(19) United States
(12) Patent Application Publication
(10) Pub. No.: US 2010/0298166 A1
(43) Pub. Date: Nov. 25, 2010

(54) CELL POPULATIONS FOR POLYPEPTIDE ANALYSIS AND USES OF SAME

(76) Inventors: Uri Alon, Rhovot (IL); Alex Sigal, Pasadena, CA (US); Ron Milo, Kfar-Saba (IL); Tamar Danon, Rehovot (IL); Ariel Cohen, Moshav Gimzo (IL); Naama Geva-Zatorsky, Rehovot (IL); Milana Frenkel-Morgenstern, Rehovot (IL); Lydia Cohen, Tel-Aviv (IL); Natalie Perzov, Herzlia (IL); Eran Eden, Rehovot (IL)

Correspondence Address: MARTIN D. MOYNIHAN d/b/a PRTSI, INC., P.O. BOX 16446, ARLINGTON, VA, 22215, US

(21) Appl. No.: 12/864,022

(22) PCT Filed: Jan. 22, 2009
(86) PCT No.: PCT/IL09/00089

§ 371 Date: Jul. 22, 2010

Related U.S. Application Data
(60) Provisional application No. 61/006,634, filed on Jan. 24, 2008.
(60) Provisional application No. 61/136,356, filed on Aug. 29, 2008.

(51) Int. Cl. C40B 30/06; C07H 21/00; C12N 5/10; C40B 40/02; C40B 50/06

(52) U.S. Cl. 506/10; 536/23.1; 435/325; 506/14; 506/26

(57) ABSTRACT

Nucleic acid construct systems are disclosed. The constructs comprise:
(i) a first nucleic acid construct comprising a first nucleic acid sequence encoding a first reporter polypeptide linked to an additional nucleic acid sequence capable of inserting the first nucleic acid construct into a genome of a host cell such that an endogenous polypeptide covalently attached to the first reporter polypeptide is expressed in the cell; and
(ii) a second nucleic acid construct comprising a second nucleic acid sequence encoding a second reporter polypeptide, linked to an additional nucleic acid sequence capable of inserting in a non-directed manner the second nucleic acid construct into a genome of a host cell such that an endogenous polypeptide covalently attached to the second reporter polypeptide is expressed in the cell, wherein the first reporter polypeptide and the second reporter polypeptide are distinguishable.

Cells and cell populations comprising same as well as methods of generating same are also disclosed. In addition, use of the novel construct systems are disclosed for identifying target agents are also disclosed.

### FIELD AND BACKGROUND OF THE INVENTION

The present invention, in some embodiments thereof, relates to cells comprising endogenous polypeptides attached to reporter polypeptides and uses thereof.

Genomic technology has advanced to a point at which, in principle, it has become possible to determine complete genomic sequences and to quantitatively measure the mRNA levels for each gene expressed in cell populations. Comparative cDNA array analysis and related technologies have been used to determine induced changes in gene expression at the mRNA level by concurrently monitoring the expression level of a large number of genes (in some cases all the genes) expressed by the investigated cell population/culture or tissue. Furthermore, biological and computational techniques have been used to correlate specific function with gene sequences.

These methods are highly effective for analyzing homogeneous populations of cells but loose their differentiation power when applied to heterogeneous populations due to large variability and averaging effects. Accordingly, the interpretation of the data obtained by these techniques in the context of the structure, control and mechanism of biological systems has been recognized as a considerable challenge. In particular, it has been extremely difficult to explain the mechanism of biological processes by genomic analysis alone.

Proteins are essential for the control and execution of virtually every biological process. Their rate of synthesis and half-life are controlled post-transcriptionally. Their level of expression is therefore not directly apparent from the gene sequence or even the expression level of the corresponding mRNA transcript. It is therefore essential that a complete description of a biological system includes measurements that indicate the identity, quantity and location of the proteins which constitute the system. An ideal measurement system would: (a) work at the level of individual cells, because experiments that average over cell populations can miss events that occur in only a subset of cells. Furthermore, averaging can miss all-or-none effects, and cell-cell variability; (b) follow cells over extended periods of time to reveal phenomena such as oscillations and temporal programs and (c) make minimal perturbations to the state of the cells.

At present no protein analytical technology approaches the throughput and level of automation of genomic technology. The most common implementation of proteome analysis is based on the separation of complex protein samples most commonly by two-dimensional gel electrophoresis (2DE) and the subsequent sequential identification of the separated protein species. This approach has been assisted by the development of powerful mass spectrometric techniques and the development of computer algorithms which correlate protein and peptide mass spectral data with sequence databases and thus rapidly identify proteins. This technology (two-dimensional mass spectrometry) has reached a level of sensitivity which now permits the identification of essentially any protein which is detectable by conventional protein staining methods including silver staining. However, the sequential manner in which samples are processed limits the sample throughput. In addition, the most sensitive methods have been difficult to automate and low abundance proteins, such as regulatory proteins, escape detection without prior enrichment, thus effectively limiting the dynamic range of the technique. In the 2DE/(MS)ⁿ method, proteins are quantified by densitometry of stained spots in the 2DE gels.

The development of methods and instrumentation for automated, data-dependent electrospray ionization (ESI) tandem mass spectrometry (MS)ⁿ in conjunction with microcapillary liquid chromatography (μLC) and database searching has significantly increased the sensitivity and speed of the identification of gel-separated proteins. As an alternative to the 2DE/(MS)ⁿ approach to proteome analysis, the direct analysis by tandem mass spectrometry of peptide mixtures generated by the digestion of complex protein mixtures has been proposed [Dongr'e et al., Trends Biotechnol 15:418-425 (1997)]. μLC-MS/MS has also been used successfully for the large-scale identification of individual proteins directly from mixtures without gel electrophoretic separation [Link et al., Nat Biotech, 17:676-682 (1999); Opitek et al., Anal Chem 69:1518-1524 (1997)]. While these approaches accelerate protein identification and assay protein modifications, they usually average over many cells and do not allow quantification of dynamics in individual cells.

There have also been advances in high-throughput quantification of protein levels and localizations at the single-cell level using antibody staining and microscopy. However, as staining of internal proteins requires the killing of the cell, it is not possible to follow protein dynamics in the same cell over time. A dynamic proteomics method in individual cells can complement antibody and mass spectrometry-based approaches.

Dynamic measurements in living cells are made possible by the use of fluorescent proteins as genetic tags. Labeling with fluorescent tags often leaves the wild-type localization intact. A library of cells containing GFP-labeled cDNAs, expressed under an exogenous promoter, has been created to investigate protein localization on the scale of the proteome [Bannasch, D. et al. Nucleic Acids Res. 32 Database issue, D505-D508 (2004); Simpson, J. C., et al EMBO Rep. 1, 287-292 (2000)]. A disadvantage of this approach is that exogenous expression gives no information about the transcriptional regulation of the gene, and potentially leads to non-physiological levels of expression. To follow wild-type regulation, homologous recombination can be used to integrate sequences of fluorescent proteins into the genome at the wild-type locus. This approach was made high throughput in yeast [Huh, W. K. et al. Nature, 425, 686-691 (2003)]. High-throughput homologous recombination is also being developed in mouse embryonic stem (ES) cells in the KOMP, EUCOMM and N or COMM initiatives. However, as yet, high-throughput homologous recombination has not been achieved in human cells.

Another tagging approach for analyzing proteins is known as central dogma (CD) tagging. This method labels proteins in their native chromosomal locations without the need for homologous recombination [Sigal et al., Nature Protocols, Vol 2, No. 6, 2007; Sigal et al., Nature Methods, Vol 3, No. 7, 2006; Sigal et al., Nature 444, October 2006, p. 643-646, Jarvik J, Biotechniques. 2002 October; 33(4):852-4, 856, 858-60 passim]. CD tagging labels genes by integrating a DNA sequence coding for a fluorescent tag into the genome. The tag is inserted in a non-directed manner using a retrovirus. It is marked as an exon by flanking splice acceptor and donor sequences. If the tag integrates within an expressed gene, it is then spliced into the gene's mRNA and a fusion protein is translated. The identity of the labeled gene is then determined by rapid amplification of cDNA end (RACE).

### SUMMARY OF THE INVENTION

According to an aspect of some embodiments of the present invention there is provided a nucleic acid construct system comprising:

(i) a first nucleic acid construct comprising a first nucleic acid sequence encoding a first reporter polypeptide linked to an additional nucleic acid sequence capable of inserting the first nucleic acid construct into a genome of a host cell such that an endogenous polypeptide covalently attached to the first reporter polypeptide is expressed in the cell; and

(ii) a second nucleic acid construct comprising a second nucleic acid sequence encoding a second reporter polypeptide, linked to an additional nucleic acid sequence capable of inserting in a non-directed manner the second nucleic acid construct into a genome of a host cell such that an endogenous polypeptide covalently attached to the second reporter polypeptide is expressed in the cell, wherein the first reporter polypeptide and the second reporter polypeptide are distinguishable.

According to some embodiments of the invention, the nucleic acid construct system further comprises a third nucleic acid construct comprising a third nucleic acid sequence encoding the first reporter polypeptide linked to an additional nucleic acid sequence capable of inserting the third nucleic acid construct into a genome of a host cell such that an additional endogenous polypeptide covalently attached to the first reporter polypeptide is expressed in the cell.

According to some embodiments of the invention, the additional nucleic acid sequence of the first nucleic acid construct directs insertion of the first nucleic acid construct into the host cell in a directed manner.

According to some embodiments of the invention, the additional nucleic acid sequence of the first nucleic acid construct directs insertion of the first nucleic acid construct into the host cell in a non-directed manner.

According to some embodiments of the invention, the host cell is a mammalian cell.

According to some embodiments of the invention, the first nucleic acid construct comprises a retroviral sequence.

According to some embodiments of the invention, the second nucleic acid construct comprises a retroviral sequence.

According to some embodiments of the invention, the first nucleic acid construct comprises a transposon sequence.

According to some embodiments of the invention, the second nucleic acid construct comprises a transposon sequence.

According to some embodiments of the invention, a 3′ end of the first and the second reporter is flanked by a splice acceptor sequence and a 5′ end of the first and the second reporter is flanked by a splice donor sequence.

According to some embodiments of the invention, the first reporter and the second reporter are fluorescent polypeptides that fluoresce at a distinguishable wave length.

According to another aspect of some embodiments of the present invention there is provided a cell expressing at least two endogenous polypeptides, each covalently attached to a distinguishable reporter polypeptide.

According to some embodiments of the invention, at least one of the at least two endogenou polypeptides has a higher nuclear:cytoplasm expression ratio.

According to some embodiments of the invention, the cell expresses an additional endogenous polypeptide attached to a reporter polypeptide, the reporter polypeptide being identical to one of the two distinguishable reporter polypeptides.

According to some embodiments of the invention, the at least one of the at least two endogenous polypeptides is constitutive.

According to some embodiments of the invention, the cell comprises the nucleic acid construct system of the present invention.

According to some embodiments of the invention, the cell is a diseased cell.

According to some embodiments of the invention, the cell is a cancer cell.

According to some embodiments of the invention, the cell is viable.

According to an aspect of some embodiments of the present invention there is provided a cell population, wherein each cell of the population expresses at least two endogenous polypeptides, each covalently attached to a distinguishable reporter polypeptide, wherein at least one of the at least two endogenous polypeptides is identical in each cell of the cell population.

According to some embodiments of the invention, the cell population expresses an additional endogenous polypeptide attached to a reporter polypeptide, the reporter polypeptide being identical to one of the two distinguishable reporter polypeptides.

According to some embodiments of the invention, both of the at least two endogenous polypeptides are identical in each cell of the cell population.

According to some embodiments of the invention, the cell population is viable.

According to some embodiments of the invention, at least one of the at least two endogenous polypeptides comprises a sequence as set forth in SEQ ID NOs: 1-164.

According to some embodiments of the invention, the cell population comprises diseased cells.

According to an aspect of some embodiments of the present invention there is provided an isolated polypeptide comprising an amino acid sequence as set forth in SEQ ID NOs: 1-164.

According to an aspect of some embodiments of the present invention there is provided a method of generating a cell population, the method comprising:

(a) introducing a first nucleic acid construct into the cell population, the first nucleic acid construct comprising a first nucleic acid sequence encoding a first reporter polypeptide linked to an additional nucleic acid sequence capable of inserting the first nucleic acid construct into a genome of a host cell such that an endogenous polypeptide covalently attached to the first reporter polypeptide is expressed in the cell; and subsequently

(b) introducing a second nucleic acid construct into the cell population, the second nucleic acid construct comprising a second nucleic acid sequence encoding a second reporter polypeptide, linked to an additional nucleic acid sequence capable of inserting in a non-directed manner the second nucleic acid construct into a genome of a host cell such that an endogenous polypeptide covalently attached to the second reporter polypeptide is expressed in the cell, wherein the first reporter polypeptide and the second reporter polypeptide are distinguishable,

thereby generating the cell population.

According to some embodiments of the invention, the method further comprises introducing a third nucleic acid construct into the cell population prior to introducing the second nucleic acid construct, the third nucleic acid construct comprising a third nucleic acid sequence encoding the first reporter polypeptide linked to an additional nucleic acid sequence capable of inserting the third nucleic acid construct into a genome of a host cell such that an additional endogenous polypeptide covalently attached to the first reporter polypeptide is expressed in the cell.

According to some embodiments of the invention, the method further comprises:

(a) selecting a cell following administration of the first nucleic acid construct, wherein the first reporter comprises a higher nuclear:cytoplasm expression ratio;

(b) propagating the cell to generate a second population of cells; and

(c) introducing into the second population of cells the second nucleic acid construct.

According to some embodiments of the invention, the method further comprises identifying at least one of the endogenous polypeptides.

According to another aspect of some embodiments of the present invention there is provided a method of identifying a target of an agent, the method comprising:

(a) contacting the cell population of the present invention with the agent;

(b) analyzing a localization or amount of at least one of the endogenous polypeptides, wherein a change in the amount or localization is indicative of a target of the agent.

According to some embodiments of the invention, the analyzing is effected in real-time.

According to some embodiments of the invention, the agent is a therapeutic agent.

According to an aspect of some embodiments of the present invention there is provided a method of identifying an agent capable of affecting a cell state, the method comprising,

(a) contacting the cell population of the present invention, with an agent; wherein at least one of the endogenous polypeptides is a marker for the cell state; and

(b) measuring a localization or amount of the marker, wherein a change in the amount or localization of the marker is indicative of an agent capable of affecting the cell state.

According to some embodiments of the invention, the cell state is a disease state.

According to some embodiments of the invention, the marker is a therapeutic target.

According to an aspect of some embodiments of the present invention there is provided a method of identifying a marker for disease prognosis, the method comprising:

(a) contacting the cell population of the present invention with a therapeutic agent;

(b) comparing a localization or amount of the at least one endogenous polypeptide in responsive cells of the cell population with non-responsive cells of the cell population; wherein a difference in expression or localization of the at least one endogenous polypeptide in responsive and non-responsive cells is indicative that the endogenous polypeptide is the marker for disease prognosis.

According to an aspect of some embodiments of the present invention there is provided a method of isolating a polypeptide, the method comprising contacting a cell population expressing an endogenous polypeptide covalently attached to a reporter polypeptide with an antibody under conditions that allow specific binding between the antibody and the reporter polypeptide, thereby isolating the polypeptide.

According to an aspect of some embodiments of the present invention there is provided a method of analyzing a localization of a first and second endogenous polypeptide in a cell, the method comprising detecting a localization of the first and second endogenous polypeptide in the cell, wherein the first and second polypeptide are each covalently attached to a distinguishable reporter polypeptide, thereby analyzing localization of a first and second polypeptide.

According to an aspect of some embodiments of the present invention there is provided a method of treating a cancer comprising co-administering to a subject in need thereof a therapeutically effective amount of Camptothecin and an agent capable of downregulating DNA helicase DDX5 as set forth in SEQ ID NO: 165 or replication factor C activator 1 (RFC1) as set forth in SEQ ID NO: 166, thereby treating the cancer.

According to some embodiments of the invention, the agent is a silencing oligonucleotide.

According to some embodiments of the invention, the cancer is ovarian or colon cancer.

According to an aspect of some embodiments of the present invention there is provided a pharmaceutical composition comprising as an active ingredient camptothecin and an agent capable of downregulating DNA helicase DDX5 of SEQ ID NO: 165 or replication factor C activator 1 (RFC1) of SEQ ID NO: 166 and a pharmaceutically acceptable carrier.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E are photographs and schemes illustrating how the library of tagged proteins was generated. Cell clones in the library were created in two steps: First a red fluorescent tag flanked by splice signals (mCherry) was introduced on a retrovirus into the genome of H1299 cells, resulting in cells that express proteins with an internal mCherry exon. After two rounds of tagging, a cell clone was selected with a red labeling pattern that is suitable for image analysis, bright in the nucleus and weaker in the cytoplasm. This clone formed the basis for an additional round of tagging, with a yellow fluorescent tag (eYFP or Venus) as an internal exon. Individual YFP tagged cells were sorted, expanded into clones, and the tagged protein in each clone was identified.

FIGS. 2A-D are photographs illustrating image analysis of the library of the present invention. Image analysis used the red fluorescent images to automatically detect cell and nuclear boundaries and to quantitate the yellow fluorescent protein intensity in each compartment at each time-point.

FIGS. 3A-D are cell images in the presence and absence of the drug Camptothecin (CPT). Cells were grown in an incubated microscope for 24 hours, and then for an additional 48 hours in the presence of 10 μM CPT. Cells were imaged every 20 minutes, and fluorescent intensity in each cell was automatically tracked. Cell divisions and morphological changes associated with cell death were automatically detected. FIGS. 3B-D show a schematic of two daughter cells of the cell in 3A. The cell labeled with the blue track shows blebbing and fragmentation typical of apoptosis.

FIGS. 4A-C are pie charts comparing protein localizations on LARC (Library of Annotated Reporter Clones) database vs. all proteins in GO (Gene Ontology Consortium). Distributions of protein localizations for: FIG. 4A—proteins in LARC with published localization; FIG. 4B—all proteins in GO; FIG. 4C—“uknown” proteins in LARC based on manual inspection. (These proteins include hypothetical proteins and proteins encoded from regions in the genome denoted as ESTs and mRNA. These proteins have no published localization).

FIGS. 5A-S are graphs illustrating the results of immunoblots against 19 selected proteins. For each protein: blue line consists of 141 fluorescent measurements taken at a 20 minute resolution for 47 hours, red line denotes quantification of immunoblotting analysis (measurement taken at 0, 8.5, 17, 24, 36, 40 and 45 hours following drug (CPT) addition. Average correlation between the two measurements across all proteins is R=0.6. Error bars denote standard errors.

FIG. 6 is a graph illustrating the rate of cell death following addition of CPT. Red line denotes the fraction of dead cells at each time point following CPT addition for over 60 hours (time resolution—20 minutes). Error bars denote standard errors.

FIGS. 7A-I are graphs illustrating examples of day to day repeats of experiment for several clones. Experiment was repeated between 2 to 8 times for 9 different clones of 9 unique proteins. Thin blue lines denote normalized total fluorescence averaged over many cells in one experiment, bold line denotes average over all days, error bars denote standard error. Mean Coefficient of variance (std/mean) over all clones and all time points of all proteins is 0.13 (mean correlation between experiments at different dates is R=0.8).

FIGS. 8A-D are graphs and plots illustrating the broad temporal patterns of protein fluorescence intensity in response to drug. FIG. 8A: Examples of YFP-tagged protein intensities of individual cells, over 48 hours after drug addition. One example is show from each of the five profiles i-v. Thin lines—individual cells, bold black lines—population averages. FIG. 8B: Normalized fluorescence shows widespread waves of accumulation and decrease in intensity. Each row corresponds to one protein averaged over all cells in the movie at each time-point (at least 30 cells). Proteins were clustered according to their dynamics. TOP1 is indicated by an arrow. FIG. 8C: Ribosomal proteins show correlated dynamics (P<10⁻³). Cytoskeleton-related proteins show behaviors either correlated or anti-correlated to cell motility. FIG. 8D: Cell motility (mean velocity of cell center of mass) declines 10 hours following drug addition.

FIGS. 9A-D are plots illustrating clusters of proteins from the same GO annotation with similar dynamics. Each plot represents a different cluster of proteins with the same GO annotation. Each line denotes the average fluorescence measured for at least 30 individual cells normalized between zero (blue) and one (red).

FIG. 10 is a graph illustrating rapid translocations in response to the drug CPT. Nucleolar levels of tagged TOP1 (the drug target) decreased in less than 2 minutes following CPT addition. Each line corresponds to a different cell.

FIGS. 11A-F are photographs and graphs illustrating TOP1 drug and dose dependency. FIG. 11AD illustrate that nuclear exit of tagged TOP1 does not occur with an equivalently lethal dose of etoposide, a topoisomerase-2 inhibitor drug. FIG. 11E is a graph illustrating that tagged TOP1 exits from the nucleus to the cytoplasm in a CPT dose dependent manner (full lines). A control nuclear protein expressed in the same cells (XRCC5-mCherry) does not exit the nucleus at all CPT doses (dashed lines). Each line is the mean of all cells at each time-point. FIG. 11F shows immunoblots with anti-TOP1 and anti-GFP showing that most TOP1 is degraded within 4 hours. In this degradation process fragments of TOP1 linked with YFP are created. These fragments are the source of fluorescence measured in the cytoplasm following CPT addition.

FIGS. 12A-B are graphs illustrating rapid translocation in response to the drug CPT. FIG. 12A illustrates tagged proteins that show a rapid decrease in nucleolar intensity and FIG. 12B illustrates tagged proteins that show a rapid increase in nucleolar/nucleoplasm ratio followed by a decrease back to basal levels.

FIGS. 13A-B are graphs illustrating localization changes in proteins in response to actinomycin-D. Localization changes of proteins in response to addition of 1 μg/ml of actinomycin-D (a transcription inhibitor). FIG. 13A: Tagged proteins that show a rapid increase in nucleolar/nucleoplasm ratio followed in some cases by a decrease back to basal levels. FIG. 13B: Tagged proteins that show a rapid decrease in nucleolar intensity.

FIGS. 14A-C are plots and graphs illustrating slower translocations in response to the drug CPT. Localization of fluorescence (nuclear intensity divided by total intensity) for all tagged proteins over time following drug addition is illustrated in FIG. 14A, and examples of two tagged proteins that show changes in nuclear (red line) and cytoplasmic (blue line) intensity (chaperon PFDN5 and thirodoxin reductase TXNRD1) are illustrated in FIGS. 14B and C respectively.

FIG. 15 is a graph illustrating that nuclear to cytoplamic ratio of TXNRD1 increases following CPT addition. Each line denotes the nuclear to cytoplamic ratio measured for an individual cell tracked over 50 hours. Bold green line denotes the average nuclear to cytoplasmic ratio.

FIG. 16 is a graph illustrating measurement of cell-cell viability over time. CV (Coefficient of variance=std/mean) of 400 proteins. In red all proteins that show CV of over 3 standard deviations from the average normalized CV of all proteins. Each line denotes CV of a different protein. Average CV of all 400 proteins is bold black and that of the 30 “bimodal” proteins is bold brown.

FIGS. 17A-F are graphs illustrating the proteins displaying bimodal response at the single cell level in response to CPT. FIGS. 17A-B are examples of proteins that show unimodal distributions, with similarly shaped profiles in each individual cell. All cells rise with time (red lines) or decrease with time (blue lines). The CV (std/mean of cell-cell distribution at each timepoint) increases slightly over time, and the distribution of slopes of fluorescence levels show a uniform behavior, all rising or all decreasing. FIGS. 17C-F are examples of proteins that show bimodal behavior. The dynamics after about 20 hours are different in different cells: some cells show increase in fluorescence levels (red) and other cells how a decrease (blue). This results in bi-modal distributions of fluorescent intensity slopes. Slopes are defined as median time derivative of the fluorescence levels, in the interval between 24 hours following drug addition to 48 hours (or time of cell death).

FIGS. 18A-B are graphs and plots illustrating that a tagged protein with a bimodal behavior correlates with the fate of individual cells. FIG. 18A: The RNA helicase DDX5 shows an increase in intensity in cells that survive the drug after 48 hours, and a decrease in cells that show the morphological changes associated with cell death. Heavy colored lines are cells that die, with darker colors corresponding to earlier cell death. Blue lines are cells that do not die during the movie. FIG. 18B: Cells that show the morphological correlates of cell death have significantly higher slopes of DDX5 fluorescence accumulation than cells that do not (T-test P<10̂-13). Slopes are defined as in FIGS. 17A-F.

FIGS. 19A-F are graphs illustrating that DDX5 shows different dynamics in response to other drugs. Response of DDX5 to Camptothecin 0.33 μM, Cis-platinum 40 μM and Etoposide 33.3 μM. Each line denotes total fluorescence measured for a single cell. Coefficient of variance (CV) is denoted for each measurement.

FIGS. 20A-B are plots illustrating that arbitrary fluorescence units can be converted to scalable units. FIG. 20A: Each dot is the measurement of the total fluorescent levels of a specific clone on two different dates. Each measurement is averaged over many cells at the time point before drug addition. Data is corrected for exposure time and lamp intensity (R=0.97). FIG. 20B: Each dot is the measurement of the total fluorescent levels of a specific protein using two different clones. Each measurement is averaged over many cells at time point before drug addition. Data is corrected for exposure time and lamp intensity (R=0.63).

FIGS. 21A-B are graphs and plots illustrating that a tagged protein with a bimodal behavior correlates with the fate of individual cells. FIG. 21A: Thioredoxin reductase 1 (TXNRD) shows an increase in intensity in cells that survive the drug after 48 hours, and a decrease in cells that show the morphological changes associated with cell death. Heavy colored lines are cells that die, with darker colors corresponding to earlier cell death. Blue lines are cells that do not die during the movie. FIG. 21B: Cells that show the morphological correlates of cell death have significantly higher slopes of TXNRD fluorescence accumulation than cells that do not (T-test P<10̂-13). Slopes are defined as in FIGS. 17A-F.

FIG. 22 is a graph illustrating that cell death dynamics in response to CPT+DDX5 siRNA increases in phase I compared to control but decreases in phase II.

### DESCRIPTION OF SPECIFIC EMBODIMENTS OF THE INVENTION

The present invention, in some embodiments thereof, relates to cells comprising endogenous polypeptides attached to reporter polypeptides. The cells may be used to analyze endogenous polypeptide localization in the cell such as in diseased and non-diseased states. Amongst a myriad of other uses, such cells may be used to test the effects of agents of interest, identify therapeutic agents as well as to determine targets of therapeutic agents and markers for disease prognosis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

A quantitative understanding of human protein networks requires the measurement of endogenous protein dynamics in living cells.

The present inventors have devised a novel approach for visualizing polypeptides in live cells and therefore have made it possible to analyze localizations of polypeptides and quantities thereof during a particular cell state and/or following exposure to a therapeutic agent. Their approach comprises tagging at least two polypeptides in their native chromosomal locations, where the image analysis of one of the tagged polypeptides is aided by the other tagged polypeptide.

Whilst reducing the present invention to practice, the present inventors have generated a library of more than 1000 cell lines based on the same parental clonal cell (H1299 cancer cell line), each clone expressing two tagged proteins used for image analysis of the third tagged protein. The third tagged protein is different in each of the cell lines of the library. Each of the tagged proteins was labeled at its endogenous chromosomal location, each undergoing endogenous regulation. Generation of the library was effected by three sequential rounds of random endogenous gene tagging as detailed in Example 1 herein below.

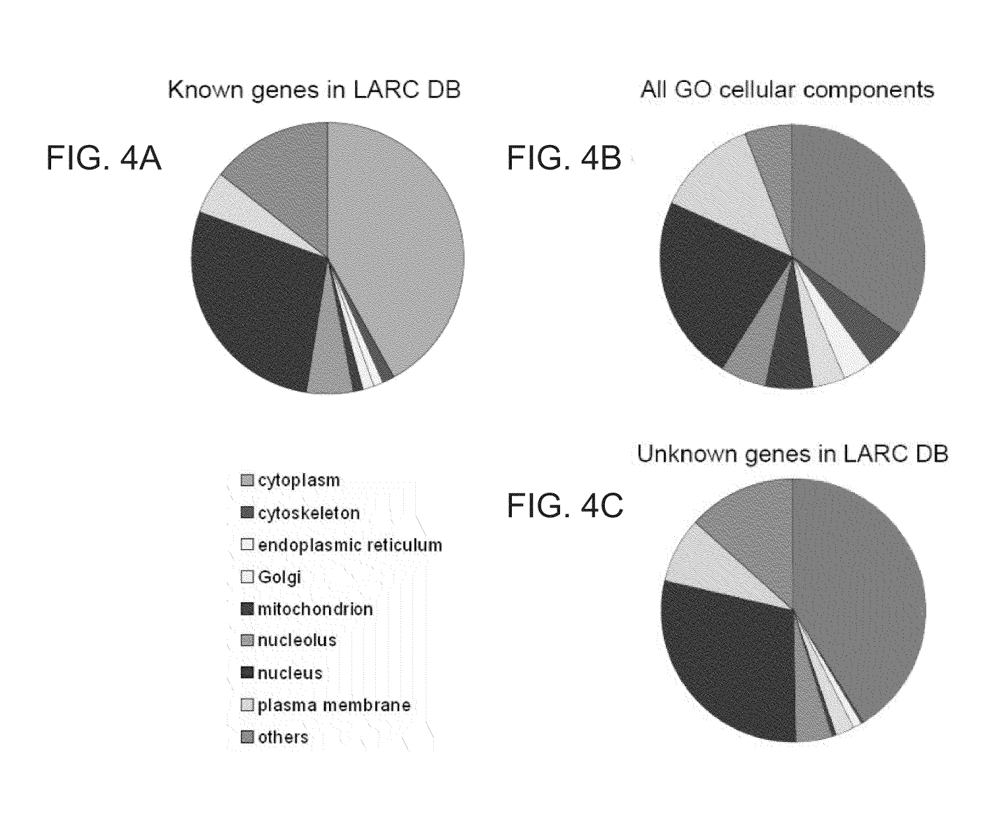

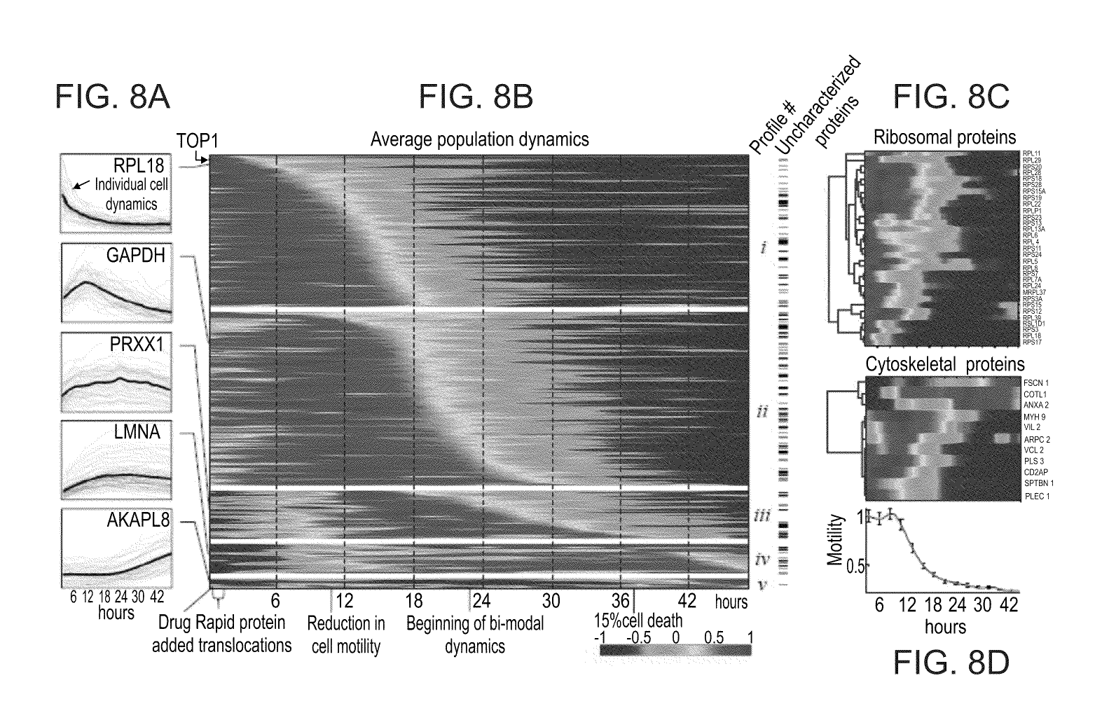

The tagged polypeptides in the library of the present invention spanned a wide range of functional categories and localization patterns including membrane, nuclear, nucleolar, cytoskeleton, Golgi, ER and other localizations (SOM) (FIGS. 4A-C). In addition, all tagged polypeptides in the library had localization patterns similar to their counterpart polypeptides without the tag. 20% of the tagged polypeptides in the library of the present invention were novel (see Table 2 in the Examples section herein below and FIG. 8B).

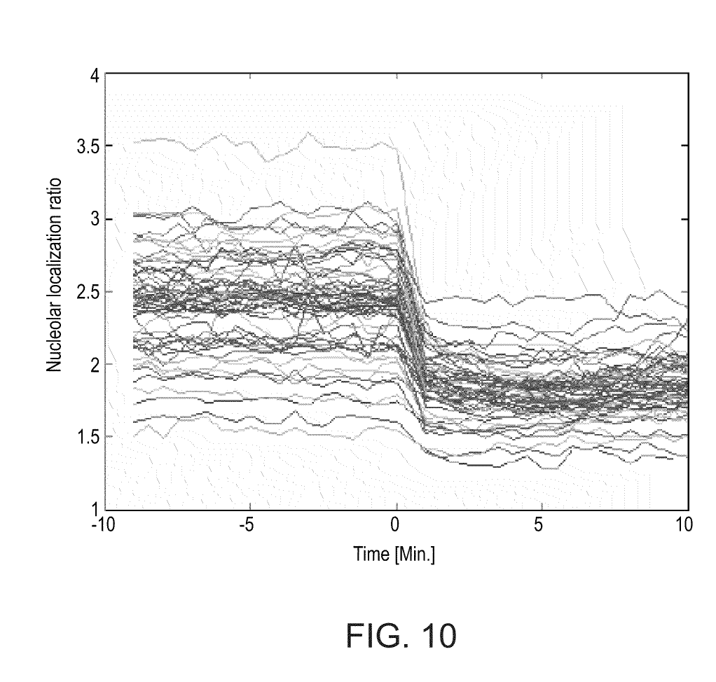

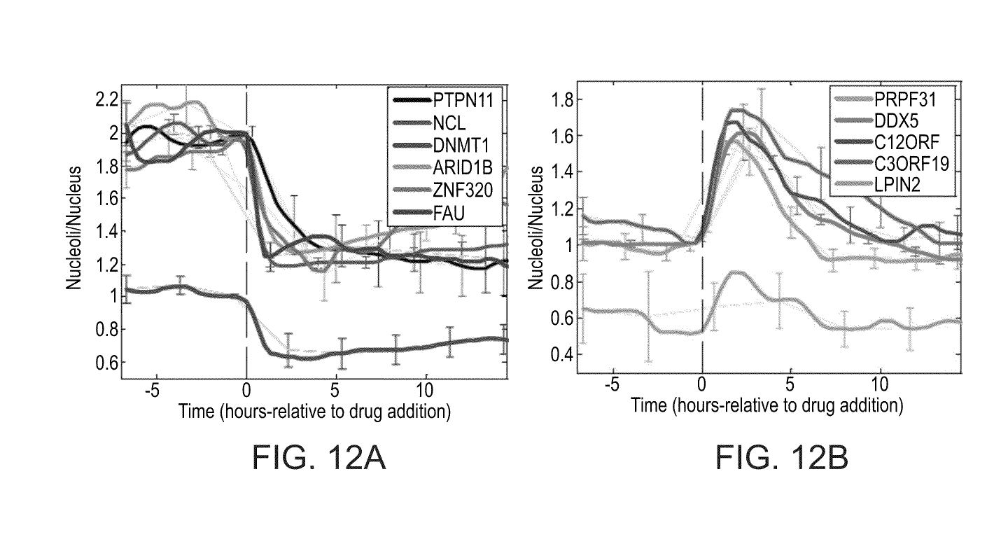

Using an exemplary therapeutic agent, camptothecin (CPT), the present inventors further showed that the present library of cell lines may be used to identify a drug target (FIGS. 8B and 10) and aid in determining a drug mechanism of action (FIGS. 12A-B and 13A-B).

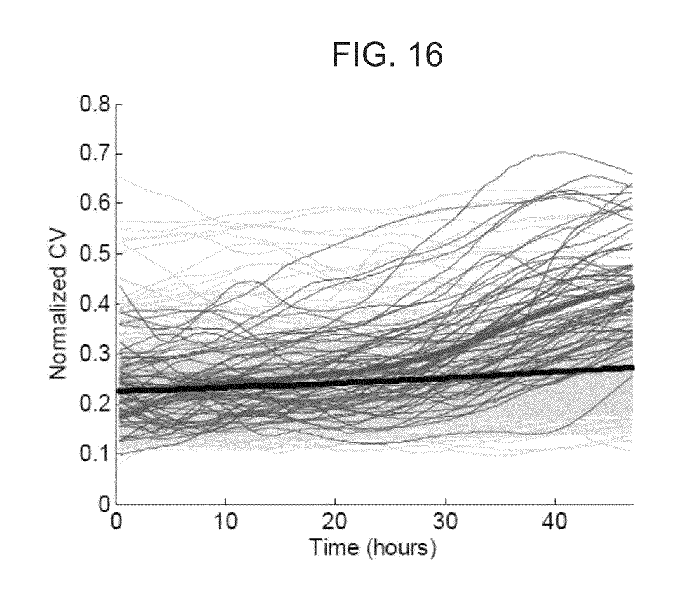

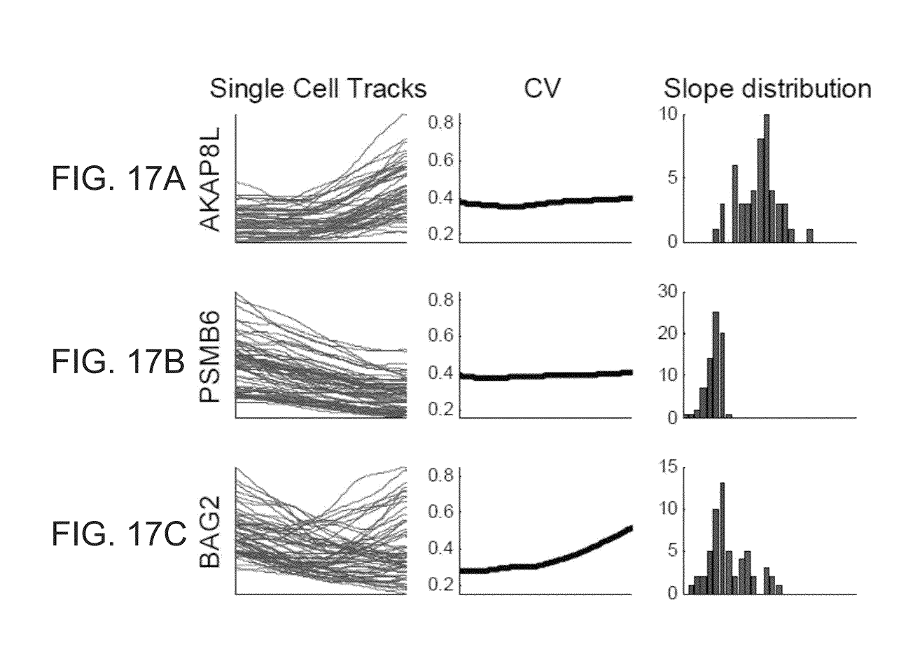

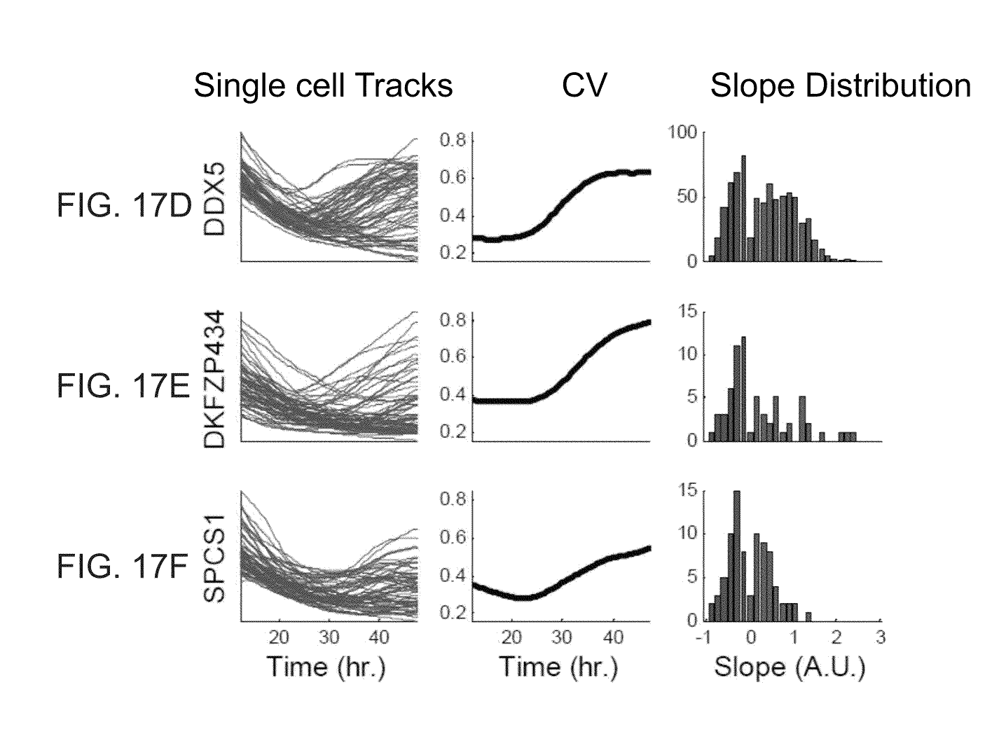

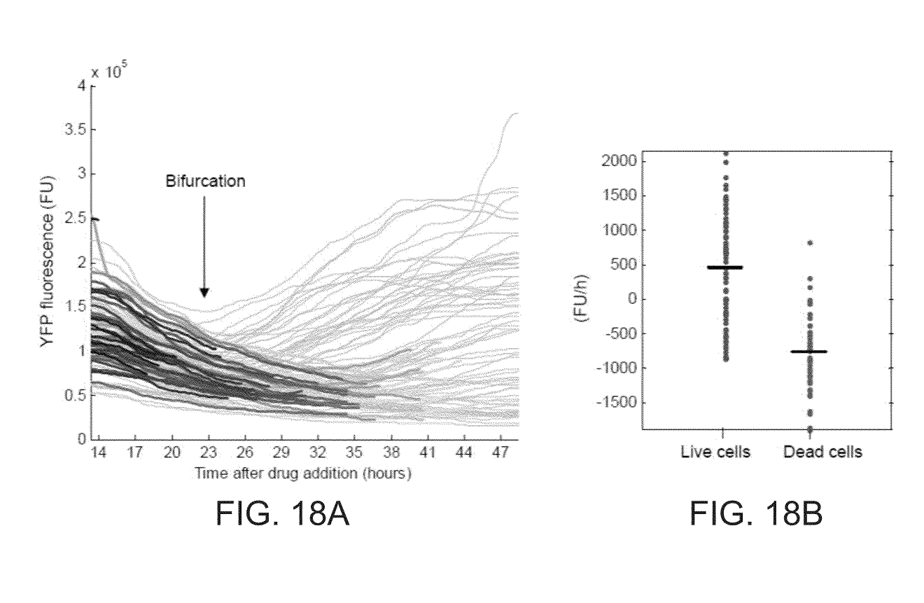

In addition, the present inventors showed that the present system allows monitoring of cell-cell variability of a particular polypeptide over time. The present inventors identified a group of polypeptides which diverged from standard cell-cell variability following treatment with CPT (FIGS. 16 and 17A-F). The present inventors further showed that the different behaviors of some of these proteins were linked to the fate of each cell (FIGS. 18A-B and 19A-F).

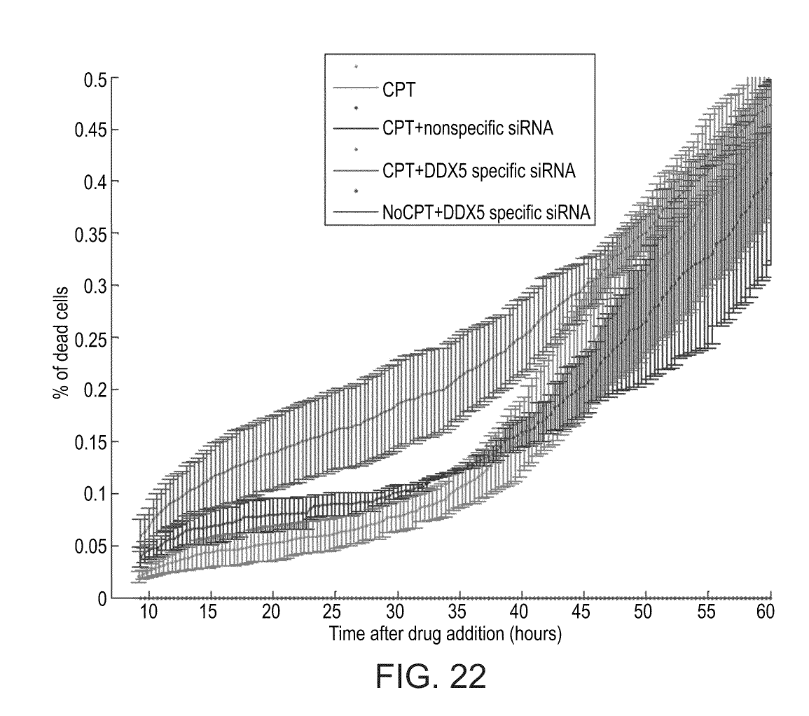

These proteins are indicative of potential drug targets, since down-regualtion of same would enhance the drug effect. As such the present system allows for identification of secondary targets (FIG. 22).

Thus, according to one aspect of the present invention there is provided a cell expressing at least two endogenous polypeptides, each covalently attached to a distinguishable reporter polypeptide.

The term “cell” as used herein, refers to a biological cell, e.g. eukaryotic, such as of mammalian origin (e.g. human). The cell may be diseased (e.g. cancerous) or healthy, taken directly from a living organism or part of a cell line, immortalized or non-immortalized.

According to one embodiment, the cell is viable.

As used herein, the phrase “endogenous polypeptide” refers to a polypeptide whose polynucleotide sequence encoding same is transcribed from its native chromosomal location in the cell.

According to one embodiment, the endogenous polypeptide is full-length.

According to another embodiment, the endogenous polypeptide is tagged internally (i.e. not on the N or C terminus) with the reporter polypeptide of the present invention.

According to yet another embodiment, the endogenous polypeptide maintains wild type functionality (i.e., of non-tagged protein) and further has a similar cellular localization pattern both prior to and following attachment of the reporter polypeptide.

Exemplary endogenous polypeptides include those listed in Table 3 of Example 2 herein below including those comprising a sequence as set forth in SEQ ID NOs: 1-164.

According to one embodiment of this aspect of the present invention, one of the endogenous polypeptides serves as an aid in the determination of the localization of the second endogenous polypeptide in the cell. Such a polypeptide is referred to herein as a “helper polypeptide”. Thus for example the “helper” polypeptide may be one that allows cell structures to be identified. For example the “helper” polypeptide may be one that localizes to the nucleus, such as XRCC5—Genbank Accession No. NP_{—}066964.1, such that the nucleus may be easily identified. Alternatively, the “helper” polypeptide may be one that localizes to the entire intracellular domain, such as DAP1—Genbank Accession No. NP_{—}004385.1, such that the entire cell may be identified. Typically, the “helper” polypeptide is constitutively expressed e.g. a house keeping polypeptide i.e. is not affected by a cell state such as a disease.

According to another embodiment of this aspect of the present invention, a combination of endogenous “helper” polypeptides aid in the detection of an additional polypeptide. The combination of “helper polypeptides” may each comprise an identical reporter polypeptide or alternatively reporter polypeptides that are distinguishable one from the other. The additionally polypeptide may serve to highlight a different area of the cell—for e.g. one of the helper polypeptides may be for identifying the cell nucleus and the other for identifying a second organelle or the cell cytoplasm as a whole.

The phrase “reporter polypeptide” as used herein, refers to a polypeptide which can be detected in a cell. Preferably, the reporter polypeptide of this aspect of the present invention can be directly detected in the cell (no need for a detectable moiety with an affinity to the reporter) by exerting a detectable signal which can be viewed in living cells (e.g., using a fluorescent microscope). Non-limiting examples of reporter polypeptides include fluorescent reporter polypeptides, (e.g. those comprising an autofluorescent activity), chemiluminescent reporter polypeptides and phosphorescent reporter polypeptides. Examples of fluorescent polypeptides include those belonging to the green fluorescent protein family, including but not limited to the green fluorescent protein, the yellow fluorescent protein, the cyan fluorescent protein and the red fluorescent protein as well as their enhanced derivatives.

As mentioned, the reporter polypeptides attached to at least two endogenous polypeptides of the present invention are distinguishable from each other. Thus, fluorescent reporter polypeptides for example may be selected such that each emits light of a distinguishable wavelength and therefore color when excited by light.

The reporter polypeptides are typically attached covalently to the endogenous polypeptides directly (i.e. via peptide bonds), although indirect attachment via linker peptides is also contemplated.

Since the polypeptides of the present invention are generated by transcription of genes present in their native chromosomal location in the cell, methods of generating cells expressing same typically entail changes to the native gene sequence of the cells.

Thus, cells of the present invention are typically generated by introduction of at least two nucleic acid constructs into the cell, both of which being capable of insertion into a genome of the cell.

The nucleic acid constructs of the present invention comprise a nucleic acid sequence encoding a reporter polypeptide linked to an additional nucleic acid sequence capable of inserting the nucleic acid construct into a genome of a host cell such that an endogenous polypeptide covalently attached to the reporter polypeptide is expressed in the cell.

It will be appreciated that the nucleic acid constructs of the present invention may be inserted into the genome of the host cell in a directed fashion (e.g. by homologous recombination or site-specific recombination) or a non-directed fashion i.e. non-homologous recombination.

The phrase “directed insertion” refers to the insertion of the construct at a predetermined sequence in the genome of the cell.

The phrase “non-directed insertion” refers to the insertion of the construct at a random sequence in the genome of the cell.

As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule will therefore have a nucleotide sequence that facilitates the exchange of nucleotide strands between the first nucleic acid molecule and a defined position of the second nucleic acid molecule. Thus, the first nucleic acid will generally have a nucleotide sequence that is sufficiently complementary to a portion of the second nucleic

As used herein, the phrase “site-specific recombinase” refers to a type of recombinase that typically has at least the following four activities (or combinations thereof): (1) recognition of specific nucleic acid sequences; (2) cleavage of said sequence or sequences; (3) topoisomerase activity involved in strand exchange; and (4) ligase activity to reseal the cleaved strands of nucleic acid (see Sauer, B., Current Opinions in Biotechnology 5:521-527 (1994)). Conservative site-specific recombination is distinguished from homologous recombination and transposition by a high degree of sequence specificity for both partners. The strand exchange mechanism involves the cleavage and rejoining of specific nucleic acid sequences in the absence of DNA synthesis (Landy, A. (1989) Ann. Rev. Biochem. 58:913-949).

Nucleic acid constructs (also referred to herein as “expression vectors”) capable of insertion in a directed manner typically comprise one or more functionally compatible recognition site for a site-specific recombination enzyme.

As used herein, the phrase “functionally compatible recognition sites for a site-specific recombination enzyme” refers to specific nucleic acid sequences which are recognized by a site-specific recombination enzyme to allow site-specific DNA recombination (i.e., a crossover event between homologous sequences). An example of a site-specific recombination enzyme is the Cre recombinase (e.g., GenBank Accession No. YP_{—}006472), which is capable of performing DNA recombination between two loxP sites. Cre recombinase can be obtained from various suppliers such as the New England BioLabs, Inc, Beverly, Mass., or it can be expressed from a nucleic acid construct in which the Cre coding sequence is under the transcriptional control of an inducible promoter (e.g., the galactose-inducible promoter) as in plasmid pSH47.

Such “directed” nucleic acid constructs typically contain other specialized elements intended to increase the level of expression of cloned nucleic acids or to facilitate the identification of cells that carry the recombinant DNA. For example, a number of animal viruses contain DNA sequences that promote extra-chromosomal replication of the viral genome in permissive cell types. Plasmids bearing these viral replicons are replicated episomally as long as the appropriate factors are provided by genes either carried on the plasmid or with the genome of the host cell.

The “directed” nucleic acid constructs of the present invention may or may not include a eukaryotic replicon. If a eukaryotic replicon is present, the vector is capable of amplification in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples of mammalian nucleic acid constructs include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, and pNMT81, which are available from Invitrogen, pCI which is available from Promega, pMbac, pPbac, pBK-RSV and pBK-CMV, which are available from Strategene, pTRES which is available from Clontech, and their derivatives.

Nucleic acid constructs containing regulatory elements from eukaryotic viruses such as retroviruses can be also used. SV40 vectors include pSVT7 and pMT2, for instance. Vectors derived from bovine papilloma virus include pBV-1MTHA, and vectors derived from Epstein-Barr virus include pHEBO and p2O5. Other exemplary vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

As mentioned, the nucleic acid constructs of the present invention may also be inserted into the genome of the host cell in a non-directed fashion, i.e. non-homologous recombination.

The phrase, “non-homologous recombination” as used herein refers to the joining (exchange or redistribution) of genetic material through a mechanism that does not involve homologous recombination (e.g., recombination directed by sequence homology) and that does not involve site-specific recombination (e.g., recombination directed by site-specific recombination signals and a corresponding site-specific recombinase). Examples of non-homologous recombination include integration of exogenous DNA into chromosomes at non-homologous sites, chromosomal translocations and deletions, DNA end joining, double strand break repair, bridge-break-fusion, concatemerization of transfected polynucleotides, retroviral insertion, and transposition.

Retroviral vectors integrate into eukaryotic genomes by a distinct mechanism of non-homologous recombination that is catalyzed by the action of the virally encoded integrase enzyme, and the mechanism of viral integration, replication and infection has been well described [see for example Retroviruses. Coffin, J M.; Hughes, S H.; Varmus, H E. Plainview (NY): Cold Spring Harbor Laboratory Press; c1997; Use of wildtype retroviruses as mutagens]. The mutagenic ability of retroviruses and retroviral vectors and their ability to enable the rapid identification of mutated genes through the linkage of retroviral tag sequences within the transcripts of mutagenized genes are well known in the art (Friedrich G, Soriano P. Methods Enzymol. 1993; 225:681-701; 3: Gossler A, et al., Science. Apr. 28, 1989; 244(4903):463-5; Friedrich G, Soriano P. Genes Dev. September 1991; 5(9):1513-23; 5: von Melchner H, et al Genes Dev. June 1992; 6(6):919-27].

Retroviral constructs of the present invention may contain retroviral LTRs, packaging signals, and any other sequences that facilitate creation of infectious retroviral vectors. Retroviral LTRs and packaging signals allow the reporter polypeptides of the invention to be packaged into infectious particles and delivered to the cell by viral infection. Methods for making recombinant retroviral vectors are well known in the art (see for example, Brenner et al., PNAS 86:5517-5512 (1989); Xiong et al., Developmental Dynamics 212:181-197 (1998) and references therein; each incorporated herein by reference). In preferred embodiments, the retroviral vectors used in the invention comprise splice acceptor (SA) and splice donor (SD) sequences flanking the sequence encoding the reporter polypeptide. Typically, the constructs of the present invention do not comprise a promoter, a start codon or a polyA signal. In this way, if the virus inserts into an actively transcribed gene, the reporter sequence is retained as a new exon after splicing of the mRNA. Owing to the large size of the first intron and viral preference for integration sites near the start of genes, the first intron is the most common point of insertion. The tagged mRNA translates to an internally labeled protein, with the reporter polypeptide usually near the N terminus.

Retroviral LTRs and packaging signals can be selected according to the intended host cell to be infected. Examples of retroviral sequences useful in the present invention include those derived from Murine Moloney Leukemia Virus (MMLV), Avian Leukemia Virus (ALV), Avian Sarcoma Leukosis Virus (ASLV), Feline Leukemia Virus (FLV), and Human Immunodeficiency Virus (HIV). Other viruses known in the art are also useful in the present invention and therefore will be familiar to the ordinarily skilled artisan.

Like retroviruses, transposons and transposon vectors can also be used to integrate sequences in a non-directed fashion into the chromosome of the cell. Also like retroviruses, transposons integrate by enzymatically catalyzed non-homologous recombination in which transposase enzymes catalyze the genomic integration and transposition of transposon DNA.

Numerous transposons have been characterized that function in mammals. In particular, the TC1/mariner derivative transposon, Sleeping Beauty, has been demonstrated to integrate efficiently in mammals.

The constructs of the present invention can be introduced into a cell and integrated into DNA by any method known in the art. In one embodiment, they are introduced by transfection. Methods of transfection include, but are not limited to, electroporation, particle bombardment, calcium phosphate precipitation, lipid-mediated transfection (e.g., using cationic lipids), micro-injection, DEAE-mediated transfection, polybrene mediated transfection, naked DNA uptake, and receptor mediated endocytosis.

Typically the introduction of the constructs of the present invention is effected whilst the cells are being cultured in a medium which supports well-being and propagation. The medium is typically selected according to the cell being transfected/infected.

According to one embodiment, the constructs of the present invention are introduced into the cell by viral transduction or infection. Suitable viral vectors useful in the present invention include, but are not limited to, adeno-associated virus, adenovirus vectors, alpha-herpesvirus vectors, pseudorabies virus vectors, herpes simplex virus vectors and retroviral vectors (including lentiviral vectors).

As mentioned, at least two nucleic acid constructs are introduced into the cell to generate the cells of the present invention.

According to one embodiment, the nucleic acid constructs are introduced in a non-simultaneous (i.e. consecutive) fashion into the cell. This may be particularly relevant if the nucleic acid construct is inserted into the cell in a non-directed fashion, since consecutive introduction of the nucleic acid constructs allows for selection of a particular clone following introduction of the first construct, and prior to introduction of the second construct.

For example, the present invention contemplates introduction of the first nucleic acid construct into the cell in a non-directed fashion, selection of a cell in which a particular polypeptide is tagged, propagation of that cell and subsequent introduction of the second nucleic acid construct into the cell. If the second nucleic acid construct is introduced into the cell in a directed fashion, a cell population will be generated in which both endogenously tagged polypeptides will be identical in each cell of the cell population. Alternatively, if the second nucleic acid construct is introduced into the cell in a non-directed fashion, a cell population will be generated in which only one endogenously tagged polypeptide will be identical in each cell of the cell population, whereas the other endogenously tagged polypeptide will be particular to each cell.

Other combinations contemplated by the present invention include introduction of the first nucleic acid construct into the cell in a directed fashion and simultaneous introduction of the second nucleic acid construct into the cell in a directed fashion.

Another contemplated example includes introduction of the first nucleic acid construct into the cell in a directed fashion and subsequent introduction of the second nucleic acid construct into the cell in a non-directed manner.

Following introduction of the nucleic acid constructs of the present invention the tagged reporter polypeptides may be identified, such as by 3′RACE, using a nested PCR reaction that amplifies the section between the reporter polypeptide and the polyA tail of the mRNA of the host gene. The PCR product may be sequenced directly and aligned to the genome.

Exemplary oligonucleotide primers that may be used for 3′RACE and sequencing are listed in Table 1 herein below.

TABLE 1

| | | | Alignment in |
|---|---|---|---|
| Primer name | Use | Sequence | YFP or mCherry |
| AP first-strand | First-strand cDNA | GGCCACGCGTCGACTAGTAC(T)17 | |
| | synthesis | (SEQ ID NO: 167) | |
| AP 92 | RACE first and | GGCCACGCGTCGACTAGTAC | |
| | nested reaction 3′ | (SEQ ID NO: 168) | |
| | primer | | |
| YFP 90 | RACE first | GCAGAAGAACGGCATCAAGG | Bases 471-490 |
| | reaction 5′ primer | (SEQ ID NO: 169) | |
| | for YFP-tagged | | |
| | genes | | |
| YFP 85 | RACE-nested | CGCGATCACATGGTCCTGCTG | Bases 646-666 |
| | reaction 5′ primer | (SEQ ID NO: 170) | |
| | for YFP-tagged | | |
| | genes | | |
| Cherry 45 | RACE first | GTGGTGACCGTGACCCAGGA | Bases 322-341 |
| | reaction 5′ primer | (SEQ ID NO: 171) | |
| | for mCherry- | | |
| | tagged genes | | |
| Cherry 46 | RACE-nested | GCGGATGTACCCCGAGGACG | Bases 456-475 |
| | reaction 5′ primer | (SEQ ID NO: 172) | |
| | for mCherry- | | |
| | tagged genes | | |
| Cherry 56 | Sequencing of | GACTACACCATCGTGGAACA | Bases 586-605 |
| | mCherry RACE | (SEQ ID NO: 173) | |
| | product | | |
| YFP 906 | Sequencing of | GGATCACTCTCGGCATGGAC | Bases 686-705 |
| | YFP RACE | (SEQ ID NO: 174) | |
| | product | | |

In this fashion, a library of cell clones may be generated, each expressing at least two identified tagged, full-length proteins, generated by transcription of genes situated in their endogenous chromosomal location. The library may comprise any number of cell clones, such as 10, 50, 100 250, 500, 1000, 2000 or more.

The present inventors using the methods described herein generated a library of cell clones comprising about 1200 different tagged proteins, of which 80% were characterized polypeptides and 20% were novel polypeptides (comprising amino acid sequences listed in SEQ ID NOs: 1-164).

It will be appreciated that libraries generated according to the method of the present invention may be used for isolating polypeptides. Cells expressing the required tagged endogenous polypeptide may be contacted with an antibody which binds specifically to the tag (i.e. reporter polypeptide). The polypeptide may then be isolated using known techniques such as immunoprecipitation and immunoaffinity columns.

As used herein, the term “isolating” refers to removing the polypeptide from its native environment i.e. cell. According to a preferred embodiment the polypeptide is also removed from other cellular components, such as other polypeptides in the cell.

Antibodies for reporter polypeptides are known in the art. For example antibodies that bind specifically to GFP are commercially available from Abcam (e.g. Catalogue numbers ab290 and ab1218) and Cell Signalling (Catalogue No. 2555).

Alternatively antibodies for reporter polypeptides may be synthesized.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Using an exemplary therapeutic agent, camptothecin (CPT), the present inventors showed that the cells of the present invention may be used to identify a drug target (FIGS. 8B and 10). The novel drug targets identified using the method of the present invention are further described herein below.

Thus, according to another aspect of the present invention, there is provided a method of identifying a target of an agent, the method comprising:

(a) contacting cells of the present invention with the agent;

(b) analyzing a localization or amount of at least one of the endogenous polypeptides, wherein a change in the amount or localization is indicative of a target of the agent.

As used herein, the term “contacting” refers to direct of indirect contacting under conditions (e.g. for an appropriate time and under an appropriate temperature) such that the agent is able to cause an alteration (e.g. an up-regulation, down-regulation or change in location) in the target.

According to this aspect of the present invention, the change in the amount is by at least 1.5 fold, and more preferably by at least 2 fold or more. A change in localization may comprise a localization to a different organelle, (e.g. from mitochondria to cytoplasm or from nucleus to cell membrane) or may comprise a change in organelle expression ratio.

As used herein, the term “localization” refers to either a localization with respect to a cell compartment (e.g. nucleus, cell membrane, mitochondria etc.) or with respect to another polypeptide.

Analysis of the localization or amount of the tagged endogenous polypeptide is typically affected according to the reporter polypeptide of the present invention.

Thus, for example if the reporter polypeptide is fluorescent, a fluorescent confocal microscope may be used to analyze the localization and/or expression of tagged endogenous polypeptide. Alternatively, the expression of a tagged endogenous polypeptide may be analyzed using flow cytometry.

Preferably, the analysis does not affect the viability or function of the cell. For example the cells of the present invention may be used to monitor a change in amount or localization of endogenous polypeptide over real-time using long period time-lapse microscopy. Time-lapse movies may be obtained as described by Sigal et al. (Sigal, Milo et al. 2006, supra) with for example an automated, incubated (including humidity and CO₂ control) inverted fluorescence microscope (e.g. Leica DMIRE2) and a CCD camera (e.g. ORCA ER—Hamamatsu Photonics).

It will be appreciated that if the analysis is effected in real-time, a sequence of events following a particular treatment can also be monitored. Thus for example, the camera or cameras may be capable of recording a number of cell populations at one time, each cell population comprising a different tagged endogenous polypeptide over a period of time (e.g. 24 hours). Analysis of the movies obtained following monitoring allows reconstruction of the sequence of events that occur after contact with the agent. The present inventors have shown, using the agent Camptothecin (CPT) by way of example, that typically the first polypeptide to respond is the direct target of the agent.

Agents whose targets are being determined, include therapeutic agents (such as polynucleotides, polypeptides, small molecule chemicals, carbohydrates, lipids etc.). It will be appreciated that the agent may also be a condition such as radiation. Further, the targets whose agents are being determined may be carcinogens or pollutants.

If the tagged endogenous polypeptide is a marker for a cell state, the cells of the present invention may be used to identify an agent capable of affecting that cell state.

Exemplary cell states include, but are not limited to a disease state such as cancer, an oxidative state and a hyperglycemic or hypoglycemic state etc.

According to this aspect of the present invention the cells of the present invention are contacted with a test agent and a localization or amount of the marker of the cell state is analyzed, wherein a change in the amount or localization of the marker is indicative of that the test agent is capable of affecting the cell state.

It will be appreciated that the cells of the present invention may be used to identify markers for disease prognosis. According to this aspect, diseased cells of the present invention are contacted with a therapeutic agent and the localization or amount of the tagged endogenous polypeptide in responsive cells is compared with the localization or amount of tagged endogenous polypeptide in non-responsive cells. A difference in expression or localization of the tagged endogenous polypeptide in responsive and non-responsive cells indicates that the tagged endogenous polypeptide is a marker for disease prognosis.

As used herein, the phrase “marker for disease prognosis” refers to a polypeptide whose expression or localization correlates with the severity of a disease. It will be appreciated that this method may also be used to select potential drug targets for enhancing an effect of a drug.

Detection of responsive and non-responsive cells is effected according to the cell type and the therapeutic agent. Thus, for example if the cells are cancer cells and the therapeutic agent causes a decrease in a particular marker e.g. a matrix metalloproteinase, cells may be generated that express a tagged matrix metalloproteinase, a tagged protein (or proteins) that aid in image analysis and a third tagged protein that is being analyzed. Such cells may be analyzed for other markers whose expression (or localization) correspond with the known marker of the disease.

According to another example, the cells are cancer cells and the therapeutic agent causes cell death. Individual cells may be analyzed using a microscope to see whether they show signs of cell death (e.g. cell shrinkage, nuclear fragmentation, blebbing etc.) in order to analyze if they are drug responsive or not. Comparison of the polypeptides in the responsive cell group with polypeptides in the non-responsive cell group, allows identification of potential drug targets for enhancing the effect of a drug. For example, the present inventors showed that three polypeptides were differentially up and down regulated in cells that survive the drug CPT, as opposed to cells that die. The three polypeptides were the helicase DDX5, the transport protein VPS26a and the appoptosis protein PEPP2. By targeting these proteins, together with CPT, one may be able to increase the efficacy of the drug by targeting cancer cells that would otherwise not be killed.

Since the cells of the present invention express at least two tagged endogenous polypeptides, the cells may be used to analyze localization of same.

Thus, according to yet another aspect of the present invention there is provided a method of analyzing a localization of a first and second endogenous polypeptide in a cell, the method comprising detecting a localization of the first and second endogenous polypeptide in the cell, wherein the first and second polypeptide are each covalently attached to a distinguishable reporter polypeptide, thereby analyzing localization of a first and second polypeptide.

It will be appreciated that the method of this aspect of the present invention may be used to analyze localization the two endogenous polypeptides to a particular cell compartment, or alternatively to analyze their localization with respect to one another. Accordingly, the method of this aspect of the present invention may also be used to detect a binding or interaction between the first and second endogenous polypeptide.

Accordingly, the present invention may be used as a FRET system for analyzing the interaction between two endogenous polypeptides.

As used herein, the term “FRET” refers to the process in which an excited donor fluorophore transfers energy to a lower-energy acceptor fluorophore via a short-range (e.g., less than or equal to 10 nm) dipole-dipole interaction.

As mentioned, the present invention identified novel targets for Camptothecin using the cell populations of the present invention.

As described in Example 3 herein below, the present inventors have shown that DNA helicase DDX5 and Replication factor C activator 1 (RFC1) both decrease in cells that respond to CPT treatment indicating that these proteins promote cell survival under this drug. Accordingly, inhibition of these polypeptides may increase the efficacy of CPT (FIG. 22). In addition, the present inventors have shown that inhibitors of thioredoxin and thioredoxin reductase 1 (TXNRD1) may also be used to enhance the effect of CPT.

Thus, according to another aspect of the present invention, there is provided a method of treating a cancer comprising co-administering to a subject in need thereof a therapeutically effective amount of Camptothecin and an agent capable of downregulating DNA helicase DDX5 or replication factor C activator 1 (RFC1), thereby treating the cancer.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein the term “subject” refers to any (e.g., mammalian) subject, preferably a human subject.

As used herein, the term “camptothecin” refers to a cytotoxic quinoline alkaloid capable of inhibiting the DNA enzyme topoisomerase I. Camptothecin is widely commercially available (e.g. Sigma CPT; C9911). The camptothecin may be an analogue or a derivate of available camptothecins.

The term “DNA helicase DDX5” refers to the polypeptide whose sequence is as set forth in Genbank as NP_{—}004387.1, Swiss Prot. number P17844 and homologues and variants thereof.

The term “Replication factor C activator 1 (RFC1)” refers to the polypeptide whose sequence is as set forth in Genbank as NP_{—}002904.3, Swiss Prot. number P35251 and homologues and variants thereof.

The term “thioredoxin reductase 1 (TXNRD1)” refers to the polypeptide whose sequence is as set forth in Genbank as NP_{—}001087240.1, NP_{—}003321.3, NP_{—}877393.1, NP_{—}877419.1 or NP_{—}877420.1, Swiss Prot. number Q16881 and homologues and variants thereof.

As used herein the term “cancer” refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells.

Specific examples of cancer which can be treated using the combination of the present invention include, but are not limited to, adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; Burkitt's lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; glomus tumors, multiple; hepatoblastoma; hepatocellular cancer; hepatocellular carcinoma; leukemia, acute lymphoblastic; leukemia, acute myeloid; leukemia, acute myeloid, with eosinophilia; leukemia, acute nonlymphocytic; leukemia, chronic myeloid; Li-Fraumeni syndrome; liposarcoma, lung cancer; lung cancer, small cell; lymphoma, non-Hodgkin's; lynch cancer family syndrome II; male germ cell tumor; mast cell leukemia; medullary thyroid; medulloblastoma; melanoma, meningioma; multiple endocrine neoplasia; myeloid malignancy, predisposition to; myxosarcoma, neuroblastoma; osteosarcoma; ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell cancer of lung; soft tissue sarcoma, squamous cell carcinoma, head and neck; T-cell acute lymphoblastic leukemia; Turcot syndrome with glioblastoma; tylosis with esophageal cancer; uterine cervix carcinoma, Wilms' tumor, type 2; and Wilms' tumor, type 1, and the like.

According to one embodiment of this aspect of the present invention, the cancer is ovarian or colon cancer.

Down-regulating the function or expression of DNA helicase DDX5, replication factor C activator 1 (RFC1), thioredoxin or thioredoxin redutase can be effected at the RNA level or at the protein level. According to one embodiment of this aspect of the present invention the agent is an oligonucleotide capable of specifically hybridizing (e.g., in cells under physiological conditions) to a polynucleotide encoding these polypeptide. Exemplary siRNAs capable of down-regulating DDX5 are set forth in SEQ ID NO:175-178.

The prior art teaches of a number of delivery strategies which can be used to efficiently deliver oligonucleotides into a wide variety of cell types [see, for example, Luft J Mol Med 76: 75-6 (1998); Kronenwett et al., Blood 91: 852-62 (1998); Rajur et al., Bioconjug Chem 8: 935-40 (1997); Lavigne et al., Biochem Biophys Res Commun 237: 566-71 (1997) and Aoki et al., (1997) Biochem Biophys Res Commun 231: 540-5 (1997)].

According to another embodiment of this aspect of the present invention, the agent is a RNA silencing agent.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl. Acad. Sci. USA. 2002; 99:1443-1448; Tran N., et al., FEBS Lett. 2004; 573:127-134].

In particular, the present invention also contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433 and Diallo et al, Oligonucleotides, Oct. 1, 2003, 13(5): 381-392, doi:10.1089/154545703322617069.

The present invention also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21mers with a central 19 by duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the sRNA pathway is determined solely by the degree of complementarity to the target transcript. It is speculated that siRNAs with only partial identity to the mRNA target will function in translational repression, similar to an miRNA, rather than triggering RNA degradation.

Synthesis of RNA silencing agents suitable for use with the present invention can be effected as follows. First, the polypeptide mRNA sequence is scanned downstream of the AUG start codon for AA dinucleotide sequences. Occurrence of each AA and the 3′ adjacent 19 nucleotides is recorded as potential sRNA target sites. Preferably, sRNA target sites are selected from the open reading frame, as untranslated regions (UTRs) are richer in regulatory protein binding sites. UTR-binding proteins and/or translation initiation complexes may interfere with binding of the sRNA endonuclease complex [Tuschl ChemBiochem. 2:239-245]. It will be appreciated though, that siRNAs directed at untranslated regions may also be effective, as demonstrated for GAPDH wherein sRNA directed at the 5′ UTR mediated about 90% decrease in cellular GAPDH mRNA and completely abolished protein level (wwwdotambiondotcom/techlib/tn/91/912dothtml).

Second, potential target sites are compared to an appropriate genomic database (e.g., human, mouse, rat etc.) using any sequence alignment software, such as the BLAST software available from the NCBI server (wwwdotncbidotnlmdotnihdotgov/BLAST/). Putative target sites which exhibit significant homology to other coding sequences are filtered out.

Qualifying target sequences are selected as template for sRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, plsl, TAT(48-60), pVEC, MTS, and MAP.

Another agent capable of downregulating the expression of the CPT modulating polypeptides of the present invention is a DNAzyme molecule capable of specifically cleaving its encoding polynucleotide. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 94:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther wwwdotasgtdotorg). In another application, DNAzymes complementary to bcr-ab1 oncogenes were successful in inhibiting the oncogenes expression in leukemia cells, and lessening relapse rates in autologous bone marrow transplant in cases of Chronic Myelogenous Leukemia (CML) and Acute Lymphocytic Leukemia (ALL).

Another agent capable of downregulating the expression of the CPT modulating polypeptides of the present invention is a ribozyme molecule capable of specifically cleaving its encoding polynucleotide. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications.

An additional method of downregulating the function of a CPT modulating polypeptide of the present invention is via triplex forming oligonucleotides (TFOs). In the last decade, studies have shown that TFOs can be designed which can recognize and bind to polypurine/polypirimidine regions in double-stranded helical DNA in a sequence-specific manner. Thus the DNA sequence encoding the polypeptide of the present invention can be targeted thereby down-regulating the polypeptide.

The recognition rules governing TFOs are outlined by Maher III, L. J., et al., Science (1989) 245:725-730; Moser, H. E., et al., Science (1987)238:645-630; Beal, P. A., et al., Science (1991) 251:1360-1363; Cooney, M., et al., Science (1988)241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer (2003) J Clin Invest; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch (2002), BMC Biochem, September 12, Epub). The same authors have demonstrated that TFOs designed according to the A-AT and G-GC rule do not form non-specific triplexes, indicating that the triplex formation is indeed sequence specific.

Thus for any given sequence in the regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and subsequent formation of the triple helical structure with the target DNA, induces steric and functional changes, blocking transcription initiation and elongation, allowing the introduction of desired sequence changes in the endogenous DNA and results in the specific downregulation of gene expression. Examples of such suppression of gene expression in cells treated with TFOs include knockout of episomal supFG1 and endogenous HPRT genes in mammalian cells (Vasquez et al., Nucl Acids Res. (1999) 27:1176-81, and Puri, et al., J Biol Chem, (2001) 276:28991-98), and the sequence- and target-specific downregulation of expression of the Ets2 transcription factor, important in prostate cancer etiology (Carbone, et al., Nucl Acid Res. (2003) 31:833-43), and the pro-inflammatory ICAM-1 gene (Besch et al., J Biol Chem, (2002) 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res (2000); 28:2369-74).

Additionally, TFOs designed according to the abovementioned principles can induce directed mutagenesis capable of effecting DNA repair, thus providing both downregulation and upregulation of expression of endogenous genes [Seidman and Glazer, J Clin Invest (2003) 112:487-94]. Detailed description of the design, synthesis and administration of effective TFOs can be found in U.S. Patent Application Nos. 2003 017068 and 2003 0096980 to Froehler et al., and 2002 0128218 and 2002 0123476 to Emanuele et al., and U.S. Pat. No. 5,721,138 to Lawn.

As mentioned hereinabove, down regulating the function of a CPT modulating polypeptide of the present invention can also be affected at the protein level.

Thus, another example of an agent capable of downregulating a CPT modulating polypeptide of the present invention is an antibody or antibody fragment capable of specifically binding to it, preferably to its active site, thereby preventing its function.

As used herein, the term “antibody” refers to a substantially intact antibody molecule.

As used herein, the phrase “antibody fragment” refers to a functional fragment of an antibody that is capable of binding to an antigen.

Suitable antibody fragments for practicing the present invention include, inter alia, a complementarity-determining region (CDR) of an immunoglobulin light chain (referred to herein as “light chain”), a CDR of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single-chain Fv, an Fab, an Fab′, and an F(ab′)₂.

Functional antibody fragments comprising whole or essentially whole variable regions of both light and heavy chains are defined as follows:

(i) Fv, defined as a genetically engineered fragment consisting of the variable region of the light chain and the variable region of the heavy chain expressed as two chains;

(ii) single-chain Fv (“scFv”), a genetically engineered single-chain molecule including the variable region of the light chain and the variable region of the heavy chain, linked by a suitable polypeptide linker.

(iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof;

(iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and

(v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds).

Methods of generating monoclonal and polyclonal antibodies are well known in the art. Antibodies may be generated via any one of several known methods, which may employ induction of in vivo production of antibody molecules, screening of immunoglobulin libraries (Orlandi, R. et al. (1989). Cloning immunoglobulin variable domains for expression by the polymerase chain reaction. Proc Natl Acad Sci USA 86, 3833-3837; and Winter, G. and Milstein, C. (1991). Man-made antibodies. Nature 349, 293-299), or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler, G. and Milstein, C. (1975). Continuous cultures of fused cells secreting antibody of predefined specificity. Nature 256, 495-497; Kozbor, D. et al. (1985). Specific immunoglobulin production and enhanced tumorigenicity following ascites growth of human hybridomas. J Immunol Methods 81, 31-42; Cote R J. et al. (1983). Generation of human monoclonal antibodies reactive with cellular antigens. Proc Natl Acad Sci USA 80, 2026-2030; and Cole, S. P. et al. (1984). Human monoclonal antibodies. Mol Cell Biol 62, 109-120).

It will be appreciated that for human therapy or diagnostics, humanized antibodies are preferably used. Humanized forms of non-human (e.g., murine) antibodies are genetically engineered chimeric antibodies or antibody fragments having (preferably minimal) portions derived from non-human antibodies. Humanized antibodies include antibodies in which the CDRs of a human antibody (recipient antibody) are replaced by residues from a CDR of a non-human species (donor antibody), such as mouse, rat, or rabbit, having the desired functionality. In some instances, the Fv framework residues of the human antibody are replaced by corresponding non-human residues. Humanized antibodies may also comprise residues found neither in the recipient antibody nor in the imported CDR or framework sequences. In general, the humanized antibody will comprise substantially all of at least one, and typically two, variable domains, in which all or substantially all of the CDRs correspond to those of a non-human antibody and all or substantially all of the framework regions correspond to those of a relevant human consensus sequence. Humanized antibodies optimally also include at least a portion of an antibody constant region, such as an Fc region, typically derived from a human antibody (see, for example: Jones, P. T. et al. (1986). Replacing the complementarity-determining regions in a human antibody with those from a mouse. Nature 321, 522-525; Riechmann, L. et al. (1988). Reshaping human antibodies for therapy. Nature 332, 323-327; Presta, L. G. (1992b). Curr Opin Struct Biol 2, 593-596; and Presta, L. G. (1992a). Antibody engineering. Curr Opin Biotechnol 3(4), 394-398).

Methods for humanizing non-human antibodies are well known in the art. Generally, a humanized antibody has one or more amino acid residues introduced into it from a source which is non-human. These non-human amino acid residues are often referred to as imported residues, which are typically taken from an imported variable domain. Humanization can be performed essentially as described (see, for example: Jones et al. (1986); Riechmann et al. (1988); Verhoeyen, M. et al. (1988). Reshaping human antibodies: grafting an antilysozyme activity. Science 239, 1534-1536; and U.S. Pat. No. 4,816,567), by substituting human CDRs with corresponding rodent CDRs. Accordingly, humanized antibodies are chimeric antibodies, wherein substantially less than an intact human variable domain has been substituted by the corresponding sequence from a non-human species. In practice, humanized antibodies may be typically human antibodies in which some CDR residues and possibly some framework residues are substituted by residues from analogous sites in rodent antibodies.

Human antibodies can also be produced using various additional techniques known in the art, including phage-display libraries (Hoogenboom, H. R. and Winter, G. (1991). By-passing immunisation. Human antibodies from synthetic repertoires of germline VH gene segments rearranged in vitro. J Mol Biol 227, 381-388; Marks, J. D. et al. (1991). By-passing immunization. Human antibodies from V-gene libraries displayed on phage. J Mol Biol 222, 581-597; Cole et al. (1985), Monoclonal Antibodies and Cancer Therapy, Alan R. Liss, Inc., pp. 77-96; and Boerner, P. et al. (1991). Production of antigen-specific human monoclonal antibodies from in vitro-primed human splenocytes. J Immunol 147, 86-95). Humanized antibodies can also be created by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807; 5,545,806; 5,569,825; 5,625,126; 5,633,425; and 5,661,016; Marks, J. D. et al. (1992). By-passing immunization: building high affinity human antibodies by chain shuffling. Biotechnology (N.Y.) 10(7), 779-783; Lonberg et al., 1994. Nature 368:856-859; Morrison, S. L. (1994). News and View: Success in Specification. Nature 368, 812-813; Fishwild, D. M. et al. (1996). High-avidity human IgG kappa monoclonal antibodies from a novel strain of minilocus transgenic mice. Nat Biotechnol 14, 845-851; Neuberger, M. (1996). Generating high-avidity human Mabs in mice. Nat Biotechnol 14, 826; and Lonberg, N. and Huszar, D. (1995). Human antibodies from transgenic mice. Int Rev Immunol 13, 65-93).

It will be appreciated that the inhibitory agents of the present invention may be administered concurrently with the CPT (e.g. by formulating them in a single composition) or may be administered prior to or following CPT administration.

The agents of the present invention can be provided to the individual per se, or as part of a pharmaceutical composition where it is mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide or polynucleotide preparation, which is accountable for the biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., a nucleic acid construct) effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

It is expected that during the life of a patent maturing from this application many relevant reporter polypeptides will be developed and the scope of the term reporter polypeptide is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, an and the include plural references unless the context clearly dictates otherwise. For example, the term “a polypeptide” or “at least one polypeptide” may include a plurality of polypeptides, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Construction of a Cherry/YFP CD-Tagged Reporter Clone Library

Gathering of quantitative information from time-lapse fluorescent movies of proteins in individual living cells is a difficult task. In order to overcome such difficulties, a system for dynamic proteomics was developed. [Perlman, Slack et al. 2004, Science 306: 1194-1198; Echeverri and Perrimon 2006, Nat Rev Genet 7: 373-384; Eggert and Mitchison 2006, Curr Opin Chem Biol 10: 232-237; Megason and Fraser 2007, Cell 130(5): 784-95)]. This system for tagging proteins in human cells, is based on a retrovirally based CD-tagging approach [Sigal et al., Nature Protocols, Vol 2, No. 6, 2007; Sigal et al., Nature Methods, Vol 3, No. 7, 2006; Sigal et al., Nature 444, October 2006, p. 643-646, all of which are incorporated herein by reference]. This allows construction of a library of cell clones, each expressing a fluorescently tagged, full-length protein from its endogenous chromosomal location.

Materials and Methods

A library of fluorescently tagged proteins was constructed in non-small cell lung carcinoma cell line (H1299) in a two stage process. In both stages a fluorescent reporter was integrated into the genome via Central Dogma tagging (CD-tagging) (Otsu 1979; Jarvik, Adler et al. 1996; Jarvik, Fisher et al. 2002; Sigal, Danon et al. 2007).

The first stage was carried out in order to produce a parental clone in which the nucleus is colored brighter than the cytoplasm and the cytoplasm is colored brighter than the medium. To achieve this, a red fluorescent protein, mCherry (Shaner, Campbell et al. 2004), was introduced in two rounds of CD-tagging. In the first round, clone H7a with tagged protein XRCC5, localized to the nucleus, was selected. In the second round (carried out on the previously selected clone H7a), clone H7 with tagged DAP1 localized to the whole intracellular domain was selected. Following these two steps, a parental clone was obtained expressing two mCherry endogenously tagged proteins (XRCC5 and DAP1), stained in the cytoplasm and brighter in the nucleus.

The second stage in the generation of the library was to use CD-tagging in order to tag different proteins with a second color EYFP or Venus (Nagai, Ibata et al. 2002) within the parental clone H1299-ul.

CD tagging described in detail by Sigal et al. [Sigal et al., Nature Protocols, Vol 2, No. 6, 2007], incorporated herein by reference. Briefly, a fluorescent protein (FP), flanked by splice acceptor and donor sequences was integrated into the genome as an artificial exon via retroviral vectors (U5000, U5001, U5002), each containing FP in one of 3 reading frames. Cells positive for relevant FP fluorescence were sorted using flow cytometry into 384 well plates and expanded into cell clones.

Results

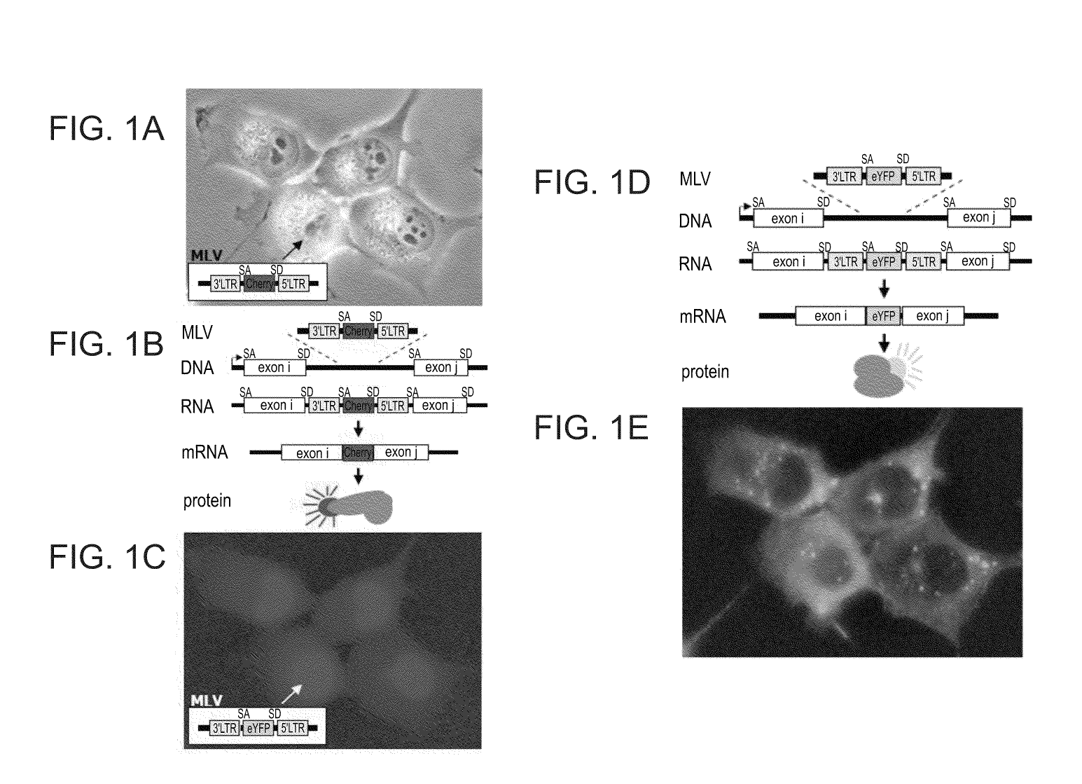

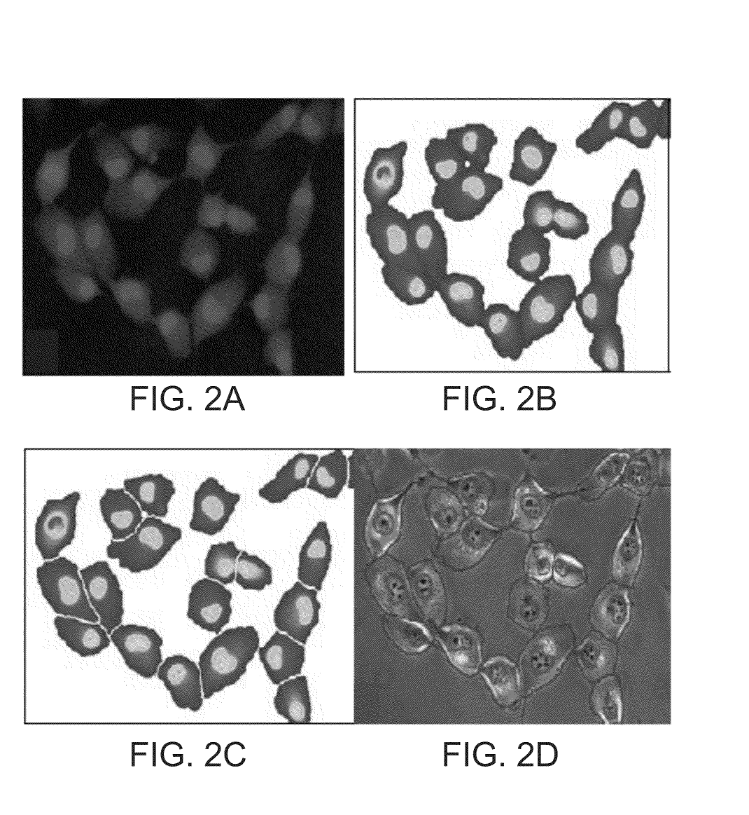

To obtain reliable image analysis of cell movies, the parental cell (H1299 non-small cell lung carcinoma cell line) was tagged with a red fluorophore (mCherry) that colors the cytoplasm and, more strongly, the nucleus (FIG. 1C). The resulting cell clone showed no growth or morphological differences relative to the untagged parental cells. Custom software used the mCherry fluorescence to automatically distinguish the cell from its background, and to distinguish the nucleus from the cytoplasm (FIGS. 2A-D). Attempts to use transfected red proteins or exogenous dyes were unsuccessful because they led to high cell-cell variability of the tag which made it difficult to analyze the images. To avoid this variability, CD-tagging was used to introduce the red tag into endogenous proteins and a clone was selected with a fluorescence pattern suitable for image analysis. This clone was then used as a basis for the present tagged protein library: A yellow fluorescent marker was introduced into the red-tagged cells by a second round of CD-tagging, following which the yellow tagged cells were expanded into clones, and the tagged proteins were identified (FIGS. 1A-E). Thus, the red tagging is the same in all cells of the library, and is independent of the second yellow stain of the protein of interest.

### Example 2

### Identification of Tagged Proteins in the Library of the Present Invention

Materials and Methods

Tagged protein identities were determined by 3′RACE, using a nested PCR reaction that amplified the section between the FP and the polyA tail of the mRNA of the host gene. The PCR product was sequenced directly and aligned to the genome.

Results

The library listed herein below includes 1200 different tagged proteins, of which 80% are characterized proteins and 20% are novel proteins.

Table 2, herein below lists the novel proteins which were tagged according to the method of the present invention. The table also provides the results of measurement the ratio of total fluorescence in the cytoplasm vs. total fluorescence in the whole cell for each of these proteins, above 0.5 is denoted as nuclear localization and below 0.5 as cytoplasmic localization.

TABLE 2

| SEQ | | | Cytoplasm/ | | |
|---|---|---|---|---|---|
| ID | | | whole | | |
| NO: | GB number | Description | cell | Nucleus | Cytoplasm |
| 1 | AA282714.1 | AA282714 zt13f10.r1 | 0.7866 | 0 | 1 |
| | | NCI_CGAP_GCB1 Homo sapiens | | | |
| | | cDNA clone IMAGE: 713035 5′, | | | |
| | | mRNA sequence | | | |
| 2 | AA479512.1 | AA479512 zv21f09.s1 | 0.779 | 0 | 1 |
| | | Soares_NhHMPu_S1 Homo | | | |
| | | sapiens cDNA clone | | | |
| | | IMAGE: 754313 3′, mRNA | | | |
| | | sequence | | | |
| 3 | AA843465.1 | AA843465 aj54c11.s1 | 0.3618 | 1 | 0 |
| | | Soares_testis_NHT Homo sapiens | | | |
| | | cDNA clone IMAGE: 1394132 3′, | | | |
| | | mRNA sequence | | | |
| 4 | AA928516.1 | AA928516 om17h03.s1 | 0.4001 | 1 | 0 |
| | | Soares_NFL_T_GBC_S1 Homo | | | |
| | | sapiens cDNA clone | | | |
| | | IMAGE: 1541333 3′, mRNA | | | |
| | | sequence | | | |
| 5 | AF086125.1 | HUMZA79D12 Homo sapiens full | 0.8349 | 0 | 1 |
| | | length insert cDNA clone ZA79D12 | | | |
| 6 | AF087973.1 | HUMYU79H10 Homo sapiens full | 0.7233 | 0 | 1 |
| | | length insert cDNA clone YU79H10 | | | |
| 7 | AI027434.1 | AI027434 ow49f09.s1 | 0.2965 | 1 | 0 |
| | | Soares_parathyroid_tumor_NbHPA | | | |
| | | Homo sapiens cDNA clone | | | |
| | | IMAGE: 1650185 3′ similar to | | | |
| | | TR: Q40462 Q40462 NTGB1, | | | |
| | | mRNA sequence | | | |
| 8 | AI208228.1 | AI208228 qg50b01.x1 | 0.7128 | 0 | 1 |
| | | Soares_testis_NHT Homo sapiens | | | |
| | | cDNA clone IMAGE: 1838569 3′, | | | |
| | | mRNA sequence | | | |
| 9 | AI434862.1 | AI434862 ti13c03.x1 | 0.7284 | 0 | 1 |
| | | NCI_CGAP_Kid11 Homo sapiens | | | |
| | | cDNA clone IMAGE: 2130340 3′, | | | |
| | | mRNA sequence | | | |
| 10 | AI671392.1 | AI671392 wc29g07.x1 | 0.3552 | 1 | 0 |
| | | NCI_CGAP_Kid11 Homo sapiens | | | |
| | | cDNA clone IMAGE: 2316636 3′, | | | |
| | | mRNA sequence | | | |
| 11 | AI733141.1 | AI733141 ol81a03.x5 | 0.5479 | 0 | 1 |
| | | NCI_CGAP_Kid5 Homo sapiens | | | |
| | | cDNA clone IMAGE: 1535980 3′, | | | |
| | | mRNA sequence | | | |
| 12 | AI801879.1 | AI801879 tx28f05.x1 | 0.2595 | 1 | 0 |
| | | NCI_CGAP_Lu24 Homo sapiens | | | |
| | | cDNA clone IMAGE: 2270913 3′, | | | |
| | | mRNA sequence | | | |
| 13 | AI870477.1 | AI870477 wl74b03.x1 | 0.7639 | 0 | 1 |
| | | NCI_CGAP_Brn25 Homo sapiens | | | |
| | | cDNA clone IMAGE: 2430605 3′, | | | |
| | | mRNA sequence | | | |
| 14 | AK022356.1 | Homo sapiens cDNA FLJ12294 fis, | 0.6871 | 0 | 1 |
| | | clone MAMMA1001817 | | | |
| 15 | AK023312.1 | Homo sapiens cDNA FLJ13250 fis, | 0.7707 | 0 | 1 |
| | | clone OVARC1000724 | | | |
| 16 | AK023856.1 | Homo sapiens cDNA FLJ13794 fis, | 0.2276 | 1 | 0 |
| | | clone THYRO1000092 | | | |
| 17 | AK024998.1 | Homo sapiens cDNA: FLJ21345 | 0.6494 | 0 | 1 |
| | | fis, clone COL02694 | | | |
| 18 | AK057505.1 | Homo sapiens cDNA FLJ32943 fis, | 0.8767 | 0 | 1 |
| | | clone TESTI2007829 | | | |
| 19 | AK091021.1 | Homo sapiens cDNA FLJ33702 fis, | 0.7426 | 0 | 1 |
| | | clone BRAWH2005533 | | | |
| 20 | AK091830.1 | Homo sapiens cDNA FLJ34511 fis, | 0.6938 | 0 | 1 |
| | | clone HLUNG2006397 | | | |
| 21 | AK092541.1 | Homo sapiens cDNA FLJ35222 fis, | 0.691 | 0 | 1 |
| | | clone PROST2000835 | | | |
| 22 | AK092875.1 | Homo sapiens cDNA FLJ35556 fis, | 0.3468 | 1 | 0 |
| | | clone SPLEN2004844 | | | |
| 23 | AK095109.1 | Homo sapiens cDNA FLJ37790 fis, | 0.7859 | 0 | 1 |
| | | clone BRHIP3000111 | | | |
| 24 | AK097658.1 | Homo sapiens cDNA FLJ40339 fis, | 0.3469 | 1 | 0 |
| | | clone TESTI2032079 | | | |
| 25 | AK098306.1 | Homo sapiens cDNA FLJ40987 fis, | 0.6876 | 0 | 1 |
| | | clone UTERU2015062 | | | |
| 26 | AK124927.1 | Homo sapiens cDNA FLJ42937 fis, | 0.1741 | 1 | 0 |
| | | clone BRSSN2014556 | | | |
| 27 | AK127572.1 | Homo sapiens cDNA FLJ45665 fis, | 0.5898 | 0 | 1 |
| | | clone CTONG2027959 | | | |
| 28 | AK127877.1 | Homo sapiens cDNA FLJ45982 fis, | 0.7119 | 0 | 1 |
| | | clone PROST2017729 | | | |
| 29 | AK130903.1 | Homo sapiens cDNA FLJ27393 fis, | 0.7623 | 0 | 1 |
| | | clone WMC01011 | | | |
| 30 | AK131516.1 | Homo sapiens cDNA FLJ16742 fis, | 0.8201 | 0 | 1 |
| | | clone BRAWH2008993 | | | |
| 31 | AV741821.1 | AV741821 AV741821 CB Homo | 0.7017 | 0 | 1 |
| | | sapiens cDNA clone CBLACB04 5′, | | | |
| | | mRNA sequence | | | |
| 32 | AW070221.1 | AW070221 xa09d05.x1 | 0.6662 | 0 | 1 |
| | | Soares_NFL_T_GBC_S1 Homo | | | |
| | | sapiens cDNA clone | | | |
| | | IMAGE: 2567817 3′ similar to | | | |
| | | TR: O15503 O15503 INSULIN | | | |
| | | INDUCED PROTEIN 1.;, mRNA | | | |
| | | sequence | | | |
| 33 | AW592040.1 | AW592040 hf37f06.x1 | 0.8192 | 0 | 1 |
| | | Soares_NFL_T_GBC_S1 Homo | | | |
| | | sapiens cDNA clone | | | |
| | | IMAGE: 2934083 3′, mRNA | | | |
| | | sequence | | | |
| 34 | AW662723.1 | AW662723 hi35g04.x1 | 0.623 | 0 | 1 |
| | | NCI_CGAP_Co14 Homo sapiens | | | |
| | | cDNA clone IMAGE: 2974326 3′ | | | |
| | | similar to gb: M60724 RIBOSOMAL | | | |
| | | PROTEIN S6 KINASE (HUMAN);, | | | |
| | | mRNA sequence | | | |
| 35 | AY054401.3 | Homo sapiens non-coding | 0.7634 | 0 | 1 |
| | | transcript BT1C (BDNF) mRNA, | | | |
| | | complete sequence; alternatively | | | |
| | | spliced | | | |
| 36 | AY176665.1 | Homo sapiens nervous system | 0.7225 | 0 | 1 |
| | | abundant protein 11 (NSAP11) | | | |
| | | mRNA, complete cds | | | |
| 37 | BC033363.1 | Homo sapiens, clone | 0.8908 | 0 | 1 |
| | | IMAGE: 4753714, mRNA | | | |
| 38 | BC034424.1 | Homo sapiens hexosaminidase A | 0.6379 | 0 | 1 |
| | | (alpha polypeptide), mRNA (cDNA | | | |
| | | clone IMAGE: 4823589) | | | |
| 39 | BC035195.2 | Homo sapiens cDNA clone | 0.6273 | 0 | 1 |
| | | IMAGE: 5266689 | | | |
| 40 | BC035377.1 | Homo sapiens cDNA clone | 0.4531 | 1 | 0 |
| | | IMAGE: 4826240 | | | |
| 41 | BC038752.1 | Homo sapiens cDNA clone | 0.7525 | 0 | 1 |
| | | IMAGE: 5269351 | | | |
| 42 | BC039104.1 | Homo sapiens hypothetical protein | 0.8318 | 0 | 1 |
| | | LOC283404, mRNA (cDNA clone | | | |
| | | IMAGE: 4828118) | | | |
| 43 | BC040610.1 | Homo sapiens ribosomal protein | 0.7936 | 0 | 1 |
| | | L4, mRNA (cDNA clone | | | |
| | | IMAGE: 3897039) | | | |
| 44 | BC042060.1 | Homo sapiens olfactory receptor, | 0.7563 | 0 | 1 |
| | | family 7, subfamily E, member 47 | | | |
| | | pseudogene, mRNA (cDNA clone | | | |
| | | IMAGE: 5590288) | | | |
| 45 | BC042816.1 | Homo sapiens cDNA clone | 0.7201 | 0 | 1 |
| | | IMAGE: 5314175 | | | |
| 46 | BC042855.1 | Homo sapiens cDNA clone | 0.8326 | 0 | 1 |
| | | IMAGE: 5313513, with apparent | | | |
| | | retained intron | | | |
| 47 | BC043574.1 | Homo sapiens, clone | 0.685 | 0 | 1 |
| | | IMAGE: 5222953, mRNA | | | |
| 48 | BC044257.1 | Homo sapiens, clone | 0.6643 | 0 | 1 |
| | | IMAGE: 6063621, mRNA | | | |
| 49 | BC044741.1 | Homo sapiens cDNA clone | 0.3626 | 1 | 0 |
| | | IMAGE: 4828106 | | | |
| 50 | BC053955.1 | Homo sapiens hypothetical protein | 0.6361 | 0 | 1 |
| | | LOC285548, mRNA (cDNA clone | | | |
| | | IMAGE: 4839316) | | | |
| 51 | BC054862.1 | Homo sapiens cDNA clone | 0.8227 | 0 | 1 |
| | | IMAGE: 4288461, partial cds | | | |
| 52 | BC078172.1 | Homo sapiens cDNA clone | 0.8116 | 0 | 1 |
| | | IMAGE: 5760022, partial cds | | | |
| 53 | BC108263.1 | Homo sapiens transmembrane | 0.8339 | 0 | 1 |
| | | protein 56, mRNA (cDNA clone | | | |
| | | IMAGE: 4801733), **** WARNING: | | | |
| | | chimeric clone **** | | | |
| 54 | BC127846.1 | Homo sapiens cDNA clone | 0.8948 | 0 | 1 |
| | | IMAGE: 40134482 | | | |
| 55 | BE745782.1 | BE745782 601579970F1 | 0.2625 | 1 | 0 |
| | | NIH_MGC_9 Homo sapiens cDNA | | | |
| | | clone IMAGE: 3928841 5′, mRNA | | | |
| | | sequence | | | |
| 56 | BE785612.1 | BE785612 601475144F1 | 0.7293 | 0 | 1 |
| | | NIH_MGC_68 Homo sapiens | | | |
| | | cDNA clone IMAGE: 3878051 5′, | | | |
| | | mRNA sequence | | | |
| 57 | BE044435.1 | BE044435 ho45d08.x1 | 0.7093 | 0 | 1 |
| | | Soares_NFL_T_GBC_S1 Homo | | | |
| | | sapiens cDNA clone | | | |
| | | IMAGE: 3040335 3′, mRNA | | | |
| | | sequence | | | |
| 58 | BF062994.1 | BF062994 7h73f05.x1 | 0.714 | 0 | 1 |
| | | NCI_CGAP_Co16 Homo sapiens | | | |
| | | cDNA clone IMAGE: 3321633 3′, | | | |
| | | mRNA sequence | | | |
| 59 | BF245041.1 | BF245041 601864168F1 | 0.7327 | 0 | 1 |
| | | NIH_MGC_57 Homo sapiens | | | |
| | | cDNA clone IMAGE: 4082368 5′, | | | |
| | | mRNA sequence | | | |
| 60 | BF594738.1 | BF594738 7o54h12.x1 | 0.2631 | 1 | 0 |
| | | NCI_CGAP_Kid11 Homo sapiens | | | |
| | | cDNA clone IMAGE: 3577991 3′, | | | |
| | | mRNA sequence | | | |
| 61 | BF688062.1 | BF688062 602067272F1 | 0.2489 | 1 | 0 |
| | | NIH_MGC_57 Homo sapiens | | | |
| | | cDNA clone IMAGE: 4066433 5′, | | | |
| | | mRNA sequence | | | |
| 62 | BG189068.1 | BG189068 RST8104 Athersys | 0.6341 | 0 | 1 |
| | | RAGE Library Homo sapiens | | | |
| | | cDNA, mRNA sequence | | | |
| 63 | BG201613.1 | BG201613 RST20954 Athersys | 0.194 | 1 | 0 |
| | | RAGE Library Homo sapiens | | | |
| | | cDNA, mRNA sequence | | | |
| 64 | BG203790.1 | BG203790 RST23181 Athersys | 0.2773 | 1 | 0 |
| | | RAGE Library Homo sapiens | | | |
| | | cDNA, mRNA sequence | | | |
| 65 | BI462136.1 | BI462136 603205131F1 | 0.3108 | 1 | 0 |
| | | NIH_MGC_97 Homo sapiens | | | |
| | | cDNA clone IMAGE: 5270983 5′, | | | |
| | | mRNA sequence | | | |
| 66 | BI559775.1 | BI559775 603252664F1 | 0.727 | 0 | 1 |
| | | NIH_MGC_97 Homo sapiens | | | |
| | | cDNA clone IMAGE: 5295231 5′, | | | |
| | | mRNA sequence | | | |
| 67 | BI825982.1 | BI825982 603076566F1 | 0.7214 | 0 | 1 |
| | | NIH_MGC_119 Homo sapiens | | | |
| | | cDNA clone IMAGE: 5168225 5′, | | | |
| | | mRNA sequence | | | |
| 68 | BM461531.1 | BM461531 | 0.4477 | 1 | 0 |
| | | AGENCOURT_6421147 | | | |
| | | NIH_MGC_67 Homo sapiens | | | |
| | | cDNA clone IMAGE: 5501266 5′, | | | |
| | | mRNA sequence | | | |
| 69 | BM690995.1 | BM690995 UI-E-CI1-aba-d-08-0- | 0.7291 | 0 | 1 |
| | | UI.r1 UI-E-CI1 Homo sapiens | | | |
| | | cDNA clone UI-E-CI1-aba-d-08-0- | | | |
| | | UI 5′, mRNA sequence | | | |
| 70 | BQ184944.1 | BQ184944 UI-E-EJ1-ajo-c-04-0- | 0.7141 | 0 | 1 |
| | | UI.s1 UI-E-EJ1 Homo sapiens | | | |
| | | cDNA clone UI-E-EJ1-ajo-c-04-0- | | | |
| | | UI 3′, mRNA sequence | | | |
| 71 | BQ233546.1 | BQ233546 | 0.6304 | 0 | 1 |
| | | AGENCOURT_7526687 | | | |
| | | NIH_MGC_70 Homo sapiens | | | |
| | | cDNA clone IMAGE: 6018551 5′, | | | |
| | | mRNA sequence | | | |
| 72 | BU533525.1 | BU533525 | 0.6682 | 0 | 1 |
| | | AGENCOURT_10197749 | | | |
| | | NIH_MGC_126 Homo sapiens | | | |
| | | cDNA clone IMAGE: 6559929 5′, | | | |
| | | mRNA sequence | | | |
| 73 | BU534173.1 | BU534173 | 0.303 | 1 | 0 |
| | | AGENCOURT_10240114 | | | |
| | | NIH_MGC_126 Homo sapiens | | | |
| | | cDNA clone IMAGE: 6561006 5′, | | | |
| | | mRNA sequence | | | |
| 74 | BU619815.1 | BU619815 UI-H-FH1-bfq-j-08-0- | 0.3354 | 1 | 0 |
| | | UI.s1 NCI_CGAP_FH1 Homo | | | |
| | | sapiens cDNA clone UI-H-FH1-bfq- | | | |
| | | j-08-0-UI 3′, mRNA sequence | | | |
| 75 | BX089034.1 | BX089034 BX089034 | 0.8095 | 0 | 1 |
| | | Soares_parathyroid_tumor_NbHPA | | | |
| | | Homo sapiens cDNA clone | | | |
| | | IMAGp998M163120; | | | |
| | | IMAGE: 1240503 5′, mRNA | | | |
| | | sequence | | | |
| 76 | BX090666.1 | BX090666 BX090666 | 0.7584 | 0 | 1 |
| | | Soares_testis_NHT Homo sapiens | | | |
| | | cDNA clone IMAGp998D014412; | | | |
| | | IMAGE: 1736400 5′, mRNA | | | |
| | | sequence | | | |
| 77 | BX100329.1 | BX100329 BX100329 | 0.7407 | 0 | 1 |
| | | Soares_NFL_T_GBC_S1 Homo | | | |
| | | sapiens cDNA clone | | | |
| | | IMAGp998H043806; | | | |
| | | IMAGE: 1503795 5′, mRNA | | | |
| | | sequence | | | |
| 78 | BX100818.1 | BX100818 BX100818 | 0.7962 | 0 | 1 |
| | | Soares_fetal_lung_NbHL19W | | | |
| | | Homo sapiens cDNA clone | | | |
| | | IMAGp998J074430; | | | |
| | | IMAGE: 1743462 5′, mRNA | | | |
| | | sequence | | | |
| 79 | BX103408.1 | BX103408 BX103408 Soares | 0.3196 | 1 | 0 |
| | | melanocyte 2NbHM Homo sapiens | | | |
| | | cDNA clone IMAGp998L01545; | | | |
| | | IMAGE: 251664 5′, mRNA | | | |
| | | sequence | | | |
| 80 | BX103636.1 | BX103636 BX103636 | 0.8348 | 0 | 1 |
| | | Soares_testis_NHT Homo sapiens | | | |
| | | cDNA clone IMAGp998J184112; | | | |
| | | IMAGE: 1621361 5′, mRNA | | | |
| | | sequence | | | |
| 81 | BX104605.1 | BX104605 BX104605 | 0.7985 | 0 | 1 |
| | | Soares_testis_NHT Homo sapiens | | | |
| | | cDNA clone IMAGp998B211795; | | | |
| | | IMAGE: 731444 5′, mRNA | | | |
| | | sequence | | | |
| 82 | BX537644.1 | Homo sapiens mRNA; cDNA | 0.7389 | 0 | 1 |
| | | DKFZp686M1498 (from clone | | | |
| | | DKFZp686M1498) | | | |
| 83 | BX537772.1 | Homo sapiens mRNA; cDNA | 0.8385 | 0 | 1 |
| | | DKFZp781M2440 (from clone | | | |
| | | DKFZp781M2440) | | | |
| 84 | BX648555.1 | Homo sapiens mRNA; cDNA | 0.6607 | 0 | 1 |
| | | DKFZp779B0135 (from clone | | | |
| | | DKFZp779B0135) | | | |
| 85 | BX648926.1 | Homo sapiens mRNA; cDNA | 0.3742 | 1 | 0 |
| | | DKFZp686O0329 (from clone | | | |
| | | DKFZp686O0329) | | | |
| 86 | NM_022895.1 | Homo sapiens chromosome 12 | 0.3436 | 1 | 0 |
| | | open reading frame 43 (C12orf43), | | | |
| | | mRNA | | | |
| 87 | NM_152318.2 | Homo sapiens chromosome 12 | 0.3186 | 1 | 0 |
| | | open reading frame 45 (C12orf45), | | | |
| | | mRNA | | | |
| 88 | CR457199.1 | Homo sapiens full open reading | 0.4427 | 1 | 0 |
| | | frame cDNA clone | | | |
| | | RZPDo834G068D for gene | | | |
| | | C14orf112, chromosome 14 open | | | |
| | | reading frame 112; complete cds, | | | |
| | | incl. stopcodon | | | |
| 89 | NM_004894.2 | Homo sapiens chromosome 14 | 0.7418 | 0 | 1 |
| | | open reading frame 2 (C14orf2), | | | |
| | | transcript variant 1, mRNA | | | |
| 90 | BC007346.2 | Homo sapiens chromosome 16 | 0.4108 | 1 | 0 |
| | | open reading frame 14, mRNA | | | |
| | | (cDNA clone IMAGE: 3689407), | | | |
| | | complete cds | | | |
| 91 | NM_033520.1 | Homo sapiens chromosome 19 | 0.622 | 0 | 1 |
| | | open reading frame 33 (C19orf33), | | | |
| | | mRNA | | | |
| 92 | NM_024038.2 | Homo sapiens chromosome 19 | 0.4308 | 1 | 0 |
| | | open reading frame 43 (C19orf43), | | | |
| | | mRNA | | | |
| 93 | NM_014047.2 | Homo sapiens chromosome 19 | 0.7672 | 0 | 1 |
| | | open reading frame 53 (C19orf53), | | | |
| | | mRNA | | | |
| 94 | NM_019108.2 | Homo sapiens chromosome 19 | 0.7063 | 0 | 1 |
| | | open reading frame 61 (C19orf61), | | | |
| | | mRNA | | | |
| 95 | NM_018840.2 | Homo sapiens chromosome 20 | 0.7255 | 0 | 1 |
| | | open reading frame 24 (C20orf24), | | | |
| | | transcript variant 1, mRNA | | | |
| 96 | NM_021254.1 | Homo sapiens chromosome 21 | 0.7483 | 0 | 1 |
| | | open reading frame 59 (C21orf59), | | | |
| | | mRNA | | | |
| 97 | NM_015702.1 | Homo sapiens chromosome 2 | 0.7598 | 0 | 1 |
| | | open reading frame 25 (C2orf25), | | | |
| | | mRNA | | | |
| 98 | NM_016474.4 | Homo sapiens chromosome 3 | 0.3994 | 1 | 0 |
| | | open reading frame 19 (C3orf19), | | | |
| | | mRNA | | | |
| 99 | NM_178335.1 | Homo sapiens coiled-coil domain | 0.7952 | 0 | 1 |
| | | containing 50 | | | |
| | | (CCDC50), C3ORF6, transcript | | | |
| | | variant 2, mRNA | | | |
| 100 | NM_032302.2 | Homo sapiens proteasome | 0.787 | 0 | 1 |
| | | (prosome, macropain) assembly | | | |
| | | chaperone 3 (PSMG3), mRNA | | | |
| 101 | NM_019607.1 | Homo sapiens chromosome 8 | 0.4354 | 1 | 0 |
| | | open reading frame 44 (C8orf44), | | | |
| | | mRNA | | | |
| 102 | NM_017998.2 | Homo sapiens chromosome 9 | 0.7684 | 0 | 1 |
| | | open reading frame 40 (C9orf40), | | | |
| | | mRNA | | | |
| 103 | CB045860.1 | CB045860 NISC_gf01a03.x1 | 0.724 | 0 | 1 |
| | | NCI_CGAP_Kid12 Homo sapiens | | | |
| | | cDNA clone IMAGE: 3252364 3′, | | | |
| | | mRNA sequence | | | |
| 104 | CD692919.1 | CD692919 EST9442 human | 0.6126 | 0 | 1 |
| | | nasopharynx Homo sapiens cDNA, | | | |
| | | mRNA sequence | | | |
| 105 | CN267986.1 | CN267986 17000531863184 | 0.6675 | 0 | 1 |
| | | GRN_EB Homo sapiens cDNA 5′, | | | |
| | | mRNA sequence | | | |
| 106 | CN280387.1 | CN280387 17000455082974 | 0.7509 | 0 | 1 |
| | | GRN_ES Homo sapiens cDNA 5′, | | | |
| | | mRNA sequence | | | |
| 107 | CN398253.1 | CN398253 17000424721764 | 0.7986 | 0 | 1 |
| | | GRN_EB Homo sapiens cDNA 5′, | | | |
| | | mRNA sequence | | | |
| 108 | CR593740.1 | full-length cDNA clone | 0.7132 | 0 | 1 |
| | | CS0DF033YJ19 of Fetal brain of | | | |
| | | Homo sapiens (human) | | | |
| 109 | CR604408.1 | full-length cDNA clone | 0.8164 | 0 | 1 |
| | | CS0DC001YF03 of Neuroblastoma | | | |
| | | Cot 25-normalized of Homo | | | |
| | | sapiens (human) | | | |
| 110 | CR623475.1 | full-length cDNA clone | 0.6816 | 0 | 1 |
| | | CS0DB006YA03 of Neuroblastoma | | | |
| | | Cot 10-normalized of Homo | | | |
| | | sapiens (human) | | | |
| 111 | CR626360.1 | full-length cDNA clone | 0.7563 | 0 | 1 |
| | | CS0DM014YM20 of Fetal liver of | | | |
| | | Homo sapiens (human) | | | |
| 112 | CR627148.1 | Homo sapiens mRNA; cDNA | 0.7868 | 0 | 1 |
| | | DKFZp779F2127 (from clone | | | |
| | | DKFZp779F2127) | | | |
| 113 | CR737784.1 | CR737784 CR737784 Homo | 0.8232 | 0 | 1 |
| | | sapiens library (Ebert L) Homo | | | |
| | | sapiens cDNA clone | | | |
| | | IMAGp998C154208; | | | |
| | | IMAGE: 1658054 5′, mRNA | | | |
| | | sequence | | | |
| 114 | CR994463.1 | CR994463 CR994463 RZPD | 0.659 | 0 | 1 |
| | | no. 9016 Homo sapiens cDNA | | | |
| | | clone RZPDp9016A109 5′, mRNA | | | |
| | | sequence | | | |
| 115 | DB049861.1 | DB049861 DB049861 TESTI2 | 0.8422 | 0 | 1 |
| | | Homo sapiens cDNA clone | | | |
| | | TESTI2039270 5′, mRNA | | | |
| | | sequence | | | |
| 116 | DB054822.1 | DB054822 DB054822 TESTI2 | 0.7785 | 0 | 1 |
| | | Homo sapiens cDNA clone | | | |
| | | TESTI2045843 5′, mRNA | | | |
| | | sequence | | | |
| 117 | DB186251.1 | DB186251 DB186251 TLIVE2 | 0.2773 | 1 | 0 |
| | | Homo sapiens cDNA clone | | | |
| | | TLIVE2006096 5′, mRNA | | | |
| | | sequence | | | |
| 118 | DB331110.1 | DB331110 DB331110 SKMUS2 | 0.2272 | 1 | 0 |
| | | Homo sapiens cDNA clone | | | |
| | | SKMUS2008761 3′, mRNA | | | |
| | | sequence | | | |
| 119 | DB514539.1 | DB514539 DB514539 RIKEN full- | 0.7233 | 0 | 1 |
| | | length enriched human cDNA | | | |
| | | library, testis Homo sapiens cDNA | | | |
| | | clone H013041M08 3′, mRNA | | | |
| | | sequence | | | |
| 120 | DB522524.1 | DB522524 DB522524 RIKEN full- | 0.7956 | 0 | 1 |
| | | length enriched human cDNA | | | |
| | | library, testis Homo sapiens cDNA | | | |
| | | clone H013076C14 3′, mRNA | | | |
| | | sequence | | | |
| 121 | DC347972.1 | DC347972 DC347972 CTONG3 | 0.6791 | 0 | 1 |
| | | Homo sapiens cDNA clone | | | |
| | | CTONG3005404 5′, mRNA | | | |
| | | sequence | | | |
| 122 | AL137478.1 | Homo sapiens mRNA; cDNA | 0.8034 | 0 | 1 |
| | | DKFZp434M1123 (from clone | | | |
| | | DKFZp434M1123) | | | |
| 123 | EF565105.1 | Homo sapiens chromosome 16 | 0.5012 | 0 | 1 |
| | | isolate HA_003251 mRNA | | | |
| | | sequence | | | |
| 124 | DB089792.1 | DB089792 DB089792 TESTI4 | 0.7495 | 0 | 1 |
| | | Homo sapiens cDNA clone | | | |
| | | TESTI4038491 5′, mRNA | | | |
| | | sequence | | | |
| 125 | NM_018011.3 | Homo sapiens arginine and | 0.3163 | 1 | 0 |
| | | glutamate rich 1 (ARGLU1), mRNA | | | |
| 126 | NM_018048.2 | Homo sapiens mago-nashi | 0.7617 | 0 | 1 |
| | | homolog B (Drosophila) | | | |
| | | (MAGOHB), mRNA | | | |
| 127 | NM_017669.2 | Homo sapiens excision repair | 0.8155 | 0 | 1 |
| | | cross-complementing rodent repair | | | |
| | | deficiency, complementation group | | | |
| | | 6-like (ERCC6L), mRNA | | | |
| 128 | NM_144726.1 | Homo sapiens ring finger protein | 0.8475 | 0 | 1 |
| | | 145 (RNF145), mRNA | | | |
| 129 | XR_040666.1 | PREDICTED: Homo sapiens | 0.4847 | 1 | 0 |
| | | misc_RNA (FLJ32065), miscRNA | | | |
| 130 | NM_001039796.1 | Homo sapiens hypothetical protein | 0.752 | 0 | 1 |
| | | LOC649446 (FLJ35776), mRNA | | | |
| 131 | NM_015168.1 | Homo sapiens zinc finger CCCH- | 0.1932 | 1 | 0 |
| | | type containing 4 (ZC3H4), mRNA | | | |
| 132 | NM_020827.1 | Homo sapiens KIAA1430 | 0.3263 | 1 | 0 |
| | | (KIAA1430), mRNA | | | |
| 133 | NM_001009993.2 | Homo sapiens family with | 0.6583 | 0 | 1 |
| | | sequence similarity 168, member B | | | |
| | | (FAM168B), mRNA | | | |
| 134 | NM_001086521.1 | Homo sapiens chromosome 17 | 0.6882 | 0 | 1 |
| | | open reading frame 89 (C17orf89), | | | |
| | | mRNA | | | |
| 135 | NR_002187.2 | Homo sapiens hypothetical protein | 0.7608 | 0 | 1 |
| | | LOC286016 (LOC286016) on | | | |
| | | chromosome 7 | | | |
| 136 | NM_001080507.1 | Homo sapiens oocyte expressed | 0.6789 | 0 | 1 |
| | | protein homolog (dog) (OOEP), | | | |
| | | mRNA | | | |
| 137 | XR_039886.1 | PREDICTED: Homo sapiens | 0.6685 | 0 | 1 |
| | | misc_RNA (LOC541471), | | | |
| | | miscRNA | | | |
| 138 | NM_020314.4 | Homo sapiens chromosome 16 | 0.7113 | 0 | 1 |
| | | open reading frame 62 (C16orf62), | | | |
| | | mRNA | | | |
| 139 | NM_024093.1 | Homo sapiens chromosome 2 | 0.7338 | 0 | 1 |
| | | open reading frame 49 (C2orf49), | | | |
| | | mRNA | | | |
| 140 | NM_001004333.3 | Homo sapiens ribonuclease, | 0.5969 | 0 | 1 |
| | | RNase K (RNASEK), mRNA | | | |
| 141 | AK098520.1 | Homo sapiens cDNA FLJ25654 fis, | 0.2283 | 1 | 0 |
| | | clone TST00252 | | | |
| 142 | NM_001093732.1 | Homo sapiens hCG2033311 | 0.6534 | 0 | 1 |
| | | (LOC644928), mRNA | | | |
| 143 | NM_015681.3 | Homo sapiens B9 protein domain 1 | 0.6197 | 0 | 1 |
| | | (B9D1), mRNA | | | |
| 144 | T85821.1 | T85821 yd57b09.r1 Soares fetal | 0.7951 | 0 | 1 |
| | | liver spleen 1NFLS Homo sapiens | | | |
| | | cDNA clone IMAGE: 112313 5′ | | | |
| | | similar to contains MER25 | | | |
| | | repetitive element;, mRNA | | | |
| | | sequence | | | |
| 145 | T85822.1 | T85822 yd57b10.r1 Soares fetal | 0.7259 | 0 | 1 |
| | | liver spleen 1NFLS Homo sapiens | | | |
| | | cDNA clone IMAGE: 112315 5′, | | | |
| | | mRNA sequence | | | |
| 146 | T85823.1 | T85823 yd57b11.r1 Soares fetal | 0.815 | 0 | 1 |
| | | liver spleen 1NFLS Homo sapiens | | | |
| | | cDNA clone IMAGE: 112317 5′ | | | |
| | | similar to contains LTR1 repetitive | | | |
| | | element;, mRNA sequence | | | |
| 147 | T85824.1 | T85824 yd57b12.r1 Soares fetal | 0.8146 | 0 | 1 |
| | | liver spleen 1NFLS Homo sapiens | | | |
| | | cDNA clone IMAGE: 112319 5′, | | | |
| | | mRNA sequence | | | |
| 148 | AI342698.1 | AI342698 qo35e04.x1 | 0.6337 | 0 | 1 |
| | | NCI_CGAP_Lu5 Homo sapiens | | | |
| | | cDNA clone IMAGE: 1910526 3′ | | | |
| | | similar to gb: L01457 | | | |
| | | AUTOANTIGEN PM-SCL | | | |
| | | (HUMAN);, mRNA sequence | | | |
| 149 | AK094352.1 | Homo sapiens cDNA FLJ37033 fis, | 0.6052 | 0 | 1 |
| | | clone BRACE2011389 | | | |
| 150 | AK094903.1 | Homo sapiens cDNA FLJ37584 fis, | 0.3903 | 1 | 0 |
| | | clone BRCOC2004950 | | | |
| 151 | AK128457.1 | Homo sapiens cDNA FLJ46600 fis, | 0.3942 | 1 | 0 |
| | | clone THYMU3047144 | | | |
| 152 | AW418496.1 | AW418496 ha19c01.x1 | 0.4929 | 1 | 0 |
| | | NCI_CGAP_Kid12 Homo sapiens | | | |
| | | cDNA clone IMAGE: 2874144 3′, | | | |
| | | mRNA sequence | | | |
| 153 | AX748230.1 | Sequence 1755 from Patent | 0.7376 | 0 | 1 |
| | | EP1308459 | | | |
| 154 | BC005233.1 | Homo sapiens pancreatic lipase- | 0.5561 | 0 | 1 |
| | | related protein 1, mRNA (cDNA | | | |
| | | clone IMAGE: 3950129), complete | | | |
| | | cds | | | |
| 155 | BC036259.1 | Homo sapiens hypothetical gene | 0.6996 | 0 | 1 |
| | | supported by AK093266, mRNA | | | |
| | | (cDNA clone IMAGE: 5271013) | | | |
| 156 | BG221753.1 | BG221753 RST41568 Athersys | 0.6439 | 0 | 1 |
| | | RAGE Library Homo sapiens | | | |
| | | cDNA, mRNA sequence | | | |
| 157 | BX648475.1 | Homo sapiens mRNA; cDNA | 0.795 | 0 | 1 |
| | | DKFZp686P11156 (from clone | | | |
| | | DKFZp686P11156) | | | |
| 158 | NM_017915.2 | Homo sapiens chromosome 12 | 0.3315 | 1 | 0 |
| | | open reading frame 48 (C12orf48), | | | |
| | | mRNA | | | |
| 159 | BC001722.1 | Homo sapiens chromosome 14 | 0.6383 | 0 | 1 |
| | | open reading frame 166, mRNA | | | |
| | | (cDNA clone MGC: 680 | | | |
| | | IMAGE: 3528725), complete cds | | | |
| 160 | NM_024294.2 | Homo sapiens chromosome 6 | 0.5592 | 0 | 1 |
| | | open reading frame 106 | | | |
| | | (C6orf106), transcript variant 1, | | | |
| | | mRNA | | | |
| 161 | NM_138701.2 | Homo sapiens chromosome 7 | 0.4211 | 1 | 0 |
| | | open reading frame 11 (C7orf11), | | | |
| | | mRNA | | | |
| 162 | NG_005982.3 | Homo sapiens ribosomal protein, | 0.7143 | 0 | 1 |
| | | large, P1 pseudogene | | | |
| | | (LOC729416) on chromosome 5 | | | |
| 163 | N68399.1 | N68399 za13b04.s1 Soares fetal | 0.6699 | 0 | 1 |
| | | liver spleen 1NFLS Homo sapiens | | | |
| | | cDNA clone IMAGE: 292399 3′ | | | |
| | | similar to SW: OLF3_MOUSE | | | |
| | | P23275 OLFACTORY RECEPTOR | | | |
| | | OR3. [1];, mRNA sequence | | | |
| 164 | NT_022171.14 | Hs2_22327 Homo sapiens | 0.6871 | 0 | 1 |
| | | chromosome 2 genomic contig, | | | |
| | | reference assembly | | | |

Table 3 lists all the proteins in the library.

TABLE 3

| Clone ID | Protein name | Protein description |
|---|---|---|
| 310505p4f1b8 | 08-Sep | septin 9 |
| 170407pl3E6 | 09-Sep | septin 10 isoform 1 |
| 200208pl2D10 | 10-Sep | septin 11 |
| 050707pl1E1 | BE745782 | heparan sulfate D-glucosaminyl |
| 200906pl2E4 | A-761H5.5 | hypothetical protein LOC440350 |
| 310806pl2C10 | AA033764 | zk19b11.r1 Soares_pregnant_uterus_NbHPU |
| | | Homo sapiens cDNA clone IMAGE: 470973 5′, |
| | | mRNA sequence. |
| 130207pl1D8 | AA282714 | zt13f10.r1 NCI_CGAP_GCB1 Homo sapiens cDNA |
| | | clone IMAGE: 713035 5′, mRNA sequence. |
| 310806pl2E7 | AA431778 | zw80e04.s1 Soares_testis_NHT Homo sapiens |
| | | cDNA clone IMAGE: 782526 3′, mRNA sequence. |
| 050707pl3H3 | AA435616 | zt74d10.s1 Soares_testis_NHT Homo sapiens |
| | | cDNA clone IMAGE: 728083 3′, mRNA sequence. |
| 150506pl1F4 | AA479512 | zv21f09.s1 Soares_NhHMPu_S1 Homo sapiens |
| | | cDNA clone IMAGE: 754313 3′, mRNA sequence. |
| 311007pl2C7 | AA758225 | ah68g10.s1 Soares_testis_NHT Homo sapiens |
| | | cDNA clone 1320834 3′, mRNA sequence. |
| 150506pl1A5 | AA843465 | aj54c11.s1 Soares_testis_NHT Homo sapiens |
| | | cDNA clone IMAGE: 1394132 3′, mRNA sequence. |
| 041206pl4C2 | AA913230 | ol41h07.s1 Soares_NFL_T_GBC_S1 Homo |
| | | sapiens cDNA clone IMAGE: 1526077 3′, mRNA |
| | | sequence. |
| 041206pl7B5 | AA928516 | om17h03.s1 Soares_NFL_T_GBC_S1 Homo |
| | | sapiens cDNA clone IMAGE: 1541333 3′, mRNA |
| | | sequence. |
| 310806pl3A11 | AA933969 | on71h05.s1 Soares_NFL_T_GBC_S1 Homo |
| | | sapiens cDNA clone IMAGE: 1562169 3′ similar to |
| | | gb: K00558 TUBULIN ALPHA-1 CHAIN (HUMAN);, |
| | | mRNA sequence. |
| 200906pl3A5 | AB051441 | Homo sapiens mRNA for KIAA1654 protein, partial |
| | | cds. |
| 200208pl2E12 | ABCA4 | ATP-binding cassette, sub-family A member 4 |
| 200906pl1E6 | ABCF1 | ATP-binding cassette, sub-family F, member 1 |
| 10704p110c8 | ACOT7 | acyl-CoA thioesterase 7 isoform hBACHb |
| 171104p42c6 | ACTN1 | actinin, alpha 1 |
| 31104p37b6 | ACTN4 | actinin, alpha 4 |
| 050707pl1B4 | ACTR1A | ARP1 actin-related protein 1 homolog A, |
| 170407vpl2B6 | ACTR2 | actin-related protein 2 isoform a |
| 041206pl4D12 | ACTR3 | ARP3 actin-related protein 3 homolog |
| 311007pl1B8 | ACYP2 | muscle-type acylphosphatase 2 |
| 311007pl3G6 | ADH5 | class III alcohol dehydrogenase 5 chi subunit |
| 150506pl2E6 | ADK | adenosine kinase isoform b |
| 310506pl3C9 | AF086125 | Homo sapiens full length insert cDNA clone |
| | | ZA79D12. |
| 310506pl3C2 | AF087973 | Homo sapiens full length insert cDNA clone |
| | | YU79H10. |
| 200906pl3G9 | AF220048 | Homo sapiens uncharacterized hematopoietic |
| | | stem/progenitor cells protein MDS028 mRNA, |
| | | complete cds. |
| 201107pl2A12 | AF339799 | Homo sapiens clone IMAGE: 2363394, mRNA |
| | | sequence. |
| 010806pl2C2 | AHNAK | AHNAK nucleoprotein isoform 2 |
| 310506pl2A10 | AI000260 | ov10b02.s1 NCI_CGAP_Kid3 Homo sapiens cDNA |
| | | clone IMAGE: 1636875 3′ similar to contains |
| | | THR.b3 THR repetitive element;, mRNA |
| | | sequence. |
| 041206pl1D9 | AI001881 | ot39c06.s1 Soares_testis_NHT Homo sapiens |
| | | cDNA clone IMAGE: 1619146 3′, mRNA sequence. |
| 010806pl2A5 | AI094227 | qa43a12.s1 Soares_NhHMPu_S1 Homo sapiens |
| | | cDNA clone IMAGE: 1689502 3′, mRNA sequence. |
| 310506pl1E10 | AI125255 | qd87h09.x1 Soares_testis_NHT Homo sapiens |
| | | cDNA clone IMAGE: 1736513 3′, mRNA sequence. |
| 160507pl3F1 | AI203131 | qr34b09.x1 NCI_CGAP_GC6 Homo sapiens cDNA |
| | | clone IMAGE: 1942745 3′, mRNA sequence. |
| 200906pl4F5 | AI208228 | qg50b01.x1 Soares_testis_NHT Homo sapiens |
| | | cDNA clone IMAGE: 1838569 3′, mRNA sequence. |
| 201107pl1A1 | AI215862 | qm35e03.x1 NCI_CGAP_Lu5 Homo sapiens cDNA |
| | | clone IMAGE: 1883836 3′ similar to contains Alu |
| | | repetitive element; contains element MER22 |
| | | repetitive element;, mRNA sequence. |
| 050707pl3E7 | AI217733 | qh15h09.x1 Soares_NFL_T_GBC_S1 Homo |
| | | sapiens cDNA clone IMAGE: 1844801 3′ similar to |
| | | SW: FTCD_PIG P53603 |
| | | FORMIMINOTRANSFERASE- |
| | | CYCLODEAMINASE; contains element PTR5 |
| | | repetitive element;, mRNA sequence. |
| 310506pl1G2 | AI310103 | qo74c04.x1 NCI_CGAP_Kid5 Homo sapiens cDNA |
| | | clone IMAGE: 1914246 3′, mRNA sequence. |
| 201107pl3F7 | AI342698 | qo35e04.x1 NCI_CGAP_Lu5 Homo sapiens cDNA |
| | | clone IMAGE: 1910526 3′ similar to gb: L01457 |
| | | AUTOANTIGEN PM-SCL (HUMAN);, mRNA |
| | | sequence. |
| 010806pl2H4 | AI434862 | ti13c03.x1 NCI_CGAP_Kid11 Homo sapiens cDNA |
| | | clone IMAGE: 2130340 3′, mRNA sequence. |
| 050707pl2E11 | AI671392 | wc29g07.x1 NCI_CGAP_Kid11 Homo sapiens |
| | | cDNA clone IMAGE: 2316636 3′, mRNA sequence. |
| 200306f7pl1C8 | AI692920 | wd42h05.x1 Soares_NFL_T_GBC_S1 Homo |
| | | sapiens cDNA clone IMAGE: 2330841 3′, mRNA |
| | | sequence. |
| 200906pl2B7 | AI733141 | ol81a03.x5 NCI_CGAP_Kid5 Homo sapiens cDNA |
| | | clone IMAGE: 1535980 3′, mRNA sequence. |
| 201107pl4A11 | AI769786 | wj26e10.x1 NCI_CGAP_Kid12 Homo sapiens |
| | | cDNA clone IMAGE: 2403978 3′, mRNA sequence. |
| 150506pl2E8 | AI801879 | tx28f05.x1 NCI_CGAP_Lu24 Homo sapiens cDNA |
| | | clone IMAGE: 2270913 3′, mRNA sequence. |
| 170407pl3F6 | AI822094 | za73d07.x5 Soares_fetal_lung_NbHL19W Homo |
| | | sapiens cDNA clone IMAGE: 298189 3′ similar to |
| | | gb: X16667 HOMEOBOX PROTEIN HOX-B3 |
| | | (HUMAN);, mRNA sequence. |
| 130207pl1C12 | AI869329 | wl68g08.x1 NCI_CGAP_Brn25 Homo sapiens |
| | | cDNA clone IMAGE: 2430110 3′, mRNA sequence. |
| 201107pl1G4 | AI869566 | wl98c09.x1 NCI_CGAP_Brn25 Homo sapiens |
| | | cDNA clone IMAGE: 2432944 3′ similar to |
| | | SW:SSRP_HUMAN Q08945 STRUCTURE- |
| | | SPECIFIC RECOGNITION PROTEIN 1;, mRNA |
| | | sequence. |
| 041206pl5F10 | AI870477 | wl74b03.x1 NCI_CGAP_Brn25 Homo sapiens |
| | | cDNA clone IMAGE: 2430605 3′, mRNA sequence. |
| 041206pl7B4 | AJ412031 | Homo sapiens mRNA for B-cell neoplasia |
| | | associated transcript, (BCMS gene), splice variant |
| | | D, non coding transcript. |
| 310806pl1C11 | AJ713761 | AJ713761 LKPD01 Homo sapiens cDNA clone |
| | | LKPD02011, mRNA sequence. |
| 160507pl2B5 | AK000451 | Homo sapiens cDNA FLJ20444 fis, clone |
| | | KAT05128. |
| 130207pl1D5 | AK022356 | Homo sapiens cDNA FLJ12294 fis, clone |
| | | MAMMA1001817. |
| 201107pl1F12 | AK023018 | Homo sapiens cDNA FLJ12956 fis, clone |
| | | NT2RP2005501. |
| 010806pl1E8 | AK023312 | Homo sapiens cDNA FLJ13250 fis, clone |
| | | OVARC1000724. |
| 200906pl1A1 | AK023856 | Homo sapiens cDNA FLJ13794 fis, clone |
| | | THYRO1000092. |
| 311007pl3F10 | AK024998 | Homo sapiens cDNA: FLJ21345 fis, clone |
| | | COL02694. |
| 200906pl2E11 | AK025325 | Homo sapiens cDNA: FLJ21672 fis, clone |
| | | COL09025. |
| 200306f7pl1D8 | AK055171 | Homo sapiens cDNA FLJ30609 fis, clone |
| | | CTONG2000480. |
| 050707pl2B10 | AK056115 | Homo sapiens cDNA FLJ31553 fis, clone |
| | | NT2RI2001178. |
| 310506pl1A4 | AK056558 | Homo sapiens cDNA FLJ31996 fis, clone |
| | | NT2RP7009253. |
| 041206pl3A1 | AK057505 | Homo sapiens C18orf2 isoform 1 mRNA, complete |
| | | sequence, alternatively spliced. |
| 170407pl1G8 | AK091021 | Homo sapiens cDNA FLJ33702 fis, clone |
| | | BRAWH2005533. |
| 041206pl7D6 | AK091108 | Homo sapiens cDNA FLJ33789 fis, clone |
| | | BRSSN2009378. |
| 170407pl1E9 | AK092541 | Homo sapiens cDNA FLJ35222 fis, clone |
| | | PROST2000835. |
| 050707pl1D5 | AK092875 | Homo sapiens cDNA FLJ35556 fis, clone |
| | | SPLEN2004844. |
| 201107pl3F2 | AK094352 | Homo sapiens cDNA FLJ37033 fis, clone |
| | | BRACE2011389. |
| 201107pl2A7 | AK094903 | Homo sapiens cDNA FLJ37584 fis, clone |
| | | BRCOC2004950. |
| 311007pl2G12 | AK095077 | Homo sapiens cDNA FLJ37758 fis, clone |
| | | BRHIP2023869. |
| 170407pl1D7 | AK095109 | Homo sapiens cDNA FLJ37790 fis, clone |
| | | BRHIP3000111. |
| 041206pl1D7 | AK097571 | Homo sapiens cDNA FLJ40252 fis, clone |
| | | TESTI2024299. |
| 010806pl3E4 | AK097658 | Homo sapiens cDNA FLJ40339 fis, clone |
| | | TESTI2032079. |
| 200906pl2D9 | AK098170 | Homo sapiens cDNA FLJ40851 fis, clone |
| | | TRACH2014997, moderately similar to Rattus |
| | | norvegicus Ca2+-dependent activator protein |
| | | (CAPS) mRNA. |
| 160507pl2G5 | AK098264 | Homo sapiens cDNA FLJ40945 fis, clone |
| | | UTERU2008747. |
| 190607pl1B6 | AK098306 | Homo sapiens cDNA FLJ40987 fis, clone |
| | | UTERU2015062. |
| 041206pl6H5 | AK123491 | Homo sapiens cDNA FLJ41497 fis, clone |
| | | BRTHA2006075. |
| 200906pl2F6 | AK123797 | Homo sapiens cDNA FLJ41803 fis, clone |
| | | NHNPC2002749. |
| 150506pl2B2 | AK124927 | Homo sapiens cDNA FLJ42937 fis, clone |
| | | BRSSN2014556. |
| 200906pl5D9 | AK127877 | Homo sapiens cDNA FLJ45982 fis, clone |
| | | PROST2017729. |
| 280305p1f2e12 | AK128282 | Homo sapiens cDNA FLJ46419 fis, clone |
| | | THYMU3012983, moderately similar to Homo |
| | | sapiens zinc finger protein 14 (KOX 6) (ZNF14). |
| 201107pl2D4 | AK128457 | Homo sapiens cDNA FLJ46600 fis, clone |
| | | THYMU3047144. |
| 310806pl1D8 | AK128738 | Homo sapiens cDNA FLJ16787 fis, clone |
| | | PLACE6013222. |
| 310506pl3G7 | AK130268 | Homo sapiens cDNA FLJ26758 fis, clone |
| | | PRS02459. |
| 311007pl3D4 | AK130830 | Homo sapiens cDNA FLJ27320 fis, clone |
| | | TMS07774. |
| 010806pl4E5 | AK130903 | Homo sapiens cDNA FLJ27393 fis, clone |
| | | WMC01011. |
| 150506pl1G6 | AK131516 | Homo sapiens cDNA FLJ16742 fis, clone |
| | | BRAWH2008993. |
| 041206pl2E2 | AKAP12 | A-kinase anchor protein 12 isoform 1 |
| 170407pl1B12 | AKAP8L | A kinase (PRKA) anchor protein 8-like |
| 310806pl2E1 | AL136790 | Homo sapiens mRNA; cDNA DKFZp434F1819 |
| | | (from clone DKFZp434F1819). |
| 041206pl6H11 | AL137366 | Homo sapiens mRNA; cDNA DKFZp434F1626 |
| | | (from clone DKFZp434F1626). |
| 310506pl3B7 | AL708335 | DKFZp686L2051_r1 686 (synonym: hlcc3) Homo |
| | | sapiens cDNA clone DKFZp686L2051 5′, mRNA |
| | | sequence. |
| 010806pl1F6 | ALDH3B1 | Homo sapiens mRNA for aldehyde dehydrogenase |
| | | 3B1 variant protein. |
| 311007pl1H1 | ALDOA | aldolase A |
| 170407pl1G4 | ALG14 | asparagine-linked glycosylation 14 homolog |
| 180504p21c4 | AMD1 | S-adenosylmethionine decarboxylase 1 isoform 1 |
| 200208pl2G2 | ANAPC13 | anaphase promoting complex subunit 13 |
| 190607pl1C10 | ANGPTL4 | angiopoietin-like 4 protein isoform a precursor |
| 280705p1f13A8 | ANLN | anillin, actin binding protein (scraps homolog, |
| 041206pl4E5 | ANP32A | acidic (leucine-rich) nuclear phosphoprotein 32 |
| 280305p1f12D9 | ANP32B | acidic (leucine-rich) nuclear phosphoprotein 32 |
| 160507pl3A1 | ANTXR2 | anthrax toxin receptor 2 |
| 200906pl5A11 | ANXA1 | annexin I |
| 200906pl4A6 | ANXA11 | annexin A11 |
| 280305p5f2E6 | ANXA2 | annexin A2 isoform 1 |
| 201107pl2G6 | ANXA5 | annexin 5 |
| 170407vpl3H9 | ANXA8L1 | annexin A8-like 1 |
| 150506pl1G7 | AOAH | acyloxyacyl hydrolase precursor |
| 311007pl1H12 | AOF2 | amine oxidase (flavin containing) domain 2 |
| 310806pl2B6 | APIP | APAF1 interacting protein |
| 311007pl1A7 | APLP2 | amyloid beta (A4) precursor-like protein 2 |
| 201107pl3B8 | APP | amyloid beta A4 protein precursor, isoform a |
| 130207p2G10 | ARCH | Homo sapiens archease (ARCH) mRNA, partial |
| | | cds. |
| 010806pl2D6 | ARHGAP18 | Rho GTPase activating protein 18 |
| 041206pl7B1 | ARID1B | AT rich interactive domain 1B (SWI1-like) |
| 050707pl3G1 | ARL3 | ADP-ribosylation factor-like 3 |
| 160507pl2F5 | ARL6IP1 | ADP-ribosylation factor-like 6 interacting |
| 200208pl2F6 | ARMC2 | armadillo repeat containing 2 |
| 010806pl4E10 | ARPC1A | actin related protein 2/3 complex subunit 1A |
| 200906pl2C10 | ARPC2 | actin related protein 2/3 complex subunit 2 |
| 050707pl3E10 | ARPC3 | actin related protein 2/3 complex subunit 3 |
| 200208pl2F12 | ASNS | Homo sapiens cDNA FLJ20372 fis, clone |
| | | HEP19727, highly similar to M27396 Human |
| | | asparagine synthetase mRNA. |
| 200906pl1B3 | ATAD1 | ATPase family, AAA domain containing 1 |
| 170407vpl2E12 | ATF1 | activating transcription factor 1 |
| 050707pl3D10 | ATG3 | Apg3p |
| 200208pl2A4 | ATOX1 | antioxidant protein 1 |
| 27073j5 | ATP1A1 | Na+/K+ -ATPase alpha 1 subunit isoform a |
| 310505p4f1c8 | ATP5B | ATP synthase, H+ transporting, mitochondrial F1 |
| 311007pl1G5 | ATP5C1 | ATP synthase, H+ transporting, mitochondrial F1 |
| 310806pl1E1 | ATP5J2 | ATP synthase, H+ transporting, mitochondrial F0 |
| 170604p17c11 | ATP6V1D | H(+)-transporting two-sector ATPase |
| 310806pl1G11 | AV702071 | AV702071 ADB Homo sapiens cDNA clone |
| | | ADBCVC06 5′, mRNA sequence. |
| 200906pl5G5 | AV703421 | AV703421 ADB Homo sapiens cDNA clone |
| | | ADBCBH03 5′, mRNA sequence. |
| 200906pl1F1 | AV741821 | AV741821 CB Homo sapiens cDNA clone |
| | | CBLACB04 5′, mRNA sequence. |
| 200306f7pl1F11 | AVEN | cell death regulator aven |
| 150506pl1A10 | AW070221 | xa09d05.x1 Soares_NFL_T_GBC_S1 Homo |
| | | sapiens cDNA clone IMAGE: 2567817 3′ similar to |
| | | TR: O15503 O15503 INSULIN INDUCED |
| | | PROTEIN 1.;, mRNA sequence. |
| 041206pl6F4 | AW070342 | xa10d08.x1 Soares_NFL_T_GBC_S1 Homo |
| | | sapiens cDNA clone IMAGE: 2567919 3′, mRNA |
| | | sequence. |
| 310506pl1G9 | AW136353 | UI-H-BI1-acn-f-11-0-UI.s1 NCI_CGAP_Sub3 Homo |
| | | sapiens cDNA clone IMAGE: 2715021 3′, mRNA |
| | | sequence. |
| 310806pl2D6 | AW241724 | xn74c07.x1 Soares_NFL_T_GBC_S1 Homo |
| | | sapiens cDNA clone IMAGE: 2700204 3′, mRNA |
| | | sequence. |
| 010806pl2B10 | AW291591 | UI-H-BI2-agk-g-08-0-UI.s1 NCI_CGAP_Sub4 |
| | | Homo sapiens cDNA clone IMAGE: 2724686 3′, |
| | | mRNA sequence. |
| 201107pl3E2 | AW418496 | ha19c01.x1 NCI_CGAP_Kid12 Homo sapiens |
| | | cDNA clone IMAGE: 2874144 3′, mRNA sequence. |
| 160507pl3A12 | AW592040 | hf37f06.x1 Soares_NFL_T_GBC_S1 Homo |
| | | sapiens cDNA clone IMAGE: 2934083 3′, mRNA |
| | | sequence. |
| 150506pl1B4 | AX748015 | Homo sapiens cDNA FLJ35934 fis, clone |
| | | TESTI2011315. |
| 201107pl3D2 | AX748230 | Homo sapiens cDNA FLJ36305 fis, clone |
| | | THYMU2004677. |
| 310806pl1D3 | AX748388 | Homo sapiens cDNA FLJ36653 fis, clone |
| | | UTERU2001176. |
| 160507pl1A1 | AY054401 | Homo sapiens trapped 3′ terminal exon, clone |
| | | B2F11. |
| 010806pl2D10 | AY176665 | Homo sapiens nervous system abundant protein |
| | | 11 (NSAP11) mRNA, complete cds. |
| 041206pl7C6 | AY480055 | Homo sapiens GKT-AML5-1 mRNA sequence; |
| | | alternatively spliced. |
| 050707pl2G4 | BAG1 | BCL2-associated athanogene. |
| 310506pl3A4 | BAG2 | BCL2-associated athanogene 2 |
| 170407pl3D4 | BAG3 | BCL2-associated athanogene 3 |
| 170407vpl2C4 | BAIAP2 | BAI1-associated protein 2 isoform 3 |
| 201107pl2D2 | BAIAP2L1 | BAI1-associated protein 2-like 1 |
| 201107pl2H3 | BANK1 | B-cell scaffold protein with ankyrin repeats 1 |
| 050707pl1G4 | BARD1 | BRCA1 associated RING domain 1 |
| 310806pl1G1 | BC000085 | Homo sapiens cDNA clone IMAGE: 3507983, **** |
| | | WARNING: chimeric clone ****. |
| 200906pl3H5 | BC011779 | Homo sapiens cDNA clone IMAGE: 3941306, |
| | | partial cds. |
| 050707pl2E9 | BC012743 | Homo sapiens cDNA clone IMAGE: 4040306, **** |
| | | WARNING: chimeric clone ****. |
| 311007pl3C7 | BC014506 | Homo sapiens, clone IMAGE: 4863312, mRNA. |
| 180504p12d6 | BC014776 | Homo sapiens hypothetical LOC541471, mRNA |
| | | (cDNA clone MGC: 17532 IMAGE: 3459303), |
| | | complete cds. |
| 041206pl2G8 | BC015412 | Homo sapiens cDNA clone IMAGE: 4393471, |
| | | partial cds. |
| 200306f7pl1F1 | BC016972 | Homo sapiens, clone IMAGE: 3896086, mRNA. |
| 310506pl1D5 | BC024924 | Homo sapiens cDNA FLJ12974 fis, clone |
| | | NT2RP2006103. |
| 041206pl4G1 | BC031950 | Homo sapiens cDNA clone IMAGE: 4838164. |
| 041206pl3G3 | BC033363 | Homo sapiens, clone IMAGE: 4753714, mRNA. |
| 201107pl4D10 | BC033643 | Homo sapiens cDNA clone MGC: 45452 |
| | | IMAGE: 5562656, complete cds. |
| 010506pl2B6 | BC035195 | Homo sapiens cDNA clone IMAGE: 5266689. |
| 200306d9pl1C6 | BC035377 | Homo sapiens cDNA clone IMAGE: 4826240. |
| 201107pl2G5 | BC036259 | Homo sapiens cDNA FLJ35947 fis, clone |
| | | TESTI2011971. |
| 160507pl1B6 | BC038752 | Homo sapiens cDNA clone IMAGE: 5269351. |
| 310506pl1D10 | bc038760 | hEST |
| 150506pl1E5 | BC039104 | Homo sapiens hypothetical protein LOC283404, |
| | | mRNA (cDNA clone IMAGE: 4828118). |
| 310806pl2C8 | BC039429 | Homo sapiens cDNA clone IMAGE: 5303182. |
| 041206pl1C3 | BC039533 | Homo sapiens, clone IMAGE: 5743964, mRNA. |
| 201107pl1G10 | BC039555 | Homo sapiens, clone IMAGE: 4249217, mRNA. |
| 050707pl2F12 | BC040619 | Homo sapiens similar to solute carrier family 16 |
| | | (monocarboxylic acid transporters), member 14, |
| | | mRNA (cDNA clone IMAGE: 5726657). |
| 010806pl3A5 | BC041444 | Homo sapiens cDNA FLJ27393 fis, clone |
| | | WMC01011. |
| 310806pl2C9 | BC042816 | Homo sapiens full length insert cDNA YN57B01. |
| 160507pl1C8 | BC042855 | Homo sapiens mRNA; cDNA DKFZp434A0326 |
| | | (from clone DKFZp434A0326). |
| 150506pl1D7 | BC044257 | Homo sapiens, clone IMAGE: 6063621, mRNA. |
| 050707pl2D12 | BC044741 | Homo sapiens cDNA clone IMAGE: 4828106. |
| 310506pl3D10 | BC048320 | Homo sapiens, clone IMAGE: 4450067, mRNA. |
| 200306d9pl1C11 | BC048993 | Homo sapiens hypothetical protein LOC285550, |
| | | mRNA (cDNA clone IMAGE: 4686377), partial cds. |
| 130207pl2A4 | BC053955 | Homo sapiens hypothetical protein LOC285548, |
| | | mRNA (cDNA clone IMAGE: 5265914). |
| 160507pl3B5 | BC054862 | Homo sapiens cDNA clone IMAGE: 4288461, |
| | | partial cds. |
| 160507pl1F5 | BC078172 | Homo sapiens cDNA clone IMAGE: 5760022, |
| | | partial cds. |
| 041206pl2H4 | BC082260 | Homo sapiens cDNA clone IMAGE: 6427299, **** |
| | | WARNING: chimeric clone ****. |
| 170407vpl3C9 | BC108263 | Homo sapiens transmembrane protein 56, mRNA |
| | | (cDNA clone IMAGE: 4801733), **** WARNING: |
| | | chimeric clone ****. |
| 041206pl5E3 | BCCIP | BRCA2 and CDKN1A-interacting protein isoform C |
| 200906pl5C5 | BE043072 | ho32e06.x1 NCI_CGAP_Lu24 Homo sapiens |
| | | cDNA clone IMAGE: 3039106 3′, mRNA sequence. |
| 010506pl2D10 | BE044435 | ho45d08.x1 Soares_NFL_T_GBC_S1 Homo |
| | | sapiens cDNA clone IMAGE: 3040335 3′, mRNA |
| | | sequence. |
| 041206pl7D5 | BE048560 | hr50f01.x1 NCI_CGAP_Kid11 Homo sapiens |
| | | cDNA clone IMAGE: 3131929 3′ similar to contains |
| | | Alu repetitive element; contains element TAR1 |
| | | repetitive element;, mRNA sequence. |
| 310506pl1G10 | BE048868 | hr54h09.x1 NCI_CGAP_Kid11 Homo sapiens |
| | | cDNA clone IMAGE: 3132353 3′ similar to contains |
| | | MER13.t3 MER13 repetitive element;, mRNA |
| | | sequence. |
| 050707pl2F4 | BE257831 | 601109413F1 NIH_MGC_16 Homo sapiens cDNA |
| | | clone IMAGE: 3350114 5′, mRNA sequence. |
| 160507pl3D7 | BE466653 | hz23g02.x1 NCI_CGAP_GC6 Homo sapiens |
| | | cDNA clone IMAGE: 3208850 3′, mRNA sequence. |
| 201107pl4A4 | BE504704 | hz31c02.x1 NCI_CGAP_GC6 Homo sapiens cDNA |
| | | clone IMAGE: 3209570 3′ similar to TR: P97346 |
| | | P97346 NUCLEOREDOXIN;, mRNA sequence. |
| 041206pl6G1 | BE505026 | hz36h06.x1 NCI_CGAP_GC6 Homo sapiens |
| | | cDNA clone IMAGE: 3210107 3′, mRNA sequence. |
| 010806pl2A2 | BE785612 | 601475144F1 NIH_MGC_68 Homo sapiens cDNA |
| | | clone IMAGE: 3878051 5′, mRNA sequence. |
| 311007pl2C3 | BF001694 | 7g91h05.x1 NCI_CGAP_Co16 Homo sapiens |
| | | cDNA clone IMAGE: 3313881 3′ similar to |
| | | TR: O60705 O60705 LIM PROTEIN.;, mRNA |
| | | sequence. |
| 160507pl2D11 | BF062994 | 7h73f05.x1 NCI_CGAP_Co16 Homo sapiens |
| | | cDNA clone IMAGE: 3321633 3′, mRNA sequence. |
| 310506pl1E3 | BF244436 | 601862730F1 NIH_MGC_57 Homo sapiens cDNA |
| | | clone IMAGE: 4080511 5′, mRNA sequence. |
| 190607pl1C5 | BF245041 | 601864168F1 NIH_MGC_57 Homo sapiens cDNA |
| | | clone IMAGE: 4082368 5′, mRNA sequence. |
| 041206pl3C4 | BF434856 | 7o74e08.x1 NCI_CGAP_Kid11 Homo sapiens |
| | | cDNA clone IMAGE: 3641967 3′, mRNA sequence. |
| 150506pl1B11 | BF509736 | UI-H-BI4-apg-b-02-0-UI.s1 NCI_CGAP_Sub8 |
| | | Homo sapiens cDNA clone IMAGE: 3087290 3′, |
| | | mRNA sequence. |
| 200906pl2B2 | BF594738 | 7o54h12.x1 NCI_CGAP_Kid11 Homo sapiens |
| | | cDNA clone IMAGE: 3577991 3′, mRNA sequence. |
| 041206pl6A1 | BF688062 | 602067272F1 NIH_MGC_57 Homo sapiens cDNA |
| | | clone IMAGE: 4066433 5′, mRNA sequence. |
| 200906pl5B9 | BF875734 | QV3-ET0103-111100-386-a04 ET0103 Homo |
| | | sapiens cDNA, mRNA sequence. |
| 311007pl3G12 | BG189068 | RST8104 Athersys RAGE Library Homo sapiens |
| | | cDNA, mRNA sequence. |
| 041206pl3G11 | BG201613 | RST20954 Athersys RAGE Library Homo sapiens |
| | | cDNA, mRNA sequence. |
| 160507pl2C7 | BG203790 | RST23181 Athersys RAGE Library Homo sapiens |
| | | cDNA, mRNA sequence. |
| 201107pl3F4 | BG221753 | RST41568 Athersys RAGE Library Homo sapiens |
| | | cDNA, mRNA sequence. |
| 310506pl3H3 | BG426583 | 602493305F1 NIH_MGC_75 Homo sapiens cDNA |
| | | clone IMAGE: 4607305 5′, mRNA sequence. |
| 311007pl3D2 | BG505700 | 602549869F1 NIH_MGC_61 Homo sapiens cDNA |
| | | clone IMAGE: 4657624 5′, mRNA sequence. |
| 050707pl1G10 | BG716117 | 602677572F1 NIH_MGC_96 Homo sapiens cDNA |
| | | clone IMAGE: 4800233 5′, mRNA sequence. |
| 310506pl2A1 | BG753571 | 602733141F1 NIH_MGC_43 Homo sapiens cDNA |
| | | clone IMAGE: 4876330 5′, mRNA sequence. |
| 170407pl1D3 | BI462136 | 603205131F1 NIH_MGC_97 Homo sapiens cDNA |
| | | clone IMAGE: 5270983 5′, mRNA sequence. |
| 150506pl1F3 | BI559775 | 603252664F1 NIH_MGC_97 Homo sapiens cDNA |
| | | clone IMAGE: 5295231 5′, mRNA sequence. |
| 050707pl3H8 | BI762388 | 603049060F1 NIH_MGC_116 Homo sapiens |
| | | cDNA clone IMAGE: 5189054 5′, mRNA sequence. |
| 311007pl3F3 | BI825982 | 603076566F1 NIH_MGC_119 Homo sapiens |
| | | cDNA clone IMAGE: 5168225 5′, mRNA sequence. |
| 150506pl2D3 | BI838110 | 603083607F1 NIH_MGC_120 Homo sapiens |
| | | cDNA clone IMAGE: 5222953 5′, mRNA sequence. |
| 130207pl2C2 | BIN1 | bridging integrator 1 isoform 1 |
| 010506pl1C3 | BIN2 | bridging integrator 2 |
| 200906pl1D2 | BM461531 | AGENCOURT_6421147 NIH_MGC_67 Homo |
| | | sapiens cDNA clone IMAGE: 5501266 5′, mRNA |
| | | sequence. |
| 200906pl1E11 | BM681834 | UI-E-EJ0-aiq-g-07-0-UI.s1 UI-E-EJ0 Homo sapiens |
| | | cDNA clone UI-E-EJ0-aiq-g-07-0-UI 3′, mRNA |
| | | sequence. |
| 010806pl2G8 | BM684766 | UI-E-EJ1-ajj-m-22-0-UI.s1 UI-E-EJ1 Homo sapiens |
| | | cDNA clone UI-E-EJ1-ajj-m-22-0-UI 3′, mRNA |
| | | sequence. |
| 041206pl3D6 | BM690995 | UI-E-CI1-aba-d-08-0-UI.r1 UI-E-CI1 Homo sapiens |
| | | cDNA clone UI-E-CI1-aba-d-08-0-UI 5′, mRNA |
| | | sequence. |
| 200906pl1D10 | BM691000 | UI-E-CI1-aba-e-01-0-UI.r1 UI-E-CI1 Homo sapiens |
| | | cDNA clone UI-E-CI1-aba-e-01-0-UI 5′, mRNA |
| | | sequence. |
| 310806pl2B3 | BM749023 | K-EST0024086 S10SNU1 Homo sapiens cDNA |
| | | clone S10SNU1-1-F09 5′, mRNA sequence. |
| 041206pl2D7 | BM905834 | AGENCOURT_6721121 NIH_MGC_71 Homo |
| | | sapiens cDNA clone IMAGE: 5556193 5′, mRNA |
| | | sequence. |
| 170407vpl3B5 | BOLA2 | BolA-like protein 2 isoform b |
| 200906pl5F8 | bpl 41-16 | Homo sapiens olfactory receptor, family 7, |
| | | subfamily E, member 47 pseudogene, mRNA |
| | | (cDNA clone IMAGE: 5590288). |
| 200906pl4B10 | BQ011346 | UI-1-BC1p-arz-e-06-0-UI.s1 NCI_CGAP_PI3 Homo |
| | | sapiens cDNA clone UI-1-BC1p-arz-e-06-0-UI 3′, |
| | | mRNA sequence. |
| 201107pl3E1 | BQ183849 | UI-H-EU0-azs-b-24-0-UI.s1 NCI_CGAP_Car1 |
| | | Homo sapiens cDNA clone IMAGE: 5852855 3′, |
| | | mRNA sequence. |
| 290307pl1A6 | BQ184944 | UI-E-EJ1-ajo-c-04-0-UI.s1 UI-E-EJ1 Homo sapiens |
| | | cDNA clone UI-E-EJ1-ajo-c-04-0-UI 3′, mRNA |
| | | sequence. |
| 130207pl1D3 | BQ230709 | AGENCOURT_7546358 NIH_MGC_70 Homo |
| | | sapiens cDNA clone IMAGE: 6025005 5′, mRNA |
| | | sequence. |
| 160507pl1D8 | BQ233546 | AGENCOURT_7526687 NIH_MGC_70 Homo |
| | | sapiens cDNA clone IMAGE: 6018551 5′, mRNA |
| | | sequence. |
| 200208pl2B4 | BRIP1 | BRCA1 interacting protein C-terminal helicase 1 |
| 170407pl1E10 | BRMS1 | breast cancer metastasis suppressor 1 isoform 2 |
| 280705p1f13D3 | BSG | basigin isoform 1 |
| 170407vpl3A9 | BTK | Homo sapiens Bruton's tyrosine kinase mRNA, |
| | | complete cds. |
| 311007pl3F2 | BU533525 | AGENCOURT_10197749 NIH_MGC_126 Homo |
| | | sapiens cDNA clone IMAGE: 6559929 5′, mRNA |
| | | sequence. |
| 130207pl2C5 | BU534173 | AGENCOURT_10240114 NIH_MGC_126 Homo |
| | | sapiens cDNA clone IMAGE: 6561006 5′, mRNA |
| | | sequence. |
| 010806pl2B5 | BU568189 | AGENCOURT_10404673 NIH_MGC_82 Homo |
| | | sapiens cDNA clone IMAGE: 6615135 5′, mRNA |
| | | sequence. |
| 310806pl1F4 | BU599750 | AGENCOURT_8827710 NIH_MGC_142 Homo |
| | | sapiens cDNA clone IMAGE: 6458824 5′, mRNA |
| | | sequence. |
| 050707pl2D5 | BU607353 | UI-CF-FN0-aeu-g-14-0-UI.s1 UI-CF-FN0 Homo |
| | | sapiens cDNA clone UI-CF-FN0-aeu-g-14-0-UI 3′, |
| | | mRNA sequence. |
| 150506pl1G1 | BU619815 | UI-H-FH1-bfq-j-08-0-UI.s1 NCI_CGAP_FH1 Homo |
| | | sapiens cDNA clone UI-H-FH1-bfq-j-08-0-UI 3′, |
| | | mRNA sequence. |
| 200906pl4F9 | BU621210 | UI-H-FL1-bfz-e-02-0-UI.s1 NCI_CGAP_FL1 Homo |
| | | sapiens cDNA clone UI-H-FL1-bfz-e-02-0-UI 3′, |
| | | mRNA sequence. |
| 041206pl2A2 | BU630466 | UI-H-FL0-bdk-a-10-0-UI.s1 NCI_CGAP_FL0 Homo |
| | | sapiens cDNA clone UI-H-FL0-bdk-a-10-0-UI 3′, |
| | | mRNA sequence. |
| 310506pl1G6 | BU753850 | UI-1-BC1p-alh-b-11-0-UI.s1 NCI_CGAP_PI3 Homo |
| | | sapiens cDNA clone UI-1-BC1p-alh-b-11-0-UI 3′, |
| | | mRNA sequence. |
| 041206pl6G3 | BU930695 | AGENCOURT_10425457 NIH_MGC_83 Homo |
| | | sapiens cDNA clone IMAGE: 6668795 5′, mRNA |
| | | sequence. |
| 010806pl4B8 | BX090666 | BX090666 Soares_testis_NHT Homo sapiens |
| | | cDNA clone IMAGp998D014412; IMAGE: 1736400 |
| | | 5′, mRNA sequence. |
| 041206pl4F4 | BX096972 | BX096972 Soares fetal liver spleen 1NFLS Homo |
| | | sapiens cDNA clone IMAGp998A01130; |
| | | IMAGE: 127368 5′, mRNA sequence. |
| 290307pl1D1 | BX100329 | BX100329 Soares_NFL_T_GBC_S1 Homo |
| | | sapiens cDNA clone IMAGp998H043806; |
| | | IMAGE: 1503795 5′, mRNA sequence. |
| 050707pl2D8 | BX100818 | BX100818 Soares_fetal_lung_NbHL19W Homo |
| | | sapiens cDNA clone IMAGp998J074430; |
| | | IMAGE: 1743462 5′, mRNA sequence. |
| 180504p11c2 | BX101084 | hEST |
| 311007pl3D7 | BX103408 | BX103408 Soares melanocyte 2NbHM Homo |
| | | sapiens cDNA clone IMAGp998L01545; |
| | | IMAGE: 251664 5′, mRNA sequence. |
| 160507pl1E5 | BX103636 | BX103636 Soares_testis_NHT Homo sapiens |
| | | cDNA clone IMAGp998J184112; IMAGE: 1621361 |
| | | 5′, mRNA sequence. |
| 200906pl2H6 | BX104605 | BX104605 Soares_testis_NHT Homo sapiens |
| | | cDNA clone IMAGp998B211795; IMAGE: 731444 |
| | | 5′, mRNA sequence. |
| 130207pl2E11 | BX108181 | BX108181 Soares_testis_NHT Homo sapiens |
| | | cDNA clone IMAGp998A194412; IMAGE: 1736346 |
| | | 5′, mRNA sequence. |
| 200906pl5B4 | BX364993 | BX364993 Homo sapiens PLACENTA COT 25- |
| | | NORMALIZED Homo sapiens cDNA clone |
| | | CS0DI038YA06 5-PRIME, mRNA sequence. |
| 311007pl1D12 | BX537644 | Homo sapiens cDNA: FLJ23130 fis, clone |
| | | LNG08419. |
| 010806pl4E8 | BX537772 | Homo sapiens mRNA; cDNA DKFZp781M2440 |
| | | (from clone DKFZp781M2440). |
| 201107pl1B3 | BX538309 | Homo sapiens mRNA; cDNA DKFZp686C09130 |
| | | (from clone DKFZp686C09130). |
| 201107pl2C1 | BX648475 | Homo sapiens mRNA; cDNA DKFZp686p11156 |
| | | (from clone DKFZp686p11156). |
| 130207pl2D4 | BX648555 | Homo sapiens mRNA; cDNA DKFZp779B0135 |
| | | (from clone DKFZp779B0135). |
| 150506pl2G3 | BX648926 | Homo sapiens mRNA; cDNA DKFZp686O0329 |
| | | (from clone DKFZp686O0329). |
| 310806pl1F9 | BXDC1 | brix domain containing 1 |
| 041206pl1F7 | C10orf129 | Homo sapiens cDNA FLJ44146 fis, clone |
| | | THYMU2027734, weakly similar to Homo sapiens |
| | | SA hypertension-associated homolog (rat) (SAH). |
| 150506pl2F2 | C12orf43 | hypothetical protein LOC64897 |
| 311007pl2D5 | C12orf45 | hypothetical protein LOC121053 |
| 201107pl1B10 | C14orf102 | hypothetical protein LOC55051 isoform 1 |
| 160507pl2A3 | C14orf112 | hypothetical protein LOC51241 |
| 041206pl2A8 | C14orf140 | chromosome 14 open reading frame 140 isoform a |
| 190607pl1A8 | C14orf2 | hypothetical protein LOC9556 |
| 310506pl1G11 | C16orf14 | hypothetical protein LOC84331 |
| 041206pl6G12 | C17orf49 | hypothetical protein LOC124944 |
| 311007pl2A6 | C19orf33 | HAI-2 related small protein |
| 160507pl1A2 | C19orf43 | hypothetical protein MGC2803 |
| 200906pl2D8 | C19orf61 | hypothetical protein LOC56006 |
| 050707pl3D7 | C1orf121 | hypothetical protein LOC51029 |
| 180504p13e3 | C1orf149 | hypothetical protein LOC64769 |
| 310506pl1F5 | C1orf62 | hypothetical protein LOC254268 |
| 010806pl1H5 | C1QBP | complement component 1, q subcomponent |
| | | binding |
| 200906pl2E6 | C20orf24 | hEST |
| 160507pl3H5 | C20orf52 | reactive oxygen species modulator 1 |
| 160507pl2B10 | C21orf59 | Homo sapiens T-complex protein 10A-2 mRNA, |
| | | complete cds. |
| 041206pl1H7 | C22orf16 | chromosome 22 open reading frame 16 |
| 311007pl1C5 | C2orf25 | hypothetical protein LOC27249 |
| 201107pl4B1 | C2orf27 | hypothetical protein LOC29798 |
| 170407pl3F1 | C2orf49 | hypothetical protein LOC79074 |
| 010506pl1E8 | C3orf19 | hypothetical protein LOC51244 |
| 201107pl3B1 | C3orf26 | hypothetical protein LOC84319 |
| 201107pl2C3 | C6orf106 | chromosome 6 open reading frame 106 isoform a |
| 310806pl1E10 | C6orf51 | hypothetical protein LOC112495 |
| 200208pl2B5 | C6orf64 | hypothetical protein LOC55776 |
| 201107pl3G8 | C7orf11 | chromosome 7 open reading frame 11 |
| 041206pl3H11 | C7orf24 | Homo sapiens cDNA FLJ11717 fis, clone |
| | | HEMBA1005241. |
| 160507pl3A4 | C7orf48 | hypothetical protein LOC84262 |
| 190607pl1A2 | C8orf44 | hypothetical protein LOC56260 |
| 050707pl3H2 | C8orf53 | hypothetical protein LOC84294 |
| 041206pl6D9 | C8orf59 | Homo sapiens cDNA FLJ20407 fis, clone |
| | | KAT01658. |
| 170407vpl3B12 | C9orf30 | hypothetical protein LOC91283 |
| 130207pl1E1 | C9orf40 | hypothetical protein LOC55071 |
| 200906pl5G7 | CA418524 | UI-H-EZ1-bbd-m-02-0-UI.s1 NCI_CGAP_Ch2 |
| | | Homo sapiens cDNA clone UI-H-EZ1-bbd-m-02-0- |
| | | UI 3′, mRNA sequence. |
| 050707pl2A3 | CA430002 | UI-H-FH1-bfp-h-24-0-UI.s1 NCI_CGAP_FH1 Homo |
| | | sapiens cDNA clone UI-H-FH1-bfp-h-24-0-UI 3′, |
| | | mRNA sequence. |
| 200906pl5F2 | CA444589 | UI-H-DT1-awl-m-08-0-UI.s1 NCI_CGAP_DT1 |
| | | Homo sapiens cDNA clone UI-H-DT1-awl-m-08-0- |
| | | UI 3′, mRNA sequence. |
| 010806pl4G11 | CA453297 | AGENCOURT_10577997 NIH_MGC_127 Homo |
| | | sapiens cDNA clone IMAGE: 6717046 5′, mRNA |
| | | sequence. |
| 200906pl3H12 | CA943566 | ir29h04.x1 HR85 islet Homo sapiens cDNA clone |
| | | IMAGE: 6546848 3′, mRNA sequence. |
| 041206pl7D1 | CACNA2D1 | calcium channel, voltage-dependent, alpha |
| 130207pl2A9 | CACYBP | calcyclin binding protein isoform 2 |
| 201107pl1H8 | CALCOCO2 | calcium binding and coiled-coil domain 2 |
| 200306d9pl1E8 | CALD1 | NAG22 protein. |
| 130207pl1A4 | CALM1 | calmodulin 1 |
| 310506pl3B1 | CALM2 | calmodulin 2 |
| 150506pl1E2 | CALM3 | calmodulin 2 |
| 200208pl2B12 | CAPRIN1 | membrane component chromosome 11 surface |
| | | marker |
| 170407vpl3B10 | CAPZA2 | Homo sapiens mRNA for capping protein (actin |
| | | filament) muscle Z-line, alpha 2 variant, clone: |
| | | HSI05568. |
| 041206pl7A11 | CASP8AP2 | CASP8 associated protein 2 |
| 010806pl1A3 | CAST | calpastatin isoform a |
| 170407pl1C2 | CAV1 | caveolin 1 |
| 150506pl2F10 | CB045860 | NISC_gf01a03.x1 NCI_CGAP_Kid12 Homo |
| | | sapiens cDNA clone IMAGE: 3252364 3′, mRNA |
| | | sequence. |
| 200906pl1D12 | CB046508 | NISC_gf05a01.x1 NCI_CGAP_Kid12 Homo |
| | | sapiens cDNA clone IMAGE: 3252744 3′, mRNA |
| | | sequence. |
| 310806pl2A3 | CB049395 | NISC_gj10f03.x1 NCI_CGAP_Pr28 Homo sapiens |
| | | cDNA clone IMAGE: 3271421 3′, mRNA sequence. |
| 050707pl2A6 | CB155900 | K-EST0214495 L17N670205n1 Homo sapiens |
| | | cDNA clone L17N670205n1-1-A03 5′, mRNA |
| | | sequence. |
| 200906pl5B5 | CB985912 | AGENCOURT_13640469 NIH_MGC_184 Homo |
| | | sapiens cDNA clone IMAGE: 30328716 5′, mRNA |
| | | sequence. |
| 041206pl1F3 | CBWD2 | COBW domain-containing protein 2 |
| 310806pl1C12 | CBX5 | chromobox homolog 5 (HP1 alpha homolog, |
| 050707pl2D9 | CCDC12 | coiled-coil domain containing 12 |
| 310506pl2C3 | CCDC23 | coiled-coil domain containing 23 |
| 010506pl1D3 | CCDC50 | Ymer protein long isoform |
| 010506pl2C10 | CCDC72 | coiled-coil domain containing 72 |
| 190607pl1G10 | CCDC74A | coiled-coil domain containing 74A |
| 041206pl3F4 | CCDC84 | coiled-coil domain containing 84 |
| 160507pl3F11 | CCT5 | chaperonin containing TCP1, subunit 5 (epsilon) |
| 290307pl1F1 | CCT6A | chaperonin containing TCP1, subunit 6A isoform |
| 200208pl2F4 | CCT7 | chaperonin containing TCP1, subunit 7 isoform a |
| 310506pl3H8 | CCT8 | CCT8 protein. |
| 31104p47c11 | CD164 | CD164 antigen, sialomucin |
| 041206pl3D11 | CD44 | CD44 antigen isoform 1 precursor |
| 160507pl3D3 | CD63 | CD63 antigen isoform A |
| 041206pl1C8 | CD641745 | AGENCOURT_14537497 NIH_MGC_191 Homo |
| | | sapiens cDNA clone IMAGE: 30416477 5′, mRNA |
| | | sequence. |
| 050707pl1C3 | CD692919 | EST9442 human nasopharynx Homo sapiens |
| | | cDNA, mRNA sequence. |
| 311007pl3H5 | CD9 | CD9 antigen |
| 010806pl3D4 | CDADC1 | cytidine and dCMP deaminase domain containing 1 |
| 311007pl3D9 | CDC37 | Synthetic construct Homo sapiens mRNA for |
| | | hypothetical protein (CDC37 gene), clone |
| | | IMAGE: 3505011.1E3. |
| 041206pl6F10 | CDK3 | cyclin-dependent kinase 3 |
| 050707pl3C12 | CDKN3 | cyclin-dependent kinase inhibitor 3 |
| 310506pl3A8 | CECR4 | Homo sapiens Cat eye syndrome critical region |
| | | candidate gene number 4 (CECR4) mRNA, partial |
| | | cds. |
| 160507pl2A12 | CENTB1 | centaurin beta1 |
| 041206pl5B7 | CFL2 | cofilin 2 |
| 160507pl1D6 | CFLAR | CASP8 and FADD-like apoptosis regulator |
| 170604p17c4 | CHCHD2 | coiled-coil-helix-coiled-coil-helix domain |
| 150506pl2F11 | CHCHD6 | coiled-coil-helix-coiled-coil-helix domain |
| 041206pl6B6 | CHCHD8 | coiled-coil-helix-coiled-coil-helix domain |
| 310506pl2E5 | CHORDC1 | cysteine and histidine-rich domain |
| 041206pl1A9 | CHURC1 | churchill domain containing 1 |
| 311007pl3D3 | CICK0721Q.1 | hypothetical protein LOC729727 |
| 050707pl3A12 | CIP29 | Homo sapiens HSPC316 mRNA, partial cds. |
| 280305p1f12d10 | CIRBP | cold inducible RNA binding protein |
| 201107pl3D4 | CIRH1A | cirhin |
| 010806pl2F10 | CK126027 | AGENCOURT_16510969 NIH_MGC_239 Homo |
| | | sapiens cDNA clone IMAGE: 30710070 5′, mRNA |
| | | sequence. |
| 010806pl4A1 | CKS2 | CDC28 protein kinase 2 |
| 200306d9pl1D7 | CLCN3 | chloride channel 3 isoform e |
| 050707pl2H5 | CLEC2D | osteoclast inhibitory lectin isoform 1 |
| 10704p110c1 | CLIC1 | chloride intracellular channel 1 |
| 311007pl3A11 | CLIC4 | chloride intracellular channel 4 |
| 010806pl1B6 | CLINT1 | epsin 4 |
| 170407vpl3B2 | CLPTM1 | cleft lip and palate associated transmembrane |
| 200208pl2F7 | CLTC | clathrin heavy chain 1 |
| 310506pl3D11 | CMTM3 | chemokine-like factor superfamily 3 |
| 041206pl7A8 | CN267986 | 17000531863184 GRN_EB Homo sapiens cDNA |
| | | 5′, mRNA sequence. |
| 200906pl5G6 | CN277269 | 17000600176551 GRN_PREHEP Homo sapiens |
| | | cDNA 5′, mRNA sequence. |
| 290307pl1D5 | CN280387 | 17000455082974 GRN_ES Homo sapiens cDNA |
| | | 5′, mRNA sequence. |
| 041206pl2B2 | CN290177 | 17000600005140 GRN_PRENEU Homo sapiens |
| | | cDNA 5′, mRNA sequence. |
| 170407pl1E12 | CN398253 | 17000424721764 GRN_EB Homo sapiens cDNA |
| | | 5′, mRNA sequence. |
| 010806pl3C12 | CNN3 | calponin 3 |
| 010806pl1F8 | COPS6 | COP9 signalosome subunit 6 |
| 050707pl1C8 | COPZ1 | coatomer protein complex, subunit zeta 1 |
| 041206pl3H8 | COTL1 | coactosin-like 1 |
| 311007pl2A1 | COX17 | COX17 homolog, cytochrome c oxidase assembly |
| 160507pl1D1 | COX4NB | neighbor of COX4 |
| 310506pl2A5 | COX7C | cytochrome c oxidase subunit VIIc precursor |
| 170407vpl3G10 | COX8A | cytochrome c oxidase subunit 8A |
| 041206pl6F11 | CR593740 | Homo sapiens cDNA clone IMAGE: 4823412. |
| 200906pl1H3 | CR599716 | Homo sapiens Shwachman-Bodian-Diamond |
| | | syndrome pseudogene, mRNA (cDNA clone |
| | | IMAGE: 4329436). |
| 050707pl3B3 | CR604262 | full-length cDNA clone CS0DC003YA14 of |
| | | Neuroblastoma Cot 25-normalized of Homo |
| | | sapiens (human). |
| 130207pl2B12 | CR604408 | Homo sapiens, clone IMAGE: 5190399, mRNA. |
| 200906pl2B3 | CR623475 | Homo sapiens cDNA: FLJ21942 fis, clone |
| | | HEP04527. |
| 200306f7pl1A9 | CR624523 | Homo sapiens hypothetical gene , mRNA |
| 041206pl6H12 | CR625980 | full-length cDNA clone CS0DC026YN07 of |
| | | Neuroblastoma Cot 25-normalized of Homo |
| | | sapiens (human). |
| 010506pl2A12 | CR626360 | full-length cDNA clone CS0DM014YM20 of Fetal |
| | | liver of Homo sapiens (human). |
| 160507pl1A9 | CR627148 | Homo sapiens, clone IMAGE: 5213378, mRNA. |
| 160507pl1D7 | CR737784 | CR737784 Homo sapiens library (Ebert L) Homo |
| | | sapiens cDNA clone IMAGp998C154208; |
| | | IMAGE: 1658054 5′, mRNA sequence. |
| 190607pl1B9 | CR994463 | CR994463 RZPD no. 9016 Homo sapiens cDNA |
| | | clone RZPDp9016A109 5′, mRNA sequence. |
| 170407pl3E4 | CRKL | v-crk sarcoma virus CT10 oncogene homolog |
| 310505p4f1c4 | CSDA | cold shock domain protein A |
| 041206pl3B4 | CSDE1 | upstream of NRAS isoform 1 |
| 160507pl2F7 | CSNK1A1 | casein kinase 1, alpha 1 isoform 2 |
| 200208pl2D1 | CXorf26 | Homo sapiens HSPC245 mRNA, complete cds. |
| 010806pl2E2 | DA336829 | DA336829 BRHIP3 Homo sapiens cDNA clone |
| | | BRHIP3037522 5′, mRNA sequence. |
| 041206pl6A7 | DA438551 | DA438551 CTONG2 Homo sapiens cDNA clone |
| | | CTONG2006372 5′, mRNA sequence. |
| 150506pl2A8 | DA691808 | DA691808 NT2NE2 Homo sapiens cDNA clone |
| | | NT2NE2011571 5′, mRNA sequence. |
| 200906pl2F8 | DA697821 | DA697821 NT2NE2 Homo sapiens cDNA clone |
| | | NT2NE2019092 5′, mRNA sequence. |
| 041206pl3H1g | DA963983 | DA963983 STOMA2 Homo sapiens cDNA clone |
| | | STOMA2001983 5′, mRNA sequence. |
| 010806pl2F11 | DAP | death-associated protein |
| 150506pl1B12 | DAZAP2 | DAZ associated protein 2 |
| 200306f7pl1C3 | DB040854 | DB040854 TESTI2 Homo sapiens cDNA clone |
| | | TESTI2027763 5′, mRNA sequence. |
| 311007pl2C1 | DB049861 | DB049861 TESTI2 Homo sapiens cDNA clone |
| | | TESTI2039270 5′, mRNA sequence. |
| 310806pl2E8 | DB054822 | DB054822 TESTI2 Homo sapiens cDNA clone |
| | | TESTI2045843 5′, mRNA sequence. |
| 200906pl4C12 | DB095008 | DB095008 TESTI4 Homo sapiens cDNA clone |
| | | TESTI4045539 5′, mRNA sequence. |
| 201107pl3E12 | DB136282 | DB136282 THYMU3 Homo sapiens cDNA clone |
| | | THYMU3007538 5′, mRNA sequence. |
| 160507pl1B10 | DB331110 | DB331110 SKMUS2 Homo sapiens cDNA clone |
| | | SKMUS2008761 3′, mRNA sequence. |
| 200906pl1G4 | DB337826 | DB337826 TESTI2 Homo sapiens cDNA clone |
| | | TESTI2027763 3′, mRNA sequence. |
| 310506pl3F2 | DB339365 | hEST |
| 050707pl2A9 | DB344099 | DB344099 THYMU2 Homo sapiens cDNA clone |
| | | THYMU2032116 3′, mRNA sequence. |
| 041206pl7C8 | DB478885 | DB478885 RIKEN full-length enriched human |
| | | cDNA library, hippocampus Homo sapiens cDNA |
| | | clone H023080L11 5′, mRNA sequence. |
| 190607pl1F10 | DB499813 | DB499813 RIKEN full-length enriched human |
| | | cDNA library, hypothalamus Homo sapiens cDNA |
| | | clone H033074L02 5′, mRNA sequence. |
| 041206pl2A6 | DB504537 | DB504537 RIKEN full-length enriched human |
| | | cDNA library, hypothalamus Homo sapiens cDNA |
| | | clone H033091O18 5′, mRNA sequence. |
| 160507pl3E2 | DB514539 | DB514539 RIKEN full-length enriched human |
| | | cDNA library, testis Homo sapiens cDNA clone |
| | | H013041M08 3′, mRNA sequence. |
| 130207pl1H2 | DB522524 | DB522524 RIKEN full-length enriched human |
| | | cDNA library, testis Homo sapiens cDNA clone |
| | | H013076C14 3′, mRNA sequence. |
| 200906pl1D3 | DB566909 | DB566909 RIKEN full-length enriched human |
| | | cDNA library, hypothalamus Homo sapiens cDNA |
| | | clone H033059N21 3′, mRNA sequence. |
| 310806pl1H4 | DB571782 | DB571782 RIKEN full-length enriched human |
| | | cDNA library, hypothalamus Homo sapiens cDNA |
| | | clone H033077H09 3′, mRNA sequence. |
| 310505p4f1c5 | DBN1 | drebrin 1 isoform a |
| 200906pl1A9 | DC347972 | DC347972 CTONG3 Homo sapiens cDNA clone |
| | | CTONG3005404 5′, mRNA sequence. |
| 190607pl1F8 | DCBLD2 | discoidin, CUB and LCCL domain containing 2 |
| 010806pl3A8 | DCC | deleted in colorectal carcinoma |
| 200306f7pl1G12 | DDT | D-dopachrome tautomerase |
| 311007pl1G6 | DDX10 | DEAD (Asp-Glu-Ala-Asp) box polypeptide 10 |
| 010806pl2C5 | DDX18 | DEAD (Asp-Glu-Ala-Asp) box polypeptide 18 |
| 311007pl1A12 | DDX43 | DEAD (Asp-Glu-Ala-Asp) box polypeptide 43 |
| 310505p7f1b3 | DDX46 | DEAD (Asp-Glu-Ala-Asp) box polypeptide 46 |
| 090505p3f12d6 | DDX5 | DEAD (Asp-Glu-Ala-Asp) box polypeptide 5 |
| 150506pl2F8 | DEK | DEK oncogene |
| 210206pl1C6 | DHX15 | DEAN (Asp-Glu-Ala-His) box polypeptide 15 |
| 200306f7pl1B10 | DHX16 | DEAN (Asp-Glu-Ala-His) box polypeptide 16 |
| 160507pl1B11 | DKFZp434M1123 | Homo sapiens NY-REN-50 antigen mRNA, partial |
| | | cds. |
| 310506pl1C9 | DKFZp451B1418 | Homo sapiens HSPC308 mRNA, partial cds. |
| 010806pl1H2 | DKFZp686B0790 | Homo sapiens clone alpha1 mRNA sequence. |
| 010806pl1G2 | DKFZp686N1150 | Homo sapiens cDNA FLJ37790 fis, clone |
| | | BRHIP3000111. |
| 160507pl1B4 | DKKL1 | dickkopf-like 1 (soggy) precursor |
| 310506pl2C1 | DLGAP1 | discs large homolog-associated protein 1 isoform |
| 041206pl6D1 | DLGAP4 | disks large-associated protein 4 isoform a |
| 170407pl3F3 | DMTF1 | cyclin D binding myb-like transcription factor |
| 041206pl7A2 | DNAJA1 | DnaJ (Hsp40) homolog, subfamily A, member 1 |
| 170604pl7c1 | DNAJC7 | DnaJ (Hsp40) homolog, subfamily C, member 7 |
| 050707pl1D3 | DNAPTP6 | hypothetical protein LOC26010 |
| 171104P31B6 | DNMT1 | DNA (cytosine-5-)-methyltransferase 1 |
| 311007pl2B12 | DPH1 | diptheria toxin resistance protein required for |
| 041206pl6F8 | DQ343132 | Homo sapiens urothelial cancer associated 1 |
| | | (UCA1) mRNA, complete sequence. |
| 170407pl3D12 | DQ578159 | full-length cDNA clone CS0DA009YE19 of |
| | | Neuroblastoma of Homo sapiens (human). |
| 130207pl1E12 | DSTN | destrin isoform a |
| 200906pl5F4 | DY654337 | ucsc5_1.5.1.L1.1.A06.R.1 NIH_MGC_331 Homo |
| | | sapiens cDNA clone ucsc5_1.5.1.L1.1.A06, mRNA |
| | | sequence. |
| 041206pl5E4 | DYNC1H1 | dynein, cytoplasmic, heavy polypeptide 1 |
| 311007pl3F5 | DYNLRB1 | Roadblock-1 |
| 041206pl6E1 | EAPP | E2F-associated phosphoprotein |
| 200208pl2B1 | ece-1d | Homo sapiens mRNA for endothelin-converting |
| | | enzyme-1c, complete cds. |
| 010506pl2D4 | ECM29 | KIAA0368 protein |
| 201107pl2D5 | EEA1 | early endosome antigen 1, 162 kD |
| 311007pl1G11 | EED | embryonic ectoderm development isoform a |
| 050707pl2B5 | EEF1A1 | eukaryotic translation elongation factor 1 alpha |
| 041206pl1A2 | EEF1E1 | eukaryotic translation elongation factor 1 |
| 041206pl3D5 | EEF1G | eukaryotic translation elongation factor 1 |
| 190607pl1E7 | EEF2 | eukaryotic translation elongation factor 2 |
| 190607pl1F3 | EF565105 | Homo sapiens chromosome 16 isolate HA_003251 |
| | | mRNA sequence. |
| 041206pl3B8 | EFHC1 | EF-hand domain (C-terminal) containing 1 |
| 310505p4f1d1 | EIF1AX | X-linked eukaryotic translation initiation |
| 201107pl4B9 | EIF2S2 | eukaryotic translation initiation factor 2 beta |
| 311007pl2C9 | EIF2S3 | eukaryotic translation initiation factor 2, |
| 310806pl1H5 | EIF3S10 | eukaryotic translation initiation factor 3, |
| 041206pl1C1 | EIF3S12 | eukaryotic translation initiation factor 3, |
| 210206pl1C3 | EIF4A1 | eukaryotic translation initiation factor 4A |
| 310506pl4B9 | EIF4E2 | eukaryotic translation initiation factor 4E |
| 180504p21e4 | EIF4EBP1 | eukaryotic translation initiation factor 4E |
| 050707pl1G11 | EIF4G3 | eukaryotic translation initiation factor 4 |
| 150506pl1C2 | EIF4H | eukaryotic translation initiation factor 4H |
| 150506pl1D4 | EIF5B | eukaryotic translation initiation factor 5B |
| 200906pl5E10 | EMP3 | epithelial membrane protein 3 |
| 150506pl2F1 | ENO1 | enolase 1 |
| 160507pl1A11 | ENSA | endosulfine alpha isoform 5 |
| 050707pl3B8 | ENY2 | enhancer of yellow 2 homolog |
| 010806pl4E2 | EPRS | glutamyl-prolyl tRNA synthetase |
| 280705p1f13C12 | ERCC1 | excision repair cross-complementing 1 isoform 1 |
| 170407pl1A1 | ERH | enhancer of rudimentary homolog |
| 050707pl1G7 | ETFB | electron-transfer-flavoprotein, beta polypeptide |
| 200906pl1B6 | FABP5 | fatty acid binding protein 5 |
| 130207pl1G3 | FAM128A | Homo sapiens family with sequence similarity 128, |
| | | member A, mRNA (cDNA clone MGC: 8772 |
| | | IMAGE: 3862861), complete cds. |
| 200306d9pl1B9 | FAM128B | hypothetical protein LOC80097 |
| 201107pl1C10 | FAM18B2 | hypothetical protein LOC201158 |
| 160507pl3E12 | FAM36A | family with sequence similarity 36, member A |
| 201107pl2H12 | FAM44A | hypothetical protein LOC259282 |
| 201107pl4D5 | FAM82B | hypothetical protein LOC51115 |
| 041206pl1A11 | FAM86A | hypothetical protein LOC196483 isoform 1 |
| 200906pl1D8 | FAU | ubiquitin-like protein fubi and ribosomal |
| 27073i1 | FBL | fibrillarin |
| 310506pl2B1 | FBXO9 | F-box only protein 9 isoform 3 |
| 201107pl1E8 | FC170787 | 1106908754941 BABEVPN-C-01-1-7KB Papio |
| | | anubis cDNA clone 1061041899735 5′ similar to H. sapiens |
| | | UQCC (UniProtKB/Swiss-Prot: Q9NVA1), |
| | | mRNA sequence. |
| 210206pl1D3 | FER1L3 | myoferlin isoform a |
| 190607pl1A3 | FEZ2 | zygin 2 isoform 2 |
| 190607pl1F1 | FHL3 | four and a half LIM domains 3 |
| 310506pl1E5 | FIGN | fidgetin |
| 310506pl2E4 | FLAD1 | flavin adenine dinucleotide synthetase isoform |
| 010506pl2D7 | FLJ10154 | hypothetical protein LOC55082 |
| 311007pl2G6 | FLJ10292 | mago-nashi homolog 2 |
| 041206pl5H11 | FLJ10986 | Homo sapiens cDNA FLJ10986 fis, clone |
| | | PLACE1001869, weakly similar to L- |
| | | RIBULOKINASE (EC 2.7.1.16). |
| 010506pl1A8 | FLJ20105 | hypothetical protein LOC54821 isoform a |
| 010806pl1D11 | FLJ20674 | hypothetical protein LOC54621 |
| 050707pl3A4 | FLJ21908 | hypothetical protein LOC79657 |
| 041206pl6G11 | FLJ31951 | hypothetical protein LOC153830 |
| 050707pl1D1 | FLJ32065 | Homo sapiens cDNA FLJ32065 fis, clone |
| | | OCBBF1000086. |
| 050707pl1E3 | FLJ35776 | hypothetical protein LOC649446 |
| 010704p19b8 | FLNB | filamin B, beta (actin binding protein 278) |
| 170407vpl2C6 | FNBP1 | formin binding protein 1 |
| 130207pl1F5 | FOSL1 | FOS-like antigen 1 |
| 010506pl1C10 | FSCN1 | fascin 1 |
| 010806pl4E4 | FUBP1 | far upstream element-binding protein |
| 180504p1ab2 | FUS | fusion (involved in t(12; 16) in malignant |
| 200906pl5F9 | FXR1 | fragile X mental retardation-related protein 1 |
| 041206pl5C4 | FXYD5 | FXYD domain-containing ion transport regulator |
| 310806pl1C6 | FYTTD1 | forty-two-three domain containing 1 isoform 1 |
| 041206pl4H8 | G36884 | SHGC-56440 Human Homo sapiens STS cDNA, |
| | | sequence tagged site. |
| 010806pl2B6 | GABARAP | GABA(A) receptor-associated protein |
| 160507pl2B2 | GAGE2 | G antigen 2 |
| 130207pl2D12 | GAGE4 | G antigen 4 |
| 170407vpl2D8 | GALNT2 | polypeptide N-acetylgalactosaminyltransferase 2 |
| 311007pl1E7 | GAP43 | growth associated protein 43 |
| 010806pl2G3 | GAPDH | glyceraldehyde-3-phosphate dehydrogenase |
| 130207pl1C6 | GARS | glycyl-tRNA synthetase |
| 150506pl1A4 | GCHFR | GTP cyclohydrolase I feedback regulatory |
| 311007pl1F11 | GCNT2 | glucosaminyl (N-acetyl) transferase 2, |
| 160507pl3H2 | GKN1 | 18 kDa antrum mucosa protein |
| 201107pl2G2 | GLO1 | glyoxalase I |
| 311007pl1C9 | GLRX | glutaredoxin (thioltransferase) |
| 150506pl1D2 | GNB2L1 | guanine nucleotide binding protein (G protein), |
| 010806pl2F9 | GNG11 | guanine nucleotide binding protein gamma 11 |
| 201107pl1B5 | GNG7 | guanine nucleotide binding protein (G protein), |
| 200906pl5F3 | GPR113 | G-protein coupled receptor 113 |
| 010806pl2E7 | GRPEL1 | GrpE-like 1, mitochondrial |
| 201107pl1B7 | GRSF1 | G-rich RNA sequence binding factor 1 |
| 280305p5f2E4 | GSPT1 | G1 to S phase transition 1 |
| 280305p1f12D4 | GTF2F2 | general transcription factor IIF, polypeptide 2 |
| 130207pl2C3 | H2AFV | H2A histone family, member V isoform 2 |
| 311007pl1C10 | HABP4 | hyaluronan binding protein 4 |
| 050707pl3F9 | HAT1 | histone acetyltransferase 1 isoform a |
| 041206pl5H2 | HCST | hematopoietic cell signal transducer isoform 1 |
| 041206pl1E4 | HDAC2 | histone deacetylase 2 |
| 200208pl2C5 | HGD | homogentisate 1,2-dioxygenase |
| 310506pl2B8 | HHLA3 | HERV-H LTR-associating 3 isoform 2 |
| 200906pl2C2 | HIST1H2BH | H2B histone family, member J |
| 010806pl2B2 | HMG2L1 | high-mobility group protein 2-like 1 isoform b |
| 031104p47c9 | HMGA1 | high mobility group AT-hook 1 isoform a |
| 27073c11 | HMGA2 | high mobility group AT-hook 2 isoform a |
| 150506pl1A11 | HMGN2 | high-mobility group nucleosomal binding domain |
| 311007pl3E9 | HMGN3 | high mobility group nucleosomal binding domain 3 |
| 290307pl1E4 | HMMR | hyaluronan-mediated motility receptor isoform a |
| 310506pl1F8 | HN1 | hematological and neurological expressed 1 |
| 190607pl1E2 | HNRPA1 | heterogeneous nuclear ribonucleoprotein A1 |
| 201107pl2F6 | HNRPA2B1 | heterogeneous nuclear ribonucleoprotein A2/B1 |
| 210206pl1E2 | HNRPA3 | heterogeneous nuclear ribonucleoprotein A3 |
| 050707pl1G6 | HNRPAB | heterogeneous nuclear ribonucleoprotein AB |
| 310506pl3H12 | HNRPC | heterogeneous nuclear ribonucleoprotein C |
| 210206pl1D2 | HNRPD | heterogeneous nuclear ribonucleoprotein D |
| 210206pl1G8 | HNRPM | heterogeneous nuclear ribonucleoprotein M |
| 311007pl3E5 | HSP90AA1 | heat shock protein 90 kDa alpha (cytosolic), |
| 050707pl3D4 | HSP90AB1 | heat shock 90 kDa protein 1, beta |
| 310506pl2C10 | HSPB1 | heat shock 27 kDa protein 1 |
| 310506pl1B9 | HSPCA | heat shock protein 90 kDa alpha (cytosolic), |
| 201107pl2D3 | HSPH1 | heat shock 105 kD |
| 160507pl3G7 | HYPA | Hypothetical protein (Fragment). |
| 311007pl1A1 | HYPK | Huntingtin interacting protein K |
| 200906pl3E9 | IFNGR2 | interferon-gamma receptor beta chain precursor |
| 311007pl3B11 | IFT20 | intraflagellar transport protein IFT20 |
| 310506pl3G10 | IKIP | IKK interacting protein isoform 2 |
| 010506pl2A4 | IL3RA | interleukin 3 receptor, alpha precursor |
| 010806pl2F6 | ILF2 | interleukin enhancer binding factor 2 |
| 311007pl1C11 | INPP4B | inositol polyphosphate-4-phosphatase, type II, |
| 130207pl1B8 | IQCK | IQ motif containing K |
| 200208pl2C11 | IRAK2 | interleukin-1 receptor-associated kinase 2 |
| 311007pl1B3 | ISOC1 | isochorismatase domain containing 1 |
| 041206pl6B11 | ITIH5 | inter-alpha trypsin inhibitor heavy chain |
| 041206pl2H6 | JAGN1 | jagunal homolog 1 |
| 200906pl3G10 | KATNA1 | katanin p60 subunit A 1 |
| 310806pl1D6 | KBTBD2 | kelch repeat and BTB (POZ) domain containing 2 |
| 160507pl2E5 | KIAA0355 | hypothetical protein LOC9710 |
| 210206pl1G5 | KIAA0802 | hypothetical protein LOC23255 |
| 200906pl2A2 | KIAA1064 | Homo sapiens mRNA for KIAA1064 protein, partial |
| | | cds. |
| 010806pl2D1 | KIAA1186 | Homo sapiens mRNA for KIAA1186 protein, partial |
| | | cds. |
| 200208pl2E11 | KIAA1303 | raptor |
| 041206pl1H2 | KIAA1430 | KIAA1430 protein (Fragment). |
| 130207pl2C1 | KIAA1783 | Homo sapiens mRNA for KIAA1783 protein, partial |
| | | cds. |
| 311007pl1G2 | KIAA1949 | Protein KIAA1949. |
| 010806pl4E11 | KLHDC8A | kelch domain containing 8A |
| 170407pl1E5 | KLHL31 | kelch repeat and BTB (POZ) domain containing 1 |
| 201107pl2H7 | KPNA1 | karyopherin alpha 1 |
| 200906pl2H3 | KRT18 | keratin 18 |
| 190607pl1C12 | KRT8 | keratin 8 |
| 010506pl1E9 | Kua-UEV | ubiquitin-conjugating enzyme E2 Kua-UEV isoform |
| 170407pl1D4 | LAP3 | leucine aminopeptidase 3 |
| 010806pl2C12 | LARP1 | la related protein isoform 2 |
| 290307pl1E10 | LARP4 | c-Mpl binding protein isoform a |
| 10704p19b7 | LASP1 | LIM and SH3 protein 1 |
| 200208pl2G6 | LDHA | lactate dehydrogenase A |
| 200306f7pl1E6 | LETM2 | leucine zipper-EF-hand containing transmembrane |
| 010306d9pl1C2 | LGALS1 | beta-galactoside-binding lectin precursor |
| 010806pl4F6 | LGALS3 | galectin 3 |
| 311007pl2F8 | LHB | luteinizing hormone beta subunit precursor |
| 170407vpl3C6 | LIMA1 | epithelial protein lost in neoplasm beta |
| 041206pl6E7 | LIN7B | lin-7 homolog B |
| 27073d13 | LMNA | lamin A/C isoform 1 precursor |
| 310131d13 | LMNB1 | lamin B1 |
| 010506pl2C12 | LOC130074 | hypothetical protein LOC130074 |
| 310806pl3B11 | LOC134145 | hypothetical protein LOC134145 |
| 311007pl1G12 | LOC283551 | hypothetical protein LOC283551 |
| 311007pl2G4 | LOC284184 | Homo sapiens full length insert cDNA clone |
| | | ZD54C08. |
| 190607pl1E6 | LOC286016 | Homo sapiens cDNA FLJ37575 fis, clone |
| | | BRCOC2003125, moderately similar to |
| | | TRIOSEPHOSPHATE ISOMERASE (EC 5.3.1.1). |
| 200906pl2G9 | LOC389072 | hypothetical protein LOC389072 |
| 050707pl2C4 | LOC441161 | hypothetical LOC441161 |
| 310506pl1D7 | LOC541471 | Homo sapiens hypothetical LOC541471, mRNA |
| | | (cDNA clone MGC: 17532 IMAGE: 3459303), |
| | | complete cds. |
| 050707pl3H6 | LOC728776 | hypothetical protein LOC728776 |
| 201107pl2D11 | LOC729416 | hypothetical protein LOC729416 |
| 311007pl2D11 | LOC751071 | hypothetical protein LOC751071 |
| 200306d9pl1B4 | LONRF3 | LON peptidase N-terminal domain and ring finger |
| 311007pl3C8 | LOXL2 | lysyl oxidase-like 2 precursor |
| 170407pl1B6 | LPIN2 | lipin 2 |
| 150506pl1H3 | LRRC50 | leucine rich repeat containing 50 |
| 311007pl2C6 | LRRC59 | leucine rich repeat containing 59 |
| 010806pl1G1 | LRRFIP1 | LRR FLI-I interacting protein 1 (Fragment). |
| 050707pl1D10 | LSM3 | Lsm3 protein |
| 041206pl2B1 | LUC7L2 | LUC7-like 2 |
| 041206pl6H8 | LYAR | hypothetical protein FLJ20425 |
| 200306f7pl1A10 | MAP2K2 | mitogen-activated protein kinase kinase 2 |
| 280305p1f12C11 | MAP4 | microtubule-associated protein 4 isoform 1 |
| 200906pl4A2 | MAPBPIP | mitogen-activated protein-binding |
| 010604p16b2 | MAPK1 | mitogen-activated protein kinase 1 |
| 180504p2ab3 | MAPRE2 | microtubule-associated protein, RP/EB family, |
| 130207pl1B1 | MBNL2 | muscleblind-like 2 isoform 1 |
| 200906pl1G2 | MCEE | methylmalonyl-CoA epimerase |
| 170407vpl2C2 | MDH1 | cytosolic malate dehydrogenase |
| 160507pl2H9 | ME3 | malic enzyme 3, NADP(+)-dependent, |
| 150506pl2C12 | MEGF6 | EGF-like-domain, multiple 3 |
| 010506pl2E1 | METAP2 | methionyl aminopeptidase 2 |
| 170407vpl2B2 | MGC11257 | hypothetical protein LOC84310 |
| 160507pl3C9 | MGC16824 | hypothetical protein LOC57020 |
| 041206pl2F1 | MGC59937 | hypothetical protein LOC375791 |
| 150506pl1D10 | mimitin | Homo sapiens mimitin mRNA for Myc-induced |
| | | mitochondria protein, complete cds. |
| 170407vpl2D2 | MKI67IP | MKI67 (FHA domain) interacting nucleolar |
| 010506pl1F4 | MKRN2 | makorin, ring finger protein, 2 |
| 311007pl1D5 | MLLT4 | myeloid/lymphoid or mixed-lineage leukemia |
| 041206pl4E11 | MMAA | Homo sapiens cDNA FLJ44706 fis, clone |
| | | BRACE3017253, weakly similar to LAO/AO |
| | | transport system kinase (EC 2.7.—.—). |
| 050707pl2H3 | MRCL3 | myosin regulatory light chain MRCL3 |
| 050707pl1D12 | MRLC2 | myosin regulatory light chain MRCL2 |
| 310806pl2D10 | MRPL37 | mitochondrial ribosomal protein L37 |
| 311007pl1G9 | MRPS18B | mitochondrial ribosomal protein S18B |
| 130207pl1G10 | MRTO4 | ribosomal protein P0-like protein |
| 310806pl1D11 | MSH6 | mutS homolog 6 |
| 27073k9 | MSN | moesin |
| 150506pl1D5 | MSRA | methionine sulfoxide reductase A |
| 010704p110d1 | MT2A | metallothionein 2A |
| 190607pl1A5 | MTDH | LYRIC/3D3 |
| 311007pl1H5 | MTPN | myotrophin |
| 041206pl3C7ag | MTX1 | metaxin 1 isoform 1 |
| 041206pl2H7 | MYEOV | myeloma overexpressed |
| 010506pl1B12 | MYH9 | myosin, heavy polypeptide 9, non-muscle |
| 310506pl1H5 | MYLE | dexamethasone-induced protein |
| 200208pl2C3 | MYO1D | myosin ID |
| 200208pl2A2 | MYO1E | myosin IE |
| 200906pl3F8 | N39715 | yx92d05.r1 Soares melanocyte 2NbHM Homo |
| | | sapiens cDNA clone IMAGE: 269193 5′ similar to |
| | | contains element TAR1 repetitive element;, mRNA |
| | | sequence. |
| 201107pl2A3 | N68399 | za13b04.s1 Soares fetal liver spleen 1NFLS Homo |
| | | sapiens cDNA clone IMAGE: 292399 3′ similar to |
| | | SW: OLF3_MOUSE P23275 OLFACTORY |
| | | RECEPTOR OR3. [1];, mRNA sequence. |
| 200306f7pl1C7 | NACA | nascent-polypeptide-associated complex alpha |
| 010806pl1G12 | NANOS3 | NANOS3 protein. |
| 010704p110d2 | NASP | nuclear autoantigenic sperm protein isoform 2 |
| 210206pl1C12 | NAT13 | Mak3 homolog |
| 010806pl4F4 | NBEAL1 | Neurobeachin-like 1 (Amyotrophic lateral sclerosis |
| | | 2 chromosomal region candidate gene 17 protein). |
| 050707pl2G10 | NCBP2 | nuclear cap binding protein subunit 2, 20 kDa |
| 160507pl3B1 | NCL | nucleolin |
| 150506pl1F11 | NDUFA12L | Myc-induced mitochondria protein |
| 010806pl1A10 | NDUFA7 | NADH dehydrogenase (ubiquinone) 1 alpha |
| 041206pl5H6 | NDUFB1 | NADH dehydrogenase (ubiquinone) 1 beta |
| 050707pl1B10 | NDUFB11 | NADH dehydrogenase (ubiquinone) 1 beta |
| 190607pl1D5 | NDUFB7 | NADH dehydrogenase (ubiquinone) 1 beta |
| 200306d9pl1C8 | NDUFB8 | NADH dehydrogenase (ubiquinone) 1 beta |
| 170407vpl2B5 | NDUFC1 | NADH dehydrogenase (ubiquinone) 1, subcomplex |
| 041206pl6F9 | NEDD4L | neural precursor cell expressed, developmentally |
| 010806pl2G6 | NEXN | Nexilin. |
| 010806pl1D1 | NFE2L2 | nuclear factor (erythroid-derived 2)-like 2 |
| 200906pl5B12 | NGRN | mesenchymal stem cell protein DSC92 isoform 2 |
| 010604p16c10b | NHP2L1 | NHP2 non-histone chromosome protein 2-like 1 |
| 200906pl5C2 | NM_001039753 | CDNA FLJ16635 fis, clone TESTI4025268, weakly |
| | | similar to 77 kDa echinoderm microtubule- |
| | | associated protein. |
| 050707pl3G6 | NM_001089591 | Homo sapiens hCG25371 (LOC440567), mRNA. |
| 200906pl2H4 | NM_001093732 | Homo sapiens hCG2033311 (LOC644928), |
| | | mRNA. |
| 050707pl1C11 | NM_001097611 | Homo sapiens kinocilin (KNCN), mRNA. |
| 311007pl2A8 | NM_015681 | Homo sapiens B9 protein domain 1 (B9D1), |
| | | mRNA. |
| 200306f7pl1F8 | NME1-NME2 | NME1-NME2 protein |
| 311007pl1H6 | NME4 | nucleoside-diphosphate kinase 4 |
| 200306f7pl1A7 | NMT1 | N-myristoyltransferase 1 |
| 180504p2ab6 | NOL1 | nucleolar protein 1, 120 kDa |
| 200906pl3H11 | NOL7 | nucleolar protein 7, 27 kDa |
| 200906pl3C7 | NPAT | nuclear protein, ataxia-telangiectasia locus |
| 160507pl1A3 | NPEPPS | aminopeptidase puromycin sensitive |
| 200906pl2B11 | NPHP3 | nephronophthisis 3 |
| 010506pl1A7 | NPM1 | nucleophosmin 1 isoform 1 |
| 010506pl2A1 | NQO1 | NAD(P)H menadione oxidoreductase 1, |
| 311007pl1B12 | NSMCE4A | non-SMC element 4 homolog A |
| 310506pl1E9 | NT_006576.400 | Predicted Gene |
| 310506pl1E8 | NT_007592.828 | Predicted Gene |
| 310506pl1A6 | NT_030059.345 | genescan prediction |
| 200906pl1C11 | nt_032977.1313 | Predicted Gene |
| 200906pl2E7 | NT_033899.591 | Predicted Gene |
| 170407pl3F4 | NTAN1 | N-terminal Asn amidase |
| 200906pl2F1 | NUCKS1 | nuclear ubiquitous casein kinase and |
| 201107pl3A10 | NUDC | nuclear distribution gene C homolog |
| 150506pl1F7 | NUDCD1 | NudC domain containing 1 |
| 160507pl1D4 | NUDCD2 | NudC domain containing 2 |
| 170407vpl2E11 | NUDT3 | nudix-type motif 3 |
| 050707pl1E10 | NUP153 | nucleoporin 153 kDa |
| 310506pl3H5 | NUP93 | nucleoporin 93 kDa |
| 201107pl3G7 | OBTP | Homo sapiens over-expressed breast tumor |
| | | protein (OBTP) mRNA, complete cds. |
| 170407pl1G1 | OSBPL8 | oxysterol-binding protein-like protein 8 isoform |
| 170407pl3E2 | OSBPL9 | oxysterol-binding protein-like protein 9 isoform |
| 041206pl2A7 | OTUB1 | otubain 1 |
| 180504p12d4 | PA2G4 | ErbB3-binding protein 1 |
| 200906pl1C6 | PABPN1 | poly(A) binding protein, nuclear 1 |
| 050707pl3F11 | PAGE1 | P antigen family, member 1 |
| 200906pl4E4 | PAK2 | p21-activated kinase 2 |
| 200208pl2G7 | PARP4 | poly (ADP-ribose) polymerase family, member 4 |
| 170407vpl2C9 | PAWR | PRKC, apoptosis, WT1, regulator |
| 041206pl3C8 | PBX3 | pre-B-cell leukemia transcription factor 3 |
| 311007pl3B8 | PCBD1 | pterin-4 alpha-carbinolamine dehydratase |
| 150506pl1C9 | PCBP2 | poly(rC)-binding protein 2 isoform b |
| 010506pl2D2 | PCMTD2 | protein-L-isoaspartate (D-aspartate) |
| 180504p12d10 | PDCD5 | programmed cell death 5 |
| 150506pl1C11 | PDIA5 | protein disulfide isomerase-associated 5 |
| 010506pl1B6 | PDIA6 | protein disulfide isomerase-associated 6 |
| 010806pl1G9 | PDZD2 | PDZ domain containing 2 |
| 160507pl3G6 | PFDN1 | Homo sapiens mRNA for prefoldin 1 variant, clone: |
| | | FCC107D06. |
| 190607pl1G1 | PFDN2 | prefoldin subunit 2 |
| 041206pl4H9 | PFDN5 | prefoldin subunit 5 isoform alpha |
| 050707pl2E5 | PFN1 | profilin 1 |
| 010806pl4B6 | PGK1 | phosphoglycerate kinase 1 |
| 031104p37b7 | PGRMC1 | progesterone receptor membrane component 1 |
| 041206pl1C9 | PHF20 | PHD finger protein 20 |
| 310506pl3C12 | PHLDB2 | pleckstrin homology-like domain, family B, |
| 290307pl1E1 | PHPT1 | phosphohistidine phosphatase 1 |
| 201107pl1C3 | PIAS2 | |
| 201107pl2H11 | PIGY | phosphatidylinositol glycan anchor biosynthesis, |
| 010806pl1C10 | PKN1 | protein kinase N1 isoform 2 |
| 171104p31b1 | PLAA | phospholipase A2-activating protein isoform 1 |
| 010306d9pl1B10 | PLEC1 | plectin 1 isoform 6 |
| 130207pl1D4 | PLS3 | plastin 3 |
| 310806pl2D4 | PNN | pinin, desmosome associated protein |
| 310506pl3E5 | POLR1D | polymerase (RNA) 1 polypeptide D isoform 1 |
| 200906pl4C4 | POLR2F | DNA directed RNA polymerase II polypeptide F |
| 200906pl1F10 | POLR2G | DNA directed RNA polymerase II polypeptide G |
| 041206pl6H10 | POLR2L | DNA directed RNA polymerase II polypeptide L |
| 010806pl1A1 | POLR3GL | polymerase (RNA) III (DNA directed) polypeptide |
| 160507pl3E8 | POMP | proteasome maturation protein |
| 310506pl2B12 | POR | cytochrome P450 reductase |
| 170604pl8b4 | PPA1 | pyrophosphatase 1 |
| 200906pl4F8 | PPFIBP1 | PTPRF interacting protein binding protein 1 |
| 310506pl4C1 | PPIA | peptidylprolyl isomerase A |
| 050707pl1F2 | PPP1R10 | protein phosphatase 1, regulatory subunit 10 |
| 170407vpl3A11 | PPP1R14A | protein phosphatase 1, regulatory (inhibitor) |
| 190607pl1H2 | PPP1R14B | protein phosphatase 1 regulatory subunit 14B |
| 010806pl1G5 | PPP1R2 | protein phosphatase 1, regulatory (inhibitor) |
| 200208pl2H5 | PPP2R2C | gamma isoform of regulatory subunit B55, protein |
| 010506pl2B8 | PRC1 | protein regulator of cytokinesis 1 isoform 1 |
| 160507pl3C7 | PRDX5 | peroxiredoxin 5 precursor, isoform a |
| 150506pl1F2 | Predicted gene | NT_030059.67 |
| 190607pl1H6 | PREPL | prolyl endopeptidase-like isoform C |
| 010506pl1F3 | PRKAR2A | cAMP-dependent protein kinase, regulatory |
| 170407pl1B7 | PROCR | Homo sapiens protein C receptor, endothelial |
| | | (EPCR), mRNA (cDNA clone MGC: 23024 |
| | | IMAGE: 4907433), complete cds. |
| 041206pl2A11 | PRPF4B | serine/threonine-protein kinase PRP4K |
| 201107pl4B8 | PRR11 | proline rich 11 |
| 200306f7pl1H4 | PRR13 | proline rich 13 isoform 2 |
| 010806pl4G1 | PRRX1 | paired mesoderm homeobox 1 isoform pmx-1a |
| 041206pl5C9 | PSIP1 | PC4 and SFRS1 interacting protein 1 isoform 2 |
| 050707pl3D5 | PSMA1 | proteasome alpha 1 subunit isoform 2 |
| 041206pl2D8 | PSMA2 | proteasome alpha 2 subunit |
| 310506pl1A3 | PSMA3 | proteasome alpha 3 subunit isoform 1 |
| 160507pl2F8 | PSMA7 | proteasome alpha 7 subunit |
| 200906pl5H10 | PSMB1 | proteasome beta 1 subunit |
| 130207pl2B4 | PSMB4 | Homo sapiens proteasome (prosome, macropain) |
| | | subunit, beta type, 4, mRNA (cDNA clone |
| | | MGC: 8522 IMAGE: 2822513), complete cds. |
| 201107pl2D10 | PSMB6 | proteasome beta 6 subunit |
| 200306f7pl1C11 | PSMB7 | proteasome beta 7 subunit proprotein |
| 290307pl1C6 | PSMC1 | proteasome 26S ATPase subunit 1 |
| 170407vpl3B9 | PSMC4 | proteasome 26S ATPase subunit 4 isoform 1 |
| 200906pl5C4 | PSMD1 | proteasome 26S non-ATPase subunit 1 |
| 310505p4f1e2 | PSMD11 | proteasome 26S non-ATPase subunit 11 |
| 310806pl2A5 | PSMD12 | proteasome 26S non-ATPase subunit 12 |
| 010806pl4E6 | PSMD6 | proteasome (prosome, macropain) 26S subunit, |
| 201107pl2G3 | PSME1 | proteasome activator subunit 1 isoform 1 |
| 311007pl1D2 | PSMF1 | proteasome inhibitor subunit 1 isoform 1 |
| 311007pl1G10 | PSPC1 | paraspeckle protein 1 |
| 280705plf13C2 | PTBP1 | polypyrimidine tract-binding protein 1 isoform |
| 041206pl7A12 | PTCRA | pre T-cell antigen receptor alpha |
| 160507pl2E10 | PTMA | prothymosin, alpha (gene sequence 28) |
| 310806pl2B11 | PTMS | parathymosin |
| 170407vpl3B6 | PTPLAD1 | butyrate-induced transcript 1 |
| 200306d9pl1E11 | PTTG1IP | pituitary tumor-transforming gene 1 |
| 201107pl2B5 | PXK | PX domain containing serine/threonine kinase |
| 200306f7pl1A4 | PXN | paxillin |
| 010506pl1B3 | RAB11A | Ras-related protein Rab-11A |
| 010704pl9b1 | RAB1A | RAB1A, member RAS oncogene family |
| 010806pl3B11 | RAB31 | RAB31, member RAS oncogene family |
| 050707pl3A5 | RAB33A | Ras-related protein Rab-33A |
| 280705p1f13C3 | RAC1 | ras-related C3 botulinum toxin substrate 1 |
| 311007pl2F1 | RANBP1 | RAN binding protein 1 |
| 310506pl3D4 | RASIP1 | CDNA FLJ20401 fis, clone KAT00901 (RASIP1 |
| | | protein). |
| 160507pl1A12 | RAVER1 | RAVER1 |
| 031104p47c12 | RBBP7 | retinoblastoma binding protein 7 |
| 010806pl1D10 | RBM12B | RNA binding motif protein 12B |
| 150506pl2D10 | RBM27 | RNA-binding protein 27 (RNA-binding motif protein |
| | | 27). |
| 010806pl3A12 | RBM41 | RNA binding motif protein 41 |
| 200906pl1F3 | RBM8A | RNA binding motif protein 8A |
| 010806pl3E10 | RBMXL1 | RNA binding motif protein, X-linked-like 1 |
| 050707pl3H9 | RBX1 | ring-box 1 |
| 041206pl2B7 | RCOR1 | REST corepressor 1 |
| 050707pl1B12 | RFC1 | replication factor C large subunit |
| 150506pl1F10 | RFXDC2 | regulatory factor X domain containing 2 |
| 010506pl2A6 | RGS10 | regulator of G-protein signaling 10 isoform b |
| 201107pl2A10 | RP11-255A11.5- | Ankyrin repeat domain 18B. |
| | 001 | |
| 170604p17c9a | RP3-467K16.1 | Novel protein (Fragment). |
| 190607pl1H11 | RPA2 | replication protein A2, 32 kDa |
| 310134b13 | RPL11 | ribosomal protein L11 |
| 200906pl4E5 | RPL12 | ribosomal protein L12 |
| 180504riboa2 | RPL13A | ribosomal protein L13a |
| 041206pl4D11 | RPL14 | ribosomal protein L14 |
| 150506pl1C8 | RPL18 | ribosomal protein L18 |
| 160507pl3E4 | RPL22 | ribosomal protein L22 proprotein |
| 200306f7pl1E8 | RPL23 | ribosomal protein L23 |
| 010806pl4D8 | RPL23A | ribosomal protein L23a |
| 041206pl2H2 | RPL24 | ribosomal protein L24 |
| 010506pl1D7 | RPL27A | ribosomal protein L27a |
| 200906pl4C11 | RPL29 | ribosomal protein L29 |
| 041206pl2G5 | RPL35 | ribosomal protein L35 |
| 031104p37b1 | RPL35A | ribosomal protein L35a |
| 031104p47d1 | RPL36 | ribosomal protein L36 |
| 200906pl1F9 | RPL36A | ribosomal protein L36a |
| 180504riboa7 | RPL4 | ribosomal protein L4 |
| 010806pl3E8 | RPL41 | ribosomal protein L41 |
| 310134c18 | RPL5 | ribosomal protein L5 |
| 311007pl2A9 | RPL6 | ribosomal protein L6 |
| 180504riboa1 | RPL7 | ribosomal protein L7 |
| 180504p11c7 | RPL7A | ribosomal protein L7a |
| 311007pl3G10 | RPL8 | Homo sapiens ribosomal protein L8, mRNA (cDNA |
| | | clone IMAGE: 3504599), partial cds. |
| 170407vpl2D6 | RPLP0 | ribosomal protein P0 |
| 010806pl2A11 | RPLP1 | hypothetical protein LOC729416 |
| 041206pl7B3 | RPLP2 | ribosomal protein P2 |
| 311007pl2E1 | RPP40 | ribonuclease P 40 kDa subunit |
| 310505p4f1e1 | RPS11 | ribosomal protein S11 |
| 150506pl1B6 | RPS12 | ribosomal protein S12 |
| 050707pl3G8 | RPS13 | ribosomal protein S13 |
| 010806pl1B2 | RPS15 | hypothetical protein LOC401019 |
| 010806pl2E10 | RPS15A | ribosomal protein S15a |
| 160507pl1B5 | RPS16 | ribosomal protein S16 |
| 010506pl1A6 | RPS17 | ribosomal protein S17 |
| 160507pl1F6 | RPS18 | ribosomal protein S18 |
| 201107pl3H11 | RPS19BP1 | S19 binding protein |
| 290307pl1D12 | RPS20 | Homo sapiens clone FLB0708 mRNA sequence. |
| 310506pl2B5 | RPS23 | ribosomal protein S23 |
| 150506pl1C1 | RPS24 | Homo sapiens full length insert cDNA clone |
| | | YB24C12. |
| 170407pl3D2 | RPS25 | ribosomal protein S25 |
| 041206pl2B8 | RPS28 | ribosomal protein S28 |
| 010506pl2B11 | RPS3 | ribosomal protein S3 |
| 310505p4f1c2 | RPS3A | ribosomal protein S3a |
| 280305p1f12C1 | RPS4X | ribosomal protein S4, X-linked X isoform |
| 310506pl1G12 | RPS7 | ribosomal protein S7 |
| 010806pl2A7 | RRM1 | ribonucleoside-diphosphate reductase M1 chain |
| 130207pl1E4 | RRP15 | ribosomal RNA processing 15 homolog |
| 280705p1f13D4 | RSL1D1 | ribosomal L1 domain containing 1 |
| 010806pl2G2 | RSRC2 | arginine/serine-rich coiled-coil 2 isoform b |
| 180504p12d12 | RTN4 | reticulon 4 isoform A |
| 010806pl1H1 | RY1 | putative nucleic acid binding protein RY-1 |
| 041206pl1F11 | S100A10 | S100 calcium binding protein A10 |
| 010806pl3E7 | S100A11 | S100 calcium binding protein A11 |
| 150506pl1A1 | S100A2 | S100 calcium binding protein A2 |
| 280305p6f2B2 | SAE1 | SUMO-1 activating enzyme subunit 1 |
| 280705p1f13C10 | SAFB | scaffold attachment factor B |
| 311007pl1B2 | SCAMP2 | secretory carrier membrane protein 2 |
| 201107pl3D10 | SEC13 | SEC13 protein |
| 201107pl2G11 | SEC14L1 | SEC14 (S. cerevisiae)-like 1 isoform a |
| 041206pl1A1 | SELM | selenoprotein M precursor |
| 200906pl2D11 | SERBP1 | SERPINE1 mRNA binding protein 1 isoform 1 |
| 041206pl3E11 | SERF2 | small EDRK-rich factor 2 |
| 010806pl4H2 | SERPINB6 | MSTP057. |
| 010306d9pl1B5 | SESN1 | sestrin 1 |
| 280305plf12D1 | SET | SET translocation (myeloid leukemia-associated) |
| 130207pl1B10 | SETMAR | SET domain and mariner transposase fusion |
| 170407pl1E2 | SF3B1 | splicing factor 3b, subunit 1 isoform 1 |
| 160507pl2C11 | SF3B14 | splicing factor 3B, 14 kDa subunit |
| 310131f6b | SFRS10 | splicing factor, arginine/serine-rich 10 |
| 200906pl4D3 | SFRS7 | splicing factor, arginine/serine-rich 7 |
| 041206pl1C5 | SH3GLB1 | SH3-containing protein SH3GLB1 |
| 310506pl3A11 | SH3KBP1 | SH3-domain kinase binding protein 1 isoform b |
| 010806pl1F5 | SHFM1 | candidate for split hand/foot malformation type |
| 160507pl1F9 | SIVA1 | CDNA FLJ46871 fis, clone UTERU3012999, highly |
| | | similar to Homo sapiens CD27-binding (Siva) |
| | | protein (SIVA). |
| 310505p4f1f7 | SKIV2L2 | superkiller viralicidic activity 2-like 2 |
| 010506pl2E6 | SLBP | histone stem-loop binding protein |
| 170407pl1G5 | SLC20A2 | solute carrier family 20, member 2 |
| 050707pl2C2 | SLC22A18AS | solute carrier family 22 (organic cation |
| 010806pl2D3 | SLC24A3 | solute carrier family 24 |
| 050707pl2D3 | SLC25A37 | mitochondrial solute carrier protein |
| 160507pl3B7 | SLC25A5 | solute carrier family 25, member 5 |
| 190607pl1E11 | SLC2A3 | solute carrier family 2 (facilitated glucose |
| 180504p1ab11 | SLC3A2 | solute carrier family 3 (activators of dibasic |
| 200906pl4A11 | SLC4A7 | solute carrier family 4, sodium bicarbonate |
| 010806pl2C11 | SLC6A7 | solute carrier family 6, member 7 |
| 160507pl2E12 | SLC9A3R1 | solute carrier family 9 (sodium/hydrogen |
| 050707pl1A10 | SLTM | modulator of estrogen induced transcription |
| 310806pl2E6 | SMS | spermine synthase |
| 090505p3f12d3 | SNRPB | small nuclear ribonucleoprotein polypeptide B/B′ |
| 010506pl1D5 | SNRPD1 | small nuclear ribonucleoprotein D1 polypeptide |
| 290307pl1B7 | SNRPF | small nuclear ribonucleoprotein polypeptide F |
| 201107pl2B11 | SNX3 | sorting nexin 3 |
| 200906pl4F3 | SNX6 | sorting nexin 6 isoform b |
| 170407vpl3B11 | SOD1 | superoxide dismutase 1, soluble |
| 200906pl3H7 | SON | SON DNA-binding protein isoform F |
| 201107pl1C5 | SORCS3 | VPS10 domain receptor protein SORCS 3 |
| 180504p1ab4 | SPAG4 | sperm associated antigen 4 |
| 311007pl3A9 | SPATA12 | spermatogenesis associated 12 |
| 150506pl1F1 | SPATS2 | spermatogenesis associated, serine-rich 2 |
| 050707pl2B12 | SPCS2 | signal peptidase complex subunit 2 homolog |
| 170407pl1F11 | SPG20 | spartin |
| 010806pl4E3 | SPTBN1 | spectrin, beta, non-erythrocytic 1 isoform 1 |
| 310806pl1H2 | SPTY2D1 | SPT2, Suppressor of Ty, domain containing 1 |
| 041206pl2A5 | SR140 | U2-associated SR140 protein |
| 170407pl1D8 | SRCAP | Snf2-related CBP activator protein |
| 200306f7pl1A12 | SRM | spermidine synthase |
| 130207pl2A6 | SRP14 | signal recognition particle 14 kDa (homologous |
| 170604p18b1 | SRP19 | signal recognition particle 19 kDa |
| 010806pl4D2 | SRPK1 | SFRS protein kinase 1 |
| 170407pl1C6 | SRRM1 | serine/arginine repetitive matrix 1 |
| 200306d9pl1C7 | SRRM2 | splicing coactivator subunit SRm300 |
| 311007pl3B10 | SSBP1 | single-stranded DNA binding protein 1 |
| 310506pl1A12 | STAG1 variant | stromal antigen 1 |
| | protein | |
| 201107pl1E6 | STAMBP | STAM binding protein |
| 050707pl3H10 | STAU1 | staufen isoform a |
| 160507pl1F4 | STK4 | serine/threonine kinase 4 |
| 010806pl4F12 | STMN1 | stathmin 1 |
| 200208pl2D12 | STXBP5L | Syntaxin-binding protein 5-like (Tomosyn-2) |
| | | (Lethal(2) giant larvae protein homolog 4). |
| 027073l5 | SUMO1 | SMT3 suppressor of mif two 3 homolog 1 isoform a |
| 160507pl1E9 | SUMO2 | SMT3 suppressor of mif two 3 homolog 2 isoform a |
| 311007pl2A4 | SYNCRIP | synaptotagmin binding, cytoplasmic RNA |
| 050707pl2G3 | T85821 | yd57b09.r1 Soares fetal liver spleen 1NFLS Homo |
| | | sapiens cDNA clone IMAGE: 112313 5′ similar to |
| | | contains MER25 repetitive element;, mRNA |
| | | sequence. |
| 170407pl1C1 | TALDO1 | transaldolase 1 |
| 290307pl1H5 | TARS | threonyl-tRNA synthetase |
| 010806pl3E2 | TBCA | tubulin-specific chaperone a |
| 200906pl3H2 | TBCB | cytoskeleton associated protein 1 |
| 200208pl2D5 | TCEA3 | transcription elongation factor A (SII), 3 |
| 170407pl1A7 | TCF25 | NULP1 |
| 010506pl2B12 | TCP1 | T-complex protein 1 isoform a |
| 310806pl2B5 | TDG | thymine-DNA glycosylase |
| 310505p4f1b4 | TENC1 | tensin like C1 domain containing phosphatase |
| 201107pl2C6 | TES | testin isoform 1 |
| 010506pl1A11 | TFAM | transcription factor A, mitochondrial |
| 310506pl1C6 | TFPT | TCF3 (E2A) fusion partner (in childhood |
| 170407vpl2B10 | THAP7 | THAP domain containing 7 isoform b |
| 050707pl1D6 | THOC4 | THO complex 4 |
| 041206pl3C6 | TIMP2 | tissue inhibitor of metalloproteinase 2 |
| 050707pl1C9 | TJP1 | tight junction protein 1 isoform b |
| 200906pl1D1 | TLCD1 | TLC domain containing 1 |
| 050707pl3D12 | TLN2 | talin 2 |
| 201107pl2C9 | TLOC1 | translocation protein 1 |
| 010806pl3C7 | TMCO3 | transmembrane and coiled-coil domains 3 |
| 050707pl3G11 | TMEM11 | transmembrane protein 11 |
| 310505p4f1d6 | TMEM123 | pro-oncosis receptor inducing membrane injury |
| 201107pl3E8 | TMEM132D | hypothetical protein LOC121256 |
| 010806pl2F12 | TMEM49 | transmembrane protein 49 |
| 200208pl2C6 | TMEM56 | Homo sapiens cDNA FLJ31842 fis, clone |
| | | NT2RP7000259. |
| 041206pl4E12 | TMEM75 | hypothetical protein LOC641384 |
| 170407pl3E9 | TMPO | thymopoietin isoform alpha |
| 160507pl3C8 | TNNC2 | fast skeletal muscle troponin C |
| 150506pl1E3 | TOMM7 | 6.2 kd protein |
| 170407pl3D10 | TOMM70A | translocase of outer mitochondrial membrane 70 |
| 310505p4f1e11 | TOP1 | DNA topoisomerase 1 |
| 050707pl1F12 | TPM1 | tropomyosin 1 alpha chain isoform 1 |
| 160507pl3B12 | TPM2 | tropomyosin 2 (beta) isoform 2 |
| 160507pl1G2 | TPM3 | tropomyosin 3 isoform 1 |
| 310505p4f1c7 | TPM4 | tropomyosin 4 |
| 010806pl4D12 | TPP1 | tripeptidyl-peptidase I preproprotein |
| 150506pl2G4 | TR | Thioredoxin reductase 1. |
| 190607pl1C7 | TRAPPC6A | trafficking protein particle complex 6A |
| 170407vpl3A3 | TRIM25 | tripartite motif-containing 25 |
| 041206pl4E2 | TRIM33 | tripartite motif-containing 33 protein isoform |
| 310506pl3H6 | TSNARE1 | t-SNARE domain containing 1 |
| 290307pl1H7 | TTC1 | tetratricopeptide repeat domain 1 |
| 130207pl1F6 | TTC26 | tetratricopeptide repeat domain 26 |
| 130207pl2A3 | TTC3 | tetratricopeptide repeat domain 3 |
| 160507pl2A9 | TTC9C | Homo sapiens clone pp8376 unknown mRNA. |
| 041206pl1B9 | TUBA1B | tubulin, alpha, ubiquitous |
| 160507pl1G1 | TUBA1C | tubulin alpha 6 |
| 050707pl3C9 | TUBB2C | tubulin, beta, 2 |
| 200306f7pl1G9 | TWF1 | twinfilin 1 |
| 160507pl1F3 | TXN | thioredoxin |
| 010506pl2A3 | TXNL1 | thioredoxin-like 1 |
| 010506pl1A12 | TXNRD1 | thioredoxin reductase 1 |
| 041206pl4H10 | TXNRD2 | thioredoxin reductase 2 precursor |
| 280705p1f13C6 | U2AF1 | U2 small nuclear RNA auxiliary factor 1 isoform |
| 171104p31b2 | UAP1 | UDP-N-acteylglucosamine pyrophosphorylase 1 |
| 041206pl2C4 | UBA52 | ubiquitin and ribosomal protein L40 precursor |
| 050707pl1C1 | UBE2D2 | ubiquitin-conjugating enzyme E2D 2 isoform 2 |
| 031104p47c7 | UBE2J2 | ubiquitin conjugating enzyme E2, J2 isoform 1 |
| 010506pl2A5 | UBE2L3 | ubiquitin-conjugating enzyme E2L 3 isoform 2 |
| 201107pl2C4 | UBE2N | ubiquitin-conjugating enzyme E2N |
| 170407vpl2B8 | UBE2Q2 | ubiquitin-conjugating enzyme E2Q (putative) 2 |
| 027073c5 | UBE2R2 | ubiquitin-conjugating enzyme UBC3B |
| 010806pl3D5 | UBE2V1 | ubiquitin-conjugating enzyme E2 variant 1 |
| 310806pl1E2 | UBE2V2 | ubiquitin-conjugating enzyme E2 variant 2 |
| 310506pl2D9 | UBL7 | ubiquitin-like 7 (bone marrow stromal |
| 201107pl1C8 | UBXD4 | Homo sapiens mRNA; cDNA DKFZp313K1023 |
| | | (from clone DKFZp313K1023). |
| 200208pl2F10 | UBXD8 | UBX domain containing 8 |
| 190607pl1A7 | UGCG | ceramide glucosyltransferase |
| 310506pl2A2 | UGP2 | UDP-glucose pyrophosphorylase 2 isoform b |
| 200906pl3C11 | UMPS | uridine monophosphate synthase |
| 200208pl2H8 | UNC5D | netrin receptor Unc5h4 |
| 160507pl1F2 | UNC84A | Sad1/unc-84 protein-like 1 (Unc-84 homolog A). |
| 160507pl1A10 | UPF2 | UPF2 regulator of nonsense transcripts homolog |
| 041206pl6A3 | UPF3A | UPF3 regulator of nonsense transcripts homolog A |
| 200906pl2F9 | UQCRB | ubiquinol-cytochrome c reductase binding |
| 290307pl1A3 | UQCRFS1 | ubiquinol-cytochrome c reductase, Rieske |
| 010806pl4F5 | USP10 | ubiquitin specific protease 10 |
| 010806pl1F11 | USP12 | ubiquitin-specific protease 12-like 1 |
| 130207pl1E5 | USP14 | ubiquitin specific protease 14 isoform a |
| 310506pl1B3 | USP34 | ubiquitin specific protease 34 |
| 310131e18l1 | USP7 | ubiquitin specific protease 7 (herpes |
| 170407vpl3B4 | UTP11L | UTP11-like, U3 small nucleolar |
| 050707pl3B6 | UTRN | utrophin |
| 280305p6f2B6 | VAPA | vesicle-associated membrane protein-associated |
| 210206pl1F1 | VASP | vasodilator-stimulated phosphoprotein isoform 1 |
| 160507pl1E8 | VBP1 | von Hippel-Lindau binding protein 1 |
| 010806pl2B3 | VCL | vinculin isoform meta-VCL |
| 010806pl3E12 | VIL2 | villin 2 |
| 200906pl3E11 | VKORC1 | vitamin K epoxide reductase complex, subunit 1 |
| 010506pl1B1 | VPS26A | vacuolar protein sorting 26 A isoform 1 |
| 290307pl1H3 | VPS29 | vacuolar protein sorting 29 isoform 2 |
| 290307pl1D8 | WASF2 | WAS protein family, member 2 |
| 010506pl2B4 | WDR12 | WD repeat domain 12 protein |
| 201107pl2B10 | WDR25 | pre-mRNA splicing factor-like |
| 311007pl1H10 | WDR43 | WD repeat protein 43. |
| 290307pl1A5 | XAGE1 | G antigen, family D, 2 isoform 1c |
| 160507pl3B4 | XRCC5 | ATP-dependent DNA helicase II |
| 310506pl1E7 | XRCC6 | ATP-dependent DNA helicase II, 70 kDa subunit |
| 310506pl1G5 | YAF2 | YY1 associated factor 2 isoform b |
| 200906pl1G8 | YAP1 | Yes-associated protein 1, 65 kD |
| 310806pl2A11 | YBX1 | nuclease sensitive element binding protein 1 |
| 010806pl1F2 | YTHDC1 | splicing factor YT521-B isoform 1 |
| 310506pl3A2 | YWHAE | tyrosine 3/tryptophan 5-monooxygenase |
| 170407vpl2D11 | YWHAG | tyrosine 3-monooxygenase/tryptophan |
| 201107pl3A9 | YWHAH | tyrosine 3/tryptophan 5-monooxygenase |
| 050707pl1C12 | YWHAQ | tyrosine 3/tryptophan 5-monooxygenase |
| 310506pl1B1 | YY1 | YY1 transcription factor |
| 310506pl1G3 | ZBTB25 | zinc finger protein 46 (KUP) |
| 130207pl1C10 | ZBTB8OS | zinc finger and BTB domain containing 8 opposite |
| 310506pl3A5 | ZCD1 | zinc finger CDGSH-type domain 1 |
| 311007pl1E10 | ZFAND2A | zinc finger, AN1-type domain 2A |
| 310806pl1A10 | ZFR | zinc finger RNA binding protein |
| 311007pl3C4 | ZFYVE21 | zinc finger, FYVE domain containing 21 |
| 280305p5f2E12 | ZNF433 | zinc finger protein 433 |
| 200208pl2A3 | ZNF646 | zinc finger protein 646 |
| 201107pl1C11 | ZNHIT3 | thyroid hormone receptor interactor 3 isoform 2 |
| 170407vpl3B1 | ZP3 | zona pellucida glycoprotein 3 preproprotein |
| 200906pl1A5 | ZW10 | centromere/kinetochore protein zw10 |

The proteins span a wide range of functional categories and localization patterns including membrane, nuclear, nucleolar, cytoskeleton, Golgi, ER and other localizations (SOM) (FIGS. 4A-C). All proteins in the library have localization patterns that match previous studies, when available (mis-localized proteins were excluded from this study).

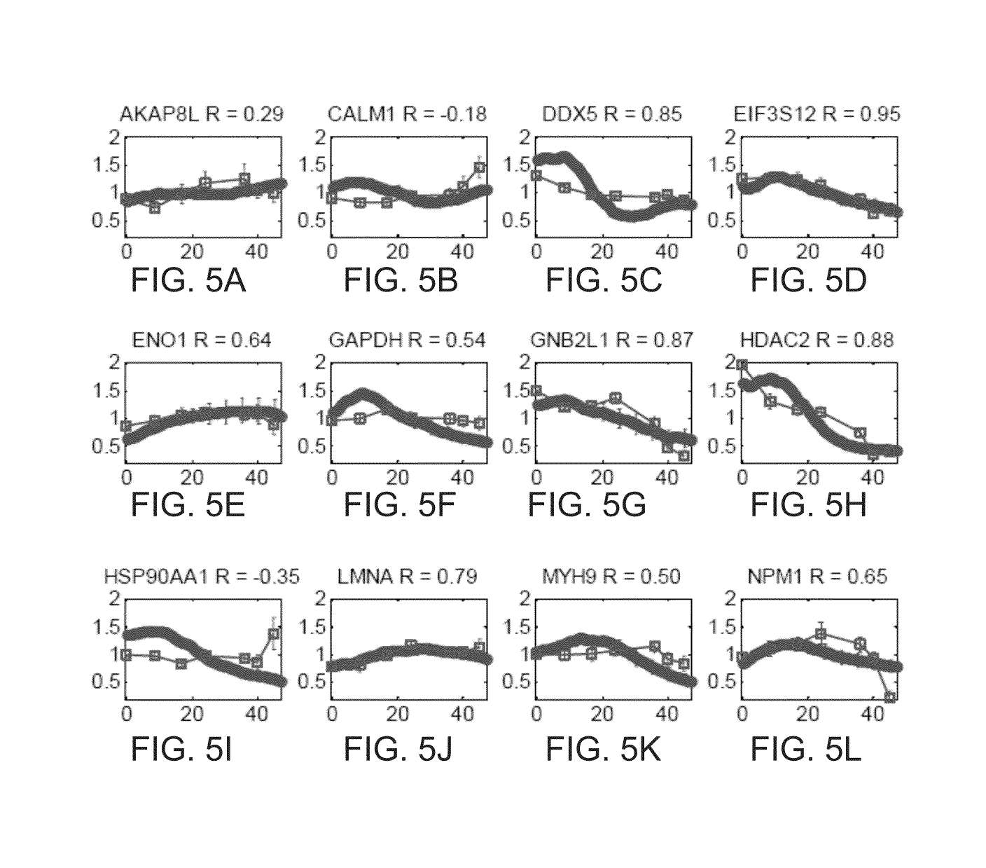

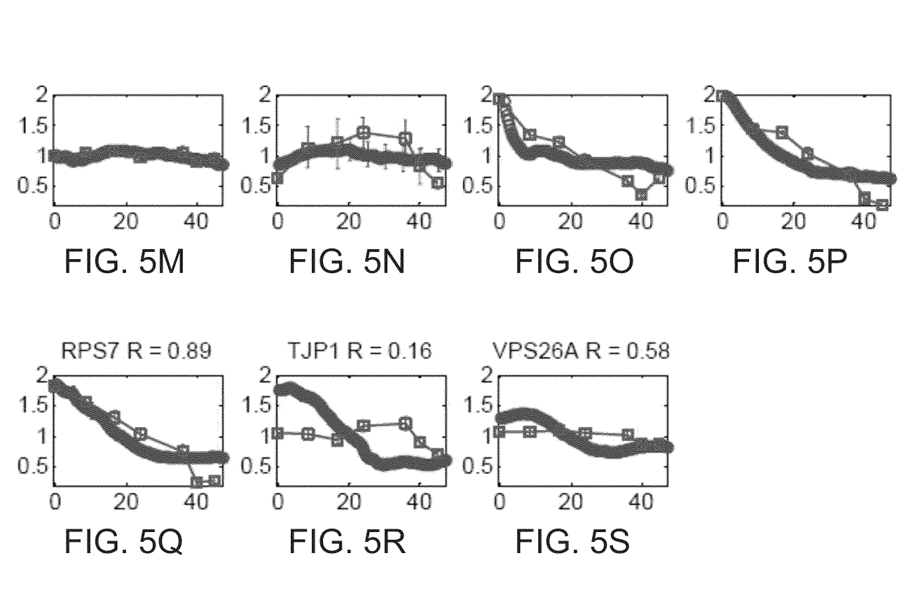

The present CD-tagging strategy tends to preserve protein functionality [Sigal, Milo et al. 2006, supra]. Note however that the present use of the library does not require proteins to be functional, but merely to act as reliable reporters for the dynamics and location of the endogenous proteins. To test this, the dynamics of endogenous protein using immunoblots on H1299-cherry cells with specific antibodies to 19 different proteins was measured. It was found that in 15/19 cases the immunoblot dynamics were correlated (R>0.5) with the fluorescence dynamics from the movies (FIGS. 5A-S). It was also found, that for all cases in which a band corresponding to the tagged protein was detected using anti-GFP immunoblotting, it indicated a full length fusion (Table 4, herein below).

TABLE 4

| | |
|---|---|
| | Size of YPF-fused protein, |
| Protein | kDa |

| name | Clone ID | Expected | Observed |
|---|---|---|---|
| CALM1 | 150506pl1E2 | ~47 (20 + 27) | ~47 |
| CKS2 | 010806pl4A1 | ~47 (10 + 27) | ~48 |
| DDX5 | 090505pl3D6 | ~95 (68 + 27) | ~95 |
| | 010806pl2F1 | | |
| EIF3S12 | 041206pl1C1 | ~55 (28 + 27) | ~55 |
| | 041206pl5H5 | | ~57 |
| ENO1 | 150506pl2F1 | ~77 (50 + 27) | ~77 |
| FAU | 170407pl2A5 | ~41 (14 + 27) | ~45 |
| FSCN1 | 010806pl1E12 | ~82 (55 + 27) | ~85 |
| GAPDH | 310806pl2C2 | 67 (40 + 27) | ~66 |
| GNB2L1 | 310806pl1H12 | ~64 (37 + 27) | ~66 |
| HSP90AA1 | 310506pl1B9 | ~120 (90 + 27) | ~120 |
| LMNA/C | 310806pl1H11 | Lamin A: | ~96 |
| | | ~96 (69 + 27) | |
| | | Lamin C: | ~89 |
| | | ~89 (62 + 27) | |
| NPM1 | 010806pl2H1 | ~60 (33 + 27) | ~67 |
| PBX3 | 041206pl3C8 | ~67 (40 + 27) | ~70 |
| PEPP-2 | 010806pl2B4 | ~59 (32 + 27) | ~58 |
| | 010806pl2D11 | | |
| PPIA | 310506pl4C1 | ~47 (20 + 27) | ~49 |
| | 031206pl3B6 | | ~47 |
| RPL18 | 150506pl1C8 | ~47 (20 + 27) | ~47 |
| RPS3A | 150506pl1B7 | ~63 (36 + 27) | ~66 |
| TJP1 | 050707pl1C9 | ~227 (200 + 27) | ~227 |
| TOP1 | 200906pl1C12 | ~120 (90 + 27) | ~120 |
| | 200306pl1H1 | | |
| | 010506pl1B1 | | |
| VPS26A | 050707pl1B11 | ~67 (40 + 27) | ~70 |
| | 211007pl2A8 | | |

### Example 3

### Assay of Proteomic Response to Drug

Drugs are used to affect the state of the cells, but little is known about the effects of drugs on the dynamics of proteins in individual human cells. The present Example illustrates analysis of drug activity on the dynamics of the proteome in individual cells. To address this, the present inventors employed, as a model system, human cancer cells responding to an anticancer drug with a well characterized target and mechanism of action: camptothecin (CPT). This drug is a topoisomerase-1 (TOP1) inhibitor with no other known targets. It locks TOP1 in a complex with the DNA, causing DNA breaks and inhibiting transcription, eventually causing cell death.

Materials and Methods

Long period time-lapse microscopy: Time-lapse movies were obtained (at 20× magnification) as described by Sigal et al. (Sigal, Milo et al. 2006, supra) with an automated, incubated (including humidity and CO2 control) Leica DMIRE2 inverted fluorescence microscope and an ORCA ER cooled CCD camera (Hamamatsu Photonics). The system was controlled by ImagePro5 Plus (Media Cybernetics) software which integrated time-lapse acquisition, stage movement, and software based auto-focus. During the experiment, cells were grown and visualized in 12-well coverslip bottom plates (MatTek) coated with 10 μM fibronectin (Sigma). For each well time lapse movies were obtained at four fields of view. Each movie was taken at a time resolution of 20 minutes and was filmed for at least three days (over 200 time points). Each time point included three images—phase contrast, red and yellow fluorescence.

Drug Materials: Camptothecin (CPT; C9911 Sigma), was dissolved in DMSO (hybri-max, D2650 Sigma) to achieve a stock solution of 10 mM. In each experiment, drug was diluted to 10 μM in a transparent growth medium (RPMI, X PenStrep, 10% FCS, w/o riboflavin, w/o phenol red, Bet Haemek). Growth medium (2 ml) was replaced by the diluted drug (2 ml) under the microscope. The same procedure was carried out for the following drugs: Etoposide (E1383 Sigma), diluted to 33.3 μM and for Cisplatinum (P4394 Sigma) diluted to 40 μM. The stock solution for ActD (A1410 Sigma) was 1 mg/ml and was diluted to 1 μg/ml.

Image analysis of time lapse movies: A custom written image analysis tool was used developed using the Matlab image processing toolbox environment (Mathworks, Natick, Mass.). The main steps include; image correction, segmentation, tracking of the cells and automated identification of cell phenotypes (mitosis and cell death). Image background correction (flat field correction and background subtraction) was carried out as previously described (Sigal, Milo et al. 2006, supra). No significant bleaching was observed (on average less than 3% over the duration of the experiment). Cell and nuclei segmentation was based on the red fluorescent images—all clones in the library showed similar distribution of red fluorescence—bright in the cytoplasm and significantly brighter in the nuclei. The main steps of the segmentation process are: 1) Differentiation between cells and background by global image threshold using Otsu's method (Otsu 1979, IEEE Transactions on Systems, Man, and Cybernetics 9(1): 62-66); 2) Segmentation of neighboring cells by applying the seeded watershed segmentation algorithm. Seeds were obtained by smoothening the red intensity image and usage of bright nuclei as cell seeds (by identifying local maxima)—one seed per cell; 3) Nuclei segmentation following cell segmentation; each cell was independently stretched between zero and one and a fixed threshold was used to differentiate between the cytoplasm and the nuclei; 4) Tracking of cells was performed by analyzing the movie from end to start and linking each segmented cell to the cell in the previous image with the closest centroid; 5) The automated cell death identification algorithm utilizes the morphological changes correlated with dying cells: rounding followed by blebbing and an explosion of the outer membrane or its collapse. An artificial neural network (ANN) algorithm was constructed that could identify each one of these morphological patters similar to the method previously described in (Eden 2005, IEEE, Transactions on Medical Imaging 24: 1011-1024). Briefly, two sets of images were constructed: The first contained 400 cell images in different stages of cell death and the second contained 400 live cell images. For each image, a collection of high-level image features was computed. An example of such a feature is a measure of object roundness, which is relevant due to the rounding that typically occurs prior to cell death. This process transforms each image into a multi dimensional vector of features. Based on these features an ANN classifier was trained in order to distinguish between live and dead cells resulting in a 96% sensitivity and specificity on a previously unseen test set.

Protein dynamics clustering: The five average population dynamics profiles depicted in FIG. 8B were generated in the following manner: The levels of each protein were smoothed using a median filter and linearly scaled between −1 and 1. The distance between every pair of proteins was measured in terms of Pearson correlation and clustering was performed using a k-means algorithm (reproducibility of results using different seeds is >99%). To choose the number of clusters optimization was effected over the average silhouette score (Blashfield 1991), which measures the dissimilarity of a protein to its assigned cluster compared to other clusters.

GO enrichment analysis: To systematically search for functions processes and localizations common to proteins that show similar dynamics we performed a GO (Ashburner, Ball et al. 2000, Nat Genet 25(1): 25-9) enrichment analysis procedure. A distance measure was devised between a pair of proteins that exploits both the protein amount and its localization changes through time. Formally, each protein i is represented by two vectors, cᵢ and nᵢ, describing the amount of protein in the nucleus and cytoplasm respectively in 141 sequential time points each.

The distance between each pair of proteins i and j was computed using the following formulas:

$${D}_{1} ( i , j ) = \frac{1 - Corr ( {n}_{i} + {c}_{i} , {n}_{j} + {c}_{j} )}{2}$$

$${D}_{2} ( i , j ) = Euc ( \frac{{n}_{i}}{{n}_{i} + {c}_{i}} , \frac{{n}_{j}}{{n}_{j} + {c}_{j}} )$$

$${D}_{tot} ( i , j ) = {w}_{1} \cdot {D}_{1} ( i , j ) + {w}_{2} \cdot {D}_{2} ( i , j )$$

D₁ is one minus the Pearson correlation between the total amounts of two proteins scaled between 0 and 1.
D₂ is the normalized Euclidian distance between two vectors that depict the protein localization at each time point. Notice that at a given time

$$t \frac{n ( t )}{n ( t ) + c ( t )}$$

may range from 0 to 1 corresponding to a cytoplasmic and nuclear localization respectively.
Dₜₒₜ is the weighted sum of the protein amount and protein localization distances where w₁+w₂=1 (we used w₁=0.5 and w₂=0.5). The larger w2 is, the more emphasis is put on localization and consequentially the GO terms that were identified (see next paragraph) were more related to Cellular Compartments terms.

The GO enrichment procedure was performed as following: For each protein a list was generated containing all other proteins ranked according to their distance. Each protein can be thought of as a cluster center and all the other proteins are ranked according to their distance from that center. The present inventors wanted to find whether a subset of proteins that show similar dynamics, i.e. reside near the cluster center, also share a common GO term. To this end a flexible cutoff version of the Hyper Geometric score termed mHG (Eden, Lipson et al. 2007, IEEE, Transactions on Medical Imaging 24: 1011-1024) was used. This analysis was done using GORILLA software [www.cbl-gorilladotcsdottechniondotacdotil/].

Quantitation of nucleolar translocations: To detect translocation events between the nucleoli and the nucleoplasm, a three step process was followed; first the present inventors focused on a subgroup of clones that showed initial nuclear localization of the YFP tagged protein (i.e. pixels of the nucleus were the source of over 50% of the total intensity). Then, for each of the selected clones, the present inventors calculated the ratio of fluorescence intensity between the top and bottom ten percent pixels in individual nuclei and averaged over the population. Clones with a max/min change of over 20 percent in this average during the experiment were inspected manually to verify the source of change in pixel intensity distribution and were classified as clones showing nucleolar translocation.

Finally, to quantify the extent and direction (nucleoli to nucleoplasm or vise versa) of the translocation, the present inventors calculated the ratio between mean fluorescence intensity of nucleoli vs. nucleoplasm (R_{ncll/nuc}) at the two time points were the max/min ratio was maximized and minimized. Measurements were normalized to 0.5, 1 and 2 at time point of drug addition, based on the R_{ncll/nuc} ratio at that time (R_{ncll/nuc}<0.8, 0.8<R_{ncll/nuc}<1.2 and R_{ncll/nuc}>1.2 respectively).

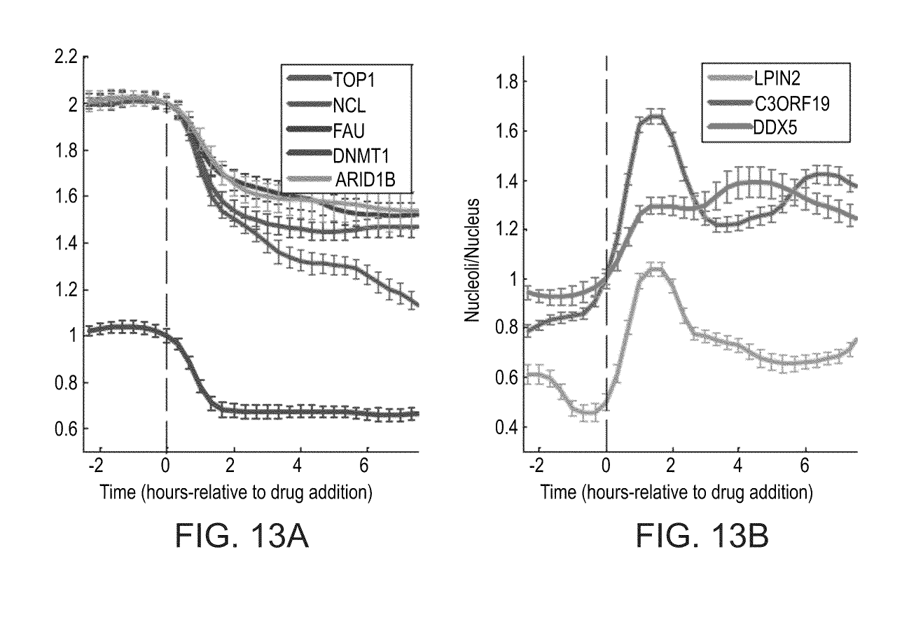

Determination of ‘bimodal’ behaviors: The coefficient of variance (CV defined as the ration between the std between cells and the mean) was measured for 400 proteins for 47 hours following addition of CPT (at a 20 minute resolution) (see FIGS. 13A-B). All CVs were normalized to average 1 (CV(i,j)/mean(mean(CV)) where i is protein number (i=1 . . . 400) and j is timepoint (j=1 . . . 141)). All proteins deviating 3 standard deviations from the average normalized CV were considered as ‘bimodal’ candidates (N=59). Following manual inspection, 30 of these proteins listed in Table 4 were denoted as bimodal.

Immunoblots against 20 selected proteins: Total cell lysates were prepared with RIPA buffer (Pierce) according to manufacturer's instructions. The protein concentrations were determined by BCA protein assay kit (Thermo scientific). Equal amounts of proteins were resolved on SDS-PAGE and subjected to immunoblotting analysis by using the antibodies listed below. The intensity of protein bands was quantified by using ImageJ software.

The following commercially available primary antibodies were used in the study: Antibodies against AKAP8L (ab51342), Calmodulin (ab38590), Cyclophilin A (ab3563), DDX5 (ab21696), Enolase (ab35075 and ab49256), eIF3K (ab50736), GAPDH (ab9285 and ab9484), HSP90 (ab13492 and ab34909), Nucleophosmin (ab15440), PBX3 (ab56239), Topoisomerase1 (ab28432) and VPS26 (ab23892) were purchased from Abcam.

Anti-Calmodulin (FL-149), -HDAC2 (H-54), -RACK1 (H-187 and B-3) and -ZO1 (H-300) antibodies were from Santa-Cruz.

Antibodies against RPL37 (A01), RPS7 (A01) and RPS3 (A01) proteins were obtained from Abnova.

Anti-Myosin IIA (M8064) and anti-GFP (11814460001) antibodies were from Sigma and Roche, respectively.

Conversion of fluorescence arbitrary units to scalable units: The present CD-tagging approach introduces a fluorescent protein into an endogenous protein, as an artificial exon. Under constant conditions (i.e. same exposure time and same lamp intensity) and under the assumption that the number of photons emitted and captured by each fluorescent molecule is similar, one can use fluorescence measurements to compare protein abundances. However, in practice, exposure times and lamp intensities differ between experiments and thus have to be corrected for. Exposure times of yellow and red channel were recorded throughout the experiments. In order to correct for differences in lamp intensity the red fluorescence levels averaged over all cells in a movie were used as a signal to align all clones. The following procedure was used to transform arbitrary fluorescent units to scalable units:

Fᵣ, F_{y}—measured red, yellow fluorescence
Eᵣ, E_{y}—exposure time for red, yellow channel
Pᵣ, P_{y}—number of proteins tagged with red, yellow fluorescence
L—lamp intensity
1. Fluorescence is a product of exposure time, protein number and lamp intensity.

Fᵣ=Eᵣ·Pᵣ·L F_{y}=E_{y}·P_{y}·L
2. To estimate the lamp intensity, it can be assumed that the average expression of the red marker, Pᵣ, is the same for all clones→Pᵣ=Const.

$$\begin{matrix}1 + 2 \to L = \frac{{F}_{r}}{{E}_{r} \cdot {P}_{r}} = \frac{{F}_{r}}{{E}_{r} \cdot Const} . & 3 \\ 1 + 3 \to {F}_{y} = {E}_{y} \cdot {P}_{y} \cdot L = {E}_{y} \cdot {P}_{y} \cdot \frac{{F}_{r}}{{E}_{r} \cdot Const} . & 4 \\ 4 \to {P}_{y} = \frac{{E}_{r} \cdot {F}_{y} \cdot Const}{{E}_{y} \cdot {F}_{r}} = \frac{{E}_{r} \cdot {F}_{y}}{{E}_{y} \cdot {F}_{r}} ( Const omitted ) . & 5\end{matrix}$$

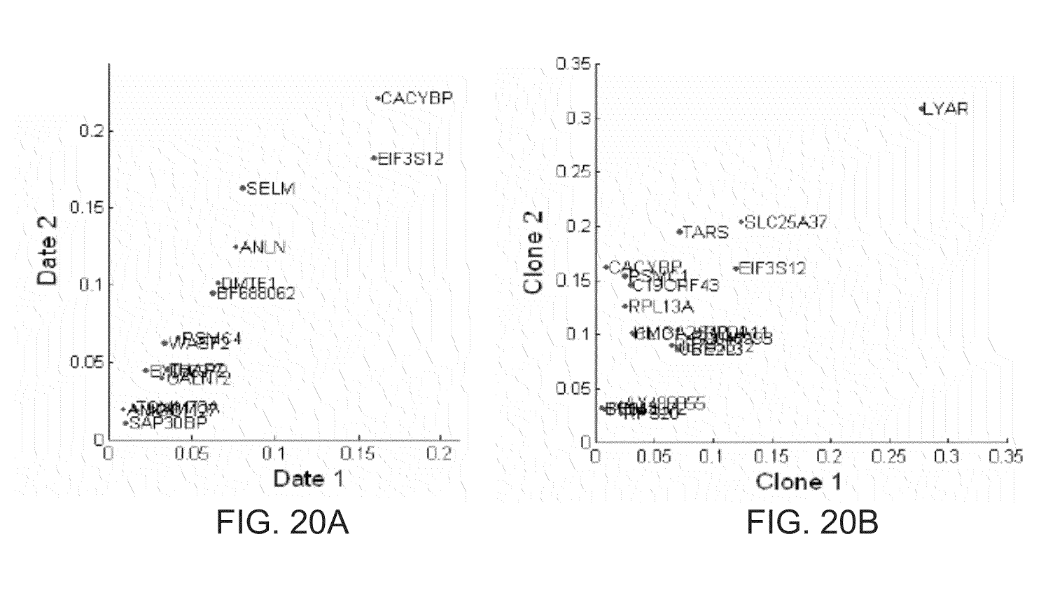

Following this scaling procedure, correlation of yellow intensity of the same protein from the same clone at a given time point, measured in two different days (starting form frozen cells) is very high, R=0.975 p<0.001. Moreover, the correlation of fluorescence intensity of a protein in two different clones where the protein is tagged at different chromosomal locations within the gene, is high, R=0.63 p<0.005. (FIGS. 20A-B). This suggests that the scaling procedure results in fluorescence units that allow determination of relative protein levels despite variations in lamp intensity and exposure times.

Identification of a drug target that acts to increase cell death following CPT treatment: Cells were plated in 12 well plate in 2 ml medium and filmed using the microscope under incubator conditions. At the begining of the movie, 1 μM of DDX5-siRNA (SEQ ID NOs: 175-178) was added. After three days, the DDX5-siRNA was removed and 10 μM of camptothecin was added. The cells continued being filmed at a 20 minute resolution for over 96 hours (whole experiment is over 144 hours). As controls, the experiment was repeated, but the DDX5-siRNA was replaced either by non-targeted-siRNA or no siRNA at all. As a further control, the identical experiment was repeated in the absence of camptoithecin.

Results

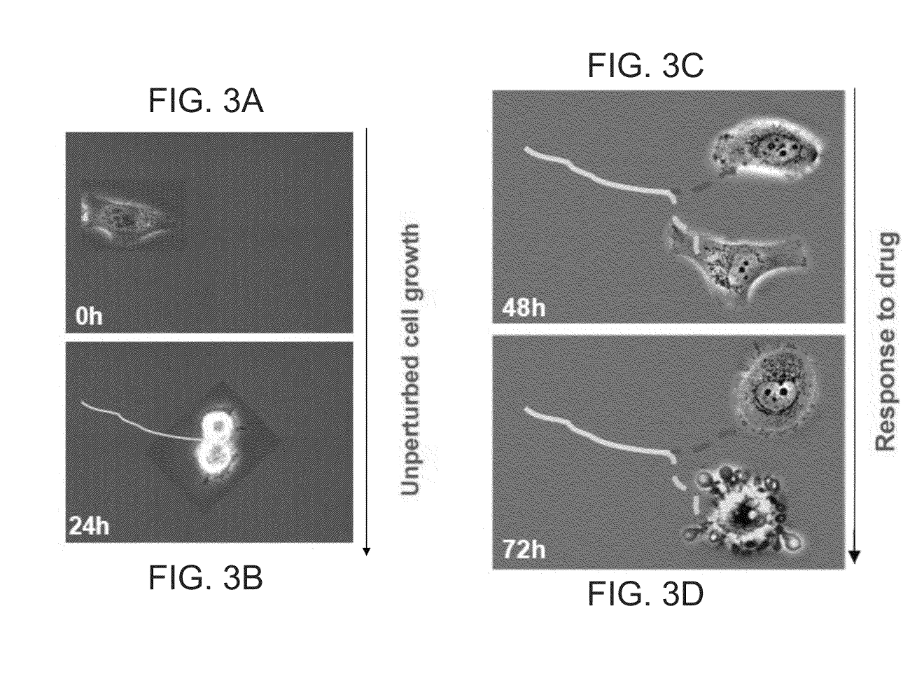

Cells were grown in 12-well plates in an automated fluorescence microscope with temperature, CO₂ and humidity control. Each well contained cells tagged for a different protein. After 24 hours of growth, the drug CPT was added (10 uM) and cells were tracked for another 48 hours (FIGS. 3A-D). Images in phase, red and yellow were taken every 20 minutes, at four positions in each well. An auto-focus system ensured that stable time-lapse movies could thus be collected, resulting in over 200 consecutive frames per protein studied, where each frame contained 10-40 different cells. Movies were stored and analyzed automatically using a computer cluster, resulting in traces of protein level and location in each cell over time.

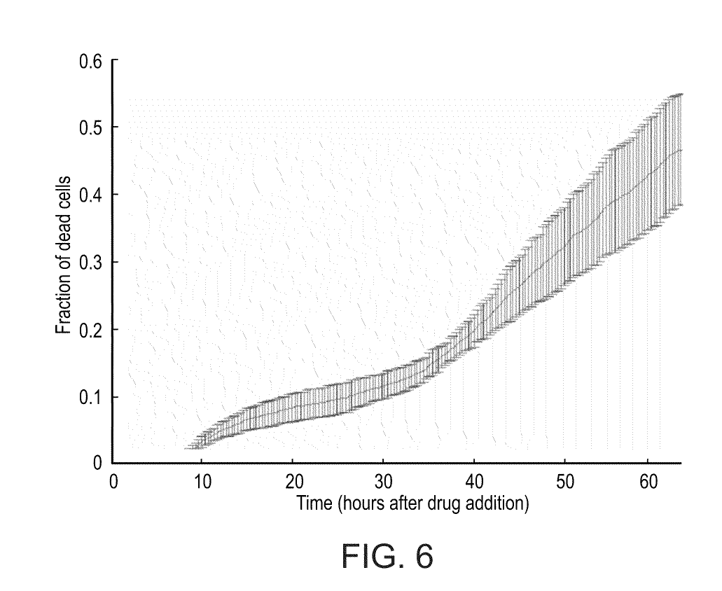

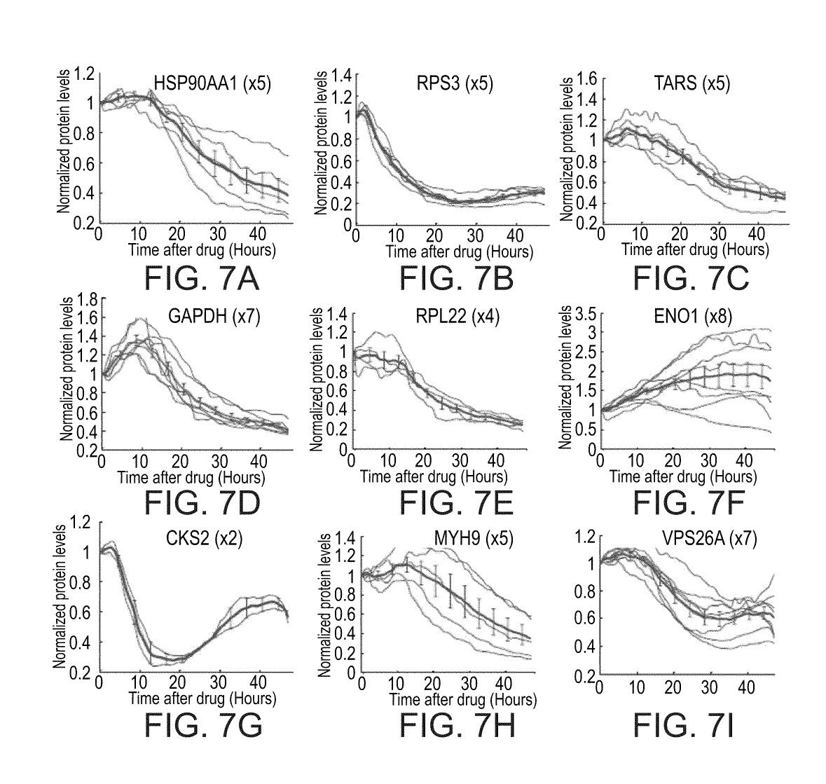

The cells showed vigorous divisions in the first 24 hours prior to drug addition, with a cell cycle of about 20 hours. Then, after drug addition, cells showed loss of motility and growth arrest after about 10 hours, and began to show cell rounding and blebbing (morphological correlates of cell death) reaching about 15% of the cells after 36 hours (FIG. 6). Day-day repeats starting from frozen cells showed a mean error in the YFP fluorescent signals of up to 15% (FIGS. 7A-I). Thus, dynamic changes on the order of 20-30% change in tagged protein intensity in individual cells are typically significant using the present assay.

Temporal profiles of protein concentration: The total fluorescence of each YFP tagged protein was measured in each cell. Overall, about 70% of the proteins show a decrease in intensity in response to the drug, on diverse timescales. The median dynamic range of this response was a 1.3-fold change in fluorescence and the largest changes were about five-fold change in fluorescence. Proteins show distinct classes of profiles, as obtained using k-means clustering (FIGS. 8A-B). The fluorescence levels of a third of the proteins decrease in the first 24 hours after drug addition (profile i). About half of the proteins show an increase followed by a decrease (profiles ii and iii). Other proteins showed an increase early (profile iv) or late, more than a day after drug addition (v). The present data includes dynamics of about 200 proteins annotated as uncharacterized hypothetical proteins or ESTs (Table 2, hereinabove). The dynamics of these uncharacterized proteins are found throughout all of the present profiles (FIG. 8B).

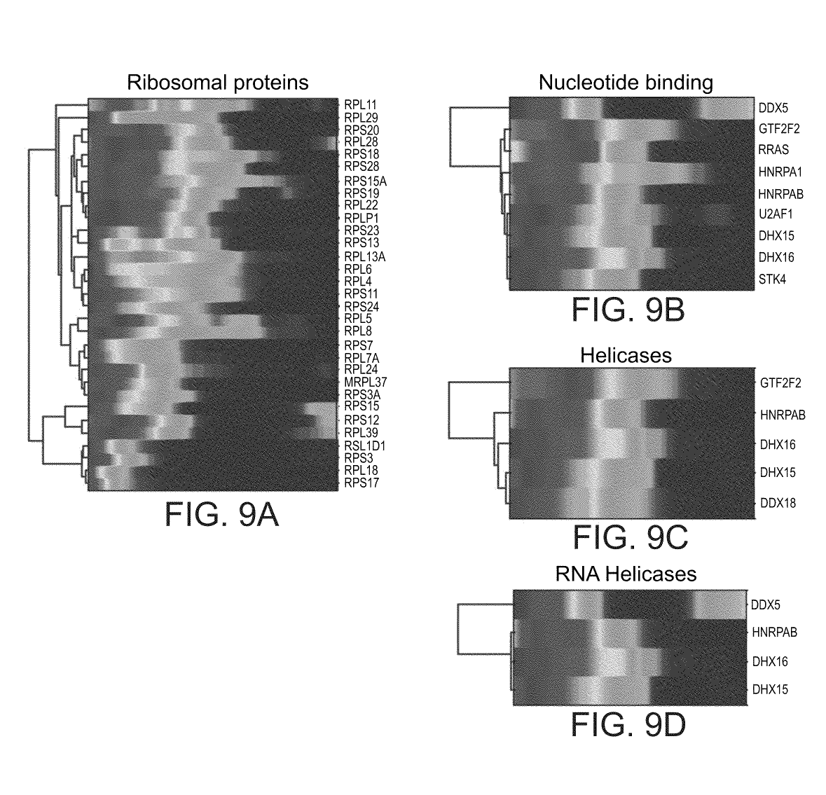

Groups of functionally related proteins tended to show similar dynamics and protein localization profiles. For example, over 75% (31/40) of the ribosomal proteins tagged in the library showed highly correlated dynamics of early degradation (p<10⁻³) (FIG. 8C and FIGS. 9A-D). This rapid degradation was also found in immunoblots with antibodies against ribosomal proteins RPS3a and RPL7. Proteins with slower apparent degradation include cytoskeleton components and metabolic enzymes. The timing of degradation of most cytoskeleton proteins correlated with the timing of the loss of cell motility as measured by tracking of cells (FIG. 8D). Proteins that rise late in the response include some helicases implicated in DNA damage repair and apoptosis-related proteins such as the Bcl2 associated proteins BAG2, BAG3 and programmed cell death protein PDCD5.

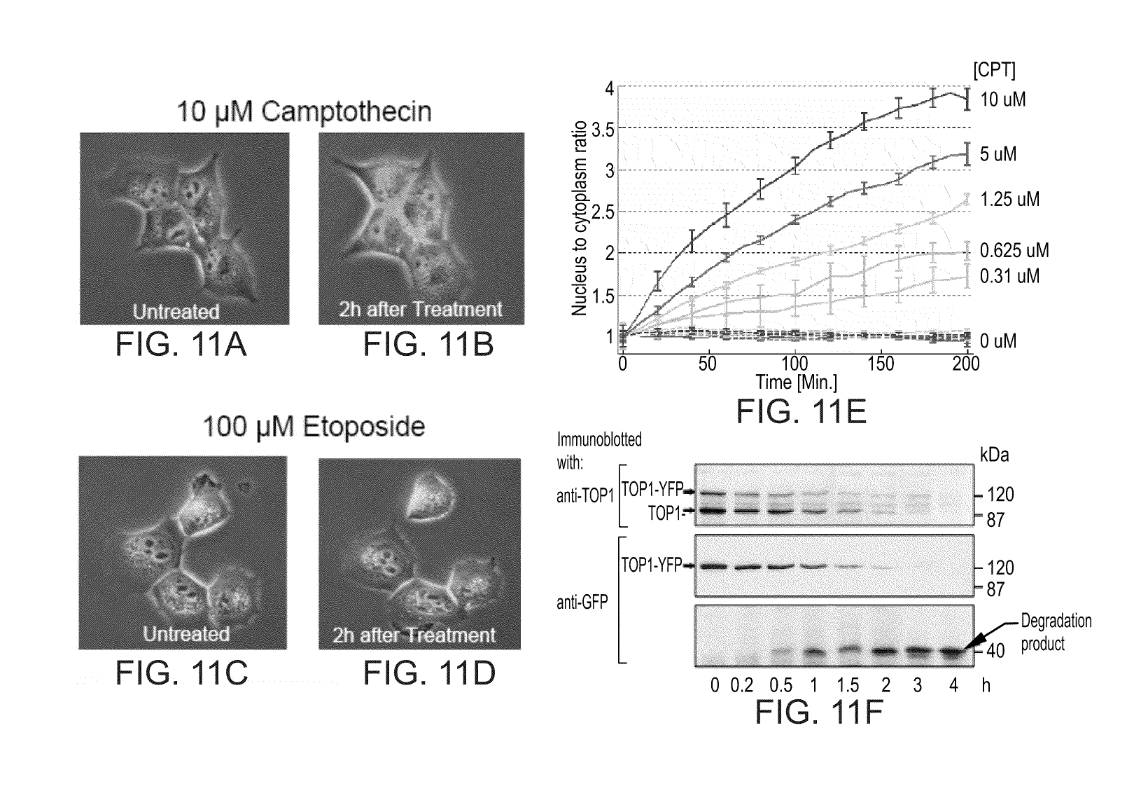

The drug target is among the first to respond: The drug target TOP1 is found in the nucleoli and nucleus of cells prior to drug addition. Drug addition caused TOP1 levels in the nucleoli to drop within less than 2 minutes (FIG. 10). The total cellular fluorescence levels of tagged TOP1 decreased on a timescale of under an hour, preceding almost all other responses in the present study (TOP1 is in the first 1% of responding proteins, FIG. 8B, arrow). The higher the CPT dose, the larger the extent TOP1 fluorescence decrease (FIG. 11E). Such rapid degradation was also found in immuoblots with anti-TOP1 antibodies (FIG. 11F).

In addition to nucleolar exit in the TOP1 tagged clone, it was found that fluorescence accumulates in the cytoplasm on the timescale of 5 hours following CPT addition, and that this accumulation increased with drug dose. Immunostaining of H1299-cherry cells with anti-TOP1 antibodies also showed endogenous TOP1 in the cytoplasm 5 hours after CPT treatment. Immunoblots indicated that as TOP1 degraded, an approximately 40 KD fragment detectable with anti-YFP antibody accumulated. None of the other 20 proteins tested with immunoblots in this study showed such a YFP fragment (FIGS. 5A-L and 11F). Taken together, these results suggest that TOP1 may be proteolised, and that TOP1 fragments exit the nucleus following drug administration. Other drugs, including DNA damaging drugs like TOP-2 inhibitor etoposide and cisplatin, did not show any of these effects on TOP1 (FIGS. 11C-D).

Rapid localization changes suggest nucleolar stress: In addition to TOP1, almost all of the other proteins that show rapid localization changes following CPT addition were localized to the nucleoli. The nucleolus is a key organelle that coordinates the synthesis and assembly of ribosomal subunits. Nucleolar proteins were identified that showed a reduction in nucleolar intensity (FIG. 12A), whilst other nucleolar proteins were identified that showed an increase followed by a return to basal level (FIG. 12B). Corresponding changes in the nuclear intensity outside of the nucleoli were found, suggesting that these are translocation events. In addition to localization changes, rapid decrease in the total level was seen in several nucleolar proteins, including ribosomal proteins. Similar results for the dynamics of most of these proteins (4 out of 5 proteins tested) were also found in response to the transcriptional inhibitor actinomycinD (1 μg/ml) FIGS. 13A-B. Similar nucleolar changes have been previously found in a study that monitored the composition of nucleoli extracted from cells responding to actinomycinD [Andersen, Lam et al. 2005, Nature 433(7021): 77-83]. In summary, these results suggest that the immediate effect of CPT on these cells is transcription inhibition, causing nucleolar stress.

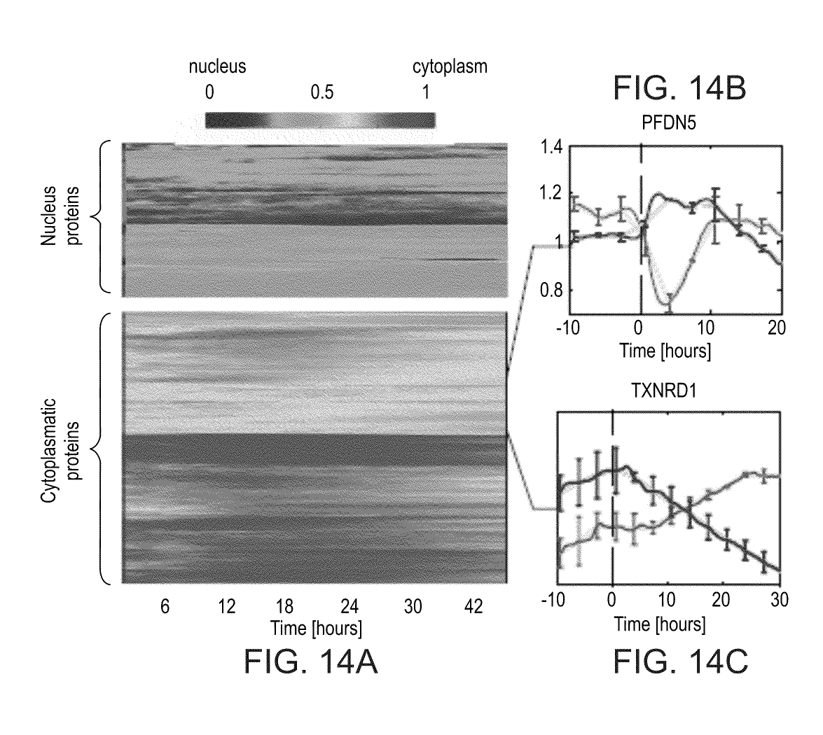

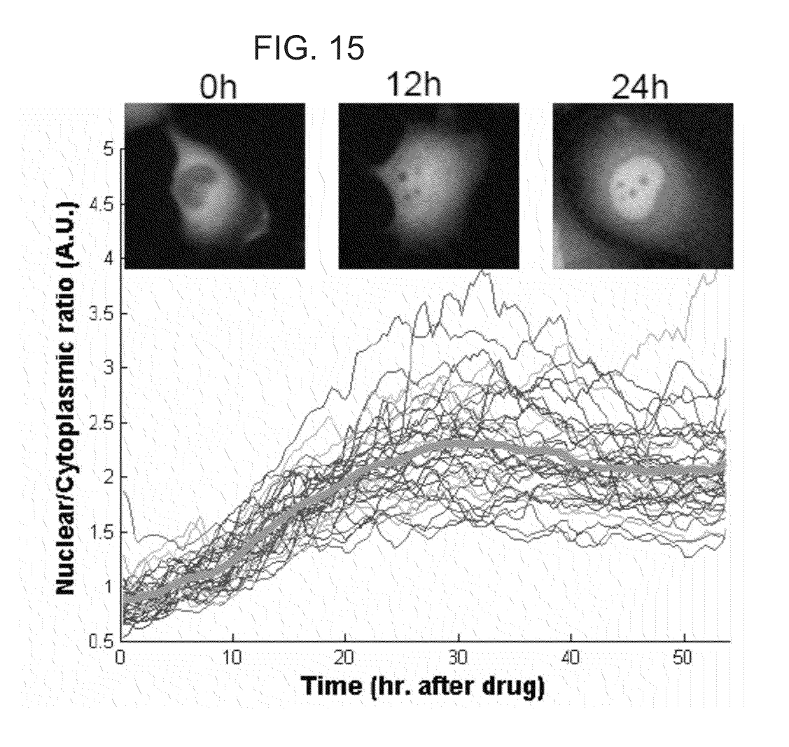

Nuclear localization changes following drug addition: The localization of each protein across the experiment was analyzed and the ratio of cytoplasmic to nuclear fluorescence was followed as a function of time. It was found that about 1% of the proteins showed significant change in nuclear localization (defined as >20% change in the cytoplasm/nuclear fluorescence ratio in an anti-correlated manner). Both rapid and slow localization changes between the cytoplasm and the nucleus were detected (FIGS. 14A-C). Among the latter are two proteins in the stress response pathway to oxidative stress: Both thyredoxin and thyredoxin reductase) showed an increase in nuclear/cytoplasmic ratio within 8 hours after drug addition (FIG. 15). As nuclear levels rise, cytoplasmic levels seem to decrease proportionally, and vise versa, suggesting that these translocations represent movement between these two compartments.

Several Proteins Show Highly Variable Behavior that Correlates with Outcome of Individual Cells:

The present system allows monitoring of the cell-cell variability of each protein over time. All proteins were found to show significant cell-cell variability in their fluorescence levels. At the time of drug addition, the level of each protein showed a standard deviation between cells that ranged between 10% and 60% of the mean. This variability is in accord with that previously found, both in microorganisms and human cells (Sigel, Milo et al. 2006, supra). Part of this variability is due to differences in the cell cycle stage of the cells. To quantify this, the cells were binned according to the time between their last division and the time of drug addition—an ‘in-silico’ synchronization approach (Sigel, Milo et al. 2006, supra). It was found that about 20% of the variability was due to cell-cycle stage difference, and the remainder was presumably due to stochastic processes.

The degree of cell-cell variability, defined as the standard deviation between cells divided by the mean, was found to show a slight increase as a function of time following drug addition for most proteins (FIG. 16) (noise increased by 30% on average). For most proteins, nearly all cells in the population showed similarly shaped profiles of fluorescence dynamics, rising and falling together (FIGS. 17A-B).

Diverging from this norm were about 30 proteins which showed a special behavior. At first, they showed the typical variability with similar dynamics in each cell. Then, at about 20 hours following drug addition, the cell population began to show dramatic cell-cell differences in the dynamics of these proteins (FIGS. 17C-F). Some cells showed an increase in the fluorescence levels, while other cells stayed constant or showed a decrease. Thus, these proteins seemed to show bimodal dynamical behavior.

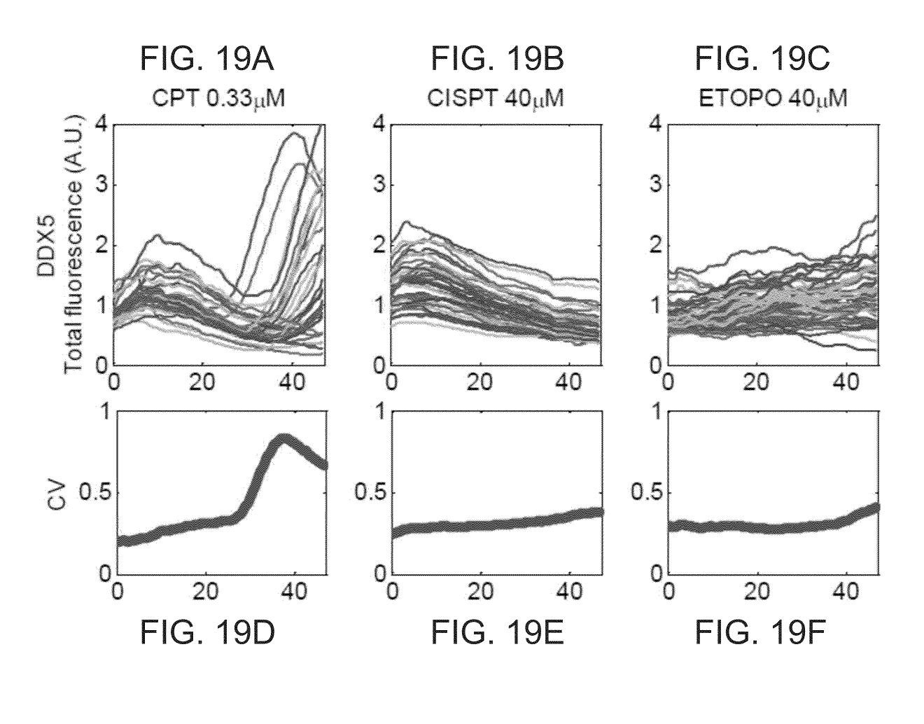

Importantly, the different behaviors of some of these proteins are linked to the fate of each cell. For example, it was found that the RNA-helicase DDX5 increased markedly in cells that survive to the end of the movies (FIG. 18A). This is consistent with its suggested anti-apoptotic role (Yang, Lin et al. 2007, Oncogene 26(41): 6082-92). Its levels decrease in cells that undergo the morphological changes associated with cell death. Thus, the fluorescence dynamics of this protein were significantly correlated with the cell fate (p<10̂-13, FIG. 18B). Such effects can not be detected in assays that average over cell populations. The bimodality of DDX5 was found to be drug specific, since tagged DDX5 did not show bimodal behavior in response to other anti-cancer drugs including etoposide and cisplatin (see FIGS. 19A-F).

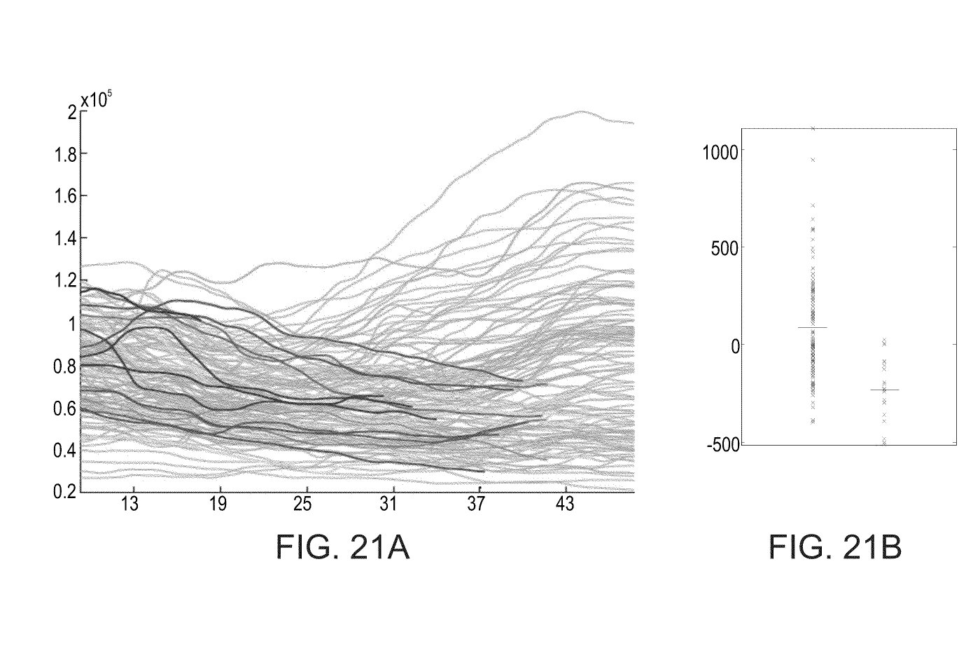

A second protein that shows similar behavior to DDX5 is Replicator factor C activator 1 (RFC1; FIGS. 21A-B). Replication factor C is a DNA-dependent ATPase that is required for eukaryotic DNA replication and repair. The protein acts as an activator of DNA polymerases.

A third protein that showed bimodal dynamical behavior is thioredoxin reductase 1 (TXNRD1). This protein is involved in the cellular response to oxidative stress. Following changes in NADPH levels, TXNRD1 reduces thioredoxin which translocates into the nucleus and eventually leads to the expression of stress related genes.

The present study showed that both TXNRD1 and thioredoxin enter the nucleus in response to Camptothecine. Previously it was suggested that these proteins are novel drug targets and that their inhibitors should be used together with ionizing radiation (IR) or H₂O₂ [Nguen et al., Cancer Letters, Volume 236, Issue 2, Pages 164-174 P].

Table 5, herein below lists the functions of the proteins with bimodal behavior, and gives reference to association of some of the proteins to cell fate.

TABLE 5

| | | | Reference to |
|---|---|---|---|
| | | | association of |
| | | | protein to cell |
| Protein name | Clone ID | description | death |
| BAG2 | 010806pl1C7 | BCL2-associated athanogene 2 | |
| BAG3 | 170407pl3D4 | BCL2-associated athanogene 3 | P. Bonelli et al., |
| | | | Leukemia 18, |
| | | | 358-60 (Feb, |
| | | | 2004) |
| C9ORF40 | 130207pl1E1 | hypothetical protein LOC55071 | |
| CALM1 | 150506pl1E2 | calmodulin 1 | O. Cohen, E. Feinstein, |
| | | | A. Kimchi, |
| | | | Embo J |
| | | | 16, 998-1008 |
| | | | (Mar. 3, 1997). |
| | | | Y. Shirasaki, Y. Kanazawa, |
| | | | Y. Morishima, |
| | | | M. Makino, |
| | | | Brain |
| | | | Res 1083, 189-95 |
| | | | (Apr. 14, 2006 |
| CALM2 | 310506pl3B1 | calmodulin 2 | O. Cohen, E. Feinstein, |
| | | | A. Kimchi, |
| | | | Embo J |
| | | | 16, 998-1008 |
| | | | (Mar. 3, 1997). |
| | | | Y. Shirasaki, Y. Kanazawa, |
| | | | Y. Morishima, |
| | | | M. Makino, |
| | | | Brain |
| | | | Res 1083, 189-95 |
| | | | (Apr. 14, 2006 |
| CAV1 | 170407pl1C2 | caveolin 1 | C. C. Ho et al., |
| | | | Lung Cancer 59, |
| | | | 105-10 (Jan, |
| | | | 2008). |
| CCDC23 | 310506pl2C3 | coiled-coil domain containing 23 | |
| DDX5 | 010806pl2F1 | p68 RNA helicase | L. Yang, C. Lin, |
| | | | S. Y. Sun, S. Zhao, |
| | | | Z. R. Liu, |
| | | | Oncogene 26, |
| | | | 6082-92 (Sep. 6, |
| | | | 2007). |
| DKFZP434M1123 | 160507pl1B11 | hypothetical protein | |
| EIF1AX | 010806pl2B11 | eukaryotic translation initiation | |
| | | factor 1A, X-linked | |
| FABP5 | 200906pl1B6 | fatty acid binding protein 5 | |
| FSCN1 | 010806pl1E12 | fascin homolog 1, actin-bundling | |
| | | protein | |
| PCMTD2 | 010506pl2D2 | protein-L-isoaspartate (D- | |
| | | aspartate) O-methyltransferase | |
| | | domain containing | |
| PDCD5 | 170407pl1B5 | programmed cell death 5 | M. Xu et al., |
| | | | Gene 329, 39-49 |
| | | | (Mar. 31, |
| | | | 2004). |
| PFN1 | 050707pl2E5 | profilin 1 | |
| NPM1 | 010806pl2H1 | Nucleophosmin (B23) | Y. Qing, G. Yingmao, |
| | | | B. Lujun, |
| | | | L. Shaoling, J |
| | | | Neurol Sci 266, |
| | | | 131-7 (Mar. 15, |
| | | | 2008) |
| PPP1R2 | 010806pl1G5 | protein phosphatase 1, | |
| | | regulatory (inhibitor) subunit 2 | |
| PTTG1 | 310506pl2C2 | pituitary tumor-transforming 1 | Y. Lai, D. Xin, J. Bai, |
| | | | Z. Mao, Y. Na, |
| | | | J Biochem |
| | | | Mol Biol 40, |
| | | | 966-72 (Nov. 30, |
| | | | 2007). |
| RFC1 | 050707pl1B12 | replication factor C (activator 1) | |
| RPS3 | 150506pl2B7 | ribosomal protein S3 | C. Y. Jang, J. Y. Lee, |
| | | | J. Kim, |
| | | | FEBS Lett 560, |
| | | | 81-5 (Feb. 27, |
| | | | 2004). |
| SLBP | 010506pl2E6 | stem-loop binding protein | Y. Kodama, J. H. Rothman, |
| | | | A. Sugimoto, |
| | | | M. Yamamoto, |
| | | | Development |
| | | | 129, 187-96 |
| | | | (Jan, 2002). |
| SPCS1 | 050707pl2F4 | signal peptidase complex | |
| | | subunit 1 homolog | |
| TOMM70A | 170407pl3H11 | translocase of outer | |
| | | mitochondrial membrane 70 | |
| | | homolog A | |
| YT521 | 010806pl1F2 | YTH domain containing 1 | |

Identification of a drug target that acts to increase cell death following CPT treatment: As mentioned, a subgroup of proteins was found that show bimodal behavior in response to drug (Camptothecin). Of these, two (DDX5 and RFC1) showed that this behavior was correlattive to cell fate (FIGS. 18A-B and 21A-B).

The present inventors then hypothesised thatt down-regulation of DDX5 may lead to higher levels of cell death. As illustrated in FIG. 22, application of DDX5-siRNA, (thereby causing a reduction in expression levels by at least 80%), caused an increase rate (approximately double) in cell death following drug addition. This holds for at least the first 35 hours following drug addition. Addition of DDX5-siRNA did not cause cell death on its own (with OUT CPT—purple line). This suggests that the effect of downregulation of DDX5 on cell death will be observed only in cells that initially respond to CPT. All of the above suggests that a drug target has been identified that when inhibited doubles the rate of cell death following CPT administration.

Discussion

This study suggests that viewing the drug response of about 1000 proteins in human cancer cells in space and time, offers insight into the drug mechanisms of action, and uncovers proteins correlated with the fate of cell subpopulations. The present inventors found rapid and specific initial movements to and from the nucleoli of a group of proteins, including the drug target. Slower, broad patterns of protein accumulation and degradation followed, as the cells stopped moving and began cell death. Specific proteins showed high cell-cell variability that correlated with cell survival or death.

The present data is relevant to the question of diversity in the response of individual cells to a drug. The present inventors found that most proteins showed variability between cells, on the order of 10-60% in their mean levels. The drug seemed to cause a slight increase in the cell-cell variability of almost all proteins. This variability is not strongly correlated with the cell fate for most proteins. However, a small set of proteins showed variability that was highly correlated with the cell fate. These proteins may play a role in cell survival and death specific to this drug, or at least may be downstream factors associated with the molecular variability that underlies differential response. This suggests a way to begin to understand non-genetic resistance of human cell subpopulations to drugs, and may point to potential secondary targets that can enhance the effects of a given drug.

These results also suggest a separation of timescales in the response, where rapid and specific responses are mediated by translocation, and slower responses that include large sets of proteins are mediated by slower changes in expression and degradation. The translocations that occur soon after the drug is added may point to feedback mechanisms which sense the immediate effect of the drug. In the present study, CPT is found to have an almost immediate effect on nucleolar proteins. This response is typical of the nucleolar response to transcriptional inhibition. Notably, the drug target TOP1 is among the first to respond. This may suggest a strategy to understand drug mechanism of action and to detect drug targets and target-associated proteins for drugs with unknown targets.

The present library also provides dynamics and localization data for about 200 proteins that are classed as hypothetical proteins or ESTs (FIG. 8B and Table 2). The library provides a universal epitope tag (yellow fluorescent protein) that can in principle be used for biochemical assays on these novel proteins. The present approach may thus offer an opportunity to characterize new proteins.

The present library employs tagging that preserves endogenous regulation and is built to allow robust image quantification. Its reproducibility, temporal resolution and accuracy allow even small dynamical features to be reliably detected.

In summary, this first broad view of the response of the proteome of individual human cells to a drug points to aspects of the drug mode of action and to specific differences in protein expression in cell subpopulations. Rapid localization changes help to pinpoint the drug target, and slower waves of accumulation and degradation provide a picture of the way the cells respond to drug stress over time. A subset of proteins showed behavior correlated with the survival and death of differential cell subpopulations. This opens the way for viewing and potentially understanding the dynamics of the human proteome under diverse drugs and conditions in individual cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A nucleic acid construct system comprising:
(i) a first nucleic acid construct comprising a first nucleic acid sequence encoding a first reporter polypeptide linked to an additional nucleic acid sequence capable of inserting said first nucleic acid construct into a genome of a host cell such that an endogenous polypeptide covalently attached to said first reporter polypeptide is expressed in said cell, said endogenous polypeptide having a higher nuclear:cytoplasm expression ratio; and
(ii) a second nucleic acid construct comprising a second nucleic acid sequence encoding a second reporter polypeptide, linked to an additional nucleic acid sequence capable of inserting in a non-directed manner said second nucleic acid construct into a genome of a host cell such that an endogenous polypeptide covalently attached to said second reporter polypeptide is expressed in said cell, wherein said first reporter polypeptide and said second reporter polypeptide are distinguishable.

2. The nucleic acid construct system of claim 1, further comprising a third nucleic acid construct comprising a third nucleic acid sequence encoding said first reporter polypeptide linked to an additional nucleic acid sequence capable of inserting said third nucleic acid construct into a genome of a host cell such that an additional endogenous polypeptide covalently attached to said first reporter polypeptide is expressed in said cell.

3.-10. (canceled)

11. The nucleic construct system of claim 1, wherein said first reporter and said second reporter are fluorescent polypeptides that fluoresce at a distinguishable wave length.

12. A cell expressing at least two endogenous polypeptides, each covalently attached to a distinguishable reporter polypeptide wherein at least one of said at least two endogenous polypeptides has a higher nuclear:cytoplasm expression ratio.

13. (canceled)

14. The cell of claim 12, expressing an additional endogenous polypeptide attached to a reporter polypeptide, said reporter polypeptide being identical to one of said two distinguishable reporter polypeptides.

15. The cell of claim 12, wherein an expression of said at least one of said at least two endogenous polypeptides is constitutive.

16. The cell of claim 12, comprising a nucleic acid construct system comprising:
(i) a first nucleic acid construct comprising a first nucleic acid sequence encoding a first reporter polypeptide linked to an additional nucleic acid sequence capable of inserting said first nucleic acid construct into a genome of a host cell such that an endogenous polypeptide covalently attached to said first reporter polypeptide is expressed in said cell, said endogenous polypeptide having a higher nuclear:cytoplasm expression ratio; and
(ii) a second nucleic acid construct comprising a second nucleic acid sequence encoding a second reporter polypeptide, linked to an additional nucleic acid sequence capable of inserting in a non-directed manner said second nucleic acid construct into a genome of a host cell such that an endogenous polypeptide covalently attached to said second reporter polypeptide is expressed in said cell, wherein said first reporter polypeptide and said second reporter polypeptide are distinguishable.

17.-19. (canceled)

20. A cell population, wherein each cell of the population expresses at least two endogenous polypeptides, each covalently attached to a distinguishable reporter polypeptide, wherein at least one of said at least two endogenous polypeptides is identical in each cell of said cell population.

21. The cell population of claim 20, expressing an additional endogenous polypeptide attached to a reporter polypeptide, said reporter polypeptide being identical to one of said two distinguishable reporter polypeptides.

22. The cell population of claim 20, wherein both of said at least two endogenous polypeptides are identical in each cell of said cell population.

23. (canceled)

24. The cell population of claim 20, wherein at least one of said at least two endogenous polypeptides comprises a sequence as set forth in SEQ ID NOs: 1-164.

25.-26. (canceled)

27. A method of generating a cell population, the method comprising:
(a) introducing a first nucleic acid construct into a first population of cells, said first nucleic acid construct comprising a first nucleic acid sequence encoding a first reporter polypeptide linked to an additional nucleic acid sequence capable of inserting said first nucleic acid construct into a genome of a host cell such that an endogenous polypeptide covalently attached to said first reporter polypeptide is expressed in said cell;
(b) selecting a cell wherein said first reporter comprises a higher nuclear:cytoplasm expression ratio;
(c) propagating said cell to generate a second population of cells;
(d) introducing a second nucleic acid construct into the second population of cells, said second nucleic acid construct comprising a second nucleic acid sequence encoding a second reporter polypeptide, linked to an additional nucleic acid sequence capable of inserting in a non-directed manner said second nucleic acid construct into a genome of a host cell such that an endogenous polypeptide covalently attached to said second reporter polypeptide is expressed in said cell, wherein said first reporter polypeptide and said second reporter polypeptide are distinguishable.
thereby generating the cell population.

28.-29. (canceled)

30. The method of claim 27, further comprising identifying at least one of said endogenous polypeptides.

31. A method of identifying a target of an agent, the method comprising:
(a) contacting the cell population of claim 22 with the agent;
(b) analyzing a localization or amount of at least one of said endogenous polypeptides, wherein a change in said amount or localization is indicative of a target of the agent.

32.-34. (canceled)

35. A method of identifying an agent capable of affecting a cell state, the method comprising,
(a) contacting the cell population of claim 22 with an agent; wherein at least one of said endogenous polypeptides is a marker for the cell state; and
(b) measuring a localization or amount of said marker, wherein a change in said amount or localization of said marker is indicative of an agent capable of affecting the cell state.

36.-37. (canceled)

38. A method of identifying a marker for disease prognosis, the method comprising:
(a) contacting the cell population of claim 22 with a therapeutic agent, the cell population comprising diseased cells;
(b) comparing a localization or amount of said at least one endogenous polypeptide in responsive cells of the cell population with non-responsive cells of the cell population; wherein a difference in expression or localization of said at least one endogenous polypeptide in responsive and non-responsive cells is indicative that said endogenous polypeptide is the marker for disease prognosis.

39. (canceled)

40. A method of analyzing a localization of a first and second endogenous polypeptide in a cell, the method comprising detecting a localization of said first and second endogenous polypeptide in said cell, wherein said first and second polypeptide are each covalently attached to a distinguishable reporter polypeptide, thereby analyzing localization of a first and second polypeptide.

41.-44. (canceled)


SEQUENCE LISTING (178 sequences)

SEQ ID NO: 1 - DNA, 364 bp, Homo sapiens
ggtctaatcaatggtgctctaactacagaacagcgaatcttaacctggggaccatgggcatgaatggactgcagaaattctgtgacctcctgagaagtgttctgggtgagacaaggcttgaaagagacaaagaaggctggagccccgtggcagatgagatggttagatctgcaggcatactaactggaagagatacaacagaggctccctggaagaaggatgccagacacagttgagagcacaagactgtaccaaaaacaagagattaaatgactttaacatattaaatgctgaatacagaagtcccccagctagaaccccaactaggcttccagaatgcaggcaaccacactgctatccttta

SEQ ID NO: 2 - DNA, 327 bp, Homo sapiens
aacagacttgtgatattactttaatggcaaacaaggctctgctgtcccacgcgcttactttgccacatggcacagtatctgtgtcacagacgcactcttcacaaggacaggtccaggcctgtgtcagtcactgcttcatcccagcacctagcacagggcctgactcatggtgagctggccccgagggtggggacagatccccgaggcgaggcgtcgatggctgcggtctgaggtctctagcggggggccgatgaagctgcttctgcgaagttgggaacccgcgcaccagttttagcagccgcggaaggaagtgctcttgccggggcg

SEQ ID NO: 3 - DNA, 347 bp, Homo sapiens
tttagcagcttaattcaaacatttattaactcttagcttctgtgagcttggtgatcctggctcagggtctttctagaggttgcagtcaagatgttagtctaaagtaatctgaagactcatttgggggagaatccatttccaagcttcttcacatggttattggcaagaagccctagttcctccctggctgttggaaggagggagggctcagttcttcagcatgtgagtcattccatgggctgcttgagtgtccttacaacatggcaactggcttctcccagagcaggtgacctggacctctcagaagagggggacgaacaggcaacagcctcgtggttctccagcct

SEQ ID NO: 4 - DNA, 478 bp, Homo sapiens
ttttttttaaataacagtggagatatattgatctcaacatttcaaggagaaaagagcttgtgaaataggcttgtgatgtttgtgataagggccctgtggcgagctggtgggcaccccatgtagagctggaacacagggacatgatcccatgtgcaggagacagctccagcctctgcatacccgtcggctcactcctgggttggaacatcctcctcattttctctggcatgtccttttgctcaatgaatcattcccggccaggccagacgtccttgctggagagccagctctccgtgcaccagccctgtgattctctaatgccagcttgaaattcggtgacccaccttgggatgctttgccataaaacttgcttctaggaagcaactggtcagtgttaactccttaatgaggctgaagtagacatggtaaaactagttgtggaaagagatcctggttacagccaggcagagttgaaagg

SEQ ID NO: 5 - DNA, 443 bp, Homo sapiens
gccgcaagttgttcaccatgtggatcctctcatcgtcctccgagatgagaaaagcaaagctcaagagaggatgagtgatgggctcgaagatgcacagtgagtgactgaacaaagatgaaaacccagggctgctgggtccctcgttcaatattgtccaaaatcactggaaagggaggaagcggattttaaatttgactctcatttgtgtgtgtatgcaagtcctctctaaaaggacttaggagaagctagggagagtgtttacctacagggaggagactggacaccggaaggttgtggaaggttgtggaagggtgtggtatggcgtgggaaggacactctttactggtactcctttgtatctcttgaatgaattaaggcatgtattacctctttaacaaccaaataaaagttgatccagttctttccttcctgcaaaaaaaaaa

SEQ ID NO: 6 - DNA, 708 bp, Homo sapiens
agggggaaaaaagcaacgcaagccaaccacaaaaacacatataccaatgaaagaaattggtttaaatttcacagcattaacattactttttaagtaaaacagttcattgaagaaagtatgtatgcagcagtggaacatgggcctgtgctttgcagtgactccaacatcctgtgcctgtcctggaaggggcgtgtccccaagagtgagaaggagaagcctgtgtgcaggagacgctactatgaggaaggctggctggccacgggcaacgggcgaggagtggttggggtgactttcacctctagtcactgtcgcagggacaggagtactccacagaggataaatttcaacctccggggccacaatagcgagatttgtaagactccagggcctcccagccgtgaataatctgatggttcctgaaatgactggggaagcagacgcttcgtatggcagttgaagagtgtgtgtctatgtgcatttaaaaccttctttctgtacttacacattcacacgggaagacaggctcattcttgtgcacacttgagagttttacaactgatgaaaattaatttaagaatcagatggagcaacttgacaccagtgggctcaggagcccggggagaaaaatacatcactaatggccagttttccatatggtctgcacgggtaaagaaagtctgcaaaaaaaaaaaaaaaaaaagatcttta

SEQ ID NO: 7 - DNA, 543 bp, Homo sapiens
tttttttttttttagcttgaatgactggagtttagtttttatttctcagaacaaaacagtttgaagcctaattaacatcctcggaaggaacttaacactaaaactcctaacagcttcagttttctgaccttgaagaaagggaaaatgaagagaccatggtgccacttccgaagcaaagcctgaagttctgtgctttagaggtggtgttgccatcctatgattgcaggagtctggccttggcttggtggaggagcattcaaagtaattaactggcatcatctgtccaagagatgggatggataagaagttaagcttccaggagatgcctcatcatttgtgccagtgaccccgcataatttcttgatgaattgtgcaaactgggaagctgatagctcttttgtgttctggaaaaaatgctgccacagaggtctgattttgaagtggctgccaacatcccagacagcggaggtgttatttttatattctactgtctccacacagaaaccttactgtaggggcgggaggcacaatctcccccagctt

SEQ ID NO: 8 - DNA, 566 bp, Homo sapiens
tttttgcacggagctctacagtttactaacagcatttgcaaagttggagccttactccagcctagaaggtggtgagtgctggaatgaatattccatttcacagaaagaaaccaaggcccccagggatgagttggtaaccaaagccatgggtagaggcacagaaattccttgactactctggaaaatgagggagttcacttccaaggaggtcaggaaggaaagcagttgctatggcaacatctttggcagctattttcatccttctctggttcagaacagcttccagcaggttctctgcctccgcctgctccaatgcaggggtgaagtcactgccaacgtttaaccctttccccacgtgggcctggctccagtcagatcgagggtctgggagggctgcagggatctctcagtcgccaatggttgcagacaatccagtaagccacagctgttacttgctgcactgaaccctgcctgcgctgagtaccgaaagcaggagggaggtagccacatctcttgagcccggcgctgcctaaactccctctcggccgaattcttgccctgaggcc

SEQ ID NO: 9 - DNA, 391 bp, Homo sapiens
  misc_feature (362)..(362): n is a, c, g, or t
aattgaactttccacgaattttcctatttaatctttgttacagccttctgaggaaatgcaggcacagaggaagacattcattctttcaaaatcatttgttcaaccaatgcagcggcctactacgagtgtgacatagttctaggtgctggagttcagcgggagccaggtaatttgcctaatagcagtggatttgaagactgttcaagcctggtagcaccagcagaccaggtcggcgcaggcctcagccccgtggcactcacaatcgcctggtgcccgctgggttagactgtggttacaaggtgctggtctggtaggacggctgcggctgggctgttctgtgggctagcctggagctccatctntgcntgcgtgcaggctagcccgctttctg

SEQ ID NO: 10 - DNA, 373 bp, Homo sapiens
ttttttttgatttgaaatgaatttactacatatgaaaaattggaatatgacaaatgctattaagtaacaaatgccgaatttaatcttgaaaaatgcattttcttactggcatattttctccttgtatctgatatcaaatcaatttctgaatgtcttctgtcaagagttttgcatgccttctttattcctttggcccaccatttgccaaataccactcttaggtgttggaaactcttgaattctctggtttgattgaagagcttcacagaagggtctattacagattgaggaatgatccatttcacatctgaagctccaaagagctacttgtgaggaatctttgagtgcatttatctcacatgtgaagctttct

SEQ ID NO: 11 - DNA, 503 bp, Homo sapiens
tttcgaatacaagacattttattgtttcacataaaagaaatccagaggataatgggtctggggttacctggccactgctcatccctgctaactcaggctccttcgacctcttcaccctgcctctccccagtggtggccacccccagctgcaagggcgataacacacgtggagttgttctaggaagaattattttgtgcccagctgaaaatcaggggcttgattatcagggaagggggagaacacatgtggggttaggcaatcagcagtcactgccgtggccatgactggagcatgtcgaatgttctgaggcctccagggggtgcatggcagtctctcagtagtgtgcaacttggcctttctcgacccagaagctcaaggcggtgggactctccagcaggtatcaggcacagcatctgcttcaaagaacacatcggcgacaggggagccccttgcactcactcaggacaggagaatcaggaaaagcagcagatctgcagggctt

SEQ ID NO: 12 - DNA, 585 bp, Homo sapiens
gcggccgctgctacttcccaacccgcagcgccccgattggcctggccgcgcgccagggccgagcctgcagcgcctccggttataagttgaagaaataagaccagtttccaaataaatgacaaagagcttggtattcctgcaggcatcagaatcacctggaggaggagatgctgctgctggtggtggcccagagaccacacattgagaaccactgctctagaaaaccatttgtctttgctgatggagaaacctggctctaatagaagggcttgtatgtgtccaggaagtctagtgaattcgaccatgaatccagacatggccagtggctaaatcctgtgggaagacactgtgcttctctctgacccatgaacactctgctagtcaagctctctgtcacaaagacaacttgaagagacagagtggacctcacagaagataccatcgtcactcttaccaatgcaactgtggtgaacaggaccactattattccttagatcaaaaggacagcacattcaacagcatcctcatggcatgccagcaatttgcataggatgttcacaattaaactttgattatctagtgctt

SEQ ID NO: 13 - DNA, 447 bp, Homo sapiens
ttaaacaatagtgatctgccatttattgactctatgtcacttgcattgtatacataatttcatttaatccttacagtaggtgtcattatttccatttttacagatcagaaaacagactcaaagatgttaaatacttgttcaaatacttctccaaataataagttgtgaagccaggatccgaacccaggtttctgactgcaaagcccaagctttctccactacgccagccagctggagatgtgtcaggggtagttttcactccaggcagagaaatagcataagcaaagatggagaagcagtaaatcgtggaggagcattcagggaagtgagcaggcaggctgttgaagcacaggggtacctgcgctgcctggctctgcgccccgcccgccggcccgccgccccgccgcttctcgggtcgccctcgggctccggcctcgccctcccttc

SEQ ID NO: 14 - DNA, 2609 bp, Homo sapiens
ttattcaaacaacacgtgccatccttcaacaccccaacccacctcccgtgccccctctatgcctcacagcacctgccctggagtagctgacttactgccgttactgtctcctccaaggagtggaagctccgtgagaccagatattttgctggttttgttcactcaagtgcctagaactgtgctgagtacaaaacagatgctcccaaactacgagtaccagtgcatgctcaggagaacaaatgagcaaaccaacggtgaatgtctactatgtgccacacgtcactgctacgcactgtgagggactgagaaggtctgcctgcaggaagttcacgttctagtatggaagggaaaatgagtgcaagggcaggtgcggcagctcacacctgtaaacccagcactttgggagactgaggagggcaaatcacttgagctcaggagtttgagaccagcctgagcaacatagcaaaaccctgtctctacaaaaaatacaaaaattagctgggtgtagtggcgggtgcctgtagtcccagtactcaggaggctgaggcaggaagatcgcttaagcctaggagacggaggctgtagtgagctgagatggtgccactgcactccagatgagtacagaagaagagcaaatgtgctaaacaccaaaccatttccaaaaataccccagtgtttcagaacacacaaaccatgctctactccacccccaaagtaccatccagccttctgtcccacgagtgtccagccccgccaagtcctgacacccaggactccccatgcctctgggtccgcgagttgtgctgctggggacagagatgtcaatgctgccagcacagagcccacccccaccgtcagcttgctgggaccacattctccacagtttttccccagggatcatgctttgcagaagcaccacacacagagggcacacgggccatctgggcaatgctggttgcggccttctgggctccaggcctctgtcctcaagcccctgtagagggtaccctggggcaggtgctggttggaccccagcagaggacacggtgggccagggctggagcccagaatggcctgtctgcagagctctctgaaagtccaggcctgctcagagacacaaaaatcagcaggctgacctggctctcccctggctgctgggagacccactccgcagaccacaccgagggacagggaagctagctcaccccaaccttcacttcccccctccctctggggccttgggccaacttacctcgagcttctcaaccactgcggcctaatccactcgagcgctgtctgtctttcaatgctcagttgaaagtgtgtccctggaaaatggccatctgcgagtgactgcgggtgcactttcccctaggagcctgtggccctgcaggcagcaccgggagcacccctggggctgggcctggggctgacaatggggctccagagtgtggaacgttttctagaactctgtggctgagaaccatgaggcagccggtgcccactcagccaagtcccccagaaggcccctgtgcccactgcccaggcccaccagccactcgcactgaatgcgaggcccccacccactccaccagcactggcactgagacccaaggggagctgggcttgggtccagctgctccaggtgagctggctggggaggaggatgaatagggctcagagtggaaggccacagctggagcgccaatcgtgctgccccttctcataggacccctatgacccacagatggcagggacctcaggagagatgctactctccataaaggcaaaaacaaagcctcacacacctacttttatcaaacaaaagtacaaaagcagttgctgtaagaaatatttcttcagttcattaccacaatttatgtacacgttcaacgccaggtttttcttcactgcttatttctaggaaatggtctatggtggaaacgtcttcagctgcctgctggactgttatttcttgaaaagcacactttaaaatgttctcatagctgagaatgggctgaaaagaaaagaagaaaagcataccaaagttaggagaaagacatacataacgttaccttattccaaagaaataattgtgatagaaatgaaatttgtaattgatcataccattagtgttactatcacttgacatattcattcctaaaaatcaccaagttagcaaaatcacacaaaaccagaggactcatgggaaaggtggggttagggcaaaacattcaaacttcatcagtgacacatgaaaaaagatgaccacccaataatgtcagtgctgttttccagtttggttaagaaattatacatgcggccgggcgcggtggctcacgcctgtaatcccagcactttgggagccccaggtgggcagatcacgaggtcaggagttcgagaccagcctggccaacatggtgaaacccccatctctactaaaaatacaaaaattagctgggtgtggtggtacacgcctataatcccagctacttaggaggctgaggcaggagaatcgcttgaatccggcgggtggggggcagaagttgcagtgagccgagattgtgccactgcactccagcctgggtgacagagagagactccgtctc

SEQ ID NO: 15 - DNA, 1868 bp, Homo sapiens
gtccggcagcgcctgcccaggtgtgctcggagcgtagctgtagcaacggggcgggccttggttcgcctggggcgtggtcgctgtctggggctcccacgtggttcgcctcatgggcctggagctgggcttctctcgcctcctgtcgtctggctgcaccgactttgcccacgccctcacgggagacgcagacactagggacccggggaggcgcccgggcggggccgccatgttggcagagagtcccagcgtcccgcgcctcggttccggaacccgcggcgcggggatggagctgggctgcccttgggcgccgtcctgggctggtgcccaccctggccgcgtggtcaccggcaagaagcccagggcctcacccaggcgtgggggccgggggaaggaccggaccctcccgaagtcgcggaccaggccgggcggcacccgggctggggtggctgctaccccgagctaagctggctgcgccgcatctcacggtccccggggcccgagcgctgcgcctggaccggcgccgagcgaggcggccaatcggccggctggacccacagtccccgcgccataggcgggtcggggctttcagacccggctcccagccctcgaactcggtaaccttggaggtcagcttgagatgactgcgcttcctgcacgcgttgtcctccccagaaacgcccaaaatggcaaagcggcgtccgtggccgcccgaggccctccctgcctgggttctccccagccgaacctcacgcccggcccctccttcattctctcctggccaggctgctcctgcccttgggcgaatccgctgcgtcccacggtcctgactgtggcatttctgcgtgtctggagagctcccccggcagcgacgtcctgcttagcacgtggctcgtctaactctgttcccctggcctcctgctcttctaggggtcatctttaggtttctgctggtctctgccccctgtgacacttgctggtacccaaatgaagtcgtttatgtcacactctaacaagaggccctgaagaagccctcacaaaaacattagcaacggagcgcctgcggcaaacccttctaacctttggtctttatggtttcaaagcagtttatgggaacttctcttttttaaaagattccccttggggccaggcgcggtggcttctgtaatcccaggactttgggaggccgaggcaggtggatcacttgaggtcaggagttcaagactagcctggccaacatggtgagacacccgtctctacaaaaaacacaaaaaaattagcagggcatggttggtggcaggcacctgtagtcccagctacacgggaggcttaggcaggagaattccttgaacccaggaggtggaggttgcagtgagccgagatggcaccactgcactccagcctgggcaacagagcaagactctgtctcaaaaaaaaaaaaaaaaaaaagaaatttcccgtgggcggggcacagtgactcatgcctgtagtaatcccagcacttttggaggccaaggtgggcagatcccctgagctcaggagtccaacctgggcaacatggtgaaacccctgtctctacaaaaaataaaaaaatgagtccagtgtggtggtgtgcacctgtggtcccagctactctggaggctgaggtgggaagaccacctaagccccaggaggtcaaggctgcagtgagccatgattgcaccactgcactccagcctgggcaacagagtgagatccagtctcaaaaaaaaaaaattccccctttcctcaatccccaccccacccccccaccatatgcctgtgacccactgaggcttgcatactctgcattgcaattatgtgttattcttgattaaattcatttattctgg

SEQ ID NO: 16 - DNA, 2345 bp, Homo sapiens
agacctgaaggctgcttccgctaacccgggctggcgctgctaacctcacccaacgatccctgctgtcggaaaatgtccagaggcaccatccctgctatgaagagggaatgccagtacaacagccacaaggggtcttcctgtggccctcaaacaaaaaatcactgcattacatcagcagactgcattggagagaaaggcgccgggacagaaaagcctgagataaaccacctgtcattcttggagaaaaggatgctgcggctttcccgatttggttgatgagatggaaaatcaacccttacaggaaggacatcaccaattccggaaaggatgctgcgccagtcatgtttccgcactctgtgagacgttgaaatctccagaagttgcccaaggttttcaagctgaacacaggagaaatgactctggatctctgaggactgttttcctcaataaggagctgcaatcttggctccaccaccctccctccaaaacatctcctgtgtctggtatctggttatattgtggcctgacagagaagtttctttcggggccagtgaatttaaaaaaaaaaaaaaaagaaaagcgggtcctaccaactcattaatactttaatactttaaatttccctctttatagtaggttgaaatgtcagtatctcttaagaacaatggctgaaccaaatgtttcctcaaaactcacattttttcccaccatttcagaagctgcctcaggactaagctttgatttttttttttttttttaatctggcccaaattcctatctaaggggcctggggagtcatgcccaacaaaccataaattctcatcagatgagttttatttaaccctatatatggtgacttgctttccagtctgactctggcatgacatgtgacaaagaagaaagtcaaaatattttaccccaaaacatgtttctttgccatattttgaaatggtcctgcaaagctgtgctttgtgggggaaaatatgcatctgtaaagaatctctattaacacagctagatctttttcttccaggccctcccaatcctgaagagactgagagtctagcattgttttaaaggtctgaataggaaacatttgtcatctatcatctctaagggtagccattataagacttcaaaagaaccttttgaagtattataatcttttatcttacctgaacatgtgcttcctattgatcccaggtcttcagacaattgtgaactcaaccatttgtcaattagaaaatgtttaaattggctgggcacagtgtctcatgcctctaatcccagcactttggaagaccaaggcgggcagattgcctgacctcaggagttcgagaccagcctgggcaacatggtgaaaccccgtctctactaaaatacaaaaatttagccgggcgtagtggcatgcacctgtagtcccagctacttgggaagctgaggaaggagaattgcttgaacccggcgggcggaggttgcagtaagcagagatcgtgccactgcactccagcttgggcaacaaagtgagactccatcacagaaaaaaaaaagaaaatgtttacctttacctatagcctggaaatccctgctttgagttgctccatctttctgaaccaaaccaatgttatttcttaaacgtatttcattgatgtctcatgcctccctaaaatgtataaaaccaagctgtgccccgaacaccttgggcacatgttctcaggacctcctgagggccgtgtcacgggtcataatcactcatatttggctcagaataaatctcttcaaacattttacagagttgactcttagtcaacatgtccatacctgatgagatgcttcatcagaattctagtggactggctagttcagaaaatggaccagccaggtttgcttaaatgcacccctgcacaactatctcactcctgcttggagccggctagatcaggagacctgtccctaaactaatgtactccatgtcttaagtctttttgttttgttttgtttttgtttttgacagagtctcagtctgtcacccaggctggagtgcagtggcgtgatcttggctcgctgcaacctccgcctcccgggttcaagagattctcctgtctcagcctcccaagtagctgggacaacaggtgtgcaccaccatgccctgctaatttttgtgttttaagtagagatggggtctcaccctgttggccaggctggtctcgaattcctgacctcaggtgatccgcccacctcggcctcccaaagtgctgtaattacaggcatgagccaccgcacccagcccatgtgtcttggtcttttttactaatattgattataaaatggatgaaactttggat

SEQ ID NO: 17 - DNA, 2529 bp, Homo sapiens
agaaaagaggacagagtcattttgctcagaagaaacagcatgtgcaaatgtgccacaaccattcaaagcaagttgctgcttaagtcttgcccacaacttttcatctgaaaagtggagtagctgaagcagccaatgctggtggttataattcttgaggatgcaacagtaagaacggaaaggaaataacccccgaagcctttgcaactaaggacatgtatccttcagacaagtgtttactgggcaacttcttcgtgctgtaattgagtgtggccgattgctcacaaagatgtttgcaaaatccctcctgtcccctaactcacttctccttgcagtgtcactctgccaacttctcctgtcgattggtgaagactgtttctcctcccccttgaatatgggctgggcttgtaacttgcttgaccaatagaatgcagagaaatgaaatgcagccttcaacattcaaggctatgctcaaggagtctaaccctgtggatatgctgttgtcaaatgagggagcttcgattagcctgttgaagacacacagacgacccgacaggcaataccaacattcagatatgcaagttatgctgtcttagaccatgctgcccaggtgaacttttagacgactgcaatttgtgagtgactctaggcaagaccagaagaaacttctagctaaatctaaaccaaatgccaactcagaattgtaagcaaataaaacggttgttgtttaaagacactaacttttggggtgttttgccacacctcaatacataactgttacactaattatttttcattgtgaaatcttcagcgtcttattctgtaatcaaaccagaatgtccttgccttcttagcactttgccacagtccagtggggtgaatagacagcagctgctcatttatgctcatgctcatgttcataatttgcatttggactaaaaggagctctgttctccaggacagaactattctaaagctcttcaggaacaggaacaggagtccatcacagacagcgaaatcaaaagcgccctacatagaggcatccagactgagagctgaaagggatctgggaaatcatttagtctcactccctcattttccattcagaccacagggatgaaagaacttgctgaagttctcaccgctctgtagtggcacagtcagaacaaggatcccaatctcctaactactaacataaaatgctttcagcatgcaagttgagcatacaatgggacagtttaaatttaaatggtgttttgagcaaggggagagtctaattaattacttcaaagtataattttggagtcaccagaaaggcgttaaagacaaaaaaatgctttggaacctaggtgcttcatatctgaaattggggcaggtacaggtgtattcctaccactaaagggtcattagttggttgggtagtcctttagtgttcttggtactaagcacaaaacatgggtaagatggttcaaggctggttgtggtggctcatgcctatagccccagcattttaggaggccaaggctggaggatagcttgagcccaggaattcgagacaagcctgggcaatatcgcaagaccttgtctctacaaataataatttcttaagaattggctgggcatggtggcacacacctgtaggctatttgagaggttgaggtgggaggattgctttagtgtgggaggctgaggctgcaatgaggtgatcacactatggtactacagcctgagaaacagtgtaagatcctgcctcaaaaaaaaaaagtttatgttctcaaagtgctcataatctagtggtagtacagtatttgagatattagagcagtttctcctccttttgcaactaaggacatgtatctttaaagcagaaggaatggcagagtcgtgtaataaaccctcaagtaccattacttagcttcaacaactatcgacactctactgttcttgtttcatttatgcctcacctccttcccatcccccacttgaatattctcatcctttttttttacagtttttaagataacaattacataactgaaatgcacaaatcttagctgtacagttttgacatatggatacacctgtgtaaccaatgactgtatcacaacatagagcatttcatctccccagcaagatccatgtgtcttttcctagttaatgcctctttatttctgagatggttattgctctgcttttgtttttcatgttaggctagtcttgcctgttctagaatttcatataactgagaacatacagaatgtactcactagtagtgtctgactttttcacaaaggataatgtctgtggtattcattcatgctgttgtatgcatcagtagtttattttctttttactattaagtagtgttctaaggactattttaatagcatcccacaaagggggtatgatatgttctatttacattattatttggtttgtaatattttatattttctcttgtgatttctcctttcactcatgaattattataataaattttttaaagtgtattataaaaaaaaaaaaaaaaaa

SEQ ID NO: 18 - DNA, 2034 bp, Homo sapiens
aattttttgtatttttagcagagacggggtttcaccctgttagccaggatggtctcatctcctgacctcgtgatccgcccgcctaggcctcccaaagtgctgggattacaggcgtgagccaccgcgcccggcctaataaaggacaatttttaatagccactggactatttaagtgatctcttcttctgtcagtttcttttaccatcagaggacacagcagcaaggtgccatcctagaagcagagagcagacccttaccagacaaccaaccagctggtgccatgatcttggacttctcggcctccagaactcaacaacaagaaacaaatttattagctaacctaaccactaatgacgcaagagacaattctaaggactttcaaaacagcaaagtaggagcagctgctacctctagggatgagggatgcaattgtccaattattggtgaaattgtcatttcatgctattggctatttgaaattcctcctctaatttcagaataaatcactgaaattgacatcggccagtctgaatttcaagaaattacctgctgaagacaagagggatctcttcttcagatttgcagtctggggaagacacagcctctactgtactttagaacctgagatatggtggtggagggagccctgggtcgagtggtaagattcacccttaggttagtattgacgtaaggtgacgaggagctgtagacaaaagattgtaaccataagaacttcatagtttttgtattttcaccgagcttatatttggtgtgttttttgtcttttctttatgattatcaataaaatgcttgaaaggagatgaggttggggaataatttttgggaataccacaaaagacacttttgtgatggaaatccttaaaaagacacaatccattacctcattgggttcaaaaggcaattgtgaactactgtggagtttggaaagaagcaatgaggtaatcaaggatactgttgacaatctagcttatcctatggatggaaggaaattgaaactaatggaggcgaggctggtaaaacaatagggtttgagacaattctgtggcattagaaatgaaagaggaaggtgaatccgggagacggagcttgcagtgagctgagatcgcgccactgcactccagcctgggcgacagagcgagactccatctcaaaaaaaaaaaaaaagaaagaaagaaaagaaagaggaagtaataatctgtgaaatttttccttaggaacttattggcaatttaaaaatgaatttgttaagccatgctggttctgacccaaaagccattccccagccttcctcactcccctctttcactactggcagagattgtctctcattttacaagctgaaaatgccagatgcttgcttttacagtcttccttacacccagagcatgtgcatatgtttaaacggtcaagaagaagtcataacatgggtgtctgggagagcttttatcccacaaaaacaacacttcactcaaaaaacaaaccaaacaaagaaaaattctccttcctgccattggatgtgaggctcagacctctagtaaccattttgtgaccacaaagcaacaagcctgaggaaaagtcctacacgctgagcaacaggcagaaatattgctatcgctgagttgcagaaacaaatctagagatgttttgcttctgtaattattttttatgggagattacaagtgggtttactgttcacttttcaaatcttatttctctatgatgtttagcttgggtaaattttaccttaaatccacttttttatgtaaggtaacatatttgtcggtttcaaggattaagatgtgggcatacttggaggccattattttgcccaccacaggtgaaaaaggaagtgttattcttaaatcatttggaaggatctctgtgtaaatgcaagagcgagacaagaaaatgctgtcattcttttgatatggactcgaatttccacttcatggttgtctgcttcctttttagagtattatttatcctcctaataaaaagaaagtgaaatttccc

SEQ ID NO: 19 - DNA, 2840 bp, Homo sapiens
tacatctatttaaggttacatgatattttgatatccattatgtgttgtgaagtgattaccacaaccaagctaacaacacatccaacacctcacctagacacctttctggtgtgtgtggtgagaacacttgagtctaattcctcagcaagtttcaagaaaacagtatgctataataattattattattttgagacggagtctcgctctgtcacccatgctggagtgcagtggcgcgatcttggctcactgcaacttctccctcccagttcgggcagttctcgtgcctcagcctcctaagtggccggggctacgggtgggcaccaccgtgcctggttaatttttgtgtttttagtggagatggagtctcatgttgcccaggctggtctcgaactcttgagctcgggcgatctgcctgcctcggcctcccaaagtgctgcgattataggtatgagccgccacacctggcctgctattattaattatagtcaccatgctgtaccttaggtctctggaacttttttcttttagagatgaggtcttgctctgttgcccaggctggagtgcagtggcgccatcaaagctcactgtagcctcgaactcctgggctcaagagatcctcccacctcagccttccgtgtggctggaattacaggtgtgtgccaccatgcttggctgattttaaatttttttgtggagacggggtcttgctatgttgttcaggctggccttgcactcctggcgtcaagcgatgctcccgccttggcctcccaaagtgctgggattacaggcacaagccattctgcctggttaaaacgtgtttatctgaaagctgaaagcttgtaccctttgacctacatcccccgccttcccctgtgccctcaccaccataaccactgctctactctgcttctacgagttcaattcttttttagattccacagataagtgaggtcatgcactatttgtctttctgtgtctggcttatttcacttagcataatgtcctccaggttcatccattccagaggggtgttttaaaagacaatcttggccgggcgcagtggctcacgcctgtggtcccagcactttgggaggccgaggagggcagatcgcctggggtcgggagttcgggaccagcctggccaacatggtgaagccccatctctattaaaaatgcaaaaattagccaggtgtgatggcggggacctgtagtctcggctgctcgggaggctgaggtaggagaattgcttgaacctgggaggcggaggttgcggtgagccaagatcgcgccaccgactctagcctggccgacagagcaagactccgtctcaaaacaaaaacaaaaacaaaagacaatgttgagtggtgggtcttctgctttaacccctccaacacctctgactcccgccttgtctccctccagccatgcgggttcccaccctccagcctcagcctgtccccatctgtgtccctcagactcagaggtgtcctccccagagacgcagacatgagcctctgccaccctgtattacagtgccctgcagtcaccgcctgcctgtgcatcccttgccatgctgtgacctcttgtgtctctgttttcttcttggtaaaacggggatacctcagagggcagctgtgcaacaggacaaattcacacacgagacactcgggatgctcctggcacacagacagggtgaagcatcccttgtccccattgtcaccttctctattagctcgggctgccataacaaagtgccatagactgcgcggcttacacagcagaaatttacttcctcctagttctggaggccagaaggccaagatcaaggtgccagcgaattcagtttctgtggaaattctcttccgccttctcactatatcctcacagggcctcttctctagacatgcataaagggagacagatccctgctgtctctctctttttttttttttctgagacgaggtctggctcttttgctcaggctggagtgcagtggcgcgatcccgtctccacaaaaaacataagagttggccgagcacggtggcacgtgcctgtggtcccggctactgggaaggctgaggggggagggttgcttgagcccgggaggttgaggctgcagtgagctctgattgcgccattgcattctagcctgggcaagagagtgggaccccatgtcaaaaatacaaaagatatgttgaagtcccaactcctgataactcaaatgtgactgtgttgggaacatctggagtccttacagagataatcaagttaaaatgaggtcattagtgtgggtcctaatccaacaactgacgcccttatacaaaggagaaacctggacacagacatgcacagaagaccatgtgaccatgaaggcagagatcagagtgatgcttctggaagccagggaagattgccagttaatgaccaaaagaagccaggagacaggcctgcaacggattctgcctgaaggctcccagaaggaaccaaccctgacaacaccttgatcttggacttccaacctccagagctgggaggcgacacaattctgttgttggctgcagtggctcacgcctgtaatcccagcactttgggaggccaaggcgggagaattgcttgagcccaggagtttgagaccagcctgggcaacacagtgagaccccagctctacaaacaaatataaaaaagtagctgggcatgatgggatgcacctgtagtcccagctgctcgggaggctgaggctgcagtgacctgtgatcgcgccagtacacttcagcctgggcaatagagcaagacctcatctctgaaacataaacaaaaaaaccaataaagtctctgttgct

SEQ ID NO: 20 - DNA, 2315 bp, Homo sapiens
gtttaaagatggcggcggaggaacctcagcagcagaagcaggagccgctgggcagcgactccgaaggtgttaactgtctggcctatgatgaagccatcatggctcagcaggaccgaattcagcaagaggtgaggggctgcagtgggcgagggaggcagtggccagcagccccattgtggaaatgcataggctgggcatgaggcctattgtctgtctctactttggaagctccctcctccctaggctatgctaggtactggtgcttttctgggggtcgctgtgccaccgggtgaccagcccagctcttttgtagttattctgtctgttaggcatgggtcactccatctccctgctaggacatgacagatccctgcctcctccagattgctgtgcagaaccctctggtgtcagagcggctggagctctcggtcctatacaaggagtatgctgaagatgacaacatctatcaacagaagatcaaggtgggagcctggccagagcgggtgggaagcaccctgggggtggggcaggagggtgcctgcttcagacttgcttcctgctgggtctgtcacctgagggagtagggtgttggaggacacttttcgttgctggttcttgaagtgcgtaggctgaggcctcaaaaacacattgattcaatgcttgaacctgggaggtggaggttgtagggagccaagatcacaccattgcactccagcctgggtgataagagcaaaacttcatctcagaaaaaaaaaaaaaaggtggggggcaggcacggtggctcacgcctgtaatcccagcgctttgggaggccgaggtgggtggatcccctgaggtcaggagttcgagaccagtctggctgatatggtgaaaccccatctctactaaaaatacaaaaattagccgggtgtggtggcaggcacctgtaatcgcagctactcgcagggctgaggcaggagaattgcttgaatccgggaggcggaggttgcagtgagccaagattgcagcactgcactccagccagggcaacagaagactgtctcacaaaaaaaaaaaaaagacattgattcaaatctagactctgctactcagcagctgtatccttggcaagtcctttagtgtctctaaaatgggtgttctcatctataaatagggacaataaaagcatcttctggccgggcacggtggctcacgcctgtaatcccagcactttgggaggttgaggcgggcagatcacctgaggtcaggagttcgagaccagcctggtcaacatggcgaaaccccatctgtactaaaaatacaaaaattagctgggagtggtggtgcgcgcgtgtagttccagctattgggaaggctgaggcaggagaattgcttgcatctgggaggcggaggttgcagtgaggcaagatcccgcactgtactctagcctgagcgacggagtaagactccgtctcaaaaaaaaaaaaaaaaaaaggcctggcgcggtggcttacacttgtaatcccagcactttggcaggctgaggcgggcggatcacgaggtcaggagatcgagaccacggtgaaaccccgtctctactaaaaatacaaaaaaattagccaggcgtggtggtgggcacctgtagtcccagctactcggagaggctgaggcaggagaatggtgtgaacccgggaggcggaggttgcagtgagccgagatcgcgctactgcactccagcctgggcgacagagtgagactccgtctcaaaaaaaaaaaaaaagcatcttcccatagggcgattgtgagattgagggaggtgcaggctgggcaggagctgatgatctcggtgcccatacgggggctgaccaggctggtgctcagtggtgtaggagggttgccagagggctgtttcccacagcttgcctcctggatctttgtggagaggcaggcagcagtggggtcaaggggccacagatttcctcaaagggccctgctgcatcactcaaacggaggcaaacttcaccacccactttctaggtctgtgagctgggaggatgacatgtgaaatggggccaggcatggtggctcaagcctggaatcctagcactttgggaggctgaggccagaggatcgcttgagcccaggagttcaagaccaacccaggcaacatagtgagaccttgtctctccaaatcaaaaaattactgggtgtggtagcacatgtctatagtcccagctactcaagaggctgcggttggaggatcacttgagcccaagaagttgagccatgattgtaccactgcactccagcctgggtgacagtgagacctgtctctt

SEQ ID NO: 21 - DNA, 1929 bp, Homo sapiens
aatgacttccagctggagcagcccagccagcctgcgcgctcactcgcttgcttgcgctgggcttcagggcgcccgggaatttgcactgtctgttaaaacgcggtcattgttttgctttcaaatcgaacttcacgacagttagcaacttcaagaacgcttccaaatggataaatctttccagaagatttcttgcttcaaaacagctgcatttttggaagaaagtccacaactgacaactaagcaaaaccctcctggtggatagcataaatctgctttgttgaagctgctcatttgtctgatctatgagtccagaagatgcaaactcccttcccagcttatgcaaaaatacttccagtaaaaacaagccctttggcccttttcttttgtcagtccagatttcagcatgttcttcgctttgttattcatcattttatgtatttggacccatcctcaaacagactttcaaccgtttaaaggcaaggagggactctttcctaaacctttaatcccctggcccagctgagtagatgcacagtgtcttttgatgatggtgatatagaatgattaataaccacacaagccattcggttggtcaagtcactgtttattggctcaaaataatccttggtttctgtatgtctacagctcacttcctaaacagttaaatccggagaagtctaatagataatagttggtgatcataaatataaataaccaatacattagaatgtgtagtcaaaataggaatacttgcttttcacttatttcccacgggtcgctgctggtggtagtcaacacaaataaaaatttgaactcattttgttattcatctaaaatgagaaactcggtgaaacattgattttctaaaattgctgcatatttaaatgtgattataattgctttttagcattttaaaattgtaacatcgataccaaatgcttaagaatctaaagaatagtctattggggctggcctattctgtgtaaaatggtctgtatcataatggatgactgattatcccatccacattatttggattcacttaatactaggttttaacgttaggagaataaaaccctcaagaaacctaacatagatttgtatatttagttttctccttccttatcatcttccaccagacttacaggtgttccacctgcttgtagtttcggtaataataccagctggcggtggttcctaactctagctacactttaaaattacctcgtgcatttaaaaaaaaaatgcagttgtttaggttcttcccctagatattttaatttacaaggtttgggatgtggcctgggcaccgatttttttaactttttatttgaatgtagacttacaggaagttgcaaagatagtacagagaggtctgatagagcctccactgttggttacatcccgcatagctagagcacaataataaagccaggacattgacactgagataaaatgtgcctgtgattctgtgtcaccttatccctgaagactcttgtaatcattaccacaatcaaaatacaaactatttcaacaccacgaagatctctctcatacggtccctttatggtcgtccctttctttcccccacaccatccctaaccctttgcaaccatcagtctgttctccatttctataattttgtcgtttggggaatgttttataaattggtcctcacagcttgtgaccttctaagattagctcaccatcccccccacccccccaccccacccaactcagcagaatgccctagagatttatccaagttattgcaggtatttctagtttgttcctttctgttactgagttctacactatgggtgtaacagtttgtttaatcattcagcattcacctatcatacattttggttccttctttttttgggcggggggagtgcctattacaaataaaactgtcttgaaaaattg

SEQ ID NO: 22 - DNA, 2294 bp, Homo sapiens
ttttggtcgtctctgccccagtcccttcgccgcgggacgcgcgagacgggagaaggtgcgggaagcgggaagcaggagcgggagcgcgcggccctggcacgcatagggcggcggagagggcacgagcagggattgagcacctgctgtgtgccttcacgctttacaaaaggattttcgttcgatgttcactacagcccctgcccgggggtactgatgccccatttacagagggacaagccggatttcggagaggtgaagtcactcgccgaaagtcgcaccgccagggtctgcgtgacaccctaaagcagtgttcagttaccccggggagagcgcgatgaacttgaaccacttgttggctggttcctgctcttgctcgttttttgcggatcgacacatagtgggcgctcaggaaaataaatgttggaagcttgagattgaacttgacagctcgaccctaggtacccgccacgaatccagcccagcccgcggggcaccgggtttctccagacctcgcagggaacatttgcggatgggctggtagaggaggctcggacatcccagttccgcacccgcactcgaccaacgcgtggtagcggaacccctgtcgtagcgaggcacagactgggttcaagtcccgactctgccgatttcagcctgagtgactttgagcgagtcactttttcccgtcgaaacctcagttgctccatccacaaaatgggaaatatgaacagccaccctaaaacggtgtggggaggattaaacgaaacaacgttcccaaaactctaaacttacaaatgttgtctcccgtccatcccataaacttaggcgacaaacctggcgcagggtgacctgagacaaaggcttcccggcccctgtctccaagtcgtccatccctggggcgtaggcacgttttagtgagccctgtccggcgaaacccgaaactgcggccaccttggcagcggtgggccccaaaaggaaatattcaaatattctgaaactcgtgcatgatttaggactgacatttttacgtttaattttcttgcagttttattcttgctagtaaaggtaattcgttagaaacccttaagtgcagtttctcctctgtgtcttgtttaacttttcgtgttagcgaaattcacaaatttgaccaaggaaccggagcgcggccgtcctcgccgggattccggtcatcgcaataatctggctctcggcccctactcccaggcacggaggtcgaagagacgggcttcccctacaccgccctgtgtagatgtagcctcttcgtcccggcagccctccgagattccctgtgtccaccggagcgaggagaggtgtggggctgcagcccagaactcagcttcctggacctccccaaactcccgcctctccgggattaagggagtaaatccctgacgccaaagaccaggtcaaggacaagttctcccccgccctactcccccctcctgggcggggattcatctcccttccggatgaaaggtactaaagagccgacggggggccgcggcgggccccaggcccttgatgttccggttgaacaggtgctggtgaaaaggaggcggacccggcggaagagtctgccagggggcagtgcgccgaaggggaggcggcctcctccacccccagtcccccggcccgtcttcccttctcctctctgtttgcccctcccccgcaggaagcgttcccggccgcgaggtctttgaagtgtcgttgaagcccccagggctgccttctcccctacgccacccgaactcccgctgtggggggcgggtgacctttagtccccacgagtcgtccccctttaggaagtttttgggggtcagatctcacccccccttgcccacacaggttgggaagaagactttgggccgacgcccctcacttctcccccagacccaattgcagggactttagtcctctggagtgctgcgtgtgagttaccttgtgtgtctgtgtgcgtgcctagaggtcaagtgtaactggtgttcgtgagcacctcgtggttgcgggtctctaactctcgtgggtctctaagcgcacccgcggggctggagcggaggttcgtgtctctgggagggtcagtggtgtgactgaagctgggagttagctcgtgtctgtgggtgcctcggtgtgtgtctctggggctctgagtccctgtgcgtgcgtgtgtgtctgtgaacccgacaggaagctccccgagggcaggaatatgttttgctctctactcgatcccagcgactggcaacgagcgtttaataaatatctgttgaatgaat

SEQ ID NO: 23 - DNA, 3931 bp, Homo sapiens
atacccacgcccggccggggaagctgcttgccctcttctgcctccccactggtcctgagccccctgctaccctctaacccaggcccagaccctcagccctcaggataggcttaggtgcagagctggctcagctgtgcgattgtaacccctccctctgagcctccatgtcctggtctgggaagtgttgtgaggtttgtaaaatgtgaggtgtggatgtggccccaggctagccggggaagaagtggggatgggacgtcctgacctagacgtggaggcggaactcagtcgccagacggatgtttcctgttggcgtgaacaccaggggaggagtcatccctaggtggatatcccccctgccaggccccaggaggcacctggagcttcctgggtgccctgtggggcctgggggggtggggacagggtgtgtggtgtgtcgtgctggtgggcctcggtggtgggcacacattgttcactcagctgtctcttgtcttctctatttctttggcttctctccccgccccccttgccctgcttttgcccggggtttggccgcgggcagtgccaacatacggcccttacggtaggtcccgctcttggtctgcatgtctgtcctgcatgtcagttctggtcactgtcactggcatgcgtctctctgtagctcttctccgtgtcctcatctcctccccaccggcaccccctgggtgggagcgcccagagcacctgtcaagggccaccctctgtagagcttgcctgcttccgtcctcgtttctcgttctggaatatgccccgccttcttctgggggcacagtgctgttcatgggatatcggggcatttcacgatgtttccaagagttcacaagctgagtcaggaatcctgggttccaggggtgggctcgtcccttacatccaggtgtcctgggccacctttctgctctgagcccctgaggatggggacagttgattagagagatgaggaggaagccctcttagagggagccagtgagtcgcatctgctcccctgggggtgcaggccagcctggtcctgatctccagagtagagggagcacagcacttcccccggtggtgggggacgcgcctggcacctgtggaaccgggcacccctggcttccaggccctctggccccacatgccccaacttcagtcagtgctgagcctgctctggcctctctggggcattctaggctcactgcccatagcttgccagtctccaccctgggtcccttccctgcagccagcccagctcccagtgggggtttatgtctgctcctcttcacagtccagcctccccttggggctccccagccaaagaagtccattttttcctgccccttctcatatcctggaccccattcgctgtcccagaagggatggttgaagggcatagctttgggttggggggtgtctgtcccaggatcacgggcaggggctgagccacctccctctcaccgtcccagcctcctcacctgcccctcccgctccctgcccggcaggccgctgtgcccccatgaagagcatctccagcagcctcaaggagaccatgaacccgcacgacatcgtgcaggacgccatccacaacttctcacctgcctaccagcagtacacgcagcagtccaccctggagcctgggcccacctggcgtggtggcgcccacggcctctcccgctcccacagcctcagtggcgcccgcgacaacgagaagactctcctgctcagctctgatgatgaattctaggtgcgggctgcagtggcggaagtgctggcgccatagccacggtcaggctgtgccccacctccagcctcaccaccaggccaggaggcagctggcacagtgctcacgccgcctttatttattggaccagaaacactcacatgtcgcttccagaggaacgggggacagccaggctcgcccatgggccttcaggaatatttatacatggcccagcctgcactgcccgggcgagggcagaggacactgggagcaaggcttatgcccctgctgcccgtcctgtgctgggggcatgctgggaccagccgcacccaggccccaatgcttgtgtgtggaccagcggctgcagccttctagcccctcctccccgcgagactctcaggctgaggtcggcaagccgtggctcccccacacaccgtgcaataccctgtctgacctgggctcttcccgcctgcatccctcccctgtccacctttgtccagtgctagattcacctcaccccgggcaggagtggggatgtgggcgctctgtggtcctcccctcctgacccaggcctctgtggcatgctgcaaggatcagagccagacaccaggagtcacaggccccacccaggaagggcattcagggcccctgggcaccgcttctgttgaagcaggggcttctgggcccctgggtatccccacctgtcgtggccacacctctgcctgcctcatgcccctttcccctggcctaccaaggacagcccacagcccgcactgccggctcacttgggtccttcctcgatagctttgggcagagcccttgcttcctggctgcttcagggctcaggggctcccagccctccttcccaggctgatgctgggtcctctctctctttggggcttctccctcccgtttcaggggaaaggtctgagtctccacgtttcagaccagcttctgggggaaggcagtccggcagggagaccgggaggggtggccacacagtggggagctgggaggtggggggaatggtcccagactcctctcggggcccctatccacacagggcctggtgttctaccccatctggcccctggcccatctcttctgtgccttagtcacatatgaaagcgcccctccctggctccccatctgtcccacacgctccctggggctcttagttcagctgctggcactcgcaggatcctgcagtgctgggcccagagcccttggacaggcctcaggagtggtcaggaccaccaagcccctcctctccccctccacacctctagacctggggcctccggaacccccagcaggctgggcttatactagctcctgacttaggaagagcctcgtgtcacaacacgtgtccctacaggcaaagtgtcctggcatttaaaacccagattatccctgggtttgggctgcagtcacctggagaagctggtagggtaagggagagggaccctgccggtgttcactggggattctttcttttggtccttcctggaatgaacaggttccctccctgccacctgtgaggagagttggggcccagccgtcttcctggcctccttcctttcctcgtggcagaggcctgcatgtgggtgccagaggccagctctccccctccatcttgggggggcggagcagttgggcccaagctgcccgggagggtgggtgcagacacaggctgaggaccagccctggccctgccccgccatctgctttcaccaagctgtctctccaccgtggcttcccttctccctccaggccaaagtgctgctgattcccactcccttggttttcgcctgcccagcgttgctgtttgcgtggagggtggggggagctcagtggcagggaatcagcggtccgtggggtcgtggggacgggaacatgtgcccgaccgctccatcccctcctcctccttaggatgcataacctaccttgtcttttttttttttaattttctttccaggtagagtagctctttgtacataaagaatacttgaaaaattaattgtatgatgtatgagaagacagagtctcctagttttgtatcttgttgtatgactgccatgagttccaccagaaagccactctattttggtctctgtgacattttaaatgcgtgacagaagtgagcaaataaagtgaggaagaaatctatatatgagataatatagattgtattgaaatctc

SEQ ID NO: 24 - DNA, 1603 bp, Homo sapiens
ggtgacgtttggtctgagctgacctttcctcacaatcgtgaatttgggctgtgaggatcaggccttgtcagaacccgagatgagaggagagagatttctggttcagtaagggtgagagccaggctccagactatagtgaattttccgaggagatatgtatgacctacagtgactctgacgctaaatctgcaagttcttttaacaccacggaaataatgtctaaatgctcaagacaaaatcacttatggtgttgtctacataccttatgacatcgtgtatggcaaggcactttaaggtcagaactacagatgtcaaactagttctcttaatttcagggatcacatggtcaggcatacactggttccaaaaatctttactatagatccgaaagcattttcctgaagaagtcctgccagctcggccacttcgctgtaatgcctcgctcctacaaaaggaaaaaaacccaaaaattccccaaaatgttctgtgggtttctcaggtaatttactttgtctccccaaaacagctagaaattacctaatcagttgttctttgaataaaaagatttgacatagagacttactttgaaattggaaccacctccaggatgtccaaccctaatctggggttgtgatttaactgcttcacaaagccaccatctaccacatatctaagaagaatcaaagcaagttaagtttctggaaaataaaagaagacttgttaattgttaaccactttggtttctccttttctagcaaactagcaggtacacaatgccttttatgttaagtgtaaaacttgaaagtcaagaaattggaaccctcgtatgttgttggtgggaatgtaaaatggttcagccgaggtggaaaattgttcggaggtcctcaagaagttaaacacaaaattaccatatgatccagcaattccatttctaagtgtacaccaaaaagaactgagaacaagtactcaaacaaatacttatagaccaatgttcatatcagcactatgaatcacagccataaggtaaaaacaacctaaatgtccattaaccattaatggaagaagggatgaacaaattgctttgcatatatgtatgtagtatatatacacacgcatatatacacacacaatggaatatatatatatatacacacacacactatgcaatattattcagccacaaaaaagaatgaagtactgatacatgctacaatgtggacgaacctcaaaaatattatgctaagtgaaagaaagcaaacacagaaggccacatattatatgattctatttgtatgaaatatccagaatagaaaatacctactgcttcctgctgacacatctaaagaattttttaaaaaagaaaatacgtacaaacaaaaagtaagactggtagttgccaagggctggggtcgagaggggaatgtggagtacctgcttaatgggtatggggttttattttgaggtgttgaaatattttagaactaagcagaagcagtggttgcgcaatactgtaaatgtactaagtactacttaatttttcattttaaatgattaattttatgtgaattttgcctcaatacaaaaatacacaaacttgaaagtc

SEQ ID NO: 25 - DNA, 2182 bp, Homo sapiens
ctttttttaatgtcttatgattatgctcttctattcctccattactgtcatcttttgtaatcaacagacattttccattgtatcttttaaaattcctttgttatttcctttgccatatgtattttaaatgtattctcttagtggttagcctggggattacaattaacatcttaaaacaatctcttttggattcatatccacttaatttcaatagtggcagtcaagagtcactccaatatacctttattctctcctcactcccttgtgctattattatcatacaaattacatctctatatattataagcccatcaacacagttttgtaattattgctttaggcagctgtcctttaaaacagaaaatttacaaacaaaactgtttatattgtctttcaccatacctacatagttacccttacctctgctctttatttcttcatctggatgcaagttaatgtctggtgttctggtcgggcgcagtggctcatgcctgtaatcccagcactttgggaagccgagtcgggtggatcacctgaggtcagcagtttgagaccagcctggccaacatgccgaaaccctgtctgtactaaaaatacaaaaattagccgggcgtggtggcgtgtgcctgtaatctcagctactcaggaggttgaggcaggagaatcgcttgaacctggtaggcagaggttgtggtgagccgaggtcgcaccactgcactgcagcctgggtgacagagtgagactctgtctcaaaaaaaaaaaaaaaatctggtgtcctttcattaaagcctgaaaaaactccctttagtacttcttttgggaaggtttgctaatgacaaattctttgtttatctggcaatgtcttcatttctccattaattctgaaaaagggtttaactagatagaacatttttggctaaccatctatttcatcctctcttctggcatccatgttttctgctgagaagtcaacagctaattttactgaagttcattcgatagatgattttcagtttgtggtgaaattccagtttattatcagaattctaaaattggtttattttagatgtattgcgcatattttcattgttttagagggagacagagttcactgaggcccccactctgccgttttggaactgatctcatttcattttaattatttaggtagcatattttaatataaaccaactttcacttaatagttattttttctaaaatttcttaactattgtctgtttggctaatttttcagttgaatgttagaatcattttcttaagctccaaaaagaaattctgatagaacttgggatgttattttgccaacagatcaccttcttagaacattcaggtttcccattctctcccacttttatactgttcagcaaacttaatgaatgttttcatgtagattgcattgtattttccttagacatttcaaatgttctgttgtgactgtgaactgggcttaaagtgctgttattttaaatatgaatcaatacatcaatattttctgggatgaaagaaaaacactcatctatcaatgagaatcaggagattgagatcacgaggtcaggagattgagaccatcctggataacacagtgaaaccccgtctctactaaaaatacaaaaaattagccaggcgtggtggcgggtgcctgtggtcccagctactggggaggctgaggcaggacaatgacgtgaacctgggagacggagcttgcagtgagctgagattgtgccattgcactccagcctgggcgacagagcgagactctgtctcaaaaaaaaaaaaaagttaatggaatcaggagggttcattctgtaggtaagaggtttgctttttttttttctttgaaacaataaaatatctttgttcaatttaaatcttgaggccactcatggtggctcacacttataatcacagcactttgggaggctgaggtgggcggatcacttgaggtcaggagtttgagactagcctggccaacatggtgagacccccccccgccatctctaccaaaaatacaaaaattcaccaggcatggtgatttgtgcctggaatctcagctaatcaggaggctgaggcagagaatcacttgaacccgggatgcagaggttgcagtgagctgagatcacaccactgcactccagcctgggtggcagagcaagactgtctc

SEQ ID NO: 26 - DNA, 2186 bp, Homo sapiens
ctccgcgcgcctgcccacgcgctccggtactcgctgctcgcggctggccggctcgggattccgggctttcttcccgagaccgcgtcccccagctgggccgaaggtggacgctcaggggctggaggctcagcggaatcccctgcgttcagtagccccgctctcccctgtcccgaaggattactctgcccctcagcggttccagtgccctcaaagcaatctgtctctgaagtactggctatcttctgagcgtgtgccagaagatccagctttgttgaaaagcgaagccgttagtcccttaatacaaaggagacaaattgatttatgcctggggcaccatcaccaaaagaagaggaaatggatgcaagccttcccagaacaacagaaagagtctcgctctgttgccaggctgaagtgctatggtgtgatctcggctcactgcaacctccgctttctgggttcgggcaattctcatgcctcggcctcccgagtagctgggattgcaggcacatgccaccacgcccagctaatttttgtaatcttggtggagatggggtttcaccatgttggccaggctggtcttgaactcctgacctcagataatccgccagcctcggcctcccaaagtgctgggattacaggtgtgagccactgtgctcagccaaaaaaacttgcattttaaagaaagttttccagaactgggtttgttccattcaataagtagattgagttacaactatgcacttagcttcatgtgacactgaagggaatatgaagaagaaagaagacaaattctgcttatactctgataggacgacctctgctattttccttctgaagctttgcagagagcagtgaattgtaatgaaaggagatttgggagtaaagactccgtgaggtattgaagtctctaggggaacctcattatagcattcctcttcccagcctggattctgaacaatttgagaaataaaaagcaaatgtgaagcacactgaggccaaagtatcacctttagaaccagtaaagatgaattggaattccaggcatggcaggccaaggcagacatcatccttagagacagagtccctggaggggaagaggaaggagataaagctgaagcaagcaagccagggcaagtcactttgacaccccagggacagaaagggaccaggagtatggtcagctgcaactaggaactggggaaagatgttcccgcatcactggttttttctgctcctcagatgcgtgacgttggatgagtccattaatccctctatccattatcatcttttctaaaccaaaggattttactagatcatctctgaaatttcttccaggtctacagtggtatgattatataaattactagacccatagtaaatcatctaagagctcatatgaccttatttagaaaggaaattacaaatcttttacacttggatctggaattgcttttgtaaatgtgaagctactatgagttgaattacacttttgtttcagagattgactttatgaagatccttaggaagttttaaagttgaataagattcttcttcttacctttaatcatcacttttacatctcatttgtggagaatcaaaagtcactggaatcaaaagtcactgacccacaaagtgtcttcctcttgcaagatgggcaaatggctccacaacaacataaaacccagcatcacactgacggttacagatctgtttctgccgggttgagtctcctggccaccagaatcccagagctctcacccaggctgagatgcaaaagccacaagcacagtggggagagaggaaaataagagaaggagcccatgactttgagatgtgaaataaaggagaaccaacaatactctgtgcctactcatgagcacctcggtgtactccagaactttcatttcaaaaagttaaataggaacctttgtccagagattggctcagatgttctcattagatcttagcttgaagcctcttctgccagttcctccctgtttttatagtaagtctcataaggcatggtcctggacccacagccctgtatcatatggaaaaatgatgcaggccgggcatggtggctcatgcctgtaatcccagcactttgggaagccggggcgggtggatcatttgaggtcaggagttcaggaccagcctggccaacatgatgaaaccccatctctact

SEQ ID NO: 27 - DNA, 3740 bp, Homo sapiens
tttgagggtggcgggcttgaggcgggcaggctgcttagttgcggcccgaggcgcctaagtggggatgaccaacccgtgggcgttggcgctgacccctaggcgccgctggtggccccgcgcgcggccctccggccagccccgcccctctcggggctcctccttggtccccgcgccatggcgcgtccgcgttgaccgctgtcttccctttcgggctgtgctgatcgcgaacgctgcgcagtccgtggtggcgtcgaggcacctttctcgtgcctttacctgtgttcactcctttgctttaaaaaacagccctagaagtacacatcgttggccccgaaggagccccagcagccatgtcggaccgcgaggtgaccttggagggcgggaggacggacgaggggcctggcggagctagactgagagggcgccgccggcgtcctgaaggccctgctccccgaatgtgtgggagtgtgtctgacggtgcgagggtggctgtggcggggcctgagcagcgtgtccgtgtcccgatgccgcccgcctgttactgagttaggcaggagtgcccgagtctggcgaacttcagcagttctcgttccagagctccacacgaggttggccaaagctttgcgggacttacatacccttcttcctccgcccagtcctgcttccttccccttcctttcacgggtgttgataccatgtcaacatcctcgtaccccaaactcagtcgcagcgtctgcttctggagaaaccattctgcagcattaaagctggtgagaagatgggattcgaggctgcatcactcaccagtggtgaagtaggagggtgaactagtgaaatggaatccacgagtgggtgaagtatcagacatttcatatatggtgaacgtagtagatgaaaggaaggaaagggggattggatagctattgcttagtgccaaccgagactcttaagaatagtggtcagctgaaggcaagcaacaaacagttgtaagccagtgaatctgtcccgttacatatagagaagcttcatttactgcagcaggtcaaagacaagaaccaggcccaggaattaacatccctgtttgactgaactccaaaaatagcaaaacacccaaacaaggcaatactcttccactaagtttggatccctgctaagaaaagatgaggctgggcacagtggctcacgcctgtaatcccagcactttgggaggccggggcgggaagattgcttgagttcaggagttcgagaccagcttgggcaacatgacgaaaccccatctctgctgaaagcacaaaaaattagccgggcatggtggccacggctgtggtcccagctgcttgggaggctaaggtgggtggatcgcctgggccctggaagtcagggctgtagtgagctgtgactgcactccagcctgggcaacaggagtgagactctgtcttttaaaaaaaaaaaagaaagaaaagaaaagcccgtcatacatgggttggagatacctgggtagatgccttcaaaggttttgactccccaaactgccttaaacctttttttcgtagacagtgtctcactctgttgcccaggctggagtgcagttgctggatcatggcccactgtagcctcaacttcctgggctcaaacgatcctcccacctgagcctcctaagtaactgggactacaggtatgtgtcaccacacctggatacattttttattacttgtagaggcaaggtcttgctatgttgcccgggcttgtctcaaacttctaggctccagtgagccatcacacctggcctgccttgaacctgaagcctgccggggtggctcacctctcctattaacctgacactactcctcctccctccacttactgctcagtagatgtataattatggttgtttcttttgcattattcagctggaaacaatgagatagaaaagagaatatagctccctccccctcagaagaactgcgttattagtttgctggggcttcagtaacaaactgggcagacatttattgtctcccagttctggaggctagaagtctgagatcaaagttttcacagggttggttccttctggggctgggagggagaatctgtctcatgcctctctcccagcttctggtggtttgctggcagtctttggttccttggcttatagaggcattgtcccagtcctgcctttatattcacatggtgatcttgttgtgtgtgtctctccagacgaaggcataagtaacatcattgacaaaggtcattcgacatgggcctctttttagaaggacaccagtcatactgattagggcccactctaatgagcgcatcttaacttgtctacaaagacccatttccaaataatgtcacattcacattgaccaggggttagggcttcagcatcttttgagagggacacacttcagcccataacaagctgtaccacccagccaacatgtactgacaggagctgggaaagtttggggctggattatgagggtgcttgatgaagggagctggaatgtaaacctgcatagatgtgtttattgaaatacgtgatttaacaccttggcaaagagtggctgcagacttcctgcaaggatggctcctagaatggcggtatagctactgctgcctaacaaattactccacacttcgtggcttaaaacaagaatcatttcttatctctaggttactgtgggtcagacatagtggggatcggtgatctctctaggttactgtgggtcagacatagtggggattggttatctctcttccacaatgtctgaggcctcagctggagcagttcagaggctagaggttggaatgagtgaaggttcatctgctcgaatgtctgacagctgatactggagattggctgcagcccagattggggatgtcagccagcacacccctacacggccggtccctgtggccgaggctttctcacaatatgatggctggattccaagggcaatctaaagacaaaggacaaaagaaagctgtatcctttttgtgaaccagcctcagaagttgcataccatcacttcggctacttcctatttggaagaaatgagtcactaaattacccatattcaaaaggagaggaattaggcttcatcttctaaagggaagaatatcaaaaaatttgccagtatatttttaaaacaccacacttggaaaaagccatgggccatggtaagcaaaattgaaatggcaaaattgctatggcagacagtggtggaagggagtaaaaggttcaggggtgtgggcatgttgaaatttatatactccgtgcatctagaagacatttgagacgatcatattccacatgaaggtgtaaataacacgtcatttataaagatcattagacataggatgatgaaaggggcactgatgtcactaaaatattcagtgatggctcatctctgtaggcaagaatgacaatagaaaggctgttccagaacttggctgggtgatagcactggggatgataggatcctaagacaaaagaggccattctgggacatagtggggaccaaagaaaaaaacccaagaagccaaaggcaacacttagctggcagaagtcagaggattgcaattatagcagccagcagggtctgagtggcagccaaggggacctcacttgtatggttatagaactggtcaataaacatggcatccctggaggcaaaacaggtgggcagctaagaagggtactactcagctggcaaaaaaaaaaaaaaaag

SEQ ID NO: 28 - DNA, 2732 bp, Homo sapiens
aaaaaagtacaaagaaaggaggtagtgtcatttggaagaaccttaaatatgcagtgtcactgaagtcaggggaagaaaagaatttcacggagaagggcgtgttcaatgcgtggaaggctgcagagtagataatgaagattcaaaaaggcaaatctgagaggggttgcagtacagtcgtgtggtttctagactacacggcgtttcgaaagtgggtaaaggcagacatcacgcgtcttcaagaagttcagaagaaagaggaagagtgttgtcatagtgggtaaggtcaagtatctttgattgcaaaacaacagtcgccagaagagaaggggctaaaagttggaacgaggaagaaggctcggagaaaggtgtcagacaaagcgggattagcaagaagctgttagggctggtcctaccgggatgagagaaaggcgcagaggccagccgagtggaaagagcagcggtgacgaaccgggttccactcagacgtccgacacttctcgccaaggggccagcgcggacagcagcgcctcccggggacctctgagaagccctgtttctgcgcggctccgcccgacctccaaggccgacctcggaggctcagagacccaggccccgttggcactcaccccactgccacgcggcgccagcgccggactggccgcacgataagcgcgtcccaggctgccgccaaccggccctcgggggagacgggtcccgggggcgcaggcgcgggccccagacacagcgagctccagagagagcgcagcgccgagcctggcagctctggctccagcaggaagacgcagcccacggccagcgccaggaagcccgcgtacgcggagccgcgccacagcgccatggggacccaggcgccgcacctgcgcgaaccaactcctttcctagcccgcgcctcttccgggctcggcgcgcgccgatgtcgacacaagcgctacgtcacaagggtgcgccacggggccccccaaggggcggggcgacgggcggcgccaggacggagcgaggggggaccccacgcctcagtcccaggcctggcactgcggtgttgccgccccggaggaggtgggacaacggcggttgtgccagtccgggcgctgcacccccttcccggaactctaatcgtatccccaaatagagggatgggaacacatttgctttcgcagtaaaacgaaacggacagattgtgaagaagcggacaaacctcgcgttaatattcgaaccagtgggtgtccccattggcacggatcacacccccatcttttaatccctccctccgcccgtgtcccctcatttgctagacttgtcctcttccaggcctagtgctcggcgcttctgagaggaataggctcacagaatagcggcgctgccgagacccctggggtacgcgaggcagggggattccgcccctttggaaggtggccgagaccctcagccactaaaggacttcgtcgagacaggagagcccgcagagatcgttctcttctggataaccagattattccacaatcaaactttaacccttttgggggcgctgttccctttaacaaactctggaaaatgtacacaatcttgtgcacaacacgagagttatggacctgggttgagaaacgctgctttcttttgttcccccttggtgacatcacttaaacccagccctctcttcgctgatacttttctgtgcatgaggctaggttgagagacagtgaagctaggctgggtaccagctcattctcatcagccacaatgcccggcctagtcttgttccctggtttgtttccacttttccaattctctcggctcctgaccttggctttgtgtccagttttccactgtgaccctgacctttggacttggcagcgaaactttatttccctaactttgatcttgggcattagtcttcattctcctcagccccacctcatcagaacttccccatcctggtcatctaccttcccgcagttcatcctacccagcctacctgaccatgccatccctttcgacaaagatattcacacaggaacagatttgggctaccttggaaaagaagccaaagagccagtcagatctttatgaagccatgaaagccatcttccctagagttgcctgtcacttctctctccttagggagacatgtcagtcagttcctagagaaactgcttcttctcacaaccctcagctgtcaggtttccctggcacccagaggggactgagccagcagctgacctgaaaacagcgagtctgctgactgtccagcgatcatttccctctattgagaattttaaccaagtttctgttgtctgtagttatttgatattggctgtggacccacaaagtcacacaaggctaaagggtggacagcacggaagaggcagtacatcttaacaaaatcagggttctggatgaagggaggggtggtgaatggttgctgcaatcaacagttcatacttcaatggaaagaaggtgggatttgattcctggctgcaaatcttcactctgtcacctgctatctgtgtgccctcagggtctcactttgtctcccaggctggagtgcagtggcgcaatcaccactcactgcagcctcacctaccgggctcaagtgatcatcccacctcagcctctccagtagctgcgaccacaggcacgtgccactaaatgattattttttcaagggagaaatcatgcctgtcatacaaataaaaaatgaacaagtgtaaaag

SEQ ID NO: 29 - DNA, 2051 bp, Homo sapiens
agtcgaggccccgggtggccgcccgacgagtcggtgctggactggcgcgagtgcgagcccgaatcaggctccttaaagaaagactccggcaggatcttcttccgccacgagctaggcttcggattcatgacagagttgaagagggcttcgaggtctgtgtctaggtcctgcgtgacgtggatcacttgctgcccaggcggcgggagcggagggggcgccgaggccggattcatcttctgcaaaaagaaggtcagatcagccttttatttaaagtcggaggaagtgggtaagagggttacagggtgagagggcgagatggtgggagaaagaatagagggaaaaggaaacgaggagacagataattgcccgcctggagatcccagacactcagcggtaagaccagcaggatgggggaggggtccctcctggcctcgagaattatgcaactttcttgaagcaaagaagttgcctggaggaggagaagatagggcgaggggtggagggaataactgcactcggggcttgctgaaccgcaggatggcaaaggaaaggtcgcacgattccaggacaggcagccccccgaaagaagttcagcccctactccaccccatttgattcaaataggagtttattaagtaaatcaaacgagacaatgtaaagcacttcgcacagcaccgggctggttacgtaagtgtttgttaaataaaagagaactgtatgttcttcaagttcacgtattgtgccttaattttttttttttttttttttgagtgacgtctccctcttgtcccccaggcttgagtgcaatagctccatctcagctcactgcaacctccgcctcccgggttcaaacgattctcctgcctctgcctcccaagtagctaggattaagacgcctgccaccacgcccagctaatttttgtattttttaaaagcaaaaatggggtttcaccatgttggccaggctggtctcaaatcctgacttcaggtgatccgcccgcctcaggctcccaaagtgctgggattacaggcatgagccaccgcgcccagcctgccttaatatttttacagggtaaaataaagtcgaagttaaaatctggagctgccttggaggagaaaagtttaaggaagagacaaggccactcatagttttgcctcggaaaaggtagaattttggggccactccctgaatggctgcatccatatccaaaacagaaccaccaaagtgagccacttcccctgttatctgtacttggaggtggctccaattccagactcctcatagactggaagaaattagggccatcttagactaaggcaggcatacacgtatcatcctttttttttttttttgagatggagtctcactctattgcccaggatggagtgcagtggcatgatcgcggctcactgcaacctctgcctccctggttcaagcaattatcctgcctcagcctcccgagtagctgggattctgtgcagcaagtcctctgcccataggactggcaaaaggaaaggggaaactagcacaggtcactccttggaaagtagaatctttgcaagctactctcagaagccatcacagttgcaacaacaggggaaataagctatcgaacaagaggaagtgactggaacctaatgatactaattcagaagtcacaaggctgacttgatgattaaaagatgaaaacttgaggccagccctactctaggaaagtcctcactcccgaagaaaggagacctgagccactaagtaagaagtccagttaccctgttggataaaccacatggagaaggaaaggccctgagatacttggagagagggaaaagtccagctgcccagcacctgagctgagcccagcctcagccaaccccaccggctgactgcaaacacatcagtgaccaccagtaagaccagcagagctgcacagccaagcccagcccagattgcagaattgtgagcaaataaaatggatattgctttaagccacaaaatattgaaatgttttttaaatgtagaatgtgcattctaagaataaaaagttgcaaat

SEQ ID NO: 30 - DNA, 2103 bp, Homo sapiens
acgtccaacgctgggccgaccccagatacacaggcagtcgggattcccgcccggtgcgcttgtctattcatccctctgcgtcaggctgggacgcgctccgtctgtaaaaggctcaaacgcatctcccgccgcggggcgggatctaggggcccaggccccggggtccagaggcgggtaactttgctaatctcccccagcggcggggaacgtcgcgcaaccgctgagccctgtccgccgagactaaacaagcagaggaacaggctgtaaacacacatcctgacacgcagggatgttgctcttggagaatgtgaagacagtttgcttcttgacaagcgagcgaacgggcgcccagatttttgcagcctttccgagtctcccccgagggagaggcggcagagaaaaccccggatttgggagccaccagggaaggatccgcgcaggggagccgccctccttggccccagacccgcctgcctggggccccctttgctcactgtcaatgtatggtctgaagctctgaggatggtgctgggactggggtcgggggaagcctcttgaataataactgcaaagaagaaagaggcgagaacgtctccctaaccttgaagcagaaaggactgtgttcttaaagctgttggctgcagtcacagggccagttgcccgcctctgttccctgagtaaagtgtaacatcttctgtcctcctggcttgcttgcaccattcagcaaattatactccttccttaccaaagtgggaatgctcagggaagtgtgtgtgtgtgtgtgtgtgtgtgtgtgtgtgtgtcccctctgcactgaggctgctgttagagatgttaccaatttaaaccttccagaatcctggaggtttacattttgtaaagggaggggacactcctggactgtcacaatcccaattctggttagagtggcagggactgaacaaagccaccaacatgcaaaagtccatctccagagtagcatgcatgcccccaggaaacccccagtgggatatgcttcctgcactttccccttctctctaacctctgtctcctgtttgtaaggcagaggaagggtgattccttgccactgcacaggaatgcagggttagggttatctccaagaaagggtggggtgaggctgagtcacagggagagcagaaaagctctgtatcttcaatgaggacccacacacacacacctttcccaggcttgtgggcctcattcagcaaagcagggagtgttttatattgatgcgagaggctgtcagtcagcagtaaatcagttcaggcatagctatctctttctttacgaaatcagctcattgccttggtcacactacacagaaaatctgcttatcaccgctatcggcaataaaaattagtggagccttagttgtttccgaagaggaaccccgtgtctgtgacattagaatagataagtggcttggcctgttgcaggcagagagaagcccaattcctcctcctcttctccctgcagcgatctgaacaattctgaaaccgcctccctgggcgtcagctgagcaggttggggaactaaccagggctctctctctagggccctgttaaatgcactgaacttaaaatgaaacacgaagtgtgaatttcaggtttgaacatgatgcatcaggaaacgtggaggttggcagcccttttcctccctcctgcttttcagtagcaggtattaatattgtattaaatgttatgagaaagtaaaggctgcggagaggaatgtgctcagatgcaattttgtcaaggtttttatctgtgattatgattccagatgtagaaactcccggaggagggaaatgaggggctgctggcatgtgacatgtgttttaaggtgtttggcagtgtttctcaaagtggtgacaaaatgttcaattttattacagggaattggtaaaagaaatatgaatactaggtcagagattgttcacctcagcaaaaggatttaccattattgattagggtgcagaaagtatgtatctaggtcctgcttaaatcacattgtcaacaatataaatctgtcagatcagatttttctgaaagaacaattgtaacaaaatacactatagctaattgc

SEQ ID NO: 31 - DNA, 166 bp, Homo sapiens
  misc_feature (4)..(5): n is a, c, g, or t
gaanncagattacatgcgctacttaatggaagaagatgaagatgcttacaagaaacagttctctcaatacataaagaacagcgtaactccagacatgatggaggagatgtataagaaagttcatgctgctatacgagagaatccagtctataaaaaaaaaaaaaaa

SEQ ID NO: 32 - DNA, 630 bp, Homo sapiens
  misc_feature (387)..(387): n is a, c, g, or t
ttttaatggttggatgtaaacatgaactcaaacacgttttatttattaagatacaacctgaaccacaaaaacaaggagatcaaagatgaagtggcttagacagaagtttcagtctctctccccagactgtctggaggtgagtggatggcgcttcatgaggttgctggggacccctgtctgctgggtctcttgctctgttgtcctgttacgacacgcggtgcatggcatggccacatggtcggcatggtagatgctgggttgctgccacacccacactcccttggggggcggcgctgccgcctggcccgggtgccggggttcgcgtcccgcggggccttcctcgctctttgtctcttctgagtgaacttgatgacccccttcttccangaagcgcctcttggacgcgtgtgaccgatgcccagattgcacgaccacttctggagctgctcctgtgcgcacagcgcgaggcgccgaggccccccgcgagccagcgccgcggggctggcggccaaagttggggagatgatcaacgttttcgtgtccgggccctccctgctggcgggacacggtgccccggacgctgacctcgcgcccaggggccgcattgctgtgatggagcggtccgaagcc

SEQ ID NO: 33 - DNA, 506 bp, Homo sapiens
  misc_feature (441)..(441): n is a, c, g, or t
tttaagccacaaaggggaatttattgactcatacaaatgagaaatccaagtgggtgattatggcttcaaacatggctgagtcaggaattcaggtgatgttatcagctctatccctcgccctccatttttcaactctgtttacctctgcctggctttattcaagccttcatgatctgtatagctcaacatcaacatggctggaagggctggaggaggcttcagcatggacctccaggatgattcccacagctgtgttccagaactggcctgccattactgccacaatcaggaagccacatagtccacaatttgacaccaaacggtcactctcctgtattactaaagtggtttcatttttgcatcattcccacttctttactccaagcaacaggaggaaaaaacaacagcaagaggtctacaaatctggggaaggtataatangggagtttntgccatttgtataaataaaagcctctgaaggaaaccattaatttcctctgtagctg

SEQ ID NO: 34 - DNA, 485 bp, Homo sapiens
ttttttagctgaaaagaacagaaatttattctttctcagttctataggccagaagtctaaaatcaaagtgttggcagggctgcattgcctctggaggctgtaggggagagtccattccttgccttcttccagcttctggcagctgtcagcattccttggcttgtggccatatatctctctgctccatcttcacatggctttcttttctgtgtctctattgcctcttttgcctctctcttataaggacacttgggatggtattcaggacccacacatatctcctcggaaaattcactatagtctggagcctggctctcacccttactgaaccagaaatctgacaggtgttcgtgggctgccaataaatcttcgaggtgatcggattttggttcaaaggaaaacttttctttcacaacttcaagtacagatggagccacatatgtaaaacccagaaagacctgattggcactttcacttggaattga

SEQ ID NO: 35 - DNA, 2059 bp, Homo sapiens
atcgcgagatcaggaaggtggccgagtgtgtcgccgcggccatcaggcacttctccttcctgcccttgtatgaagaaggatgtgtttgcttccccttgtgccatgattgtaaatttcctgaggcctcctcagccctgcggaactggctagagcaatgtatcttaggctcacttaaggaagctgtagagatgagcccaaggagggaaaccagaagagccccccaggctcaccagttgtttgttggctccctacaaacatgtcattcaagtggctaatcttacaacagcacaaattcatctaaccagaaagagaagaggaggctccaaaggcacttgactactgagcatcaccctggacgtgtacaagtctgcgtccttattgttttcttcattgggccgaactttctggtcctcatccaacagctcttctatcatgtgttcgaaagtgtcagccaatgatgtcaagcctcttgaacctgccttgggcccattcacgctctccagagtcccatgggtccgcacacctggagatactctattatagcaaagaagaaagataatttcattgagccatcctgttttacagcacccaacagaatcccttcaaagcctcgtggtctgacaccctatgctacgtgacttgtgacccatccatttgtcatgttcttcgggaatgtggctaaggggctaagatgtgacttgaaaagaaaggtagaacaagatcatctcaaatttattatcaaggaatagttcagaaaacgacttcagaccacagagacagcagaacagatggtccggcatggatagagcatcagacactcacagactgtgccaacaagagccatcgagtcaaaacagccaaaggaaggagggtcatggaatgggttctctcacaccaaactgatgcccagaggccctcagcatgaataacaaaggcaaccagacccacaagccatactgagtggatacaaaacctatacctaagctgacatcccaaatgtgtgtggcaagttagatgatgatggcacaaaagacagaacaccttgctttctggccattgtcagctcttggaagagagcacacttttagaggagcagctgcaaggaccctgagaacaaaactggaaatgtctgttatgaaagccttcacaggaaattctgcaagtggcaacgtgggtccattccgtgtgtgtcactagagctggcgcaagcccatggccatggtgaggcagcgtttccactggaactaatctgatacctgcaccagctcttgcaactgtgcagtgttcccactgcaaactacggatgggagaggataaagaacttcaatctttaaaaaagagaggattttccctcctggtgagtcaaaatgaacaagaaataccccaggacctcccttccctccttggccattaatgagatgaaggcaattaactcacatagtataaatgaatcatttgaggtgatgactgcattttaggcaaatgatgactttcttggttccattggtttgcaagtaaaagttacacacattgaaaagacactgaaacagatttcctaaatgcttcattttctggatgcaccaatgttgacctactatacatgttaaatggttttaaaatatcaccttaaaataaaggaaacttccagctactaactcagctctgaatgggctatgaaaggctccaaaggtatgtgaaaaattactgttattttgctttaaaaaatgtgatgtctaagagtgtctgcaatgttctaatgcttcaaaacatgtacgtaagccttgtttatctggaaatcatttctttctgcttatatcatttataaatagaaaatgttctgtaataacttaaaatagttccacatacataatgcttttcgtgtcataatacttactactggtctatatttaccaacatttatcacattttacaaaatgaagtagaagaaaaaaaagacaacgactttatggccctggaattccagtaatggtgaccaacatgttttaaattccagtaaaggttatggttacatttcaaaaaaaaaaaaaaaaaaaaaaaa

SEQ ID NO: 36 - DNA, 2077 bp, Homo sapiens
cccaaaccccaggatctgagggcaaaaacaagcctctatttaaaaaagacaagatgaagaaaaaaattcatactgaggagaaaggttcatctgtcaatgtgaacaaggagtttacatcactagcattctactggttttcttggggaaagaagtctctgcagaaaagcccacaatccacatcccactcggagcaaaggcccttctcacccgaagagccctagtgattcatgtcatcgccatctgtcccaccggacacccctgaaaactcacttctggctagcaaaacaaggcaccacttcttttgagatattttgactatgaaatgtttccgtgggacagctctaaaaaatctgcatttattccacaatcacacaacaataaaatgaggacctgaccttctaggtatgaaggtaccacagcacaatcatcaacactgctaccaaatctcatctgctaatatgtaaaatgtctccttctccagctgacaagcgatattctaccacaagccccactattgtgaaccacgataatataaaatgaaattaggaaaaagataagatgaccgggatgaagtaccttgcctacacacgccccctccctatcaccaccatcttctgcccttgactcttcaaccaacgaaggagagaaaagaaagaagagaatgaaacgtgcaatgcagactatagcatcacggacagcagcgttggttacaacatcttattagggtcctttaaaaatacacaaagagaacaaatgaaggagaacctagaaccaccacttactgctttttcttatgacttttggctcaatgtatgttttacacaaaaagaaatgctacaaggatttggtaccaggtaaacaatatataaattgttagggaaaaaaggaaaatctctttttttaaaaaaatgagaggttctactttttagggtcataattgtataagttcaatgtttctagcatatctttctagaagaaagactagaacagccacaggtgaaaaaggaaactgataaatggagggggtaatacagtagatcctgtgacgacatcctttatcctgcactaaaagtgcaatgctgcagaatgccatccccctcttgaaatcctgactctttgagaatagcaaatggtggtattactcatgcatgacctttgccaaaaagtggctggcagatgggtttgcccagcaaagtggagatgtgatgagattattctgagtccctatgcaagtagcccagttgagcctggacaagaatttcactggatcagaggctttttactttcacagaacgtagacaaatgtgccatgtcacaatggcttccactcacgtcctggtttatcatcattcctccctccactcctcaacagaaaccaaaaaaaaggcaattatctttcttccagctacagatgttaatcctcaactaacatcccataccggcagtgtctgcaatgtagtagaaaggctttgaagcctgcaaatcatttaggaccaaaaggaggaaatccaagtccaattatgggaaaatgaaaaaaagaaaaagagcagaaaagcccttcaaatcagaccacacagaaggccaaaaccacagatgaaaggaactgagaagctatgggtttttttttgctgccttttaaatgcagacccctaggcaaaggcagcacttatttaacatatccacaggtattaaagtctataacatataaggtgctagaaaaataatttcagtgattgttacttatgggtctagataattcttaatattgacaaacattggtatccatgtaaaagacaaaagtagaaaatatcactaaacttggaggtagaaaccagacttgaattctgcctctatgtatagcctttggaaaattactttttgtttcaaagcccaagtttctccatctataaaaatatggattaaaaataccttcattcaaagagttgtactataacttttcaatgaaattcatgtatatgtctgaaaaacagtatctaataaatataaactgtgtatgattctgatctttaatacagattaagactcccaagtaaaaaaaagaaaaaaaaaaaaaaaa

SEQ ID NO: 37 - DNA, 1208 bp, Homo sapiens
caacaagactgcaacatcccctcacgcctagtctgtaaagaactttgtttaacccggaggcggggccagccagctccgcccctccagctggacgcgggagcgaggttgaggttcatacccctgggttctctccaggcctgaggcggagagccagccgcctgcctacctctgggctttgaaccccgggtctggtgacttcgcttaaagacctcgggccgaaggcccgccaggctcaacctcggttcacaggacccccgacttgtgtgagtcgtacaccgctctttaccgtctctggacctaggtcatctcggcagtaaaatggggtgaaatgacccgacctccccggaggatgccgcgcacgatgtgtgcctcaagttggcttccactgacctgaactgttaagaaagccctcctcagtgacgccttccctgaatccacagtggagattgccgttttcctggaactaattcacccacacttacccatttcatccctttccaccttttatttctacctttctgccccactgatctctagaataggtgctgggagacgttagagtatttgatctcccactccccagtcatcccacttaccgagcagtaattctctgcctggctgcacattgcaatcacttgggaagcctctaaaaatactgatgccagggctctaccccaagccagttaaaatctccagcgctggacctgggcattggtactttttgtaaagctttcaggtcaaggaagagcaagactgaaaatgtttaccagatttataatcgaggaggtcctggatgaccttggcaaaagcaaccccaatggcatgggaatgaatgtttaccaagatgatgccagtatagctagtgagatgcagcaccccatcctcagccccctcgcctctggaagagacaccagactgcaaagggcaccgcgtacagaagctaacggaacttgaatgacaagacaaaaagagcagaatcagttagtgtgacacaacattctaacatgcctgattcttacatcaaatatggtaactttggggttggtagggggagacaaacaaggagaatccacttgggaacaatttgatgaagtttgcaaatcagcactttacccccaaattaacaacagcttgtatggaaaaaaaaatgctcttttaaaagtatatggtttggacaggtaaaaaaaaaaaaaaaaaaaaaaaaaaaa

SEQ ID NO: 38 - DNA, 3163 bp, Homo sapiens
gggttaatgtgctaagaggatatgagattgaagctgctggcacccagctctccctgccacagggcagagaatggggccaaggcataaagagaagcagagctctgagaaatggaatgagagagacctgatgacattgtttgagcccctgagatgagccttgcctgaggggttccccagccctggatctggcctgctccgccttgcgaaggccccacagcttgcttacctcagcaactcacagcggaagtgtgacaaccgttcataggcaaatgtcaggtcagatgtcaacttgttgctccacagcctttcggcaacagcccctgctctgggcctggaggaaaaggggcatgtgccagattgggccctgtattccctgcaaaggtgctggacatccacaggctgctacctcttgtctaggcaccctggactcactcaggccaaaggaagtgatcaataccttgttatgagccacagccaggttggatggctcccagagagttctacggctcagctccaccttcattaaaatgtgggtagcaatcccactctcttcctctctcaaagatacgggaataagaataaaccttaaagccatcattaaccacaatgacagtggcagcagcagaggtccttaaaatgtaaactttgcacacccaaccatacttggtaaggctgcagtgaaaacagcagagggaagccttattacaggaatcatggatgcttgtgctgggtcttgagcaactcacctatgttctccaggcctcattatctctctgggaagtaaggttaacaatccctatcccacataccaaagggcagctggagggatatagacggaagtcatgtggagagtgaatattgcacacaaatcttcagaaggctcgttgcacggcctgctctctctcaggcctgaaagaagggtactagtccagaggtggctagatgggattgggtctctaagggagaactcctgctctcaggcccaaccctccacctcccccccgaaaacccttaccagagcctggggaccaggtttgtgttgtccacatattctccccacatacaagcctctccaccaatcaccagagccttctgctcaggggtacctgagggaaaacaagcaacaacagtctggtgatggtggggtaactccagggtccctctcaaccaccttcccaatgtggccctcaccccagctaagttgtttcatttactaactggaaatgtctggcccagaccttcctgcctcccatcctgtgccccaacccagcctcctttggttagcaaggagagctctctgctttcaccttcaaatgccaggggttccactacgtagaaatccttccagtcagggccataggatatacggttcaggtaccagggggcagagagaagggcccggaagccggccttggtgaccagttccagctccttcatatagttcactggaatatcctctcgccacacctgtatgattgtgtctggctgaatctgttataaaaggtcaaatggcagtaaggacacaaagctgaggagattcctgggccttattcatacacaggcaacatgggacaacagattctaccctggtggtcagattcctcagcatccactctaggcacaactacctgcaacggcatctgagaaaaatgtgcccttacatagtctaacagtacatattttaatagtaagaagcagcctccattgtccaaagtgagttcttcctatctaaattcccaggtggaagaagtcgatggaaaacattcttctaaggaccaaggctgggatatgccactcccatgagccagtgccctgaagcttcactctgagcataacaagcagagtccctctggtcccagacatcattcttacctggtccccaggacaaagtgtggccaggagtgtcaaactctgcaagcacacggataccccggagccgtgcgtattcaatgacctccttcacatcctgtgctgtgtagatgtgggtgacagggttgtaggacccctgaaaggcacaagacacccttcaggttcacacttcctgaaagctagcagagtagaagatactcaaaatgcccacaagactccccagatatcagaaaacctgcccatagccctttggtgtcagggactatcttcaaaaaacttgatcataatttcccagaagttatcacatctgttttatctgagtatcataatgccagtgagataatcatggtaggtattaatactatgtccatttaacagaatataaatacataaaaagggaataaggccagaagagattctctcctgaaggtcacaaggcaaattatcggcaaagttttggacttgaactcaagtctcctgactccaaatccagtgtccttcccctatattggtctaaaactggctggttaggatgagagaccctgttcttgccagcagggccacagccagattcagacattgacccataaacttggtctgagtgaaacgggaacatacctttctcatgagctctggaaaagtgaagctctcatatgggaaggaaggatcatctaccagatgccagtggaacacgttcaatttattgtacgccatgacatcctgtaggttaaagtgcacactgtgaacccatcacagtctctccggtttcagcctcaaacttgcgatgttgggcgagctctcaggccgctccacacacccctacaggcttgacctgcctcagctctcaattaagtatttatggggtctatcaaaccttcccatcagggagggatggcatggagggaaggcccagcacacttctacttttcccagaacacatccaaagatggatgatagaagtggtctttcctcttcttgaaataaattctggccacattaagaggagggctgcagctactgtggtagcctggaatcttccaaaagcctgaagatcaatacttcctcttgccatttgtgttcggttgtctgactgatgttaagcccaactgtgagacctcctgggaacttagtagcctcttaactcataatctcagaagcaaaggctggcagatgtgtggcctcctttggttccgtcacaggagcaaaggaaaaggcagacacaggaactggattgggaactgtcagataagactgcacattaaactcaagagagttaggaaaaaaaaaaaaaaaa

SEQ ID NO: 39 - DNA, 2871 bp, Homo sapiens
agtggggagcagccagcagtcttatgctaccactgtggctcatcctgcgacctgagagaaacctgggcaactgtgagcaccagaggccaagtcctggacagtctttaggccatgttggaactacatttggaaaggtggttgttaaacatttcccagcacacacctggatctgcatctgctgtgtcaattgaaccactcactggttctgtgacctcaggcaagtcatttaaattataagccttggttttatcctccacagaatacttcctcctaaaatgtgttgtgaaaattagaatagatgaaggcacccttgaaaagatgactgatgcggttggttgccccaccacttcctactacttggcagagcctcctcgttggcagatccttctctcactagagcatgtatatggatgcatgctctctcaccctcccttgtagcttagagcagagcataccaatctatgtgccagcataccctggtatgtctcagatgggtcatagatgtgataaggtatggatcccttaagcccttgggacattcaagtgaggctgagctgctgaagccctgggatggtatgactggccacaagcaacattacccaggtacccctctgtgtcacaaaaacgttatctttgtatttgtgtcatggtgtgaaaaaattgagaagcactgcctggggacatagtgaggtagcccaattctggataaaggaaattgagaagtcttgagggggtagagaggttttcttccctaataagaagaatcacaggagagaaaatgccttcctcagccatatgttgttatttctatatgtgagcctggaatttcaaggttatcttgcaaagaaccaaggcaatgtagagaaaagaattgatagcaagtggctggatctttcacattgtaaagccattgagccatccgtgcaattgcccaccttgggaatttatttttaagagtgataataaatgctcctcattgtttaaactactttgatttttgtatttggttattcataattaaaaacacccaaatacagtagaagaactggtacatccctgacacctaatgagtgctcaatatggttagttaatcaccaaattagatcctttgcctttagaaaagctgccatggtcaaatgtgtgttgatcttatttttcgtaaatatttaagaaatttgttcagtaactagcttaacttttaatatttatcttgagtctcttctaaatatgtttttactgtataataaaagcaataattgaaaaatgacatcattttaagcttgagagcaactagatacaaagatgttgatatgcattttctcttcttgaaggatctagacaaactggagcaccattacatgaaaaaggttaaagaatcaatgttttgcaaaatagaactggaattaattcatgggaattacagagaagatcatattagttcaatataaaaagagggcctagaatgtgccaagctccatacaatgcccctcatgagtgttatttaattctcacaacaactttgcaaaaaagaaactatttttttttccattttacaaaggaggaagctaaggctgtgaaaaagagctcttttgcgagcctaggacctgattccaaaattcccactctttctgttgcaaattctggctaagctttccagagatgacttgctttccccttgaggccctttcagtgcattgtgccatggaaaacaaaaagcccaacaatgacatctttaccaaagatgccttggggagatgggagaagagttacaatgcacattacacagcaaaatcttttttaagctccaagtaaaattggttttttcttagagttttgcttccagcagtcacttcctccattctcctgctctctccacgtgaggaaaacttctatcctgccttctcctaatacactttcctgttttttttttcctgcacatttcttaaaaatcccacataaggcaaaagccaagacaatatccatcaacacctccacccccaccgcatgtccatggagagcttcaagtcatcctgccgctggtacaaacacggcagaggcctctttgtaaagaagtggtaggtagaattcaggttagcaccagaatctgaaaactccagctcctttgactgggaccaggctggtaatataaatgttagatcaggctgggtctgacacaatggggatcagtagagcatacttgtcacatttgaaggcattcagaaaaataataaccttaaaacattctaaacatgttgcaatcaaagaggataccccttgaccactgcacgaattggagttcaaagatcatctctcttttgcctaggctgtagactagttatttaacctctctcagacttggtttcctcttctggaacagggtatatcaactaccttgcagaaataaatgacatcctgtacacaatgcacctaccacgttgcctgacccaataatgtgtacatacataagaaaatgttcatgccctttgctcactttaaaatttttttttcttgtatatttgtttaagttcttcgtagactctggaatggagctggaagctgtcatcctcagcacactaacgcaggaacagaaaaccaagcactgcatgttcccacttataagtgagagctgaacgagcagaacacatggacatatgaaggggaacaacacactctggggcctgtgaggtgcagggagagcatcaagaagaacagctaatgggtgctgggcttaatacctgggtgatgggttgatctgtgcggcaaaccaccatggcacacatttacctatgtaacaaaccttgacatcctgcacatgtaccccggaacttaaaaataaaagttgacaaaaagaaagcaaaaaaaaaaaaaaaa

SEQ ID NO: 40 - DNA, 784 bp, Homo sapiens
gtgctggtctgtggcggtggttgtcggctgtcctgccaggaaaactcggcactgactatcccactgctgatgctgtaaaagaggtccttctgctatgacgcaactgtggttcctctggttctggggacacttaggcctcgcgtgccgattttcatagttccctgtggctccgagccgagggagccgcggtgtggaacggagggaaccagtgatctcagtgctgaagaaaacagggagtgtgtgtcttcatacttgatttgccttaccgcaggggcataatatgagatatctgggcgtgttaagtcgttcttgatgaaatgtgtcctctgtgaatgtagtctcactgttagactaggaagatgctgttttgctgtgcccagttcctcttaaaagtacagatgctccttgggttgcagccgtataaacccatcctaaatagaaaacgcgttttaatacccttataaacgaaaaggataaataagcctttgggttctgtaatgtgagccagcattctttgagtgcgtatgtgacaggaaccaaagagaagtgaaggaatgtgtccaagggctcccaagttggcggtggaattagaattgaagcctcagctccaaagcctgtgttctgaactacccggttgcagttcacaaaatacactttggcagtactttcataacgaaaacaaatgattacaaccagtttacaaatgctttgctttactggaggatcgacatgtttgactaattttcaaaaataaatctcagaagcagcaaaaaaaaaaaaaaaa

SEQ ID NO: 41 - DNA, 1417 bp, Homo sapiens
agagagactcaggggaaattagagcatgatggcggccgaggtcgcttggcgcaaccatcgtcttttttctttatttttagctcattagtgccttgcctcggcgctgtttccctaagctcctctctccaaccagtactgccctataacggaatccatacgtgccggctccttgatttatcccctccgaagtgctgagtgtgggctggttgtgcgtcattggactgtcaggatgtcactccccagtcaaatcttcaagctattggtggaacagcaagaaagcccgtccatcaacaagggctaataccattggtttgactccactgtgttatttcctggcaccttattgggcaaataggtgcgtcagatccggattcgctctgctacggttaattgtttactggggatttggggtcaactctggttgtttggccagtagttgacgataatagtgactcccttgcctattttgtgccaagtattgggctgctttcttcacaaacattatctggttaaacctgggagtaatgcagtatccagtttttatagatgggaaattaaggctggcgagagcttagaaaacttgcccaaggtcatccagatacagagtcgttggtgggcctggcatctggtcctgggtcaaactgcagagacttcactctttttattatgctttagtgcctccaccttagacgatgtacttgtcagccaacctagcatcttaaaaacgtttcatttggaactcaaagtcttcagtcagtttctagaagtgactttgttggggacttactatggaaaggttgtcattgcctccaccacatcttcctccccaggacccattcctctcccctttaaggtagacatttctgcctccttttatgcgaagcctaggcaccactcgcaacccctgaccaaccatctacaactcattctttggagagagtcctcaagagctggaaaactaggagaaatcaagtctaaagagagagacagaataaatcgtcttagaaatgaataatttattgcatgcaaagagaacagcccttcagctcctggaaatcttccaggatagttgatgctgagcaagacgtgtgctcctggctggtggccactgggttctttccctatagcagcattcatcgaagtgaatccaacatttcagccagaggagggcaggacgcttaggacccattgccagtgagcttttgcgaccgaaatgatctccaggagcacaacgctggactctagcccacattccatcttgggtaattaacattttaccgtcccaaatccccatcccctgtagcttcagtgggatcaccgcccttgtttcacttcctgatccaagctgttgtgtaacctccttggcactgctttaataataaaacaaccattgtaacatagaaaaaaaaaaaaaaaa

SEQ ID NO: 42 - DNA, 1076 bp, Homo sapiens
aggtaggtggagaggggatgggggaggtctcaggtcctcttgctcacccatcagctgtgtagtgtctgaagtgattaaggagattggggaggcctcccaggctggtctgctccactggccctcagaaagacttgtcgcctcgccgagcctcagtgttctcatctgaagaatagagtgggcaaggcctttctcctacacactcagagccctgaatgggaagaaatgggttccatgtgcagcaagccaggagagatttaggcaacgagaacctccagttctgtcttagggttcccagctttctccttgtcctgagggagccgctgagcctgtgggagatgaggggtgcccattccccagtgcttctcaccctcccccacctgccctcctggggtacctttgtctgccacttgcatcctattggaagctgtccccaagtgcgtgttaggcaggtggcaggtgctggagcagagtgcaccaccctccggcgatcactgtgagttggggtaagagtggggtgttcctgccagccccaggagaggggaaagcaagcccccgtccacccttgtgggtctctgctcctggaaaaggtgctctagcaggctcatctgtgtgagtcactgtcttccatgtggggggctgtgctgaagaagccttggtgatatgggtgcaaatgccaaaaacatggaatgctggaagccaagcttgcacattctaggcagccatttggaagattcttctctgcgcatgaccagctgaaaaggaaaaaccccgaaacattggcactgtgatttctccatgtgaacagtttagttcgccgaggtggacatgcctcattcatgcttatggagtgaccgaccagcctttagtgacaccccctgaaatgtgagtcatccatgggtcactagtcattggagaagagccccttatacaaaacttggagctcagaccacaaacaacaacaacaatccaaaaaataaaaaaatgaaaaattaggagcagagactaaatggaaagatggaaatattaaaataagaatattattttaaaaattaaaaaaaataaaaaaaaaaaaaaaa

SEQ ID NO: 43 - DNA, 1483 bp, Homo sapiens
cagccgggctgagaggagcgtggctgtctcctctctccgccatggcgtgtgctcgcccactgatatcggtgtactccgaaaagggggagtcatctggcaaaaatgtcactttgcctgctgtattcaaggctcctattcgaccagatattgtgaactttgttcacaccaacttgcgcaaaaacaacagacagccctatgctgtcagtgaattagcaggtcatcagactagtgctgagtcttggggtactggcagagctgtggctcgaattcccagagttcgaggtggtgggactcaccgctctggccagggtgcttttggaaacatgtgtcgtggaggccgaatgtttgcaccaaccaaaacctggcgccgttggcatcgtagagtgaacacaacccaaaaacgatacgccatctgttctgccctggctgcctcagccctaccagcactggtcatgtctaaaggtcatcgtattgaggaagttcctgaacttcctttggtagttgaagataaagttgaaggctacaagaagaccaaggaagctgttttgctccttaagaaacttaaagcctggaatgatatcaaaaaggtctatgcctctcagcgaatgagagctggcaaaggcaaaatgagaaaccgtcgccgtatccagcgcaggggcccgtgcatcatctataatgaggataatggtatcatcaaggccttcagaaacatccctggaattactctgcttaatgtaagcaagctgaacattttgaagcttgctcctggtgggcatgtgggacgtttctgcatttggactgaaagtgctttccggaagttagatgaattgtacggcacttggcgtaaagccgcttccctcaagagtaactacaatcttcccatgcacaagatgattaatacagatcttagcagaatcttgaaaagcccagagatccaaagagcccttcgagcaccacgcaagaagatccatcgcagagtcctaaagaagaacccactgaaaaacttgagaatcatgttgaagctaaacccatatgcaaagaccatgcgccggaacaccattcttcgccaggccaggaatcacaagctccgggtggataaggcagctgctgcagcagcggcactacaagccaaatcagatgagaaggcggcggttgcaggcaagaagcctgtggtaggtaagaaaggaaagaaggctgctgttggtgttaagaagcagaagaagcctctggtgggaaaaaaggcagcagctaccaagaaaccagcccctgaaaagaagcctgcagagaagaaacctactacagaggagaagaagcctgctgcataaactcttaaatttgattattccataaaggtcaaatcattttggacagcttcttttgaataaagacctgattatacaggcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - DNA, 1422 bp, Homo sapiens
ccgacgcagccgccggcccgcccgccagtctgggctcctgcacatctggcgatcccgccacatctgggcagccggcgctggagcatgaacggctctcaggcgggcgccgcggctcaggccgcctggctgagctcctgctgtaaccagtcggcgtcgccgccggagccccgcgaggggccgcgcgcggtgcaggcggtggtgctcggcgtgctgtccctgctggtgctttgcggggtcctgttcctgggcggcggcctcctcctccgcgcccagggcctgacagcgctgctgacccgcgagcagcgcgcgtcccgcgagcccgagccgggcagtgccagcggagaggacggcgacgacgactcctaggcgcccggctgcgctcggtggtcgcggcctccaggcagcccctgactccgagcggtccggagcatgcccgacggctgctgcggtcccgaccccttacccgaagcggcgcgccccacacagagaggagaagaagagaagaggagaggagagaagagaagaagagaggagagaagagaagaggagaggagagaagagaacctcagaggatccagaacggcagctggtccttgctggactgttcctgtccatgtgcctggtcatggtgctggggaacctgctcatcatccggccatgagccctgactcccacctccacacctccatgtacttcttcctctccaacctgtccttgcctgacatcggtttcacctccaccacggtcccccagatgactgtggacatccagtctcgcagcagagtcatctcctatgcaggctgcctgactcagaagtctctctttgccatttttggaggcacggaagagagacatgctcctgagtgtgatggcctatgaccggtttgtagccatctgtcaccctctatatcattcagccatcatgaacctgtgtttctgtggcttcctagttttgctgtcttttttttttctcagtcttttagactcccagctgtacaacttgattgccttactaatgacctgcttcaaggaggtggacattcctaatttcttctgtgacctttctcaactcccccatcttgccgttgtgacaccttcatcaataacataatcatgtatttccctactgccatatttggttttcttcccatctcggggacccttttctcttactataaaattgtttcctccattctgagggtttcatcatcaggtgggaagtataaagccttctccacctgtgggtctcacctgtcagttgtttgctgattttatggaagaggtgttggagggtacctcagttcagatgtgtcatcttcccccagaaagggtgcagtggctgcagtgatgtacacggtggtcacctccatgctcaacccctttatctacagcctgggaaacagggatattaaaagtgtcttg

SEQ ID NO: 45 - DNA, 867 bp, Homo sapiens
agtaaaattctactttccatctgcttggctgcggaggaccttggggtgggctcagcgtgggggctgcaggcagaaatgggtccagaaggcaaggtcgaggttagagccaagcctgtgggcagggctacgggcagggggcggggaggaatgtgggtccaggcccagaggaaaaggttgaagttatagccaggcctgtgggcgaggccgcagcagggcggccataggggaaggagccaatgtgttatgcatgtagaaaatagacaaaatctcactctgtcaccaaggctggagttcagtggagccatctcagctcactgcaacctccacctcccaggttcaagcgattctcctgcctcatcctcccaagtagctggaattacaggtgcatgctgccacaccctgctaatttttgtatttttagtagaggcagggttttgccatgctggccaggctggtcttgaactacttgtcaggcctctgagcccaagttaaatcatcataaaccctgtcacctgcacgtatacatccagatggcctggagcaactgaagaaccacaaaagaagtgaaacagccagttcctgccttaactgatgacgttccaccattgtgatttgttgctgccccaccccaactgatctcttgaccttgtgacattcttcttctggacgagtctcaggagttccccaccgagcaccttgtgacccccggccctgccagcaaaagataaccacctttaactttccactacctacccaaatcctataaaactgccccacacctatctccctttgctgacccctttctcggactcagcccacttgcacccaagtgaataaacagccttgttgctaaaaaaaaaaaaaaaa

SEQ ID NO: 46 - DNA, 1376 bp, Homo sapiens
gcctccaccggcggactcgtgagcgcgccgctgccgggaccgctcctgggccttagagaagacgcggataagggccaaggaaagagggaggtagcggttgctgagctccttcggcgcttcggctcctgtagctctgactattcggaccgtcaaggtagaataggaggcggccagttccccgctctaagaagttgcctgcgctctgagagacaggtgccgctgtgttgcctaggatgatctcaaaccactaagctcaagcaatcctcctgcgtcgactttccaaagcgctggaattatgggcgtgagccactgtgcccagactcgttcaaaacaagccatgactcctcagcaaaacagctccatcaagcgtctcgccaacctctcccttggggctcagtcaagggaatgaaaaaacgaggctagacacctgtccccaacggaaaaaccaagaacacacctggagagcagctcagacaaagggagccaggcggggaaaacagtaattggagaggagaccgtgcttccagtctgttgctggtttacaaggtaaatctattcctggacgaaaggggtacaggaacagcacccgaaaaccggcgacaggctgtggcaggcccgacgtctttcaagcccagctcctagcgtcgacgcccctccttccaagacgtttcccagcaggccctgcgcccagtttggatcaagacaatctatgcaggaagaatgaatgggtgatgctggcatcttgaaaagttgaagctgatagactgaagaatgcggattacataccctgaggctgcagaggattttttctcaggcagccaagaagatggtggattgagactgagcatgcctaccacaagcagctgacaatcattcaaagcaagaagagggtactgctaaaggaaactggcaagaagcagctcctatggtacttacaagaacatcggtctaggcttcaagaggtccaaagagactattgagggcacctacattgacaaggaatgcctgttcactggtaacgtctccatctgagggcagatcctgtatggcatggtgaccaagatggagatgcagaggaccactgtcatccagagaaactatctccactacatccgcaagtacaattgcttcgagaagtaccacaagaacttgtccatgcacctgttcccctgcttcaggatgtccagatcagcgacattgtcacgttggataagtgccagtccctgaacaagacggtgtacttcaatgtgctcaaggtcaccaaggtcgcagacatcaagaagcaattccagaagttctgaggctgaatgtctgcctgctccccaaaatgaaataaagttattttctcattcatacacaccaaaaaaaaaaaaaaa

SEQ ID NO: 47 - DNA, 839 bp, Homo sapiens
gattatccaactgagtggacaaagatgggcaagtggcctgcggggatcgggcctgctggatgcttgatgcacgactgcttgatgcttgagtgctgtgggagacaggtgctcagatattgctggtgagagtggaaaccaaaggtggtctttttggaaggtaatttggcataaccatccaaattgaaactgtatacatactatacatatgctgacccagaaattctacttataaagaatttacggtaatcataggacagtgtctgtaataaaacactggaaacaacctcaatgtttaaagtattaaataaattagagtacattcatattcagactatatagctgttacggtagatcttggtgtggaaatgggatgatgtcaagtatttattaagtgagggagaaacgcaaattacaaaacagtacctctaattgctccatgtatatgattaatatatatatatatatgtcttttcctatatgtatatatataaggaaactgccttatttttgaagttgctggttgggatttctctgttctttcagcccctgggggacacctcagttaaagcaagatcagtctgttggccgagagtggtggctcacgcctgtaatcccagcactgtgggagtccgaggcgcgtggatcacctgaggtcaagttctagaccagcctggccaacatggcgaaaccccatctctactaaaaataaaaaataataataaattaaaaaaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 48 - DNA, 894 bp, Homo sapiens
cacggaagcagaagggcattgacctgtgagatttccaatgcacagacatggcttgctctttctcaagcagaggcaaaggatgtcggtgctattgacagtcctgggcatgaatctttcggactgcaaatgcccgctgaatatggactccaacggtgccctaggggatggtggagccaccagatggaaggagcctggtgatcccgagaaggagagatgcctcgctgacctgaacatctttccagaactgttaaatgaagcctgcaggacaccagctgacagtggaaggactgaggaaggttgtataattttgtgcaaggtccaccacaaatccagctgaagaactgctccaaagtttaggtcatggcaagaacaagatgtaaacttgactggattaaggattccctaaagtgtcaggcctctgagcccaagctaagccatcatatcccctgtgacctgcatgtacacatccagatggccggttcctgccttaactgacgacattccaccacaaaagaagtgaaaatggcctgttcctgccttaactgatgacattatcttgtgaaattccttctcctggctcatcctggctccaaagctcccccactgagcaccttgtgacccccactcctgcccaccagagaacaaccccctttgactgtaattttcctttaccttcccaaatcttataaaatggccccaccccatctccctttgctgactttcttttcagactcagccctcctgcaaccagttgattaaaagctttattgctcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - DNA, 1021 bp, Homo sapiens
agttgcttgtgtcgagggagggagggagggaacagagggtgcgcgtgtgaaagctccgcccccagccctagctcctccttcccgcttcagcaggtccaggctctgcgccagtgcatccttctccaagagtgcgctgcctgggcccgcttgccctggagttaacttcagcagtcaacggagagaagagtggaaaccttactggatgcggacaggagagccagttactgaaagcagatataacgcggatcctgtaaagagtgaaaggagaagaccacttttagttgcctccctgctagcaccctgacttgctctgcttgaataagaatccaaggacacaagctaagacatttgcactgggtttagatctactgcgattcacaaaacacaaaagaactttcagtcagagggtgacacataatgattttagacaaaactcagtttcttctggtggaagctattcagattgtttggagagtgggaggaaaaaggagtgaagcatcgcttaacaacaaaagtaataatgcaggcagaatattaaatggagaagccagactttcaacacatagaaaagaaagctagttcagaatgttgagagacttgggagctgattgattgaaggtggcacttagaaggttgccctttttaaagataaaggctgatactgtgactctgtcccaggggacctgagtaaagaactcctcagagatgtggaaccactattcagaacaagatttggagagctggggcagagaagattttaccttggggatttcttcagctgcgagtagtaaattggttatgccttaatgaatgaagtttgtcaaattttaagataggatgttattacgccttttaaaagaatgaggtagagtataacatctgatttggagttctttccagcattttaaggtctttaaagaaaacaaattggagaaaaataaataaatatagatcatgctgtacatataaaaaaaaaaaaaaaaaaaaaaaaaaaaaaaaaaaaagaaaaaaaaaaaaaaaa

SEQ ID NO: 50 - DNA, 1759 bp, Homo sapiens
agtttactgcgcgcccccgagggccggggcttccccttccccgcccccttggctttcgcagtcaaatcccgctctccgcccgccgcccgcggaagtctcaccgcgcagcagcacccccagcccgggcggttccggggaccgaagggcgcggcacgtagggggcccgtaggatccaaactgcggaggagaagcaggaggagggaggaggaagaagacaaaggtgtggaggaggaggggagggaggaagaaggaagagaggcagaagactaggaaaaaatgggcccgtgggcgcggagggggcgcggcggggtagagaggcgcggacgctgagcgagcccggtgcctctcgaaagcctcaccggccgcccggccgggccgcgcagctccgccaactcccgggccggcccaagccggagcgtggcgcccgtcgggaggttgcagagttcctggagctcacagctgagcccgaaggaagtcgccctcgatgggcaggaagcgggacccaggcacgcccgcgcggtcgcgtccagatctgccgtcctccgacaggcgcaggcctgctgcgccttcgccacacaccgccactctgggcctggactccgtagttacgccgggatatctctgggcatccaaccacctccccttccccacagtcttttcatctccacccgaccccttgctaggagcagcttctagttgcagctcaagacccgcgagttcaccctccgcgccaagcactcaactcccggctctgcctacagtcacccacagacctaatgatcgactaagcgccgtaatggcccgagatgacaccaggagactcccttgctgtgcgcctccggaagacgcggaacgtagcccctttctcacactcctgggacacaaaaatacccgacaactcaaagtcgccaaaccacatcttttcatgctttgaagacttctgcccaaggctgtgacaggagtatgtccaactgtgatttgggattctttttaaacatcgaatcaaagggaagaggggagggattcgcaactaaatggtagttgttttcaagcaagtctttacgtagccccaaacctactctagtattcggtgattcagccccaaaacatctctgcctgcctttatgttcggatctcactccattatttgtcagcaccaagtactaaacccacaaagcgatcgctgtacgggacaccaaacccatacctgcccgtcccctaccttccaggcaaccagtgtcaatctcctcactatacataattcccaacatccctgcccgaagtgaggagaacggtaatttaaataatacagcgggggaggagaggacacactgtttttgaaagactgtaagcattctgctgttccactctgcgtccttacacttgctgagcactgacaccaaatttaaatggcgttccttcaaaactgatgtaattgtaaagagctgtaaagggcaatgacctcaagtggatgttttttcccaggtaccctacaaggtttgcacaaagcaagaaagcatcatgtaaccgaaatgctgaaaagtatagatgctgcaaaacctcattctgagactgagaactatggatagtcagtgtatttccttctgcggtgcagaaaaagaacaaccaaaaagaaagcaacaaggggagatgttatttattttaatgacaccaaaactaccaatcctagcaatttaataaattaaaatcatagtcctttaaatgtaaaaaaaaaaaaaaaagaaaaaaaaaaaaaaaa

SEQ ID NO: 51 - DNA, 975 bp, Homo sapiens
ggggacagacgtcgactccagcaaaaatgtcctgactgacttcaggagagaggaattattgctacgtgcctctttcccagtgaagaaaatctctgttggctgcgagcagagaagcggcggaaggattcccgactccgggaagccaggtgtcggaaggaggaagtacgcttgaagggggtccggctggccgactctgcactcgtccgttcgtggcccaccgtggtggtcaccaaattgagggcttcctggaaggacctcctcacggtgcccatccactgggtcatgtgggtctgggccactgtgagtttatctcccagctgtctctccctccctctctggaaggcaacactgatccaggatctcactctgttgcccaggctggactgcagtggcatgatcacagctcactgcagccttggcctcccaggctcaagcgatcctcccacctcagcctcctcggtagctaggactacaggtggagcaacaccgtcagtcagaaggcagcggatggcacagggcttccgcgtaaggcctgcatcgcctgagtcatggttttctcatcttgtatgaggcccacccccttcaaaaggttgtgacaaaaaaaacaactgagataatttgtgccgctggcacatagttaacactcaataaggccgggcgcagtggcttacacctctaatcccagcactttgggaggctgaggcaggtggatcacttgaggccaggagttcgataccagcctgaccaacatggtgaaaccccatctcaactaaaaatacaaaaattagccaggcatggtggtgtgcacctgtagtcccagctacttgggagcctgaggcaggagcatctcttgaacccgggaggtggaggttgcagtgagcggagatcgcaccactgcactccagcctgggcgacagaacaagactctgtctcaaaacaacaacaacaacaaaaaaaaaaaaaaaaaaaaaaaaa

SEQ ID NO: 52 - DNA, 1810 bp, Homo sapiens
tctcccggaggcgcatgatgtcctcggccaggttgtcgcgctccacctcgacgcgggctttgtcgttggttagctggtccacctgccggcgcagctcccgcatctcctcctcgtagaggtcccccaggcgcgacttgccttggcccttgagctgctcgagctcggccagcaggatcttattctgctgctccaggaagcgcaccttgtcgatgtagttggcgaagcggtcattcagctcctgcagctccaccttctcgttggtgcgggtgttcttgaactcggtgttgatggcgtcggccagcgagaagtccaccgagtcctgcaggagccgcaccccgggcacgctgctccgcaggcgcacggcagaggagcgcgtggcatacacgccgcccggggacgaggcgtagaggctgcggctggtgctggggcgcagcgcgctgcccaggctgtaggtgcgggtggacgtagtcacgtagctccggctggagctcggccggctcgcggtgcccgggccgccgaacatcctgcggtaggaggacgaggacacggacctggtggacatggctgcggagggtggcgatggcctgggcggcggcggtggcgcggactggctcccggagaagaggcgaacgagggcgcgacagcaaagctccctttggatgacatagatttattacttagtagtatattatgtattggctgtcccacattttgaaatttaatggactcgtggtgatcagaataaaagaagccttcgatgtgaggcccaggcattgagtaccatgtgtgcgattcacaagccttggcatctgaacaattttttggaaggatccatcaggacaacacgtcgggggtgtactagtgaagtgattttccaaatgtgctactcagacctgtagcatcagcatcaccggatatacaagacttaccaggtgattctgagaaccactgtgctagtgaattgttcctgtctgtggccacgtagaataagaaaaccatagggttggaaaatggggaaactggtgagtgttcgcttatgaggaaatgaagaatagatagaaaagaattagttaacttttggaattcaaaagagaagcagtttgtaaaagccaggaatttgatttgaaggactaattgcttgcagaatctttgctttctcagagaggggcaatccagatcactaggttaccgtgaaatatgttggtatggctctaaaattctagaataatctctctagtgagaaaaggcatacctttccatattaggacaaaacttcaaatcaggtttgtaaaatcctataagattattgtatcccattgccatggccaacttgtttgtctctggagatcccagtttctacatctgaaaaccatatgcattcctgctcaccaggaattatctcgctgaatgaagcaagagattcaggatgtgtaagagaatttatgaagcatttggattaccaagaaatgtgaagtataaagatcaagaatgattattacaaacactgatggtaaagaggtgtttcgaagtcgatgcaaaaaatgagttgcttatttcagtctctctttgatatatctgcctttttagtgctgactctatcatgtattcactatttgattttcagtgaatcacattttttaaagcttttaatctgtttcctcaaaaaatatattttttaaaaatatttactcaggattgttgtgagaataaaattgattcgattgatatttcaaaaaagaaatatatttttaaaaaataaagcaataccatttttggaaaaaaaaaaaaaaaaaaaaaaaaaaaaaaaaaa

SEQ ID NO: 53 - DNA, 1619 bp, Homo sapiens
gcggaagctttgttctttcgttctttgcaataaatcttgctactgctcactctttaggtctacactgcttttatgagctgtaacactcaccgcgaaggtctgcagcttcactcctgaagccagcaagaccacgaacctaccagaaggaagaaactctgaacacatctgaacatcagaaggaacaaactccagacgcgccaccttaagagctgtaacactcaccgcgaaggtccgcggcttcattcttcaagtcagtaagaccaagaacccaccaattccagacacactaccttggctcacggcaacttctgtctcctgggttcaagtgagtcttgtgcctcagcctcccaagtagctgggattacaagttgaagaaatatggagatcaacacaaaactgctcatcagtgttacctgtatcagctttttcacctttcagcttcttttctactttgtaagttactggttttcagcaaaagtttctccaggtttcaatagtctcagcttcaaaaagaagattgaatggaactcaagggtagtatccacatgccattctttggtggttggtatttttggcctgtacattttcttattcgatgaggctactaaagctgatccactttggggtggtccatcacttgcaaacgtgaatattgctattgcctcaggctacctcatttctgatttgtccattataattttgtattggaaagtgattggtgacaaattttttataatgcatcattgtgcgtccctgtatgcatactaccttgtactgaaaaatggagtgctggcatacattgggaattttcgcctgcttgcagagctttccagcccgtttgtgaatcagcggtggttctttgaagctctgaagtatcccaagttttctaaagctatcgttatcaatggaatactcatgacagtagtattcttcatcgtgcggattgcctcaatgcttcctcattatggcttcatgtattccgtgtatggaacagaaccctacataaggcttggagttttaatccagttatcctgggtcattagttgtgttgttttggatgtgatgaatgtcatgtggatgatcaaaatttcaaaaggttgcatcaaagtcatctctcacatcagacaagagaaagccaaaaatagtcttcagaatggaaaacttgattaaaagagtgctaccgataagcaaacttcattactacccagcatatctgctgataggatgaattcttggcatgttcttgtgtacctttcttaattataattgttattcaggatttcagtgtcatttttttttaaaccttagaaaagagaaggccgggcacggtggctcatgcctgtaatcccagcactttgggaggccaaggtgggtcgatcactgaggtcaggagttcgagactagcttggccaacatggtgaaacctcatctctactaaaaatacaaaaaaaagtagctgggcgtggtggttggcgcctgtaatcccagctactcgggaggctgaggcaggagaatcgcttgaacccgagagacggaggttgcagtgagctgatattgtgccactgcactccagcctgggcgacagagcaagactctgtctcaaaaaaaaaaaaaaaaaa

SEQ ID NO: 54 - DNA, 341 bp, Homo sapiens
acgatgcggggacatttcggcccgtcaccctggcaaagcgctcgcagggctggagggacagagttctcagatccaagtagagaaaaccgggaacggttccggctctggggactgacattcatcgcggcagtttagagaaaacggagcaagtgtacaagcacgccaacccccccggtgcccaagctcggcgctcacgcggctaggatgacgcccgtgggacgccccaggggccctgctcgcagccactctgctcagggtcatttatagtctctccgttctttgttaaataaagacggtgagacacggacgggctggagccggcaggggtagtggagggcaga

SEQ ID NO: 55 - DNA, 983 bp, Homo sapiens
gcagatggcaggatttcccaaaggtttctggctgaaacatattccgtggtgtatctgtacagcagtttcctcatccctgcagctgtgtttgaacaggtcatttaccatgctgtcctccaggttcaacagtatggctccaaatgatgaaatttcattctgattttctggctgaagactattctctttgtgtatgtccaccacagttactttatcccttcatctgtggatgggcagtccagcctgggtgacaagagcaactccgtctccccaaaaaaaaaaagaagaattgtcaacaagagggagtggcaattcagaagcatatttaagccaagtcctcaagactagaaagcatgaagcaggggaggcgttttgaaagcgtaagaacaatagaccatgggcatggatggccgagtctggggatcagcatcgtaatttgttgagaaggaggccgtgctgtgctgccagttattaatggtttaatcggttgatacacagccctactggcctaaccagtagcccaggccctggaggatttgcaggtcgtgtcagaatttgattgcagttccttccacttggcataaggaagacactatcagctgattgggagggtgatggtgggatggaacctgccgaggtgggggctgagtgaacacaccaccggcatagagtgggagcctttcctggcccgcttaatgcggtaatatcaaagcactgtatgggtgtctatctgtgctggacctgagttcatcttgtctgcaattacgatctctgggttattggcagtcatatcaggccctggattgggctccaatttgcgtgcgggggggccctaggggtttggggcgcggggaacgccttccctcgtctggtgccgggtgcgaaatgggttgtcgggccactgtgagggagaccccgttgccattgtgacttgcgcatgtttcacacaccgctctggcgggggggtctccctctgaggcctaacg

SEQ ID NO: 56 - DNA, 578 bp, Homo sapiens
tcggttggcgcagcagccacaccacaaccaggcgaagcaggtagtggcgctgaatcgctcctgccgcgggagatagagtttgctcttgttgcccaggctggagtgcaatggcgcaatcttgactcaccgcaacctccacctcccgagttcaagcgattctcctcccacaacctcccgagtagctgggattacaggcacccgccaccacgcctggctaattttgtatttttattagagacggggtttctccatgtggtcaggctagtctcgaactcctgacctcaggtgatccgcccacattggcatcccaaagtgctgggattacaggcgtgataaattgaccatcttataccaggaagtcaaatgagaagacatcagtaaaaatattctggccaactcatgttttatttatcatatcttcatcattaattgcttctcataatgccacagtttgtcatggtatttgtggcagcaggcaaagtccttgttcagactttgattaaattgctgtggcatttttctattaaaaagatctcctgtttcaatctaaaaaaaaaaaaaacaaaaaaaaacaaaaa

SEQ ID NO: 57 - DNA, 434 bp, Homo sapiens
tttaatagctcctagtactttaattcttgttgttccatgggaaaaaaaaatcacaaaaacaaaaatccgacaaacacgtggctgggtagaacaaaacgctcattggggagggtgggctctaaggtggtggaggagatagagaaaccgagttggaagcccttccccgcccctaagtcccagccccatttcttttcagcgcgccgggaaaacggggagggggacaaaggtgctgcgtgctgtctttcaactcccgactttttgaatggcatacaatcgtccggccgcagagcggtgagccaaagtcggagtcagctcagactctagggcctagagagctgccagcagtgtcccgggtggtgcaggctctggaaactccacctgtctgtccccgactcagccctctcggaggggtttcggaccgaagggaagaagct

SEQ ID NO: 58 - DNA, 532 bp, Homo sapiens
ttttttttttttttcccattacaaaaccactttaatacaagttccagtgacaaaaaggacatctcaggagacagtgatcgaggagctcatggagttataaacaggaattaaagcaacagaaaagtattccctcacagttctagagggcagaagtccaaaatcagtatcactgggccaaaatcaaggtgttagcagggccattctccctccagaggctccaaaggagaatccattcattgcaccttcggccactggcagctgccaacaatccttggtttgtggccacatcactcccctctttgcctccatggtcacattgccttcccctctcctgcatgtgtcagatcttctgcttcctacttagaaggatcgcaatgtggtgacatgtagggcccactggataattcaggatcatttctccacctggagattcttcattacaccagtcatacagcaaggtcacaaaaaaaagtctcagaaataccctcatgagcaacagcttcccagtgaatctgcctggatgaattagctg

SEQ ID NO: 59 - DNA, 887 bp, Homo sapiens
atgtgtggctgctcctgagctgacactaatctgctcatgactcctctgtgggagaagtaggtggttcctagaggaagaagttgggtaatgaagccacagagatttgctgatacatttgctaggcacgacttctggtcatttagaaagctatttgtggcttcatcaataaggtattccccagctgtgttactccttcgcatgttatctctttccctggaattgaaggcttcctgtctgaggcaaggacaattattccctcatgtcaaccctgatctctggctgcagtaatgagtagaggaaatgaagaactaagaatggaagcataatcatttgtccgaggtcacagagggagatgattacacagctggaatgtaaagcctcagtactttactgaatccttggctttgtccatgggcccagctacaggcataaagcttttctcttccccagcagtgacttcgagtaccagctttcaaatttattttgacattgaatctaagctttgtgaccagtatgtagaaaggagacgaagggaggaattactttatccttggaatacattagcatgcaaataaatcttttcttctgcattcgtggaagggtctgcgattatcaccatataccatgtatcttaatcagtggcctggttacaatttacttggagatcagattagggtctcttcaacacttgatggccttttctagatcagaatttacctaggatttaagaaaagtctgactacttcttttcatatttacaccgtatatcttccaggttaggggacctgctcctgtcacacattttttgcagtccacgaagtgtacccccttctccaaagagacaagcacatcggggggaaacacacgcactagcggcgagcacacgc

SEQ ID NO: 60 - DNA, 292 bp, Homo sapiens
ccagtcaaagtttatgatgtgtcttttattttaattgaatttctttgtaatggtcctgattcccatttcacttggctgaggccaactcggccttgatctccaaatttccttccagcgctggaaacctctttggcaattgggggtaatcacccctctgatgttgctgcatccaactgggatgggtttgcaaaagttctgccagaaacaggaatggatgtgtttaggagaatgaggtcttcggaagctgcctggctgcctcgggtgcagtgttttgtgatagaatcaagagttg

SEQ ID NO: 61 - DNA, 544 bp, Homo sapiens
gatttgggtaaagacattcatctcagtcgtttctctctcccagcttgaccttaggttaatatttcattggggtcaagaaaagaatatgtaggagagctatgtggctttaacaagaaaatggacaaaaatggatagttggcctcattaaagaacacgcatgctcgcagaagccattacaatcccgtcgtgcagcagcctgtgctatgtgaccagcctagaagtcatttttcgactcaaggggagaagggtgggtttctgctctttcctttgcaccatctgaaggacacggcagaagataaaggaaaatggccatgagatgctccagcggaggctgggtccaaggcctgggctcaccaaagatggcactactctcagcatggtacaggaagacaatgatgggacaatgcaagctgggacaatctccaacccaaaggtatctcagaaaacagcactagagtctgaaatgattccaccgtgtcttagcagaaagctgcccggaagttgtaatacttgaagatccaaaagcaccagtgaccaagagaag

SEQ ID NO: 62 - DNA, 761 bp, Homo sapiens
  misc_feature (467)..(467): n is a, c, g, or t
aagtgcagcacatgaataattcacagacagacaagatgggttcctatcagcccggcggtgccagacggtcccttggagtccttcctccatgccggcctgttctgtttagacccccttccacttcgggatgaaatcacagctgttggtgagagaacccccgtccggctgggaatccctcgccttctgctctggctcagcccagcttggagtctccgcctgctcactctccggtgaccggctcttcccccacagcgctttccacacaccctgtgcccttgtgacccagcccctggtgaagtccctggcccgggcctccttgtgcacacacccaggccttcccagaacacgagagcaccgggagctgcagtgggagcagaccctctatggcgtgggccccacttcccactggagtgagtgggcaacatggaccagccccacagctgaggccatctcctcactgagcctgngaccaggtccacgtaatgtgtcttcagtccccgtgggaatggtggcatctcagccttctccagagtgtttgcgactcacggtccacatgtcgcaacanacgcaagggtgttctgctctttactaataacgtgatgcgccgccccgcgtggagctcagcttttgtccctgtatgaggtntaattcgagctggcgtatcatgtnctagctgttcctgggtgaaatgtttncgctccattcccacaatacnaccgaagcataagtgaagcctgggtgctatgatgactactacttat

SEQ ID NO: 63 - DNA, 695 bp, Homo sapiens
cagtgtctcttctgtcacccaggctggagtgcagtggctcagtctgggtcactgcaacctctgcctcttgggctcaagtgattctcccaactcagccttctgggtagctgggattccagatgggggagtatgaacattgaaaaccaaggaactgggaccctttcctctcttccaaaaaatgttgttgagcacctgtgtcagacacccatcatactaaaatgaataagatagagcccctgtcctcaaggatctgatgttctagtgaagaagacagattaacagattttgctgctgctgctgctgctgctaagaatcctttcttctcacctctgggtgcctgaaagtcagcaggagttacaagcggaaatttggccaaaatgcattgcactctgaaagaaaatgcttaatttctctaaaagtgaaggacttgctgagaaaaaaatgttgaaaatataatgagcaggcatttcaatgcaaaaggtgatacaatatacccattacgttgctattctcatgggacagaaccaacctttgacttttgtataaactatctggcaatattacgtaagacacctcctatttatagttctgtttcagaacatagtatataatctggtatatatcataatgaataaagtcataatgaataaaatcaaaatagggtgccgccctatttgataataaaaaaaaaaaaa

SEQ ID NO: 64 - DNA, 601 bp, Homo sapiens
  misc_feature (1)..(1): n is a, c, g, or t
nctgccgatctgtttttatttgatatcgaggctaacggagaaggggctagagggcgtcagagctgctccctggctctggcagagggaggaagtttagggtcttgggtttacagtagcccctgctgcacagcccagggtcttgggtactggcacctgcctctaccccaagttggaatgatattctatcttccttagggtagaacaggaaagtgaaacgagggctcattttggttaagataagcaaacatgaggcctggcagggtggctcacgcctgtaatcccagcactttgggaggccgagttgggcggatcacgaggtcaggagatcaagaccgtcctggcaaacacggtgaaacccctgtctctactaaaaatacaaaatagccgggcgtggtggcgggcgcctgtagtcccagctactccggaggctgaggcangagaatggtgtgaacccgggaggcagagcttgcagtgagccgagatcgcgccactgcactccagcctgggcgacagagcgagactccaccttggcatataatctcccgtgttgctcatgcacgccttggnttgtttctcctcctcagaggaccttcggggagag

SEQ ID NO: 65 - DNA, 903 bp, Homo sapiens
agcggaaagggaagtcgaatagtccctgcgagttaatgagtgattattatttatggaaggaatggtatttgcagatgggaatgactgcgaaaagtgcatacgaaggacacaggcatgccctttccacggaagtcacagagcacacctcaacacactgagaagacgtccacgacagagaaagatgacgaaggtgcttctgtgtacagcgtcatccacttcctcatccgtaaactggatcccccacggttgtcagcagcgtctctcaggtaatcttccgtgaatggtgcctgtagcttcttcatcaaagcaagcaaaagcatttctgatgacgtcgttcaggatctgtgccatttaacttcgttaccaaacatggagagcaagatggtgaagttaatgggccctggtgcctcattcatcacgacatcaaggcatgcatcagtgggattcttccctagagaagcaagcgataccatgcaaatcttccttgctgatgaaaccatctctgttctgatcaatcatgtcgaaggcctctttgaactcctgaatctgtgactggtcaaacgtggcaaacaccttggacgttgcacactgagggcgcttcttggtggtcttgcttgtttgctcaacatggtggttgttaattccggcaccaataccagaaacggaaccaccctgtgcgagatgacaagcgacaggcgaggagcgcggggctgtgggcagacaggtgggggctatagaggtaatgatttaatactgcatagttaaattgtctttagatggcccagtctgtatgttttcgtaaaatttgaagttttaataagaaaagagaaaacaaaagagaaaaaagaagaaagaaaaaaaaatgctgggggggtagcaggcaaaaggggaaaaaaagagggg

SEQ ID NO: 66 - DNA, 727 bp, Homo sapiens
agcgggaatctggagctgacttagtcatgtttctgccttcagagagctacccagtgggggagatggacatgcaggggatgaggcaccagagagaacacaacactctctgccctcaaggagcttggagtcaagagaaaagggagacaactaagcaaaaatgcatgaggagatgaagagcatgggggaaatgaggaagcccctgtctagcctgggcaggagggagatggtaaggaagtgataatagcagaggcctattctgaggagtccactgtacctgtgaagagccttgtctggcacatgctccagaagtattccaggtgtacatggacagaggccccggatactcgtaccctgtcgactggtggtccctgggcatcacagcctatgagctgctgcggggctgggtaagacaggcacctgtgcggtacacacgaggggctgtgcagtgggggctcacgttgtacctggacgggcagagtcggcagggcccgcagtgcaggaaggagcactgggggagtcactgccccccaggtttcagtcctgatgcctgtgtgccgcctatgaacgccctgaacttctggtccaacccactcattgtacagatgggaaagaggcattcagtggggggagggtcttacacaaaggcccacagcggatcagtgacagtcaaacatcaaagcggcttgggctcgtggtttcaaacactgtcctctttgaacccaaaaaa

SEQ ID NO: 67 - DNA, 801 bp, Homo sapiens
  misc_feature (375)..(375): n is a, c, g, or t
ccactctgctgaaattagaggagaaaaaaatagggagtctcgctgtgtcgcgcagactggagtgcagtggtgcgatctgcacactgcaacctccacctcctgagatcaagcaattctcctgcctcaacctcacgggtagctgggattacagacgtgcatcaccacgcccagctaatttttgtatttttaatagagacagggtttcaccatgttggccaggttggtctcaaactcccgacctcaggtcatccacccgcctcggcctcccaaagtgctgggattacaggcatgagccaccgggcggtttttccatgtgtgatctttaaaaggcagaaacaaataagcaaaaatcattactaaaggcccacgagggtngtttctatcttagaaaatgtgtaaagaaaagtgatacaacaataaatggcttgatttaatgagaagtcaaaaatcataagatctgttgaaaataacaaaatcacttctaaaaaagataatatgttttattgaaataaagccttagaagtcatctggttttaccttttacaaagaagaaagcagtctaagagaggccaaatgacttatcccaagctatacggctaggtagtagcagattcaaatttatactacccaggcttcctaattcttagcccagagctgtgtccaccctaccaccaccttttaattcagtgcgagctcaattattgctaacagttttattgtttgaccttccatggactcgatagtcaaatcaagtcttccattacaataaatgggatttattgtatttgcaccataaccccc

SEQ ID NO: 68 - DNA, 983 bp, Homo sapiens
agcttgaaaaggcaaggaggattctagcctagagtctccagggagagtgcagccgcctggctgacacacgccagtcctgccaacaccttcattttagacttctggccttcagaacagaccaggacgcccctacagggccatagatttctcacatcaaggacctgcatttgttacctggcaccggtaccatttgttgtgtctggaaagagatctccagcgactcattggcaatgagtcttttgctttgccctactggaactttgccactgggaggaacgagtgtgatgtgtgtacagaccagctgtttggggcagcgagaccagacgatccgactctgattagtcggaactcaagattctccagctgggaaactgtctgtgatagcttggatgactacaaccacctggtcaccttgtgcaatggaacctatgaaggtttgctgagaagaaatcaaatgggaagaaacagcatgaaattgccaaccttaaaagacatacgagattgcctgtctctccagaagtttgacaatcctcccttcttccagaactctaccttcagtttcaggaatgctttggaagggtttgataaagcagatgggactctggattctcaagtgatgagccttcataatttggttcattccttcctgaacgggacaaacgctttgccacattcagccgccaatgatcccatttttgtgggtcttcattcctttactgatgccatctttgatgagtggatgaaaagattaatcctcctgcagatgcctggcctcaggagctgggccctattggtcacaatcggatgtacaacatggttcctttcctccctccagtgactaatgaaagactctttgttaacctcggacccaacttggggtacaagctatgcgcgtcgaatctgcccagtgttcagtttgaagggagactccaggggtggggccacacaacggtctccttaaataaggccatggggaaaccccc

SEQ ID NO: 69 - DNA, 513 bp, Homo sapiens
cacgaggccgagtcttggccttaactggctggcaacgtaaaacctgccctccacaaggacaagatgaaaacttggggatttggaggggacaacaaattagactcaatcacaatttcaaacttttggatgtcaatatatccatcaagaaattgaaaagaggcctggcatggtggccgatgcctgtcatcccagcactttgggagaccaacgtcggaggatcacttgaggtcaggagttccagaccagcctgggcaacgtggtcccaggagttgcagaaacgccaccaggaccttcagagcaccagttagacaaggagagccaggaggagctgggccccaacctcatccaaaagcacagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctg

SEQ ID NO: 70 - DNA, 461 bp, Homo sapiens
ttttttttttttttttttatcttttgaacaaacgtccttttcattttggtttagaaatagaaacgtggtaaaaggaggcgactgttttctgaaggcttgtagcacgaaaacaggccaactggccagcaactagttgtctatcagtttctctactcacatacatctgccttttccagactgccagcgcggagcactagtttgactttgggtgctcttcaaatagctggcattgataaggcattcccagaagactgtgttacttagtaatcttgtcggtacattttttcactgggcagagattttccaaacagagagctgccagctttcaagacgtcagagtgctttttcatcccaggggaggagctgcggtggctgagccgacgctgcgcgcacagccgcctgtggttttccgcgcattgtgagggatgaggggtggaggtggtattagacgccctcgtgcc

SEQ ID NO: 71 - DNA, 991 bp, Homo sapiens
  misc_feature (991)..(991): n is a, c, g, or t
ctgcgcctctggcccagtcgcaacagctgagcagccatacgccagtcagcaggagcagcagcataatccagcatgttgggctgcccttagcgccaggcacacacagcggagggcaagtctaccacagtctgacctaagccagtttcaaactcagacccagcctttagtcgggcaagtcgacgatactagaagaaaatcagaacccctacctcaaccaccactttctctcattgctgaaaataagcctgttgtgaagccgcctgttgcagattccctggcaaacccccttcagttaacacctatgaacagtctggcccacctctgtattcagcatagctattcctgttgatggtgatgaagacaggtgctgcaacctgtactggatgtttggggatagaaagatgaatgggacatgatttgtgtgctccaatgagctcagtccagtgcagctctgtgattcaagagatggccatttgggatgggcaaaacagaacaatatatgacaatatacaccccttggaatgaagaaaggctgagtgcggtggctcacaccctgtaatcccaccactttgggaggccccacgcgggcgaatcacccgaggtcaagagtttgaaactagcctggccaacatgggggaaacctcggtctgctactaaaaatacaaaaaattatcctggcgcggcggtgggccgtacacccgggaatccccggttatttggggaggggccgaaaaaatttgcttggaaccctgcggagggcagaaagtctgctacggtagacccaacgaattgccaacccactggaacttccaaagcctgggcacagaacaagaggcggaacacctccgtccttcgaggaagcgttgacgagctgtcctgattcaagagtcattcccccttttgtacaaataattttgcacaatccagcctttctaccttcgagggggaataatattctctcttccgcctcaaaagagcgcaccactttctn

SEQ ID NO: 72 - DNA, 558 bp, Homo sapiens
  misc_feature (523)..(523): n is a, c, g, or t
gggaaagttgggggagggcatcttaggccagaagaataacaagacaccagtggtggaaatggaaaacatgtagtgaagatagcacatggagcctgaacggggaagtcttacaagacagggttggaatgcaatggtgcgatctcagctcaacgcgacctctgcctccctggttcaagcgattctcctgcctcagcctcccgagtagctgggaccacagaccagagtgaagacaaatgtgtattacttggtagcttatgaacagcaaggaaaaactgactggcaaccgccatggaaaggtgtgaaaccgtaaccacgaggactctcacatttacatgttactgactagcgaatgtctaggcctaaaacatctgccctcttatagctgttttattattatgtaaacatggctacaagatttctgacataaaatagtagatgactcagtgtcttcaaatgattaattgctggtttttttgcctgacctctttcttcatttcttcaataataaatctattgtcatgcnaaaaanaaaaaaaaaaaaaannnnnnnacatgtcg

SEQ ID NO: 73 - DNA, 575 bp, Homo sapiens
  misc_feature (481)..(481): n is a, c, g, or t
ggggcagcgatgacgtaacgcctggcccaatgggcgccagcgaggaagcgttaaagagtcaaggcagtttgtgggagtcgcgctggggacgttcaaggtgtctcctagccgatggagtctcactgtagtccagatggagtgcaatggcgtgatctcggctcactgcaagctccgcctccccggttcacgccattctcttgcctcagcctcctgagtagctgggactacaggtgcccgccaccacacccggctaatttttttgtatttttagtagagacggggtttcaccgtgttagccaggatggtcttgatttttcgacttcatgatccgcctgcctcggcctcccaaagtgctgggattacaggcgtgagccaccatgcccggccaagcacttccttgaacacagaggtgaccatgaggagggaggcgtgaaccaggatgacggggcagcagatggagcctgcctccctgagacctcangtgaccgagtgctggactgcctcctcctgcctacatagctgagaaaataacttcttttaccannnnnnnnnnnnnnnnnnnnnaaaaaacatgt

SEQ ID NO: 74 - DNA, 536 bp, Homo sapiens
  misc_feature (518)..(518): n is a, c, g, or t
ttttttttttttttttttgatcctcataaaaattttattggagggacgtaaaaacaagctacttgcccacgaccgaggtcgctcttttgccttgcccgctctcaccctggctctctgcagacagagcccgggagcgactttttggggggagtgggggtggggaagtgtcgcagagatggagttgggggcaaaggagggtgcgcaggaagatgggatggggacgccccacttaagttgtgtagatctctcctacacttcttcaagctcggtcggcctccccgcccctgcggggctcccgcgcccagctcggccctgggggtccgagaaccctggcttcggggactggcattttcaccccattaaaaaatcctcatggttggggggaggccatctgcttatgggtggacatgggcacaggggcttctcagatgagctaggagccgtcctgagggggtgaccggtgccttgggtccagagttccggacccccaggaaatcgcaggtcgcggggagcagcanagagaagggggtgggaga

SEQ ID NO: 75 - DNA, 681 bp, Homo sapiens
catgcgccggttctgaaaccagactttgacctgcctttcggtgaggtccagcaaggccgcgatctcgacgcggcgtggccggcacaggaaaagatgagtgaggatctccgcgttggaggaagagggagtcgaaagaggcttccactctttgaaaaaggtcagtattaaggtgaaaataagaaagagatttacaatcatcacaccactatccctgttaccctccaaaaacaacagttaaatcttgggaaatggacaggaactcccaggcagacacataggagtgagatggagcataaggggcatttttgggcctctggcctccatcgctgtatggtctgggttctcttctctgcccagtgtttgcagcactgcctctgccccttatgtacccctgctttcctcactttcaggcttccatgcttctctgtgacatcccccaaaaacaggcagagtctaagatgtgcatgtacccactcaagcctcaagcttgtatctgtatccatatccagggacatgaacaagaactgaggtagccattggtttaacttagagcactctcaaagttcagatcagcgttatgtgtgtgcatgagtgtggtgggggagattttcccctaggacaaagagactttccctcccatttttgccattgtgctagttaaaaaagaaaaaaaaaaaaaaa

SEQ ID NO: 76 - DNA, 461 bp, Homo sapiens
ctgacttttggcaaaggaaggactgaaattagccccgccccgctgcggctgattcgtctagttaaaccctggtgttcctgacacaaacttcaggaaaggattttgcacttgtgcagaccgggcgagcagagtaagaagcagttggttaaaaccagagatgcggagcgcatggatacggagggctgactgcgcttggaacatttctgtcaccatgtgaacaagcctgctctagccaggtggatgctgtgaaacacgtggcccagtcctctccaacatcccagtcaacagccggcactgccagagacatggcaatccagctctctgcagctgcctatagattcacacgcaagcccagtcaagaccaatagaatggcccagttgagccctgttcaaactgtaacccatagaactgtgaccaaaataaatggtagtcattttaagccattaaaaaaaaaaaaaaa

SEQ ID NO: 77 - DNA, 430 bp, Homo sapiens
catatgttaacttattgaatgctgacagcagccctctaaggtagatgccattaatatttccatttacagatgaggaaacagaagcatagattaaataatttgcccaatgtcacacagtaagtggcagagttcatatcccttgaagacatgaccaccacgcttcactccctgcctgtgacacagaggaagctctgagatgaagagtcctctccgccaccagaagcagcagcgaaaacccatccaaagggttcaagaaaaactttcattctgagaagaggaaccgggtcggggaggcacgccggactccacatcatttcttccttcaattttgaaggagagtcgagggctggaccatcaaggctttagtctgaattcaggcctaatgtgagaattaaagaacagaaaccgccagactaaaaaaaaaaaaaaa

SEQ ID NO: 78 - DNA, 453 bp, Homo sapiens
ctggactgtggggttcatcctttcctagggagcttccaagggcccacacagcctgctccccgcaccccatggaggcgcaatgatagcagaattactggcactgttaacctgctggagtgggaacctggagtcctggaaggtgggacttgctcagggactcactgctggccttggggagcagagaactcaggtctttgtgattccgaagtccaggttcttccacctgcaactgcagtttccagaattggtcccaccccaggaggatcagccaggcagatgcaacgccaggagcagcatcagccacgctgtaaacaagggggaaacgccaagcgcattacagaggacgtcagccctgccatcactgggctggggaaacaatgccagccatggctggtctccgggttcacagtgataggggaaataaacccttatttgtctaaaaaaaaaaaaaaa

SEQ ID NO: 79 - DNA, 641 bp, Homo sapiens
aagattagctgagtttttctgctgacttaactgttgaatagcatcatcctcaggcactaccaaaagtgtttagagcactctgttgaaaatggcagtatttgaagaaactggatgtcacttaaaatatgatccttggataaacatcacatgctgctggccttcttacaaaatgagtgtgagtgaagtgcttggctctgcgagatggtcctggaatatcttcaccactcttcacaattacgtcctcttgcattcccactgatccttcagaattgtgtggcctttcaactttctgaaaggccttcactgtctcttaattcaaattctccacatgtaaatacaagtcagatcaccttacaatttaatctttaagcaaaactgtaaatcatcatttcccgcgacattacaattgttgggggaaaggtgtttggtgtcacatctaatggcttgaaaactcggagcatctctggacacagtcaatatccaggtctcaaatatgcatttactgtgtttattataatgactgcctttgtattcactgcaatcattttctactgtttcttcctttgtgtaatcatttcttcccttcccctccattgtatttttttagagtaattctatattctggttttgaatacacactt

SEQ ID NO: 80 - DNA, 479 bp, Homo sapiens
  misc_feature (364)..(364): n is a, c, g, or t
ccttcccctcctctgcatgcctttagcttcccaccaagctgggcctgagaaacaagggacccgaacctggagaggccggtgttcctggactgctctgcagtcacagattgtcacggagcccctggttgtctgaggaccccaggcctgaaccttccttgaggaagactgatttctttcagactactttgctcagtttgattttaaacagaaggctaataattatccagaagagacaggatgaggcttggacaaaggaggtggagggtatcaaaagatatttagaagataaaggaagtctggacttggtgctgtgggtataccctccctcatggcacacaccaggttattttgaagcaaatcctanacgtcatcaaacagttcaaattgcccaagtgtctcctacacacttcttttttcttttacagtttgtttgaatctgggtccaaataaagtccacacattgcaaaaaaaaaaaaaaa

SEQ ID NO: 81 - DNA, 578 bp, Homo sapiens
ttcaaagaaaggaacgatgtggacctgacctcaaagaaatccattggagaatatgacagatttagatttattgatcaactttacttttcctatacagatgggaaaacagcctgcaaaaatgttaagtagcttcctcaggatcaagtactattggagccagcattggtggtcaatcctgtatagaaatgacttcagttgtagatctgtgaccttccttacttaccttcctctaccaaagtgggtcaaccaaaaccgcatggcgtactactctctgaagcctctactaccctgctcctccgtgttgacatgtggtcaggcaagccaggacttactcacatcagctacatcagttactgggatggagaaaattgaagcctagaaagatcaagaaactttctccaggccataaatagaggaatcaggattcaaatcagatagaccccagggcttgttctcttcaacaccacattaccctacattattattcaattattaaataaaaccttgcattagtggcatttccaaatgcataacaaaaaaaataaaaaaaagtaacactggtcaaaaaaaaaaaaaaa

SEQ ID NO: 82 - DNA, 3587 bp, Homo sapiens
gggcagaccacaatgcaccgctcacggcgtcggttgcccgggcaatgggccgcacgctacgaggcccacacacccagaaggtggagccccggccgggttacgaggaccacccagctgtctggagagatgaagaaaatgaggttcaaagagatgaagtctcttgcctaaggtcagtgacagaaagtgacagagctgggatgtgaatcctggtctgactctacagtcccacatggtagatggaacctccgagcaacacctaaacaaagggagttgatgcctccgaacagagtaatgtcgctggagacactaagcatcgtcggcccctggcaggagctccctaaatatgtgttggatggatggaagaaaggatggatagttcagagtacttcagccttgattcctccgcaacagacacctgaaacttgaccctacattggttcttggccatcgtgatcttccttggacagaatcacctttcagctccggtcatcagactttcccagggccctcagaaagcccttcagagttgttactcacaggcaggctgagggattccttacggggtctgcagctctcctcacctcatccacaagtaggaccgtggcctgttcctcactactgccccaggatcactctgttcccagcccagtccagcaatcacttgtctagctttctggaaccttgagtactttcttgaaccatgagtcctgtgaccaccctagcagccctaaccctcccttatctgaaaggaagtgtgaggtgaccttgcaggtcccagagttgattgaagaccccatccagaaagaaggcaccctgtgggagagattgcaaggcctaggtctgaatccggaagcttccaccccatggagaagggctggcagtggagctgagctttggagccaaggactgtactgcagtgcagggagagtgaggccagaaaggctgagacaactcagggaaagaaaacctcccttctggctaatagtcaagcaccgcctgagtagaccaacactctcctgtccacaggggcagcagatgaagacacaaccagagaggactaacagcccccctcagctctcagtcagagggcagagcaacacagaatagacattaaaggaacagactttgaggccaggcagccttgggtgtgcatctgtccctactaagccatgtgacattaaacaagtgagtccacctctctgagcctcaggttcctcatctgtaaaatggggattataagagttcttgtttctcagggacaatgtgaggattaagtgagatgatacacatagagaatgtggtgcagtactgggcacatggcaaagatcagtgatgctagctaccacttatcattagtgttcctgtagaccaggacttctcaaccttggcactattcaccttttgggccagaaagttttttttgttgttgttgtggggagctgtcctatgcatttgaggatgtttaacagcacccctggcctctacccactagatgccaatcacaccacacccccagtttcaacaaccaaaaatgtctgcagatggtgccgaatgtcttctggagggtgaaatcactccccagcagttgaaaagcaccaccctaaacaatctggactgaatttgaatgtcacagatcccaagctcacagctccatgtaaaggccacaaggcaggcaggcctacttgcagatgaaggaacggaaagagcaacaattacagatcaagcggctagctccggtgtagactaagaggcgggatctgtagtctgctctaggagctaacaaggcctctgtgactcagtaggtgacctcagtgtcactctttaattatatgcaaagtggtcttgtgttaactcttaattattctagggctccaaacaagaaaataggtagttggtatctgatttctggcatcaagatcctgattcattgactgggagaaaatctgactctccaaaactctttcagagttcatttagcccttcatttatgactctggggagatttctgagcgagagctaggtgtcaggccctgttccaggtgctggagatgcacaagagaacaaaataggcaaagtttcctttttatggaacttatagcaggagaataaagataaaaacaagcaaaaatatttatctctgataagggcaatggataaaaataaagtagaataggccaggcacggtggcttacgcctgtaatcccaaggtgctgggattacctgcgtgagccaccgcaccctgcatcattttttcagcagacatttgttgagcacctgcttgatacaaggcacccatctaggcacaagggacatgaaaggaagcgagtcagacacagttcctatccttttggagtttagattttatgtgtggagacagacattgaacaggatgcacacaggatcacaggatgtgacaaatgcagtggaggagaaagataggctggtgtgagagctgttaatgggggctctgctctagcctgagaggcaaggaaggcttttccaggagtcacattagaggaacaatctatacaagcagggaactaggctgggggaagtgcagggagctttttaggtggaaagaacagcatgttcaaaggcctgaagctggaaggaggctggctgacttggctccagagaagctgttgggggagtgtgggagagacaagacagggagtaggggtggggagaagccagacctttggagtcttgtaagcctggttaagacacgtggactgtcttctgcggccaccagtggctctgttgaggtgggaaattaaagaaaagcaaaattaaaaagaaagagaaataagtttttctgtatgagggtgacttgtcccagaggcagcaataggcacaggccagacccaggaaaattcttcatgatattatctaatgtgctctggagattctcccagtactccctcaacatagggagaagaaaaacaaattttcctttgttttatggaatgagtttatagattcctattctctgtaaccagtgacttcaagtattgttttatctaagcagtggagtgaaggtcatgagcctctgagctggcctgagttacggccacctgggcgccatagtgaaggttatgggataagtctgtgcctgggcaaacctagataacggacatctgggttgcttggcaacggtcacgtgcaatcctgagtttgtcctgcctctatatccctgctttcatgccactgtaagcttgcttcaagctagcccacccccttttgtgaagtgtgtatagaagtcaagtgctgtctttcttccgggcccagtcttttggacgttgagtcagctgggcctgagtgcactcaataaatgattctcctgttttagattgagaccatcctgactagcacggtgaggccccgtctctactaaaaatacagaaacaaaattagccgggtgtggtggtgggcgcctgtggtcccagctactcgggaggctgaggcaggagaatggtgtgaacccagtaaatactgcacatgtgtggtgagccgagatcgcgccactgcactccagcctgggcaacagagcaagagtccgtctcaaaaaaaaaaaaaaaaaaaaaaaa

SEQ ID NO: 83 - DNA, 2712 bp, Homo sapiens
accaacagacacagacatttacacttctaggccaggaaagcgctaaccagggccctgtgactctacgcaggttccagaacacgccttctacatttgttactgaaccgatcagcgaacacagacaaacgtgccaacacttaagtctactggctggacttcatctccatggcaacaagcatggaaggcaaagagttgattccagaaggaactgtgaagagccacaacaatgtgccagtgaataatgagtagtacctactgtggcaactcttcagctaagatgagtgtcaacgaagtatcagctttctcattgactctggagcaaaaaactggctttgcttttgttgggattttgtgtatcttcttgggacttcttattatccgatgcttcaaaatcctgctagacccatatagtagcatgccttcctctacatgggaagatgaagttgaagagtttgataaagggacatttgaatatgcactcgcgtgagagttccagctatatggtttttatggttgtgccatcgggacacattctggatacaattgtaactaccttgagggtgtgggagagaggctcattttgttcagtgaattcaataaacatctgtgactaatttcttcaccatgctgtgtaaatgataaactattgttgggattcctcactttcctctgttcccacttagaggtttccaatgaatagagcataaggatggctgccgccatgtttaccatctttattcttatcttgaacaactgccttgaatttctgcttcctttcttgtgctaataaactgtgccaaaggcatcttgctctctcccctctgaaaggcccaggcatcccctccacattcctgactcagacccctttagctttagtgagtcaggtcttaaacagagaatttagtccaaagaataatcaaaatattatttgtctaataataaccataggcccaaaaggttcacaggtgaaaaaatatctcaaaaatctaacacccaaagcctacaagtagtcacaggccctaaagctccccaaatttcacagagccctgaccctcctatctcttctgtttgtcgaatctcttgcagtaaaatcccatagcttggaatccaaaccagatctgcctcatactgatgctccctcccctaccccttgcataaatagaatttaaaagggagtttccagcatgacctagtggaaagagtactctaagcagaaacaaagacagtctcagctgtgccaccagtagagctatgctctaatgggtaagtcatctaatctctgtgagcttcagtgaagagtagtgttaacatccttttcaattctagtgccctatgaatgctacaaatgtatctgaaaacccagagatctgaaatgaatgaggatttaagatatcatgaaaagtttttcagattaggagcctaaactcagagatttgaattctaatgttagcttagccaagaactagctttatgacctagaaagttacctaaagggaacctatattgcacaacaagaattaagaaacccatcctgcatgtcttcagcaatattgagagggccaattagatataatgaatgtgaaagcattttgtaaaccataaaacagtatctaaacataccttgctatcaatatttattgtcctcatcagtagcagtaatagagattctcaaaccctaagaagcaatggcttagtgtaaattattcaatctctctagtatttcattctcttcaattgagaaataaaaagataaaattatatagttctaaattctttccaactacaaacttctgactctctatgagtcagaatggaggaacatagcaggagataaatacaaaaatatttatgtcctttgaaaacacagaactgcaaaacataccatgaaggaacaagactaatattaccaagaggtagcactaagttagttactttcaatttatttgatactcttataaattctttgagttggagactactatttccattttatttttaatcttaattttgcagaagagaaactgaagctaagagaaattaactagcttcccaaggtcaccatagctagggaatggttatgttagggattgaacttgggcagtttgatttctgctgtatccctagacactgtcctcaacacttctacatagcttataggcatggttactgctcaggtctatggaattccaaaaccagctgtctctgtgagattccttgatgctttccatctcataatagatttagaacatatttgttattagctatggatcttctggaccaaatcctcacctcaaggaggctctctgtaccccagttggttcaagagtgtaggcctggtacacctcatagccagttagtaacttggcccaagaacaggccactgggccaagttcaactgaacatactgaaaattgtgtgtgccaggaaactaggtctctttgttgaccaaggcttttaaagtaccagatgtttcatttgtctcccagagaagacacccaccaagaccctccattttgatgactagaacaaaccaaggccagatctttatttcaaaggaagaaagacctctacaaccaagaaaactttatgaaatatgagcattgtttctggaagttcaactcccataagcttttcaagtatattaactctaccataatattatttccaaatcccctaaagaaaaattctattaaccgaaaaaaaaaaaaaaaa

SEQ ID NO: 84 - DNA, 3478 bp, Homo sapiens
ggaaagtctttgttccttctttacttttggcttcttcttctgaaacatcttctttatccgcctcttgttcttcctccagttttttcaaaggttgagcagattctgataatttttttgaagggtatggctgtggtgtgtgcctttctgaaggacaaaggcaacctgccacaaatatcatacagagtcctagtaacagtccagaatgcagagttgctaaaggaaaaacagcatatgatccccacactggcaatccaatgtcttcaataaagtagttatggcaagtcctgatccacgtagatagctgaaagagtgctgacgtactactcatctgaacagaacctgaaacaaaccatgatgaaacgggttcaatactcttccactctttatcacttataaagtttatgaagtccttctgagtccttggaccctgaggatgcctaaattcaccatctttacaatgataaatagtaggaagaacagttattgtaaactgtccagtcagtcctggctgctctgtgacatctactttcacaacattaacctcagatcttctccccattcagcaaaactttcccattccggttaatggttttgacaagcagggcaccacggggcataaaattgtatcatccagcctccttccagcagctctctccagttcttgtctgtgatgatgcgtacgttgctctgccgcctgtgggtccagggagcacccccaagcaacagtaccaggactgccagcggaactgcaagactcctggagggtgccatgtctgccgcttgcccacctcacagcaagcgtggcggcccaacactaggttttttaaaaactgtgactatcagtgttttaaaaattgcccggtaactctagacttcaaaagtgggataagtaatgataaaccaataataaacttaggagaagcatagtctgctttagttatatcgttatgccgtattatggtcagtaacacagtaaaggcctaattcatctgctttctaaattggttctgtacttttctagaaaagcctacatgtatatacttagttacagctgcacttctccattacttatttttaggaaggttatagagatggaatagatgctggcaaagcagttactcttcaacagggcttcaatcaaggttataagaaaggtgcagaagtcattttaaactatggacgactccgaggaacattgaggtaatttttaaagtctaaatgctgaatcattttaacctcaatactactggaggatgtttctgtataaataaagtgtttaaactgaaatgcttttcctggtgctaaatacactaaagcgtgtcgcagatcatagaattatattgccttcaaaaagtcaaaatctttatcagctcatctactttaatgtgtgaactacatattgtcttttcgtgcaaagaaatggtaagaagatgtatacttctgctacctgaacaattatctatctcattgaaaggtcttcagattttgaataaaacttgtagtacttccacacagtatgacagacctctagactagaagtacatgatgaaaatagttggtaattaagataaaattgatttaatttactttagtcctgaacattgaatacttgtcaggacgccattgcaataatggcatttatcggagccaaatggtcaaatgatacacagagccaggagcctagcagccttgtccagtttgatgctctataccaagcttgtccaaccagtggcctgcatatcacatgtggcccaggacggctttgaatatggcccaacacaaattcataaactttcttaaaacaatatgagcattatgaaatttttttcatgatattttttcttttttcttttttttttttttaactcatcagctatcattagtgttaatgtattttatgtgtggcccaagacagttcttccaatgtggcccaggaaagccaaaagattggacacccctgctttataccctttacactgtccttggtagagaaaaaaaaaatgcttcaaagaatcgctaattttaaagaagagtagatgataaaagttgccaaaacaaaccgaaaaatttattgtatttgggattttagaaaatccaactattaggaaccagaatttagtctgctacagtaggaaaacaatgtgaatattcacatcatcaagttgatgttacataaccttagaaagctactgctgaatcttttatatcaatggattatatttttaaatacttttcataataatcattattttatgacatgactataatattaaatctgttaggactagaagaatttttacctttttcaaggaaattgttagtagttcagcaaacagtttctactctgtaacataagcccaggaaagtgaagtctcttgaaaactttttttctctaaccttcattcttgatggcaagcaactatgtgcttagaacgatggttttcaactttggttgcaccttaactctagaacttaaaaaaaagataccccctgagattctgatttaattggtgtggagtataatctgggccttgatagggttcagagctcttcaggtgattctaatgtgcatccatgattgagaattgctagttaagaagctgtttaatgtccttaaagaagaaactaatttttctttctcggagttgtattcatcttcaacagatattacatagtcataagagaaaaatataaaatcaggaaaagcgtatatagagttatgaaagaggggttatgaattataaacagttttatgattaagtccaatcgtttaattgttattgaaagatagtcttatatttttaagtcctattttgctatttaacccttgtttatacttttgttcagtgctttgctctcctggtgtcaccttcataataataattcaactttgatcaataaaataaacaatcttctggatgcagttggccagtgtgaagagtatgtgctcaaacatctgaaattaatcactccaccgtcccatgttgtagatttattggactccattgaggatatggacctttgtcatgtagttccagctgagaaaaagattgatgaagctaaagatgaaagactctgtgaaaataatgctgagtttaacaaaaactgtagcaagagccatagtgggatagattgttcatatgtagaatgttgtagaacacaggagcatgcacattcagaaaacccaagccccacatggattttggaacagacagccagtttagttaaacagctgggcctatcagtagatgtattacaacacctcaaacaactataaaattaccttcccttttctaatgaaaataatgttcagaacatttggtttcctaacaatcgaaatttgtactggtttctgcatcaaacacctcaactgtagggttaccctttatggaagtttgaaattaacactattgtcttcaaaattaacactattaaatgtaatataagcctttaaaagaaaaaaaaaaaaaaaaaaaca

SEQ ID NO: 85 - DNA, 5657 bp, Homo sapiens
ggtggttccccaatgcccccacccttccgtggccccagccagtggggctcagctctggccaccctgacatgaaggggaaggtccagcagccaaaccccaggggctctgaaggaatcggcctgtgggtaggagatgccgggtacctgcttttgtttctttttaaatttcccccaggtgattctgttgtgctaccaggatgccaaatcccccaggccccttaacctttgtaggagcacacgctcgaaagcaatcgctgcagtcagcctgcacacggctcccccacagaggcccaaacacacctggccacacactgcacacgtgctcacagcaccccacactcacacccacgccgggacctggctccctgaggacctggatgcacaaggagggacatgcacgtgaccttcaagctgagggtgggatcgcactgctcgaacatgaagtcccagatatccagggttccgtccatcctggtggtaaagaaaacggtcggcctcacggggctccaggcagcatcagtgaggaggaggacaagggagacccaggttctatgacacaggtccaccaagtccctctaggaaccagccctgcgaaaggcctatgggtgctgtgctcgacaccctgctcctttcacacagccctggcagcctgcaggacctgaagacattgggaaggctcctaaagcctgtaaagggcttcacatctactgggaaccccctacttgctatgaggcctggaacaatgcactgcccttgtcagccagcgttcccacccctgtagaatgaggacatcgaaccaggtttctgctcccaaagcaaggactggctgagtcacaagcctggggttgctccttagcaaacccattcctgggctaagcccaagattctatgttagtacccccagggttggggctgagaatctggattttaaaaaaatcactcctccaccaggcacggtggctcatgcctgtaatcccagcccttcgggaggctgaggcaggcgaatcacctgaggtcaggagtttgagaccagcctggccaacatggggaaactctgtctgtactaaaaatacaaaaattagctgggtgtgctggcgtgcgcctgtaatcccagctactcaggaggataaggcaggagaatcgcatgaactcgggaggcggaggttgcagtgagccgagatcgcgccattgcacttcagcctgggtgacagagtaagactccatctcaaaagcattttaacaaacaaacaaaaactcctcccaagatattctgatgcaaccagatttgggaagcactggaaccaaagatgctcattagggttgctggtggctttgacgttgcaggagtctgtggttctgtgggcaggaaaggaaccccaggatgttttttttagcaagagaaaaaagtaattatttatacatgcaatgcacaatacacatggcagtgctcggtggtgaatgactcaagggggtggttagaattcgggcttattacctaatttaggagatgaaggggaggaggagaaagggcacttttagacaaggtaaatgggccctaagaagaatggagggggatgtgatagtccaagcttctctggtctaaccattgtgctcatagctctgtctcaagctccctgtctgttgctctcctcagaggagtggcactgagcaagacaggctatgcctcactgctgaatcgtgccaaggcacccgcatttcccagccaagcttcctgggttcacacccaatctcggctcctcactagctgggagagcaggagcaagtggcttggcctttctgtagcccagttccctaacatgaaaaatgagatgaagagaagagcaatgctgcctcctcctggggatagtttgaggattatgaaaattaagccagccagacgcggtggctcacacctgtaatcccaacgctttgggaggccgaggtgggcggatcacctgaggtcaggagttcaagaccagcctggccaacgtggcaaaatcccatctctactaaaaatacaaaaattagttgggcatggtggcaggtgcttgtagtcccagctactcaggaggctgaggcaggagagtctcttgaacctgggaggtggaggctgcagtgagccgagatcacgccactgcactcagtctggggaactaagcgagactgtctcaaaaaaaaaaaaaaaaaaaaaaaggaaataaaattaagccatggccattatttttagtaactgagtgtttgaagcttatcactttgtggagtggctggagcaagtcacttgctaacacgagcttgggtgtttgtggaaaggtttttctccattttacatatggaggctcgggactctccggggcccagctgggctccctccatcccctcctgtctcctgcatcccctcctgtccccagcatcgcctcttacttggtccacatgatggacgattcccggctgtcttcagaccaaatgcgggctgtccagtcgccaaccgtcaggaagttcttcgggtagaaggggtttctctggagggcgtagatggggccatgatggcccgggaaggtgcacacaatcttctcagctgacgtcttggccttgcggttgcaggagatgacgatgccctgctcggtccccaccatgaacttggtgggctgtggaaggagtgacacaggaggggaagactccacgaaccatcaattctgggattagcgggcatggacaggagcttgcttggggggtctgcaggcgctagaaaggccctcagatcaagccttggcaagggctgtggcccctcctggagagcagtccccactcttcccgggctcctcagaccccatctttttggaagctgctcaaggagcctgtgttgaatgaagccctccctggctcctccagtcctggagggttgtcaggtcactagccccctctgcagagcaccactgtgtcctcgatgcatttggccataaagatcctgaatgtttgtttacagagagagtaaagaagacccagggaggggacgcagggtggcccacccagatcactcatgggatgggcagagccttccctggcaagacagtgacatttgtcatagcattgtccaggctggtgtccctgagaactcagagcaggtgaccaaccagcactcaggaagctacctggcagtcgatgatgtaactcgtccccctaccactggtgggtgagctctttgaggggcacgagcttgtctgacatgggtccatcgcccacgaactgagctttagtagttattcacttcactgcggactgaatgcacaaaggctggtctcctgactcctggtccagtgctcccctcgcgtctctgagcatgaccccctccatcacgcaatgcccttccttgtttactgttctgtcttaacttctgggctagactgaacaacctccaccagcagggcctgagcctacaatggtgctgtgtcctcagcattccaaatggaaagaacagctgagtgaggtaggggaggggcacactgaggggagggcctttgtcaacgggctcagatgtcctctgcagctggtgaacctgcccctatagaactcccaggttttaggcagaggttgcagtgagccgagatcatgccactgcactccagcctggcctacagagcaaggccctacctcaaaaaaaaaaaaaaaaaaaaaaaaagaaaaaagaaaaagaaaaaagaaaagaaaagaaaaaagaactcctagtttatttatttatttctcgagacaggttctggctccattgcccaggctggagtgcagcggtgcgatctcagctcactgcatccttgaccttctgggctcaagcagtactcccacctcagcctcccacgtagagaccacaagcacacaccaccagggtcagctaatttttgtatttttgtagagatggagtctccctatgttgtccagactggtctcaaactcctaagctcaagtgatctgcacacatcggcttcccaaagtgctgggattacagacctgagccactgcacctggccccccagcttttttatttttatttttttaaaaaacatattgttttattatgcagttttcttctaccagaaaattactaaaatgataaatataaaatctctagaaaatacccagaacatggccaggtgtggtggttgaggcacgagaatcacttgaacctgggaggcagaggttgcagcgagccaagatcatgtcactgcactccaggctggtcttgaactgctgggctcaagtgatcctgccacctcagcctcccaaagtgctgggattacaggtgtgagccacggcgccttgcctcacattgatatttttcttctttttctttccttccttccttccttccttccttccttccttccttccttccttccttccttccttctttccttccttctttccttttctttccctctctctccctctctttctttctttctttcttggtaaagactgggcgggcggtggggggggtctcattatgtacccaggctggtcttaaattcttggcctcccaaagtgctgggattacaggcgtgagccaccctgcccgacctgaactttgatatttctttgtcattctcagtgactgaacaccgagaccatgcttgtgctctggttttccaaggagcctgctttgacctcattttcctctccagtacctcttccttccccagaggaaggcaggtgaccagcctagggaccccgtgccaggcagggccaggacctgggttggagtccaggcctcccgatgttggtcctgggcgtgaccttccccacaaaacagggcttttctggaattgttctgtcaaaagattgccctctgtgttcagctgggccactggccaaaagatgacttggtgccatgatgactatggtgtccttcttcgagaagctctgctgccccctgctggaaagttgttgtaaaggccacacacgtctaggatggctgcagtgagggtggcctccctgggctggtgctgggttccacgagcacctgatatcctagcaatggcctgtctttagccctggctgccctgtcaatgcaagtcggggaagggacagcaagggacactcaccaaagtagattcgaactccagggagatggcccccaaggcattttccaactgttccttcttggtgatgtccaagatcacaacttcagtgggctcgctcatctttcggatgtcccaccacatgacctggcgggaggaggggcgacagtgagacttcgaggccctgtatttcccctggcttttgccttctccgccctcaccccaccatcctggggtcagccactgggccacaaaggatggagctcccggcaattcgtgggagcaaatacactttgaggattgctgttctgtccacagttcccacacctggctgatcatcagagtcgccggcaagcttcatacaacccagatgctgaggcctcgccctcgacctactgaatcaaaacctccaggtgcaaggccaagaatcggtatatgccaagactccccacaataaggcaatatccggcaatgttaaaaatgcatgtgtgctccgacctagaagtcccactgccagcaatgtctgctgtagatatatccccgcatgtgtgcaacaatctgtgtataaagaaaaaaaaaaaaaaaaaaa

SEQ ID NO: 86 - DNA, 1920 bp, Homo sapiens
acaatcctttgcggtggttcaagatggcggcgcccagtggcactgtgagcgattcggaaagtagtaacagcagtagcgatgcggaggagctggagcggtgccgcgaggcggcaatgccggcttggggcttggagcaacgcccgcacgtggcagggaagccaagagccggtgctgcaaatagccagttgtcaacctcccaaccgagcctcaggcataaggtgaatgagcatgaacaagatggcaacgagcttcagaccacccctgaattccgagcccacgtagccaagaagctgggagccctgctggacagcttcattaccatctcagaagcagcaaaggagccagcaaaagctaaggtacagaaagtcgctttggaggatgatggtttccgccttttcttcacatctgtccctggaggccgtgagaaggaagagtctccccaaccccgccgaaagcgacagccctccagctccagtgaggacagtgacgaggagtggcggcggtgccgggaggcagctgtgtcggcgtccgacatcctacaggagtcagccatccacagccctggaactgtggagaaggaggcaaagaagaaaaggaagttgaaaaagaaagccaagaaggtggccagtgtcgactcggctgtcgctgccaccacccccaccagcatggccacagtccagaagcagaagtcaggtgagctcaacggggaccaggtgtcgcttgggaccaaaaagaagaaaaaggcaaagaaggccagcgagacctctccattcccaccagcaaagagtgctacagctatacctgcaaactgaacccagccatgggcacagggctcagccagctccaaggacaaggtgtcccccccaccctggggacaaggcatttccaagccccacctccctctccaagttcaaggactgggctggcaaacccagactgcccatgagaccctgatggtgatgaaggcttgctcgagagttgggccaaaaaagtggctgtagggtgagaagaccaatcaaggcctgcccctctgtgctcccacggagggctggcgggggcgctgtggttccagaacatttcatgacctcaggaaaaaaggaaccttccagttatttgagactagtggccaagtggtgaaacctccatctccctagaactgtctgaggtgggcaggggaagggagaccttccccaccacctccttagcctggtgtgagaaacagtttttagaaaatgagagaagggatccccaagaggccaaggcccagagaaacttttgttcttctctccttggcccacatagacttcacagaatcgtctgagaaaggagagctttttcacccctggctttctagaattttctttgtctgcatttgtgaatataccacacatgatggtgtctctgagccgaccagattatggaaactcaattgtcagaggacccaaaacaaaacttagagtgatttggatattgcctctctgtcaatgctgaaccttaagacattttaagtaaacatcctcctcctctctacacccccaggatttgtgcgttcaccccacccaagacttcaggaagtgtcatagcactcgtggactaggtttcatgggacaaaggcattctgcaagaagtagaagcttgtggccgggtgcagtggctcatgcctgtaatccaagcattttgagaggccaaggaggtggatcaattgaggccaggagtttgagaccagcctggccaacatggcgagaccccatctctactaaaaatacaaaaattagctgagcatgttgacacacgcccgtaatcccagctactcaggaggctgaggtgggagaatcgcttgaacccaggaggtgcaggttgcagtgagccgagattgcgccactgcactctagcctaagcaatggagcaagaccctatctc

SEQ ID NO: 87 - DNA, 622 bp, Homo sapiens
atccgggcctgagagtgcaggcttgagggaagcatggaggtccatggcaagcccaaggctagcccgagttgttcgtcgcccacccgggattcctcaggagtcccagtgtccaaggagctgctgacggcgggaagcgacggccgcggaggtatatgggacaggttgctcatcaactcccaacctaagtccagaaagacctccactcttcaaacagttcggatagagaggagtcccttattggaccaggtacagacatttctcccacagatggcacgggcaaatgaaaagctaagaaaagaaatggcagctgcaccacctggtcgtttcaatattgaaaacattgatgggcctcatagtaaagttatacaaatggatgtggctttgtttgagatgaatcagtcggattcaaaagaagtggacagttcagaagagagttcacaagacagttcagagaacagttcagaatcagaagacgaagatgacagcatcccatctgaagtcaccatagataacattaagcttcccaattctgaaggtggaaaaggcaagattgaagttttggacagtccagcaagtaaaaaaaagaaatagtcaaataaattatctgaaaagaaacaggtga

SEQ ID NO: 88 - DNA, 321 bp, Homo sapiens
atgtttgcacccgcggtgatgcgtgcttttcgcaagaacaagactctcggctatggagtccccatgttgttgctgattgttggaggttcttttggtcttcgtgagttttctcaaatccgatatgatgctgtgaagagtaaaatggatcctgagcttgaaaaaaaactgaaagagaataaaatatctttagagtcggaatatgagaaaatcaaagactccaagtttgatgactggaagaatattcgaggacccaggccttgggaagatcctgacctcctccaaggaagaaatccagaaagccttaagactaagacaacttaa

SEQ ID NO: 89 - DNA, 673 bp, Homo sapiens
cttcccggcatcccctgcgcgcgcctgcgcgctcggtgacctttccgagttggctgcagatttgtggtgcgttctgagccgtctgtcctgcgccaagatgcttcaaagtattattaaaaacatatggatccccatgaagccctactacaccaaagtttaccaggagatttggataggaatggggctgatgggcttcatcgtttataaaatccgggctgctgataaaagaagtaaggctttgaaagcttcagcgcctgctcctggtcatcactaaccagatttacttggagtacatgtgaaagaaaacgtcagtctgcctgtaaatttcagcaagccgtgttagatggggagcgtggaacgtcactgtacacttgtataagtaccgtttacttcatggcatgaataaatggatctgtgagatgcactgctacctggtactgctttcagtgtgttccccctcagcccctccggcgtgtcaggcatactctgagtagataatttgtcatgcagcgcatgcaatcagaatctcactgagccacccatcattgtgaaataattacctcagttgtacaggacttggtgatcaggatccaggcactcacttgtattctactgctcaataaacgtttattaaacttgatcctgctacttaaaaaaaaaaaaaaaaaaaaaa

SEQ ID NO: 90 - DNA, 766 bp, Homo sapiens
gcgagccccggcgcaggggccggatctggccgggggccggcggcggtgtgggagcggcgcgtcatgtacaccatcaccaaggggcccagcaagctggtcgcgcagcgccgcacaggtcccacgcagcagcaggtggagggccggctcggcgagctcctgaaatgccggcagcccgcgccgccgacctcgcagcccccgcgggcgcagccctttgcgcagccgccgggaccctggcccctgtcgagcctggcagcaggtgcaacagcagctggatggtggcccagccggtgagggcgggccaaggcctgtgcagtacgtggagaggacccccaatccccggctgcagaactttgtgcccattgacctagacgagtggtgggcgcagcagttcctggcgagaatcaccagctgttcctagtggctgctgggagggggcgctgctacacggccgacctgtcgccaggagagaagcatggcgccctgcccacccactgcgcctggctgggtgccggccacacctgaagtgccagcatttggacttttgcacctttttttcccttggcccggctgtcccaaccaagctgccatggccaagggccgaacccgtctgacctcagccctgctcactgtgcccagggaccagcgaccagcccctggggctggcagggaggagctccaggctaataaagtggagaaactgtcaaaaaaaaaaaaaaaaaaaaaacaaaaaaaaaaaaaaaaaaaaaaaaaaaaaaaaaaaaaaaaa

SEQ ID NO: 91 - DNA, 461 bp, Homo sapiens
attcctccaacgggcaggtctcagcgctcctccccctgctccgctcctctgcagggcccaggcgcccttggccttaggacccaacttctcttaccgccatggagttcgacctgggagcagccctggagcccacctcccagaagcccggtgtgggggcgggccacgggggagatcccaagctcagtccccacaaagttcagggccggtcggaggcaggggcaggtccgggtccaaagcaaggacaccacagctcttccgactccagcagcagctccagcgattcggacacggatgtgaagtcccacgctgctggctccaagcagcacgagagcatcccgggcaaggccaagaagcccaaagtgaagaagaaggagaagggcaagaaggagaagggcaagaagaaggaggctccccactgaagggccctggacagggctcattaaaccttcctctctgccttc

SEQ ID NO: 92 - DNA, 945 bp, Homo sapiens
ccaagcgcggggccggagcggccttcccggagtcctttgcgcggcacctggcgacaaaatggctgcccgagggagacgggcggagcctcagggccgggaggctccgggccccgcgggcggtggcggtggcgggagccgttgggctgagtcgggatcggggacgtcgcccgagagcggggacgaggaggtgtcgggcgcgggttcgagcccggtgtcgggcggcgtgaacttgttcgccaacgacggcagcttcctggagctgttcaagcggaagatggaggaggagcagcggcagcggcaggaggagccgcccccgggtccgcagcgacccgaccagtcggccgccgccgctggccccggggatccgaagaggaagggcggtccgggctccacacttagcttcgtgggcaaacgcagaggcgggaacaaactagccctcaagacgggaatagtagccaagaagcagaagacggaggatgaggtattaacaagtaaaggtgacgcgtgggccaagtacatggcagaagtgaaaaagtacaaagctcaccagtgcggtgacgatgataaaactcggcccctggtgaaatgacgcccctcccccacctgcccatggcctgggactctctgcgatgtacataactatttaatgcagcggcagcggcgacagccttccctgagaggacttaaaagcagaaggaaaccgagatgcttcccgcagccgtggacgattctccaggactctttttttaccttgagcacttgcctcgtgagacttcatagaacagtggtttactgtcccccccttctcacctcctcattctctctggctctttctgtcttcctcttctcaccctcctccctccccttagccatcacttctgggaagtaaagaacttgacttagtgccggaaaaaaaaaaaaaaaaaaaaaaaaaaaaaaaaaaaa

SEQ ID NO: 93 - DNA, 934 bp, Homo sapiens
ctcacagtcccgcctcttccgctgcgtgccggaccatggcgcaggggcagcgcaagtttcaggcgcacaaacccgcaaagagtaagacggcagcggcagcctctgaaaagaatcggggcccaagaaaaggcggtcgtgttatcgctcccaagaaggcgcgcgtcgtgcagcagcaaaagctcaagaagaacctagaagtcggaatccggaagaagatcgaacatgacgtggtgatgaaagccagcagcagcctgcccaagaagctggcactgctgaaggccccagccaagaagaaaggggcagctgccgccacctcctccaagacaccttcctgaggacgctggccccagtgcaggccaacatcccaccccctacctccatatgggaccttgcaagtcatcccacaggctgcactgtcaggaagaggaccctgtcccccagcactgggcttcacctagaacttcagtgggggccaagggtgctgagaacccagcaatgaccaggaagatacagtcactaacttcatctgtccccgtgccccttcccaggtcctgcctccacaggtttaacccagaacaataaacctggctttgtcatccctcttgcagtcctgtgttcgggtgagcaggccaggtgagcccacaagtctccatgagtgacgtggcctggcgtgctccaccccaccccaccgcctttagcaaccatgtgcccaggggacagctgggcttctacacctctggccctgagcctgagagccgggaaagagtcttttctccatttaaccccgggtgactcactccctggccagtcctcacccctggggacacaaccagagtcaagctggacatcagtaggtcagatgccacctcacaggaccaaggtgccgattaaaccggaatacattcagaaaaaaaaaaaaaaaaaaaaaaaaaaa

SEQ ID NO: 94 - DNA, 2371 bp, Homo sapiens
atccacgcacgctcagcccggcgagcgcattcagttctcgagctccagccctcagcgcatgcgcaggacgagtcgcctgagggaactgatctcagctcgggcccgcgttacatcctcctcctcttcttccttcggcccagctttccttaggggctgcaacccggacgccgaggccggtttcggagtggggagtgcccattttctctccttcccacgttcctggcccccagacgccatttgcaggcgggtggcttgggtcagcctccccgcccccacccgactcccgtcacgggagagcgcacaccgcgccccgagaaccaatcagcagccgcgttaggtaaccatgtctgagtctggacacagtcagcctggactctatgggatagagcggcggcgacggtggaaggagcctggctctggtggcccccagaatctctctgggcctggtggtcgggagagggactacattgcaccatgggaaagagagagaagggatgccagcgaagagacaagcacttccgtcatgcagaaaacccccatcatcctctcaaaacctccagcagagcggtcaaaacagccaccacctccaacagcccctgctgccccgcctgctccagcccctctggagaagcccatcgttctcatgaagccacgggaggaggggaaggggcctgtggccgtgacaggtgcctctacccctgagggcaccgccccaccaccccctgcagcccctgcgccacccaagggggagaaggaggggcagagacccacacagcctgtgtaccagatccagaaccggggcatgggcactgccgcaccagcagccatggaccctgtcgtgggtcaggccaaactactgcccccagagcgcatgaagcacagcatcaagttggtggatgaccagatgaattggtgtgacagtgccatcgagtacctgttggatcagactgatgtgttggtggttggtgtcctgggcctccaggggacaggcaagtccatggtcatgtcattgttgtcagccaacactccagaggaggaccagaggacttatgttttccgggcccagagcgctgaaatgaaggaacgagggggcaaccagaccagtggcatcgacttctttattacccaagaacggattgttttcctggacacacagcccatcctgagcccttctatcctagaccatctcatcaataatgaccgcaaactgcctccagagtacaaccttccccacacttacgttgaaatgcagtcactccagattgctgccttccttttcacggtctgccatgtggtgattgttgtccaggactggttcacagacctcagtctctacaggttcctgcagacagcagagatggtgaagccctccaccccatcccccagccacgagtccagcagctcatcgggctccgatgaaggcaccgagtactacccccacctagtcttcttgcagaacaaagctcgccgagaggacttctgtcctcggaagctgcggcagatgcacctgatgattgaccagctcatggcccactcccacctgcgttacaagggaactctgtccatgttacaatgcaatgtcttcccggggcttccacctgacttcctggactctgaggtcaacttattcctggtacccttcatggacagtgaagcagagagtgaaaacccaccaagagcaggacctggttccagcccactcttctccctgctgcctgggtatcgtggccaccccagtttccagtccttggtgagcaagctccggagccaagtgatgtccatggcccggccacagctgtcacacacgatcctcaccgagaagaactggttccactacgctgcccggatctgggatggggtgagaaagtcctctgctctggcagagtacagccgcctgctggcctgaggccaaggagaggaatgtcatgcaggggacctcctgggtccgcagtgtactgcgagggagcacagatgtccatcccccgctggggtggagagcggcagcaggcctgatggatgagggatcgtggcttcccggcccagagacatgaggtgtccagggccaggccccccaccctcagttggggctgttccgggggtgactgtgagcgatcccaccccaaacctgagatggggtagcccgtcctgtgtcctccacagggacaagcagtgggaggagtctgaatggtcaccaggaagcccgggctccatcttgacctcctttttcagggacaggagcaacaggcccctcttccctgactctaagcccttccctgtaaggtgaggcagggtctggagagctctttattggaacagatctggtggttcaaataaacacagtcatgcaagcctgaaaaaaaaaaaaaaaaaa

SEQ ID NO: 95 - DNA, 1091 bp, Homo sapiens
cgcggcgcctgctctgtagagccggcggaaccgggtagcttggccaggttgtgaggaaccgcagcgcgccgcaggaccgggccgctgagcctgcagccgccccgcgccgtgacctgcgaccctagaccccgactccctttggctcagcccgcgcgccccaggcccggcccgggcggcgcgacgggaggatgagcggcgggcggcggaaggaggagccgcctcagccgcagctggccaacggggccctcaaagtctccgtctggagtaaggtgctgcggagcgacgcggcctgggaggataaggatgaatttttagatgtgatctactggttccgacagatcattgctgtggtcctgggtgtcatttggggagttttgccattacgagggttcttgggaatagcaggattctgcctgatcaatgcaggagtcctgtacctctacttcagcaattacctacagattgatgaggaagaatatggtggcacgtgggagctcacgaaggaagggtttatgacctcttttgccttgttcatggtcatttggatcatcttttacactgccatccattatgactgatggtgtacagctcccaagtgctccctatccagtccaaaggaccctcttgattacagcacaggaacttgatcgttggggaaccccagccccttggaacttggaagacccgtgtttcctggaccgcgaatcagtgtgttgggcatcagtgttttctgcaagggttgtgacctgaaactttttaaaaaccacccacctttggggaagcatttctgaatttatccatcaccaaccatttcttcttggataccatcaagtaacagctattatttgccaagtggagctgtcatttaatttgatgcacctctggattcagatgaaacattaaattgtcttcctcgattctccatcgggtgtagagtttttaaactatcaatggcatttcaagtcttctgaaacagcatggctgtatgtgcgtggtccatagcacagtacatgcagcatctaataagagtttccattgtagaatgttttcacatacttgaataaatcaaatctttaattgagaaaaaaaaa

SEQ ID NO: 96 - DNA, 1064 bp, Homo sapiens
agcggctgttagtgcgtcgcagctgctggcgatccggcgaccctcggccggcaggacccgcgggccacgcagccggggccttctcaacgcctcagtacctcggcgggaccgccatggttctgctgcacgtgaagcggggcgacgagagccagttcctgctgcaggcgcctgggagtaccgagctggaggagctcacggtgcaggtggcccgggtctataatgggcggctcaaggtgcagcgcctctgctcagaaatggaagaattagccgaacatggcatatttctccctcctaatatgcaaggactgaccgatgatcagattgaagaattgaaattgaaggatgaatggggtgaaaaatgcgtacccagcggaggtgcagtgtttaaaaaggatgatattggacgaaggaatgggcaagctccaaatgagaagatgaagcaagtgttaaagaagactatagaagaagccaaagcaataatatctaagaaacaagtggaagccggtgtctgtgttaccatggagatggtgaaagatgccttggaccagcttcgaggcgcggtgatgattgtttaccccatggggttgccaccgtatgatcccatccgcatggagtttgaaaataaggaagacttgtcgggaacacaggcagggctcaacgtcattaaagaggcagaggcgcagctgtggtgggcagccaaggagctgagaagaacgaagaagctttcagactacgtggggaagaatgaaaaaaccaaaattatcgccaagattcagcaaaggggacagggagctccagcccgagagcctattattagcagtgaggagcagaagcagctgatgctgtactatcacagaagacaagaggagctcaagagattggaagaaaatgatgatgatgcctatttaaactcaccatgggcggataacactgctttgaaaagacattttcatggagtgaaagacataaagtggagaccaagatgaagttcaccagctgatgacacttccaaagagattagctcacctttctcctaggcaattataatttaaaaaaaaaaaaaa

SEQ ID NO: 97 - DNA, 1416 bp, Homo sapiens
attcggcaccgcagcgtaggtgctaccaccgctgccgtcgccgccgccattttgatggcaggaagagtccggttctgggacagctggagacagtggtggtgactgaaataactttaccaaaggaaagctattttgcgaactatcttctccagcggagatggccaatgtgctttgtaacagagccagactggtttcctatctcccaggattttgctctttagttaaaagggttgtcaatcccaaagccttttcgactgcaggatcatcaggttcggatgagtctcatgtggctgctgcacctccagatatatgctctcgaacagtgtggcctgatgaaactatgggaccctttggacctcaagatcagaggttccagcttcctgggaacataggttttgattgtcacctcaatgggactgcttcacagaagaaaagcctggttcataaaactttgcctgatgttctagcagaacctttatcaagtgaaagacatgagtttgtgatggcacaatatgtgaatgaatttcagggtaatgatgcacctgttgaacaagaaattaacagtgcagaaacttactttgaaagtgccagagtagagtgtgcaatacaaacatgtccagaattgctgcgaaaagattttgaatcactgtttccagaagtagctaatggcaaactaatgattctgactgtaacacaaaaaactaagaatgatatgactgtttggagtgaagaagtagaaattgaaagagaagtgctcttagaaaagttcatcaatggtgctaaggaaatttgctatgctcttcgagctgagggttattgggctgactttattgacccatcatctggtttggcattttttggaccatatacaaacaacactctttttgaaactgatgaacgctaccgacatttaggattctctgttgatgaccttggatgctgtaaagtgattcgtcatagtctctggggtacccatgtagttgtagggagtatcttcactaatgcaacaccagacagccatattatgaagaaattaagtggaaattagcagaaatatccattcatttgctgtactatttgtatgtaatatttgggttgatctataaacactgtcagactaaagtttttaaaatatacttatttctaagtatttatttcagcatttatgaatttgcaacattggcaagtgatttgggattttaaaattgcaaatgttcatttattcatatcattgaatacacgttgaacacatccacattgtataggatgtggtaattagcttgtaaccagggtatgatctgctattgttatttctcctctttattggaaaaaggcctcagttttaattattttcttcccaaaataaatcacacatttggttacaaaaaaaaaaaaaaaaaaaaaaaaaaaaaaaaa

SEQ ID NO: 98 - DNA, 2975 bp, Homo sapiens
gaagacacttccggttgcgacggaggtaggcttacgaggcctgtgtcgggtagaaagggtccttcctggaccgggaccctctgccacgaccatggaccgtaggaaaaagcctttggacgtcacggcctcctcgttggtagatcttaaggctgaactcttccgaaagcaagaagaattcaaacaagaaaaacttctaaaagattctggagtttttggaaaaccaaaaacaactaacaagaaaccaagtatctggagcaaacagaatgtaggcgtttcaaatcgagctgagaaggatgctgaacagaagattgaagaacagaagactttagacaaagcaagggaaaaattggaagaaaaagccaaattatatgaaaaaatgactaaaggagactttatagatgaagaagtagaggatatgtaccttgtggatttcacacagaagatcatagacaagcgcaaagaaatggaggcatctggtgcccatagagattctcaaaaggcaggagaaagggacgacgatgaggaaaaccttcctgagggagagatccctcctccccaagaccccagtgaagaatgggtggattacgtggactctttggggcgttcccggcgctgtatgagaaaggatttgccagatctgctggagatggataaaaatcttcaggggagactttttattagtcctgctaatgaaaaaaccctattatctgaagatatgagaaaagaacttcagcgccagcaatgggaggaagaagaaagagaggccctgaagaggcccatggggcccgtacattatgaagacattcgggaaaatgaggcccggcaacttggtgttgggtattttgcctttgcccgagacaaagagttgagaaacaagcagatgaaaaccttagagatgctgcgtgaacagacaacagatcagagaacaaaacgagaaaacataaaggaaaagcgaaaggctatcttagaggcaagacttgccaaacttcgacaaaaaaagatgaaaaaatcaaaagaaggtggaacagaagaagaaaatagagatggagatgttattgggcctttgccaccggagccagaggctgtgccaaccccacgtcctgctgcccagagtagcaaagtagaagtcattgtccaggagaggaaggacaccaagcctggagtgccacacatccgggagtgggaccgcggaaaagaattttcctttggatactggtcgaagaggcagtcagatctccgggctgagagagatcctgagtttgccccgccgtcagattactttgtgggtcagaagagaactggtttttccagcagccaggcatggagcagacctgggccagcacagagtgacccagggcagtgccctgaccagagccacggacctagccctgaacatacgtcacccactcctgcccccgacaacccaccacaagcccccacagttactttcaaaactctggatgacatgatttcctattacaaacaagtgacatgatctttcaaagcacgctgacttgggtttgtactttgacagtgcctttctctcccagagggagaaataactttaggaactgaattgtacctttgtcctgtcctttccctaggaggcacagacttcgggttggatttgtcagcaaggaggaaagttatggaaactttggccacttggctgttcattttattctaagtgggatagggacatacctacctggatttacatgtgagctgcgatagaatagaagtatttattctgtaaaattagacactgagatgtgcttataaccctgtttcatatctactcccacgacttactcatatttaagggttcttttccattccttttgcaaatccgagcatgcaggtgtctttattccaagggttcagcttccagatcagccgatggaccataggtcacgaggaatttctccctgtcaagcagtggaaaactgcatgggaggcaaaatgctctgttctccaagaggacccggaagtaatcacataggaaatgataaggaagaccaggaggagctcttcgtagtccagaaaggtagaagtgggagttgtttacttaattttactgtcataccatgctattacctacactcctgtgtgcagtgggcattcagtaaatgtgtgttgaaggactgggacgtacgtggaggctgctggacctggtcagagactgatgtgccttagcggcaatggttagagcttttcagtgcatcccacctccctgtcgcccccatgctcggcttcctcacattcaggagcctgacttggatcagacttggggctgcacagtggagcaggtgggttcccgtgtcattagtaataaggagagggttgggggtgggcagggctccagaaagtcagcagtgtgcctgggcacccaccccatcctctacctgccacacctcagagggttcctacagctgcacacaagcagttgagagttgatgaccaggcccatagggctcccacagctggttcccaggccagtgagtgctgtgagaatacagtagcacaagtccttgttctctgaagagtgggaaggagaggagtgagtgaagtagcctgtcccctgcaggtcctctgcgatggcattgtctcggttcccgcagtgctgcagtgtggaagggagtgccccatcctcattacagatgacacactggagtgtggaggggtcgatgacttgtgcagggtcatatggtacctaaggggcagatctcagacttaaacacaattgatgtctaacccctagacagtctttttagtgccctctgctctcagtcttgttgccctagtatcaagcaatcttagacaaacatcctgaattcttacaaacttacctctaaactctgaggataaagttgccagtccttttaatggtcagcctaatcattctgtcagcctaatcgggtaattgctttttttaataaatacacataaaaaccaactaaaaaaaaaaaaaaaaa

SEQ ID NO: 99 - DNA, 2454 bp, Homo sapiens
ggaaagactatgttttaggtgacccgtgtggcctttttgttgaggcctttaggatacaaggcccccacctaaagacgcgaccctcccgtaggaggggggcagggcccgggggcgggagcacagcggggccccagcctcaggcggcgcgtcactgagcacaaaggagacaacagcgaggcggcagcgggcgctgatcttcgctcgccagccactcgcaattgcggttacagacctgcagctccccctcccccagccggcccgcccgcctttctgtctcctctctccctccgtactggacggccccggtccatttccgggctccggatatttggtatcgattggggccggggacgcggagcaggtggccgcggcggggcagctgggccgccagcttggtgcctcggggaccgtctcccgctgctttggtcaccagcccctgcccgcccgacccgctccgttctccggcctgcgagccctgccggccggactttgcgccgcgtccggcgctgctgctgcgctcggggccccgctcggcgccggcggtgaccgggaagcccgcgttaaaggggcaaccgggaccctggcccggtatggctgaagtcagcatcgaccagtccaagctgcctggagtcaaggaagtatgccgagattttgctgtcctggaggaccacaccctggctcacagcctgcaggaacaagagattgagcatcatttggcatcgaacgttcagcggaaccgtttggtccagcatgatctccaggtggctaagcagctccaagaggaagatctgaaagcgcaggcccagctccagaagcgctacaaagaccttgaacaacaagactgtgaaattgctcaggaaattcaggagaagctggctattgaggcagagagacgacgcattcaggagaagaaggatgaggacatagctcgccttttgcaagaaaaggagttacaggaagagaaaaagagaaagaaacactttccagagttccctgcaacccgtgcttatgcagatagttactattatgaagatggagaccaaccagggtcaaggagggccagggaattgggttctggattctcaagaccttgtagactccaaagagatggaaagactgtgaagcacaagaaagagaaaccagaacatccactggagaacttggaagagccagaacaacattgttcatcgaagagatccctgtcatcctctagctcgggcaaagggagggacaatccccatattaacaatgagcagcatgaaaggaaacggtccactcaggagaggcctcggagacctctgcttcccacgatcagtggtgaagtgtttctgagcactgaatgtgatgactgggagactaagattaaccatcagactcgaaattgggaaaaacagtctcgacaccaagatcgactttcacccaagtcctcacaaaaagcagggcttcactgcaaggaagttgtatatgggagggaccatgggcaaggtgagcacagaaaaaggagacacaggcccaggactcctccattctcagagagtgaggagcagctccacctccatgacgcaggaatgaagccaagagtgatgaaagaagctgtatctactccatcacgaatggcccacagggatcaggaatggtatgatgctgaaattgccagaaaactgcaagaagaagaacttttggctacccaggtggacatgagagccgctcaagtagctcaagatgaagaaatcgctcgacttctaatggctgaagaaaagaaagcttacaaaaaagccaaggagcgggagaaatcatctttggacaaaagaaagcaagaccccgagtggaagccaaaaacagctaaagcagcaaattccaagtcaaaagagagtgatgaacctcaccattctaagaatgaaaggccagcacggccaccaccacctatcatgacagatggtgaagatgcggattacactcattttacaaaccagcagagttccacacggcatttctcaaaatcagagtcctctcataaaggttttcattacaaacattaaaaacctaggaatctgccttgaaaatggactcactatagcaaatattactgggtgatacagaatgaattctacacttactttttttctcctgtgtttgcattcctgggatttatcctcaagtgcatttctgaccataagtaattttaattcatttcaaatgttttggttattcatgatcacttgggcagtataagaaaatgtagcttctgaatattggccacctctatgctgcatatacttcttgggatatagtatctaagacctttgtaaactgccattttgttaggtatggagtttggtatctagggagtaggccttatttagcaattcaaattttatggagatgaatgatcaaagtgaaacaatgtttggatgcaacgcagaataaaagaatataagaaatagctttttgttg

SEQ ID NO: 100 - DNA, 1408 bp, Homo sapiens
cgcgggcttccccgggcggctgcgtccccagtagcccggccggcctcggcaccgcgtgtcgtgggggtcccgggccgcggctgcagggccggggcggcggcgaggccgaggggcgggaagccactgcccggcctggcagtgtgaacgtgcaagtcgatcccctaacccagaaagccccaggcgcggtctctatgggcggccccgctcctgcttctgttttatttttttacggacagggtctcgctctaccgcccgggttgtaatgcaatggtgtgatcacggctcaccgcagcctcgacctcccgggctgaagcgatcttcccgcctcagcctcctgagtaactgggaccacaggcgcgccctgctggttgttttttttttttttttttggtagagatgggggtctcgctatgttggccaggctggtctcgaattcctggcctcaacgatcgtcctgtctcggcctcccaaagtgccgggatgacaggcatgagccaccgcgcctggccccttcttttgaatgggcctccttgcgtttccgtttcaatgccccgtgctacttttttgggagccccaaggctgtactgtttgattgactcctttttttcccctttctttttaacctaaattaaagctgccactgcagagccccgccatggaagacacgccgttggtgatatcgaagcagaagacggaggtggtgtgcggggtccccacccaggtggtgtgtacggccttcagcagtcacatcctggtggtggtgacccagtttgggaagatgggcaccctggtctccctggagcccagcagcgtggccagtgacgtcagcaagcctgtgctcaccacaaaagtccttctggggcaggatgagcctctcatccatgtctttgcaaagaacctggtagcgtttgtgtctcaagaagctggaaacagagcagtcctcctcgccgtggccgtgaaggacaaaagcatggaggggctgaaggcgctgagggaggtgatccgggtgtgccaggtgtggtgacctggaggcagccgccccgcgctgcttagcaggacacgtgaacacccagacacccactcagggactcaagtctcacctccctccccggtggagggaggaactttggcactagcccttggagccaggaaaaaagactcgtgtctcaggcagactcttactctggttactaagatcatctgtgcatgacggggagggtggaacaggtcccggaggagtcgtgaatggttctcaccaggacctgaatcgttgcttgtgtttgagaattggaggaatgagtcagcaggcgtggctcatggcccccccgtgtgcaggatcaattgtaggaggaaatttcttttttattaaaagcgaatgtgtatcccaaaaaaaaaaaaaaaaaaaaaa

SEQ ID NO: 101 - DNA, 1817 bp, Homo sapiens
aaaaatcagaataagaagtacctgacatactttctacatctgtagttgcggaagacattttaataggtcttctcatagcctttctttgctaaggacattgtgactctccagagagcaacagtgatggctctagaatgtctaggaaaaagaagggcttaatgtcaggagtctgcttggggcacacaacactagaagatgtccttctgcacattgtttcatatcgagtatggaacccttcagatcaaagcttaccaataaattcagtatgtagaacagattaacgtagttgaaatgaggaagaatgagagttatctcaaccagccagcaccccctatccccattcccacactttccctcatgggaggctgtcgggagcacttcgaaaaccactggaaaggccgggcacggtggctcatgcctgtaatcccagcactttgggaggccaaggcaggcagatcacctgaggtcaggagttcgaaaccagcctggccaacatggcgaaaccccatctttactaaaaatacgaaaattagccaggtattagaattatttctgaattatcagtctctcatttgtgctttggagaagcagaaaaggcaaaaggggtctttggccatcttctgctggagcttccagggaggatgtgtctccaagagaccagatgtaccgagtttgaaatcccagaagcccaagaggaaaagaatcacagggaggaaaagactgtccaaaggcttctggagtcttctgttctctaaccttggaaggttttgaacaatatttctcagaggatagcctttcacttattcatctgtccagcatgactcatccccgggagtgttgagtaagtgaaattttgctgtattcatgtttttgtgacttataaaataggatgataaggagagaacatgaactctggagtcagacctgttacctcggacatgatactcttagctttgtcatttagtatttgagtaattttgggcaagctaacatctctggtcgttctcatctgtaaaatgagaataaatgaaacccactaaccagaattggtatgaaaatgaaatgtggcagaaaaaaaatgaaagtgaatagtatcaccactgacacacaagcactaaaggcccttcctgtctccatcaggtatggatttggggcaacatttggccagatcttgtttatctttctgttcatctattctgtctaattcagtgctttgtttacaacgaatgtcttacaaatgctgactgaacactagcatacctgcatgaacaacaggtaaataaattttagatgtgttttaatgtttattaatctatcctgtcagagaagaactgccagttatagataaatatgatgccaggtcagggctgaagagttgggcaggttgttatctgcatggggtcactaggttccagtggagaggtgggggctaagctctcacccgctctgcagccacctggcacccggtttcagtttcctgaaagggagccttctacttgctgacgactgcctcatctcttctgaggtttcctctgataaacaattttctctgctttttttttttaattatgaaatacttaaaatgtaaaggagataatgtgacacacatttacccataattgagattgccataattgctataactttttcaaaattttgacttaatttcagacttttagaaaaattggccaggtgtgatggctcatgcctgtaatcccagcactttgggaggccaaggtgggtaaattacttgaacccagcagttcgagacttgcctgggcggcatagtgagaccttgtctctactgaaaacaaact

SEQ ID NO: 102 - DNA, 2335 bp, Homo sapiens
aggttcgaatctccgccgcttcgcggttgcttctcaacgtccgggccgcatctcggcggcggcgagggctgagcgcgggagctgcctccgagccggagccccagccctaggccctgcgcgagctgccccgccctaccccctccagcgtcctgtcgcctcctcgcccgacttcggcctgtccctctctcacgcgctcagtcctcgctcttcgccccccgcagctatcggcactcggtctcccgcgcctggcgggctccgcccgagcctctgggcccatggccaagcggcgtgcggccgagccggtgacgttccacgtgccttggaagcggctcctgctttgcgacttcgctgagcagccgccgccaccgcctctctggatccggccgcccggggtcgcgcatgctgggcagctcctcggcgtcccagagcagcaccgaaagcgcaaaatcgacgcagggaccatggcagagccctcggcttcgcccagcaagcgccgtgacagcggggacaacagcgccccgagcggccaggagcgtgaggaccacggtctggagacaggcgatccgccgctgccgccgccgcccgtactgccggggccgggggaggagctcccgggcgcccggctcccggggggcggtggcgacgacggggcggggcgcgcaggacccccgcggggagactggggggtcgcatcgcgccagcacaatgaagaattttggcagtataataccttccagtactggaggaatcctttgcctcctattgatctggcagacattgaagatttaagtgaagacaccctgacagaagcaacacttcagggcaggaatgaaggggctgaggttgacatggagtcctgatgtaaggagccgaagcagtgggattggctgatttgaggagatgtctctaagtgaattctcgtattcttaagggaaaagttattttccatacttgaagttatatttccaaacctgagaaatgaagaaagattgttctgacattaaatacctacagttactactgaacctcttaataaggatttgtcaaggatagagtacagttgtaggggaagtattttatgtatgcattcttagagcaaaaagttttgtttaaattctagaattgaaggtactgatcttataaaaagaaattctagcagttttagaaataggtgggaaaaactcaaatattcctcctatctgcaccaaaaagtttatttgtggtatataaaatgaatattgttttataataacttgttaataaagtactttctaatacattctattgactctgttagttgaacaaatagctgacttgaacatctatgcaaacttaagatgggcgggattgttgtaaaagctattgttttaaaagagctttctaaatgtaaagtagtgataatttcaatttgggtagcgtgtttgcaaagcttccaatatttgatgttggttaagctctactatgggcaactgaagatggataaagaaaaatgaaaactgaatcggtgcctgcttccccctgttttcccaggattagaggaaaaaatttattgtataatcagcttcttggttttgaattgcttcgaggcatggttttattccttattactttagacctgtagttttcaacactgacagcactttaaaaatctttgcctgggcctcactcttgagagattctcatttaatagttctgaggtgggtcttggatataactatttttttaaacacctgtcctgtttccctccattcctctcatgtgcagacagggttgagaaccagtagactaatggtcgtttttcctgtttaaaggagataactaatttgagctgaagcaatgcttcttaattagctttgtttttgttttgctctgttggtggctttgttacaactgaattattgtgttattactatttcattgttaaagaaataaagtaagcaatttgtgatgtgagtatcagtgattaagttaactaacttttgtactgcatccagaatgttggttttgcaattgagtaactggttcttgcttgcattttttgttgttgatgacattagatccaaaattcaagacaaatggtaaatgccattgagagggaaagagaaaaacttgattttttttgtgtaatgaaggatttaagaatgggttgacattaataagaatgctttagaacagaagacaaactgtattgcattgtggtcagacatggttcaaagtcttgtactgccacttcctacctatgtatctttaagccagttatttttcatctccaagcctcaaatttctcacctgtaaaatgagaaataataaatagtatctacctcaaaaaaaaaaa

SEQ ID NO: 103 - DNA, 666 bp, Homo sapiens
ccgcattcttttttttttgaaggtgaaaaggaggtttatttagtgttcgaacagctcagaggagacccacagtgggtagcttctctctgtaggcaggtcatcccatggagtgttcagctttcagcaaagaggtggccctggggagggtggctcctctctgcccactgctcattccactgtctgctgctctcagcagagaggaggccctggagagcgtggctcctctccgcagcaacttgttcagacatctctgcatgtctctgaagctctcagcagggagggtagttcctctctgctgctggttgtcccatagtctctctctcttctgccctgctctgactgagccccagggcttttatggacttggtatctgttggagaatgaggtcctgtcctgctttcacccatgacccatctagcttcagctgtatccattttcttctgagcccatcttccattgtcctcaacgagtttctttggtctttactgcttgctttccatctcgccatgtggcctgcaggcttgtcagtaacacatttctttagctgtgccttacttggcctgtcggcacagtctgtcagaggcgaccacttcctcctttggctgccgagacacctgactccactccttttcattctcctttgcaggttcctcctgttctccccat

SEQ ID NO: 104 - DNA, 486 bp, Homo sapiens
ttactgctggtcctgggagccattttccttcggagcagcagccctgtccggcatctgtcttgagctcccagcaaggaaagtccatcagcttgataatggaggagaacaatgactccacggagaacccccaacaaggccaagggcggcagaatgccatcaagtgtgggtggctgaggaagcaaggaggcttagtcaagacttggcatactcgctggtttgtgctcaagggggatcagctctattatttcaaagatgaagatgaaaccaagcccttggtgagtaggagaaaatgtaaagcattaagggcctaagaaagccaagaaatagagggatttgctagaaaccgattgggactgagaccacccagagctccctggtctccttcagttcattgtcatctttcacccttatacccattacttgctttgagtcggagataataaaatcgctacttgaggccaagaggcaaattatacttgtctaata

SEQ ID NO: 105 - DNA, 731 bp, Homo sapiens
ttcttgccaaatgaacctcagagacatacatctaaggaccttgcgtgcaccaaagagggcctaggaggacagcagctgtccctacttgctgcactgtggggaggcgcctgtgctcactgctctgctgttgaagagctcagtgaagcatatgatgtgactgtggatatccaactgctttagctgacatcttggaaggaaagcaggagagagacctgggccctgaccttccttcgtcaccatcagcttgtgacatcttctcaggccagccctagccaactctgtgactgtttccacaggtgtcgatgttggcatcatgtctaacttctgctgcttcccaagttcgccatgatcatctcattagtgagcagatgtaaagtatttgtaaaaaggcatcatacaaaaacaaagttcattgttattcatccttttccttcccaaatacactagacttctttaaaacattaaagcaaagtgaggccaagctgtttacttgtttaacctaatatgaagggccactgcaaagcctgaggctcaaaggtaagattcacagccaggtgcagtgcctcccgcctgtaatcccagcactttgggaggccaaggcaggaggattacttgaggccaggagttcgagactagcctgggcaacatagtgagaccctgtctctacaaaaataaaaaaaattagccaggcatggggggtgagcgcctgtagtttccagctactcaggagg

SEQ ID NO: 106 - DNA, 652 bp, Homo sapiens
ggcctcggagagccgaggatcaaaaccgagaagccaagtcggcttctagggccacgttaagagagggggctgctctgttaagcacagagaccaagcgtcttgcaccttttaacaggctcggggcaggaagcaggagccggaatctgggccgggagacgaagggatggtctaggacctgctcctggatggtggatgcgtgagatccaggttagacttcgttcctaacgtcaaggctcccaggctccacttccggttccgagggggccgtcccgtcacccccggaagttcctcctccacgctttagggccgggccacttcttctgccacgtctgcatttcggggacccggatgccgcgcttgcgcctctttcatcttcccatcatggccgccgcctgtgcgcctctgctgagtcgtatgtatttccctcctgacattttttttcagatgttccagtcactttatggcctcaccaacagaaatgagattaaaaagaatttgtcaaactatctttaataatgccccttcactctgcctgtgacgtattagtgacctctgagctagagtcttgtagtcacttcctggtgacccctgaccccgttgatttccgtccgctaggttgctctcacccatggcgtttgctggttatgagatt

SEQ ID NO: 107 - DNA, 599 bp, Homo sapiens
cccacgcgtccgcccacgcgtccgcccacgcgtccgggagccctgtagcggaggggctggggggctgctctgtccccttccttgcgcgctgcggcctcagcccacccagaggccggggtgggagggcgagtgctcagcttcccgggttaggagccggaaaattcaaatccgaaatattccaccccagctccgatgggaagtactggacagcctgctggctcagtatggtacagtagagaactgtgagcaagtgaacaccgagagtgagacggcagtggtgaatgtcacctattccaaccgggagcagaccaggcactcattagaagaattcctcaattgctgcttcaacacccgccacgatggcgttcaacctggcagattaatttaacaactctctgatgggttgccctgaaatttgaaaaacagtgccttgggccgggcgcgatggctcacacctgtaatcccagcattttgggagaccgaggcgggcgaatcacctgaggtcgggagttggagaccagcctgaccaacatggagaaaccccatctctactaaaaatacaaaattagcggggcatggtggcgcatgcctgtaatccc

SEQ ID NO: 108 - DNA, 1397 bp, Homo sapiens
tccgaatgctgaaggaaaaacgctcaaaatctcattcttcagggggaagcgttgccactccgaggtgcccactgggaacgaatcccaaagccacgagcgcctgcctagtggggaatgtgaactgttatcctgagagtcgtccttctctctccctggtccaggacagaaaatactgaatagacaggaatttctgaagtctaaacgcctccaatgataacaggagtgttattggaaagggaacaagcgagaagacacagtcttcgaggagttaagttttgctaatctaatgatgagactgcattcatgaagactgagtgaagactttattgcaccatcacatcactaaggtttttctccaacatgaacattctgatgaagtcgaaggcttgaggcctgactaaagcacatatcacactccctacacttccatgttttctctcccatgtggaccctctgatgcatatcaagattcaagcgcctgttgtagcccttcccacagtcctcacatttgtatggcttttctacactgtgaactttttcttgcactttagagaatgaattctgtacaatgttcttcccatgctgctcacatttgagaggtgtttctctgctgtggcgtctctgatgggtcagacgagttgaggaccagctgaagcccttcccacactcatcacatttgtatggcttttctccagtgtgcactctttgatgagaatgaagctgtgaattctgagtaaatcttttcccacactcttcacatttgaatggtttttctccactgtgcagtctctgatgtttcaaaagacacgaggcccagccaaaactcttcccacattccttacaattgtatggtctctctcctgtgtggaccttctggtgcatatcaagattaaacttactattgtagccctttccacacttctcacatttgtatggcttttctccagtgtgaactctctgatgggaatgaagatgtgaattttgagtaaatctcttcccacactcttcacattggaatggtttttctccactgtggagtctctcatgtttcaaaagacatggggcccgactaaagctcttcccacattccttacaattatacagtttctctcctgtatggacgcgctggtgaaagtcaagatccaacctccttttgtagcccttcccacactccacacatttgtatggtttttctccactatgggatctctggtgggaatagcattgtgaatttgtgtaaaatcctttcccacattcttcacatttgaatggtttttctccactgtggactcgctgatgtttcaaaagacacgaggcccatctgaagctcttcccacactctttacaattatatggcttttctcccgtgtggaccatatgatgcgtataaagatctc

SEQ ID NO: 109 - DNA, 939 bp, Homo sapiens
gtcccccgcctgagggaggggagcggtgcagcagacatccgagggcagctgggaccccctgactcagccgacgggtgagtcaggctccctgcaggccacaccggacccccccagggcggggatttccccaagatgagaaatcagccaccggaagtcacgccggaccttggacgggcagacagaggctgggaggagttctgggtgcagagccccccaacctgtgctctcatctcttgctctggggtaagccagtggccatgctataaggacactcaagccaccctatgaagaagcccacatgaagaggaactgagatatctggccaacagccagccagtcactgagcctgccaaccacgctgtggcaggtacctccagccccagacacctgcagcctccactgaaagctcaatggcagcctcatgagaccctggaccggaaccacccaacgaagcggctcctgtattcctgattgacagaaactacgggatcataaatgcttgctgttcagtctgccaagtgttggcgtgatttgttaaacagcaaccagtaactaatacgccacccatggctgccgcgttcctgctgtggggccagcactattccatgcttagaggctccatcaatacctgtgatggactaaatggcacggtggctcacacctataatctcaccactttgggaggccgaagtgggaagattgcttgagcccagaagttggagaccagcctgggcaacacagcaagacccctgtctctaccgaaaatgaaaaaaattagctgggcatggcagtgtgcacttgggagctactcaggaggctgaagcgggaggatcacttgagctcaggagttcaaggctgcattgagctatgatggcaccactgcagtccagcctaggagacagagctagaccctgtctctaaaaataaataaataaataa

SEQ ID NO: 110 - DNA, 1015 bp, Homo sapiens
cgggaggctggagggagctgagcccccggggagggggcccgattccgcctcgccgcgcctctggctgctgggccgtgggtttttctcttctcctgggagtaaggaggacgacggcccctaacccctgaattagccttctatttccattagtgacttagaagctacccggcgcctcatctgggctcacctgagctgaggatcaggaaggggagggggcacagtcattccctcgcggacgcggcgggaccccagcggacggctttgtgcggactttcggcaccgttatgcccccctagcccgacaccacctggggctggcgcgccacgttactgttcagggccagacgcaccgccctgcctgctccggggagccggacctccgatcccggggatgggggaccccgagacctcagacccaacggaggggactctgatacctcagaccccacgcggggaccccgaggcttcagacccccacagggtgaccccgagatctcagacccccgcaggagtacctccagacctcaaatccccagtaagggaatgaaagagacttcggatccctgaggcagcaaatccctgtaggggcatcccaggactcaggtcaccgcggggtgaacccagacctcacattcaggcaatccccaggacgccgatcggctggcactacccactgtcccggccaccccccctggagctcaaaggcttggccctccagctgctccaccctgccggcaaccaggctcaaacctgcagcccgcggatcccctgccccggaagcaaccagattcgcgggaggttacctgactgatccaaggcagtttctcactccgttgcccaggctggaatgcagtggtgtaatcacagctcactgcagcctcaacctactggactcaaaggataagatctgcttttaattaatttttgtatacggtatgggatagggaccaaagttcatttttttgcatgtatacatatccaaatgttccagcatcatttattaaaaagttattttt

SEQ ID NO: 111 - DNA, 2142 bp, Homo sapiens
agacactacaacagagctctgtcacctctttctaaaagctgggaaatggttattgtcaacgccagccccattttggaggcacctggttgcaggggaccacagctagctgggtgcggtggctcaggactgtaattccagctacatgagaggattgcctgaggccaggagtttgagaccagcctgaacaacataatagactgggagctccatgaggtccgagacaacctgaagacgcccagctcagcagagactggctacggcaatcactcagtaactatttgttgaattggacataatagaagagctgaagaattttaaacaagtcaccatcagtgcagtggattcctcaaagcaaacctggaaatctcggcatgagttggatttaaggcagtacattttcagctcctgaaaatttgaacagtttctcaagagaacaacttgaaacagaaaggcaaggatgtagatgtggttgtagaagatgatgtctgcggccaccaaactgtccactgtgaagacaaaccatgcatgtttagtaggcagtggctcctcttccctgtggggagtcccactgcttccctggcctgggtgttctctgccggtagatgttattgacaaaataagcaaagtcctctgattctgagaattagagttaatgatagctctagattgcctctgccatgctatttaaagtcctgctttattgtctgacatgagttcggcatctatgaggacttgtccagtgcaaagtacaatctccactgaaagatatcaccagcacatcttgtcactgtgctgtagcaagctgggcagtacaatgatgttggcaggatcctgtttaggtggaggatggccacctgggcatcaagaatcttctaccctgattccacattgactccccaaagtgggcttcagacagctctttacttacgtcttctgattaaagcagcagacagggtcccagctagagaaaagttctgggagcaggagaggtgttggtggctttaaaaccaggggtgaagattgaattaggaggttaggcaggagtaggatcaaagacttagaaaatgcttgtttcctcctattattcttatgagagctgatacggggctttcagttttgggggagagccttggaagagaatgagggaatttaatcaactagcgggacctttccaacttacatttcccagtgtttgttggatgatacgtttgcctaataagttaaagcttccaaaaatagaaaggaaagcttggtgacactaagctctggcatgctgtcatcttctggctaggggtcttgtgcttctgtttttctatttctgaccaaagacataaaatgtgttgtgacctctcaggtaatgctgtggtatcatgtccaagcagtaactaaccagagtggagtttttacaggtaaaaaagtattgaatagctcagcaacagggaggacttaatgataaattgctttttcagaaaatgacaaggggacagacacttgcggttcttccattgagagccttaaggaagttaagcgaagcctctgctcaccccttcaagtaacgatgacccatcagatcgtggtcataaccagtagcatctgaggcacccctaagctctctgggccccagattccttgttaagttaaatgaggtgacttcccagtttaccattctaggggttcctgaggagttaaaaatgaaggagaaaatgcaacatgtaccaagtgagcaagagctcaaggcttatctctggcacgaaagtgaccgctgtccgttagagatgtgattgccctagatttcctttaggaaatgcaaacattgaactctggccttcaggacccaagtcccggcttgcatctattctatacactttctgttatttgttagggctcacaaggaacagttatttatcttttattttctattttttttttagagatggagtctcactctgtctcccaggctggagtgcagtggtgccatctcagctcactgcaacctccacctcccgggttcaagcaattctcctgtctcagcctcctgagtagctgggactacaggcgtgtgccatcatgcccggctaatttttagtatttttagtagaggcagggtttcaccatattggccaggctggtcttgaactcctg

SEQ ID NO: 112 - DNA, 2894 bp, Homo sapiens
aaagggagagggtgagggagttgtggagcgacagcgacagagccttggagagggaggctctgctcaggatctatccccagtgccttatttagttcacttggtgaggtcatgttttcctggatggtgttgatgctagtagatgttcttcagtgtctggacattaaaatcttgggcatgcagcaccatatgagaagtttaaaaaataaaatttaaagagagaaaagtggtgggattgctgaatcagacggagccaatagatgatcagtaatggtggccaatcatcagctagaaagaaagtgcttagcagggcttgaaaacaccaaaactctgagaccactgaccttcaaaaacttcaacagccctgagtgaaaagtccaaacacatttatctaccattcgaagacgctttattgtctgtcctctgcgcatctcagtagtctctttttaccacactgtctatatactgcatgagccatttatatgaaactatctgacactccaaggcatcttatactatatattcaaccatgtatccctagtacctagcacagtcctggcatatagtttgctactaaacttttacagaatgaaggaattatcttgtatccagtttccaagttttaaggtgattcttcactaaaaaaaaagtattacagttcacaaataacctacttccctttttacaaatgggatcaattttaatcttatctcctaataacattactttcatttactctgatctaaatatactgtcctaagagagcaataagaaagagagttgaagctggagtttgaagaattgtacatggtcctgtgataccctaccttgttttaacctgagtgactctctcctagcggagagagagccggacagactccattttagtttcttcacgtgcagccccctttaccttccacccttaattgcataactagtataaactgactcaaagcaggtccagaatgcacttactgataagatattgaggcaagctgcaccagctgctcctgggtacgcactcggtgaatgtcacgcaaaacccctgcatttctctctttgtgatagtttaagcccctgcacctggaactgtttatttgttttgtaactgctattgtaaccaattaatattttaactatttgccagctctgcttctgtaaaacttgtttcagctaaactcccccctcccctatttagaccacggtataaaaacaaaaccagccccttcctcggggccaagagaattttgagcattacatgcctctcggttgccggctaataaagcactccttaatttgtctcaaagtgtggcattcctctataactcgcttggttacaacagtccacactgtggcctgaggtgcattgcccacctgagcttcatctgttatgtatgtcagggaatataagcagggtgagagtggcctcatcagaggaccccagatctctggcttacccatctggcaagtgcacctctgtgagcaaagacttcagagccagatgacaagaatggcccaggcagtccaccagaaaaacctgggcccagtgtacgtcaatgcagagcatcaagcatggtgtaacaggtgcacagttgcctactcctgttcagagatgactgcatcccacataccgtaaaatgaggaaatgcagagaagcagatgtaactgaagaagacagcagaagcaacaaggagggacaaccaggacctaggagggcaccatgccagagacgcctggaccccacgctaggctcagtgcctgttatactcttgggagccagcactttccctctccatcacatggcatacttgccattatttgttgtgtaaaatattgtccttagttttcacctttcctaggagacacaggcagagcctgtgacactacagcttctggcacgcagtaggtaggtgcatcacaaacatctgctgagttcacacactcttgccttctcaaaacttcttgtcaagtcttcagtgaaaaggaattgctgattgaacaagaattaaacttctagagactcctggatccactgaagtttgagacaagctctagtcaggaagtgttagagagttctaaacaggaaatattccaacacatcatctaagcatgaagcaggagacacccataattgagtttgctgccaccaaccccaacagcaagaattccagtcctgctgctgcaaattctcttttggagtctttcttgcagttgcggttggttggctgctgctgaagtcacctgaaagctcaattaggctaaagagtctagacgggtcacttacatggctggcagtgcatattggctgctgagtgtgatgcctatctgacctctccatgttacctggagtgctcacaacgtggtagtttgtcccaagacacacaggcaaatactacaaagcttcttaggacctagccttggaggtcctagagtacgacttctgctccatcctattggaaaagcaagtcattgtgaccagcacagattcaagaaatgggagattcgactctacctgtcaatgtaaacagcagcatgtgcatgcagggaggaaagaaattgagggcatcatcttgaagactatcatatcacaccattattccaactaatgaacattgtgttttagatgggtagtactagctactcatctgtcccccagaaacccaagctaagcatggacatattgaagagaatgtcagcaccattaaaaaaactctagaaaaatcacatgtgatgacttaggttaattcagtctgtcaattacatcaatataactgccttcttgtaaccctaagtatggtgaagcagaattgaattctacaaaagtctttcatctgttttcctatggaataattaacaaacccaataaatgtataaatagcaaaaaaaaaaaaaaaaaa

SEQ ID NO: 113 - DNA, 698 bp, Homo sapiens
atctctactgtcccatatccatccctgctgaccaaagcaaaactaggtttttcttgcttttcctgggctgagcctcttattgttcctagacacagccagcctggtgagcacaggcaaactggacattggaagacccagctgcacaaagactccttcctgtgagaggcttggagaagactttgctctagacacacaagggagcctgtgggaaggtgccaggggagccaagaagagcaaaaccaaggaggcattgtttcctccagagcctcattcatcaactcgctctgaacagttagcacgctcagacagtcatcttctgcaccttgcctttccctgtgtcttgactgagggcttatctgagagccttttgttcaggctcataattattcagtgactcaggagcccacaagcattacccacggagccagacaagaccagcaagctctgaggaccacctgttccaagtcatttcctgtgtgggcggcaacttcacagggctgaaaatacgagttacggtaaaaatgtcttcaaccctggcgcgctggattgttgaaactcgatggaatcttgcttgattatgttcaggccagacacatttcattatcattctttgcatatatattaaaaactctaacccctttacaaacaaatgttctcaaggggcagacagcacacccttgcctcatgacattgctcta

SEQ ID NO: 114 - DNA, 677 bp, Homo sapiens
gccagcaggaggctgatgaaggagcttgaagaaatccgcaaatgtgggatgaaaaacttccgtaacatccaggttgatgaagctaatttattgacttggcaagggcttattgttcctgacaaccctccatatgataagggagccttcagaatcgaaatcaactttccagcagagtacccattcaaaccaccgaagatcacatttaaaacaaagatctatcacccaaacatcgacgaaaaggggcaggtctgtctgccagtaattagtgccgaaaactggaagccagcaaccaaaaccgaccaagtaatccagtccctcatagcactggtgaatgacccccagcctgagcacccgcttcgggctgacctagctgaagaatactctaaggaccgtaaaaaattctgtaagaatgctgaagagtttacaaagaaatatggggaaaagcgacctgtggactaaaatctgccacgattggttccagcaagtgtgagcagagaccccgtgcagtgcattcagacaccccgcaaagcaggactctgtggaaattgacacgtgccaccgcctggcgttcgcttgtggcagttactaactttctacagttttcttaatcaaaagtggtctaggtaacctgtaaagaaaggattaaaaatttaagatgttctaaaaaaaaaaacaaa

SEQ ID NO: 115 - DNA, 537 bp, Homo sapiens
  misc_feature (311)..(311): n is a, c, g, or t
agaaatgtatgtcctggtcttcggagtcgggggacactttaataatgatcattaaatttgatcagccgacttaaacttgttgtctactggaaaccaattaactggatggagtctcactctgtcaccaggctggagtgcagtggcatgatctcagtttacctgcaacctctgcctcctgggtttaagcgattctcctgccttagcctcccaagtagctgggactacaggcgcgcaccaccacgcccagaaaaattggaacagaaaaatatctaacttgctgagcatttgatgggaaaaagtaaaagataacnttccatttggtacacaacttattgtacatagagctatgatttgaggaggcatctaatttctgaacaaattcaccaagaaataccatcacttaaagtcattatcgcaatcatgctgcagtgaacactctatacaaaatggccaggtcattaaacatcaaagatggaaaacaagccagcaatctcttctgttctcttcaaagtgaatgcaaaattgttaaggtaataa

SEQ ID NO: 116 - DNA, 565 bp, Homo sapiens
gctcggattacagacgtgagccactgcacccgaccaatctgtctttttgtagaggggcctcaagcatgaacttactgatggctctcaccatatgatatgcctactccctcttcaccttccaccatgattggaagtttcctgaggacttgccagtagcagatgcctgcaccacacctcctgtacagcctgcacaaccgaggtgatggccggaagaacatggcagagggcaaaacaaaacagcattgggaacaagctctgtttaaaaggagacttgtgaacagcaaagattagaaagggttctcttacaactgaagcccatggaagacaaatgtgtactgcgtgagttttaaggcaataggagtagtgggacctagggcacaccagagagcatattaactctcaaacttttaaaaacattatatctgctggacacagtggctcacaccttaatcctacaactttgggaggccgaggcgggcgggtgtagcttgagcccaggagttcgagaccaacctgggcaacatggcaaaatcccgtccctacaaaacaaacaaacaaaaaacaa

SEQ ID NO: 117 - DNA, 589 bp, Homo sapiens
acttgctgggaggcagggccgggagagcccgacttcaggacaacttgggcctgcggcggtcgccgggaggcccaaccttggcgtggaggagcccaccgaccggagaccatttggggcctggagatgccatcggagggcaggagctcatcctggagaggccaccgtgaggcctgacctgggcctggggagcttggcttgaggaagctgtggaccgaccaaggccgccaggagatggctaaagaaacaggctcagagaatgttatttgattggaccgtgttgcatttctggacagtgcagctgagatcagactttgtgtgtaactccactagcctaccagggtgcctctcataaagcattcctttcagctacgatacaaaagaagcaaatatttgccactggaaaaaatattcaaagacactcttaggttaatctatagctgatgacagtcagtctagtctacatagcaagcagcttcaagatatgattacttagctaagcgggaaatgggacgtgactgctgcctcattcccacgcctctctggacctgataatttagaggaagctcacattcgcaagataaaaattttc

SEQ ID NO: 118 - DNA, 540 bp, Homo sapiens
  misc_feature (470)..(470): n is a, c, g, or t
gtggaaggaaagggctttattcagctgggagcaccggcggactcacgtctccaaaaaccgagctccccgagcgagcaattcctgtcccttttaagggcttacagctctaagggggtccgtgtgagagggtcgtgatcgattgagcaaccagcgggtacgtgactgcgggctgcacgcaccggtaatcagaacagagcagaacaggccagggattttcacgatgcttttccatacaatgtctggaatctatttgggaagctgaggcaggagaattgcttgaatccaggaggcggaggttgcagtgagccaagatcatgccattgcactccagcctctctgggcctcaggctcctcatctataaaatggggacatgcaagtccctaacccagaaggtcagtgtggggatcgaacaggagatagcacatgaagagcacaggttgagtttgtggggtgcgggggcgtttggtangacagtcantggtttaccaaaccaaagtgcaagctgaaagtttcggaccccagggatcagttgaaagagc

SEQ ID NO: 119 - DNA, 429 bp, Homo sapiens
tgggggaatccactgaagacctacatttcccggaagtttaacagcccccttctcagttttcctgatataaatttaattcacacttgggcagggcatgttgcctcacacctataatccgagcactttgggagcccgaggcacccggatcacctgaggttaggagttcaagaccagcctgggcaacatggtgaaaccccgtctctactaaaaatacaaaaattactactcgggaggctgaggtaggagagtcgcttgaacccgggagacagaggtttcagtgagcccagatcgccccaccatactccagcctgggcaacagagcgatgctccatcttaaaaaaaaaaaaaatcaaaataaaatacaataaaaaataaaaataaaatttaattcacagttgtaaccagcctaaattaaaaatatttcttaag

SEQ ID NO: 120 - DNA, 462 bp, Homo sapiens
ctgtaaagattgtaagaccaggtgggagagtgcttacgtgtggtgcatttctggaacgaacaaggtttatttcaaacaactctgttgtaaatgccaaaagagttttaacccttatcgagtagaagcaatccaatgtcagacctgctcaaagtctcattgttcctgtcctcaaaagaagagacacattgatctaaggaggcctcatcgacaggaactgtgtggtcgctgcaaagacaagagattctcctgtggcaatatttacagctttaaatatgtgatgtgacttgtacagtgtgacttgtaatggacccctgagctcttcttgtaacttactgtgctgtcttccttttttgcaacttggctctgacctggcatcggaaaatggctaggcttttgtactttttgtagattgtgtaacaattgtacaatgtgaatagaataaaataaatgcatgtgaactag

SEQ ID NO: 121 - DNA, 563 bp, Homo sapiens
gtcgcgtccgcacttctcctgcccgagagagactgagccgcgctggcagctcgcgtcgagtcggtctgccctagccgcatcccgcggcgcccggtcgggctccgggcaccaggcaacacctaggccgttcccttcagacagccccgggccagcggccccctcgggaaatgtccagcggccgcagaaggggcagcgccccctggcacagcttctcccggttcttcgctccccgaagtccttcccgggacaaggaagaggaagaggaggagaggccggggacgagcccgcctccagctccaggccggtccgctgccagtgttgaaaatgagcccatgagcacaagtcagaaaaaggaaaatgtactttcatcagaagcagtaaagattcgccaaagtgaggacaaaaggaaccatgctgagaagccagtcactcttccagtgcaggaagatcccaaaaaggcatatgatctttccagttccacttcagataccaaaataggagaaagtgacagacagccaaaagaaagcttttttcagtttcttggtaacttattcaatatctcg

SEQ ID NO: 122 - DNA, 1555 bp, Homo sapiens
gagaaactaaaaaaatcgttaaaagtaaagacacgttctggacgggtatctcgacctcccaaatataaagctaaagattataagttcataaaaacagaggatctggcggatggtcatctgtcagattctgatgattactcagaactctgtgtggaagaagatgaagatcagagggagaggcacgcactctttgacttatcgagctgctccctgaggcccaaaagctttaagtgtcagacttgtgaaaagtcatatatagggaaggggggactggcccgacattttaaacttaacccaggccacggccagttggaccccgagatggtgctgtctgagaaagccagtggaagcaccctccgggggtgcacggaggaaaggacgctcagcctgacctccctggggctgtccatgccagcggatccatgtgagggaggggcccgctcctgcttggtgacagagtcagcacgcggtggcctgcagaatggtcagtctgtagacgttgaagagacattgccatctgaaccagaaaatggagctcttttgcgatcagagagataccaaggacctagaagacgcgcatgctcagagacccttgcagagtcccgcacagctgtcctccagcagagaagagctgctcagctacctggtggccctgctgcggcaggggagcagagggcgtcgccaagcaaagccaggctcaaggagttcctccagcagtgtgaccgggaggatctggtggaattggctctgcctcagctggctcaggttgtgaccgtgtatgagtttcttctgatgaaggttgaaaaagatcatctagcaaagccttttttcccagctatatataaggaatttgaagagttgcataaaatggttaagaaaatgtgccaagattacctcagtagttctggtctgtgttcccaggagaccctggaaataaacaatgataaggttgctgagtcattaggaatcacagaattcctacggaagaaagaaatacacccagacaaccttggacccaagcacctcagccgagacatggatggggagcagctagagggagctagcagcgagaagagggaacgtgaggctgcggaggagggactggcctcagtgaaaaggcccagaagagaagccctgtccaacgataccactgaatctcttgctgccaacagcagaggccgggagaagcccaggcccttgcatgctttggccgctggtacaatagtgtctcaggaggaggacattgtcacagtgactgatgcagaggggcgtgcctgcggatgggcccgctagaaggagttcctctagaagctgtggagtcggtcgtcaccgtggagccagagccctcacagtgaagtggagtcagatcctagattcgtctgattttatccagagaaggtctatggcaagcaatgtatatttttctaatgtgaatattgcacagatgaaccttttatttataaagaataatgtctttcaaaaaaaaaaaaaaaaaaaaaaaaaaaaaaaaaaaaaaaaaaaaaaaaaaaaaaaaaaaaaaaaaaaaaaaaaaaag

SEQ ID NO: 123 - DNA, 1260 bp, Homo sapiens
agggccttattccaggagtaaaaaaactgaaaaaccaaataaacccagatgcttggcagggcatggtggctcacatctataatcccagcactttgggaagccaaggtgggaggattgctttgagctcaggagtttgagaccagcctgggtaacagagtgaaaccctatctctacaaaaaaatatatatataaaacttaaccggcattcctgggtgtggtggctcatgcctgtaatcccagaactttgggaggccaaggtgagcacatcatggggtcaggagtttgagaccagcctgaccaacatggagaaaccccatctttgctaaaaatacaaatataaaattagccaggtgtggtggtgcatgcctgcaatcccatctactcaagaggccgaggcagaataaccgctcaaacgcacgaggcggaggccgcggagagcctagatcgtgtcattgcacttcggcatgcgcaacattagtgaaactctgtctcatagattaattaattaattgattaaaataaaaagtaaaaccaatgcttaatggtatcaacacaatatcagaactcaattagcttccaaccatcagcaggttggcacctcagcgacataagcagtctcaggtgtgcatacaatgacgagtcaagggtcccagtcccaggcagtgagggcttcagctgaatctgcgttgctcctccatttctgatagtaccctccaaccaaggaccaccaggaagcacctgggcaacttggtgaatccccgtctgtactaaaaatacagaaatgatctgggcatgatggtgggtgcctgtaatcccatatactgtggaagctgaggcaggagaatcgcttgaacccggaggtggaggttgtggtaagccaagattgcatcactgcactccagcgctgtgggagagaggattgtgttggtaatttttgtggttcggacaatgttcatgtttttgtattgaggttttattatggaatggtctttttgtagatcagtcagagagtgacattgtctgatgttttttgtgaaattattttgctcaacaggacaccaccatggccgtgctgttggtgccaggtcagctcctagcaacatcaaggttttcggactgagggtgcaaaccagctcccagctggcagcagttgcttttctctcttacaccgccacactcgaaatcagaaaattggcaacccctttggtagaacatgacgctcccggtcactacaggcccacttataccaagggcgaattc

SEQ ID NO: 124 - DNA, 570 bp, Homo sapiens
actaacagcctgagagaaaaggacactgcatatcacagagagatgcatagatgttacactcagaaagagagtgaacaactagaagatgtggaaggcaagctttttagtattaagaggaaccagtttggcctctggagtcaagaattctcaaaacagctttctgaacctgaatcgcagtaaagctggtgttctcctgaagccaacatttatggtatcaccttgctgatccaggcaaaatggcaaacgtgtagtgaacataaagccaggggctgagattgtggatgtcctctgtaccaagtttcttatcatggcaagggtagaagttcaccagatcaataaaccaaactccctgtcacctcagactctgcacctgttaaagccctgtaaagagccgacatgtaaggacttatactgactggatacagagaatatggagcaaatcaaacgtatttaaatagtaaattatcatattgttataaataaaaatttacattgcatttccttttagaatgttacttggatatatattcttcaacatttacaaatgctgatgcttcatgtacttaaaaa

SEQ ID NO: 125 - DNA, 1772 bp, Homo sapiens
gcattgtgggaagggcggccggtgcagccgcagctgccatcttaggggcgcctggcgctacgggtttctcgttggaggcggccttcgtggcagctgtagacgccgggaaaaggcataaagtccgttggccgacacctttctttcctccggcctcggtagaaccgccagcccgcgtccgaaggcggaggcgaggggaactggccgcgtgaggggcctgaggcgagcggttagagcgtctcccggaaggatgggccggtctcggagccggagctcgtcccgctccaagcacaccaagagcagcaagcacaacaagaagcgcagccggtcccggtcgcgatcccgggacaaggagcgcgtgcggaagcgttccaaatctcgggaaagtaaacggaaccggcggcgggagtcgcggtcccgttcgcgctccaccaacacggccgtgtcccggcgcgagcgggaccgggagcgcgcctcgtccccgcccgaccgcatcgacatcttcgggcgcacggtgagcaagcgcagcagcctggacgagaagcagaagcgagaggaggaggagaagaaagcggagttcgagcggcagcgaaaaattcgacagcaagaaatagaagaaaaactcatcgaggaagaaacagcacgaagagtagaagaattggtagcaaaaagggtggaggaagaactggagaaaaggaaggatgaaattgaacgagaagttctccgaagggtggaggaagccaaacgcatcatggaaaagcagttgctcgaagaactcgagcgacagagacaagctgagcttgccgcacaaaaagctagagaggaggaagaacgtgcaaaacgtgaggagctagagcgaatactggaagagaataaccgaaaaattgcagaagcacaagccaaactggccgaagaacagttgagaattgttgaagaacaaagaaagattcatgaggaaaggatgaaactagaacaagaacgacaacgtcaacaaaaagaagaacaaaaaattatcctgggcaaggggaagtccaggccaaaactgtccttctcattaaaaacccaggattaaattgcaaactctgaactttttacaaagaaaaatggaaaaactttgtatggtagcttcatgttgaagtggttttttgtttttgtttttgtttttttaatttgtaaaatctggaaagttagcttgttctaataggggctatgctctgcaattccctttttttttttttttttccttccactaagtcaaatccttatcagatcattgttgtattctaaggagtgacgtatttttcacctgtttggattctatattagtggtctgaggaagagcagatcacattgtaaaactatggatggtctgataaggcttttactgaccccactgacttcagagttatactctgtttgctacatcataatgctggttttgctgactttttgtttttttatatatttataaaaaaagaaaaagttggtgattgcattgggaaattcccagggtattactggacctatgtggtgtattgttaaaccagtgtccttgtgatactgttgctcttgatgttcctgatacaggtaaggaaacagttggtcaactctgatacaaagtatatatacagttcagtattgtctctgttcattttgtttttatttcattgacaaaatcaaaccagcattccccattgtgtaaataaatgattttgctgaataaagtaaagtcttaaattcatatgttgaagcaaaaaaaaaaaaaaaaaaaaaaa

SEQ ID NO: 126 - DNA, 2579 bp, Homo sapiens
ggcgccgggggacacgttggctgcgttttcggcgggcctcccgggtacaaaaatggctgtggctagcgatttctacctgcgctactacgtagggcacaagggcaagtttgggcacgagtttctggagttcgaatttcggccggacggaaagcttagatatgccaacaacagcaattacaaaaatgatgtgatgatcagaaaagaggcttatgtgcacaagagtgtaatggaagaactgaagagaattattgatgacagtgaaattacaaaagaagatgatgctttgtggcctccccctgatagggttggccgacaggagcttgaaattgtaattggagatgagcacatatcttttaccacatcaaaaataggttctcttattgatgtaaatcagtcaaaggatcctgaaggccttcgagtattttactatttggtacaagacttgaaatgtttagttttcagtcttattggattacacttcaagattaaaccaatttaaattgtatgttttcaggctgtttgtatatttaattaagggatgggaggggttatttgtcatttacagtattggggtttttatgaatgtgaagcaaacaaaaaaaatttgtatgtaaactgaaaataagaaaatacattagcaagcttaatggttatccttacttgagtccacatgggttggacagtccccacacacattaaattctgtaaatgaaagccaccttttgttaaaaatttgctctaataaaacataccaaatcctggttgcagagtagttttttgttttttccaggaggctatgtctctaattcactttagagataataagaaattgttctggtagatacatcctgtgacagaagatactttaggtggaactatgtagccagattcccatccatgaaaggcaagtgtagattgtcccttatttccttcatacatgattggatttaattttggggggcttatacaaggtctagtttttttttacagttatgacaaacccctcagggattattcacatttaaatattttcagttacaagcagtgaggtcctaaagtgttacaagagtacagtctaccccatgttaggcatatctttgattatgtctttattccttatttcacaatgtatttggtgtgtaggggaggggggagaactaaatgagttttcagctttataaattgttaaacatttagacaaacatatatgtatgtatgaatgtacataaatatttttaactcctattgaccacgagtctcacttcagtttcccagttcctttcaacctctttctgatagatttcctctttcattacttttagtaaccatgttccttgtttccttttattcttccatctgaagccccactcttaaaaagttgcactgttccagtagttataatccacttgccctaggaacaagttagcactgaattttgggtggaataattagtttctgaaggcttgccaggacccctgagcaggtaggctctagagtcgggcagtccaataacttttttgcaataatggaaacgtcctatgtgcagtccaatagggtagcaactggccaaatgaggcctactgattacttgaggtgtgccttgtataactgaatttatggtgctatttaaacaatttttttctaacgtgaaaaggataaaacataaaaaactcttgagaactataaagtgaacacctatatgcctacccctacctagattctatacttaacatcttttttactgtaatatctctattataataaatcttggtttttcacttaactggtgtaattggtgccaataaactactttttttgtagtgctatttaattttgattaaatttagatagccacgtgtctagcggctaccgtttggacagtatagctctagagcatggcttggtaacctgtttgccatggagcactagatggtccttttcactcctcaaaatgcatgcccattgccttcaggtttgccatggaaagtcaaatgatttccacttcattatgcaagtacgctatcatcttcaggtcttttgtatgtaaaatgtttctgttccagttgtagaccttgatgattgtgcagtatgaaatcgtattgtaattttcttgcatttagatgtcaacctcagaaacaggaacaatcgtcttttgaacttccagtaggcccacagttgttggttgttcctcaaaacaggttgtggctcctgttgaataagatgatccattaaaaactgaacaaggttgaggagaaatagtgcttacgttgaaaaatctttaagtctttgtccccgttctctaacttccttacgttttcgtttatttagctccatccccactatctactagaatttctcatatttaaaccaagatgggagactaggtcattaggaaaatattaccgtctacaattttcttatactttgatctgtcttttatttgattgtaagttgctgatggacagtgatcattagaaactgaattttgtataatactagttttatatgaaactagatatttattgcgctcaggttatgttccttttacctccttccttaataaagagaccacttgaaat

SEQ ID NO: 127 - DNA, 4224 bp, Homo sapiens
gcgaaattcaagctccaaactctaagctccaagctccaagctccaagctccaagctccaaactcccgccggggtaactggaacccaatccgagggtcatggaggcatcccgaaggtttccggaagccgaggccttgagcccagagcaggctgctcattacctaagatatgtgaaagaggccaaagaagcaactaagaatggagacctggaagaagcatttaaacttttcaatttggcaaaggacatttttcccaatgaaaaagtgctgagcagaatccaaaaaatacaggaagccttggaggagttggcagaacagggagatgatgaatttacagatgtgtgcaactctggcttgctactttatcgagaactgcacaaccaactctttgagcaccagaaggaaggcatagctttcctctatagcctgtatagggatggaagaaaaggtggtatattggctgatgatatgggattagggaagactgttcaaatcattgctttcctttccggtatgtttgatgcatcacttgtgaatcatgtgctgctgatcatgccaaccaatcttattaacacatgggtaaaagaattcatcaagtggactccaggaatgagagtcaaaacctttcatggtcctagcaaggatgaacggaccagaaacctcaatcggattcagcaaaggaatggtgttattatcactacataccaaatgttaatcaataactggcagcaactttcaagctttaggggccaagagtttgtgtgggactatgtcatcctcgatgaagcacataaaataaaaacctcatctactaagtcagcaatatgtgctcgtgctattcctgcaagtaatcgcctcctcctcacaggaaccccaatccagaataatttacaagaactatggtccctatttgattttgcttgtcaagggtccctgctgggaacattaaaaacttttaagatggagtatgaaaatcctattactagagcaagagagaaggatgctaccccaggagaaaaagccttgggatttaaaatatctgaaaacttaatggcaatcataaaaccctattttctcaggaggactaaagaagacgtacagaagaaaaagtcaagcaacccagaggccagacttaatgaaaagaatccagatgttgatgccatttgtgaaatgccttccctttccaggaaaaatgatttaattatttggatacgacttgtgcctttacaagaagaaatatacaggaaatttgtgtctttagatcatatcaaggagttgctaatggagacgcgctcacctttggctgagctaggtgtcttaaagaagctgtgtgatcatcctaggctgctgtctgcacgggcttgttgtttgctaaatcttgggacattctctgctcaagatggaaatgagggggaagattccccagatgtggaccatattgatcaagtaactgatgacacattgatggaagaatctggaaaaatgatattcctaatggacctacttaagaggctgcgagatgagggacatcaaactctggtgttttctcaatcgaggcaaattctaaacatcattgaacgcctcttaaagaataggcactttaagacattgcgaatcgatgggacagttactcatcttttggaacgagaaaaaagaattaacttattccagcaaaataaagattactctgtttttctgcttaccactcaagtaggtggtgtcggtttaacattaactgcagcaactagagtggtcatttttgaccctagctggaatcctgcaactgatgctcaagctgtggatagagtttaccgaattggacaaaaagagaatgttgtggtttataggctaatcacttgtgggactgtagaggaaaaaatatacagaagacaggttttcaaggactcattaataagacaaactactggtgaaaaaaagaaccctttccgatattttagtaaacaagaattaagagagctctttacaatcgaggatcttcagaactctgtaacccagctgcagcttcagtctttgcatgctgctcagaggaaatctgatataaaactagatgaacatattgcctacctgcagtctttggggatagctggaatctcagaccatgatttgatgtacacatgtgatctgtctgttaaagaagagcttgatgtggtagaagaatctcactatattcaacaaagggttcagaaagctcaattcctcgttgaattcgagtctcaaaataaagagttcctgatggaacaacaaagaactagaaatgagggggcctggctaagagaacctgtatttccttcttcaacaaagaagaaatgccctaaattgaataaaccacagcctcagccttcacctcttctaagtactcatcatactcaggaagaagatatcagttccaaaatggcaagtgtagtcattgatgatctgcccaaagagggtgagaaacaagatctctccagtataaaggtgaatgttaccaccttgcaagatggtaaaggtacaggtagtgctgactctatagctactttaccaaaggggtttggaagtgtagaagaactttgtactaactcttcattgggaatggaaaaaagctttgcaactaaaaatgaagctgtacaaaaagagacattacaagaggggcctaagcaagaggcactgcaagaggatcctctggaaagttttaattatgtacttagcaaatcaaccaaagctgatattgggccaaatttagatcaactaaaggatgatgagattttacgtcattgcaatccttggcccattatttccataacaaatgaaagtcaaaatgcagaatcaaatgtatccattattgaaatagctgatgacctttcagcatcccatagtgcactgcaggatgctcaagcaagtgaggccaagttggaagaggaaccttcagcatcttcaccacagtatgcatgtgatttcaatcttttcttggaagactcagcagacaacagacaaaatttttccagtcagtctttagagcatgttgagaaagaaaatagcttgtgtggctctgcacctaattccagagcagggtttgtgcatagcaaaacatgtctcagttgggagttttctgagaaagacgatgaaccagaagaagtagtagttaaagcaaaaatcagaagtaaagctagaaggattgtttcagatggcgaagatgaagatgattcttttaaagatacctcaagcataaatccattcaacacatctctctttcaattctcatctgtgaaacaatttgatgcttcaactcccaaaaatgacatcagtccaccaggaaggttcttttcatctcaaatacccagtagtgtaaataagtctatgaactctagaagatctctggcttctaggaggtctcttattaatatggttttagaccacgtggaggacatggaggaaagacttgacgacagcagtgaagcaaagggtcctgaagattatccagaagaaggggtggaggaaagcagtggcgaagcctccaagtatacagaagaggatccttccggagaaacactgtcttcagaaaacaagtccagctggttaatgacgtctaagcctagtgctctagctcaagagacctctcttggtgcccctgagcctttgtctggtgaacagttggttggttctccccaggataaggcggcagaggctacaaatgactatgagactcttgtaaagcgtggaaaagaactaaaagagtgtggaaaaatccaggaggccctaaactgcttagttaaagcgcttgacataaaaagtgcagatcctgaagttatgctcttgactttaagtttgtataagcaacttaataacaattgagaatgtaacctgtttattgtattttaaagtgaaactgaatatgagggaatttttgttcccataattggattctttgggaacatgaagcattcaggcttaaggcaagaaagatctcaaaaagcaacttctgccctgcaacgccccccactccatagtctggtattctgagcactagcttaatatttcttcacttgaatattcttatattttaggcatattctataaatttaactgtgttgtttcttggaaagttttgtaaaattattctggtcattcttaattttactctgaaagtgatcatctttgtatataacagttcagataagaaaattaaagttacttttctcaagtgttttcaaaaaaaaaaaaaaaaa

SEQ ID NO: 128 - DNA, 3362 bp, Homo sapiens
agactccctgtctttgcggtttgggagatgatgagaaaccacagaattgctagtagtttatgtggagatcaggtcttctccaagaaaaaaaaaaagaaaaaaaaaaacaacatggctgcaaaggagaaactggaggcagtgttaaatgtggccctgagggtgccaagcatcatgctgttggatgtcctgtacagatgggatgtcagctcctttttccagcagatccaaagaagtagccttagtaataaccctcttttccagtataagtatttggctcttaatatgcattatgtaggttatatcttaagtgtggtgctgctaacattgcccaggcagcatctggttcagctttatctatattttttgactgctctgctcctctatgctggacatcaaatttccagggactatgttcggagtgaactggagtttgcctatgagggaccaatgtatttagaacctctctctatgaatcggtttaccacagccttaataggtcagttggtggtgtgtactttatgctcctgtgtcatgaaaacaaagcagatttggctgttttcagctcacatgcttcctctgctagcacgactctgccttgttcctttggagacaattgttatcatcaataaatttgctatgatttttactggattggaagttctctattttcttgggtctaatcttttggtaccttataaccttgctaaatctgcatacagagaattggttcaggtagtggaggtatatggccttctcgccttgggaatgtccctgtggaatcaactggtagtccctgttcttttcatggttttctggctcgtcttatttgctcttcagatttactcctatttcagtactcgagatcagcctgcatcacgtgagaggcttcttttcctttttctgacaagtattgcggaatgctgcagcactccttactctcttttgggtttggtcttcacggtttcttttgttgccttgggtgttctcacactctgcaagttttacttgcagggttatcgagctttcatgaatgatcctgccatgaatcggggcatgacagaaggagtaacgctgttaatcctggcagtgcagactgggctgatagaactgcaggttgttcatcgggcattcttgctcagtattatccttttcattgtcgtagcttctatcctacagtctatgttagaaattgcagatcctattgttttggcactgggagcatctagagacaagagcttgtggaaacacttccgtgctgtaagcctttgtttatttttattggtattccctgcttatatggcttatatgatttgccagtttttccacatggatttttggcttcttatcattatttccagcagcattcttacctctcttcaggttctgggaacactttttatttatgtcttatttatggttgaggaattcagaaaagagccagtggaaaacatggatgatgtcatctactatgtgaatggcacttaccgcctgctggagtttcttgtggccctctgtgtggtggcctatggcgtctcagagaccatctttggagaatggacagtgatgggctcaatgatcatcttcattcattcctactataacgtgtggcttcgggcccagctggggtggaagagctttcttctccgcagggatgctgtgaataagattaaatcgttacccattgctacgaaagagcagcttgagaaacacaatgatatttgtgccatctgttatcaggacatgaaatctgctgtgatcacgccttgcagtcattttttccatgcaggctgtcttaagaaatggctgtatgtccaggagacctgccctctgtgccactgccatctgaaaaactcctcccagcttccaggattaggaactgagccagttctacagcctcatgctggagctgagcaaaacgtcatgtttcaggaaggtactgaacccccaggccaggagcatactccagggaccaggatacaggaaggttccagggacaataatgagtacattgccagacgaccagataaccaggaaggggcttttgaccccaaagaatatcctcacagtgcgaaagatgaagcacatcctgttgaatcagcctagaggagaagcagcaggaatgatgctttgatactctggaggagaagttaactcaagatggaattcatgttctgatttgaggaatgaaaatgagatgatcaggcaggaaactgacattccaaggatctaatccaggaagtactctcagtggggaccacctgctttcatcccctgacattgtgggagaaattttgcaatgtatgctaatcaaaatgtatttatatgttctctgctgatgttttatagaggtttgtgaagaaaattcaacctcagcaacttcagaaactgcccctgatacgtgtgagagagaaataaaatcagattttgagtgttgaagggactgaggaagtgaggataaagagcatgaggacagcatggaaagaaggaggcagaagtggaactgaactttcactctccatgggacagatcaatctcattatcaagtctgaatagcaaccagccctctcctccaccccgtttctcctcagttaattggagctcagtcaggtgattattgagtcttgtacagcactgaaatgaaatcaaagatgaagaagcattgattgtattcgaagattgaagcacgctcatactttgtatgtgctttagggaaggggtgggtgggcacttgggccttgcgggtgcattcatgtaatctgagactcttgaactttatgacggagtcttcaatattttgatgtatatgaaacttttgttaaatatgttgtatacttcgctggctgtgtgaagtaaactaaaactctgatgaacactttggagtctgctttagtgaaggagaccaaagtgggaagggctttagggcactgatagaggccctgggtgtacttttcaatcctgtgtaatgtttaattcttgcaactgaatcaaaacagtgttaaattatggcaatatttgcactttgggaatgagtacataactgtatgatcacactctgcaaatgccacttttaaagctgttaatagactttgcaccttttctttgacaaggatgtgtcatatttaaatttttacattcatcatggctacaggtagaactggggaggggggaatgtaattttttatgggaattttgatatgaaaagaaactagtcatttatttatacaataggcttggctcaaaaagtgtttttcagacctcggtattcctaatgtgggatgtgactttattttatttttagtagcaaatttggatgtagactgacagacatagctgaatgtcttaataaatttaaatttgaagat

SEQ ID NO: 129 - DNA, 1963 bp, Homo sapiens
actagaaacgaggagtatttttcatgtggcactaatgagttaattcataatatgattttttgagacaaagtctctgttgcccaggctggagtgcagtggcacgatcacagcccactgcagcctcgacctcctgggctcagatgatcctcccaccttagcctcccaagtagctgggactacaagtgcacaccaccatgcccggctaatttttttgtagatatggggttttgccatgatgcccaggctgatctcaaactcctaggctcaagtaatccttctgccttggcttcccaaagtgctgggattataagcatgagccaccatgccaggccaatattattccccaaaagaaggaaattgggtgtgaggtcctgctcctatggtcaccatgagatttttttgtttgttttggactcttgcccaggctggagtgcagtggcacgatcatggctcactgcaacctcagcctccctggtagctaggaccacaggtgtgcaccactatgcccagggaatttttaagttttttgtagagacagggtctcaccatgttgcccaagctgttctcaaactcctggcctcaagcagtcctcctatcccaaagtgctgaaattacaggcatgagccaccacgcctggccaccactagtttttgtaatgggagcaggttccatatgagatggaggaatggatttcatgattgtctttgtaatttcttaggtccccaagaaattgattggacatgaggaaaccactctaagtgtgaccactctaaagcattagcagtcagtcatttcactctagggagaaatcagagctgtatatggagaataggtaaagtccccaatatggatatgtatttttatatttgactgcttgtatttttttgttagatgcaaatagtacggtattccgaacagacactaaaaatagctgtcatctcaaagaatccagtgcttgtgtcacagtatgagaaagtagatgctggggaacagcgtttaatgaatgaagcattccagccagccagtgatctctttggaccttgcattctccatcagattggatcacctcccaccctgaggccccccaagactttgaacagttcttcagtcatccttacagaaagataccctctccagacaaacgcagtatttatatacggtccattggatctctatgaagcaccagaattatcagtgaagaatatattaaatggctcacgggctactgtaaagcatatttctatcgcttgagagtaaaactgctagaaccagttcctgtttctacaacaaggtgttcctttagagtcaatgagaacacacaaaacctacaaattcatgcaggggacatcctgaagttcttgaaaaagaagaaacctgaagatgccttctgtgttgtgggaataacaatgattgatctttacccaagagacttgtggaattttgtctttggacaggcctctttgacagatgcaaaggattttgatagggaaatctggggcttcttccacttggaagaagctgaccggcgccctctaaacctttgccctatctgtttgcacaagttgcagtgtgctgttattggcttcagcattgtagaaagatataaagcactggtgaggtggattgatgatgaatcttctggcacacctggagcaactccagaacacagtcgtgaggataatgggaatttactgaaacccgtggaaggaagcctttaaggaatggaaagagtggataataaaatgcctgactgttctccaaaaataaggaccttcaaataggagtgattgaaataaatgactacttgcatgttatgctttcatttgggtggaatacttcatcagaataaactattgatcttgtgctgtgtcaaagtaacagactagaaccttctttcaagtacctgaattgaaatgaaactcattttgaataataaaaactctagaaactctttatcttctca

SEQ ID NO: 130 - DNA, 1966 bp, Homo sapiens
agcagctgcccctgcaaatgtcagcgccagcccagtcaaaagagcttgaaacctaccaagccggaggactgtgctgtgcctctctcgcccacattttccccaagcactctcaggaacctggcaacagtgtccccttgtggccaagcccggaacatcacatctgtacgttgcaatctgtggatcagctacgagactgagagaaaggaatgaaaggatggaagaattacaagatcaggcactgctgtctgtctgttccacggatgtaaccacagcacacgcgtggctcacggtactagtgtgataaatgcttgttacatgaaggcgtgaacagggatgagaagagacttcctggagaaacaaaaggactaacaatcaggaaggggaggtgatcggggcaggagtaaagtggacacctcagcaaagccattcgctgtgatctctgattgtgcagtgtcatgtcctgtcaccagagccccctcgtgtttgatgttggccaatgccgccagcatgatctagcaggccaaatcctaatctaccattctctgacaccagctggtcccctgggtcgtccacccgatgtcccccattctccccacttggcctcccccacaggctctcggcaaaggaccgtgggaggcacctgtgacactgcccttttcctgtgcagctgtttttcttcttcattcttttcactcctcgttactcttttttttttcactctcagcccacacaaaactaggaactttgttattctacttatttttctgtactctgtctgtttgcacacagatggatatctgagagccagcgaactttctttacctcctagtatcatttcatgaaaattagtagcacctgcacaatggggccttggagacaggaataaaaggaaaaatctggaatggaatcacatgacgcaacaggctatgaagactccctgcccggctgctatatgtctggtaaacagaataaatagtacttgagcatccctgactctgagactgtggggtagctggttgttttaagatgggaccagggtattgtgcacttattgccaattcttgcttcagtaagtggggtggctgtgcagagtgagtggcctgtgtatgaaccagggtccgtcccatctaatgagagtccggtcaggctgagtcctcactaagacttcagcgtggattcatcagtaaccttggttcactggcaggcttgctggactttggagaaaaggctgaccctcccccaaagcagcccattgctgccactgccctaggaacagagggcctggagccagggcttgacctggtacaggagcttttgcacaaggccggctcaaacctccaaacctggggaaacaggatgcatggtgaagtgaagaagaaagaaaatgcatccaccgttccttcctgttggtgtcagcacgatgaggaataattacctggatctggaaagcaggcgtgtgggaggctgcaggcccccctggtctgtgtagttaggcacttaatttttcatcctcctccttctctctgccctcccgaaaccgccgttttcaaaggaaaaacagaaaaacgtatctgggtaacccgtgtaatactgtttacatcagcccacttggcccagaaaggataccagctccttactgatgctcagataacagggttagtgcttaaaaaaattatgccttgggccgggtgcggtggctcacgcctataatcccagcactttgggaggctgaggtgggtggatcgcagggtcaggagatcaagaccatcctggccaaccaacatagtgaacctgtctctgctaaagatacaaaaagtagctgggcgtagtggcgcatgcctgtggtcccagctgctcgggaggctgaggcaggagagtcgtttgggcctggcgggcggaggttgcagtgagctgagattgcgccacactgcactccagcccaggcggtggagtgagtctccatctc

SEQ ID NO: 131 - DNA, 6131 bp, Homo sapiens
ctccgactctcggcacctggcctccagctttcggaactatggaggccgcgcccgggacccccccgccgccgccatcagagtcgccgccgccgccatcgccgccgccgccatcaacgccttcgcctcctccgtgttcccccgacgcccgcccggccaccccgcacctcctccaccaccgcctcccgctccctgacgacagggaagatggagagttggaagaaggtgaattggaagatgatggggcagaggagacccaggatacctccggagggcctgagagaagccggaaagaaaagggggagaagcatcacagtgattcggatgaggagaagtcccacaggagactgaagcggaaacggaagaaagagcgggagaaagagaaaaggaggtcgaagaagaggaggaaatccaagcacaaacgccatgcttcttctagcgatgacttctctgacttctcagatgactcggatttcagccccagtgagaaaggtcaccgcaagtacagagagtacagccccccatatgcgccgtcccaccagcagtaccccccatcgcatgccacgcccctgcccaagaaggcatactccaagatggacagcaagagttatggcatgtacgaggactacgagaatgagcagtatggggaatatgagggcgacgaggaggaggacatgggcaaggaggactatgacgacttcaccaaagagctgaaccagtaccggcgtgccaaggagggcagcagccgcggccgaggcagccgaggccggggccggggctacaggggccgaggaagccgtggaggatcgcgaggccgcggcatgggcaggggcagccgaggcaggggcagaggctctatgggaggagaccacccggaggatgaagaggatttctacgaggaagagatggactatggagagagtgaggagccaatgggagacgacgactatgacgagtactccaaggagctgaaccagtaccgccgctccaaggacagccgaggccgagggctaagtcgaggccgtggcaggggctcccgaggtcgagggaaaggaatgggtcggggccgaggccgaggtggcagccgaggagggatgaacaagggcggaatgaacgatgacgaagacttctatgacgaggacatgggcgacggtggtggtggaagctaccggagtcgtgaccatgacaagccccaccagcagtcggacaagaaaggcaaagtcatttgcaagtacttcgtggaagggcgctgcacctggggagaccactgtaattttagccatgacatcgaactcccaaagaagcgagaactgtgcaagttttacatcactggattttgcgccagagctgagaactgcccttatatgcacggtgatttcccgtgtaagctgtaccacaccactgggaactgcatcaatggtgacgactgcatgttttcccacgaccctctgaccgaagagacgagggagctcttggataagatgttggccgatgatgcagaagcaggtgccgaggatgagaaggaggtggaggaactgaagaagcagggcatcaaccccctgcccaaaccgccccctggtgtgggcctcctgcccacccctcctcggccccctggcccgcaggctccaacctctcccaacggcaggcccatgcagggtggccccccgcccccgccccctccccctcccccaccgcccgggccccctcagatgcccatgccggtgcatgagccactgtccccgcagcagctgcagcagcaggacatgtacaacaagaagatcccctccttgtttgagatcgtggtgcggcccacgggacagctggctgagaagctgggtgtgaggttccctggacccggtggacccccagggccaatgggccctgggcccaacatgggacccccagggccaatgggcggtccaatgcatcctgacatgcaccccgacatgcacccggacatgcaccctgacatgcacgcagacatgcacgcagacatgccgatgggccctggcatgaatcctggcccacccatgggccctggcggccctccaatgatgccctacggccctggagactccccacattctggaatgatgccccctatcccgccagcccagaacttctatgaaaacttctaccagcagcaggagggcatggagatggagcccggactcctgggggatgcagaggactacgggcactacgaagagctgccaggggagcctggggagcacctcttccctgagcaccctctggagcccgacagcttctctgagggagggcccccaggccggccgaagccaggcgccggtgtccctgacttcctgccctcagcccagagggccctgtacctgaggatccagcagaagcagcaggaggaggaggagagagcgaggaggctggctgagagcagcaagcaggaccgggagaatgaggaaggtgacaccggaaactggtactcaagtgatgaggatgagggtggaagcagtgtcacctccatcctgaagaccttgaggcagcagacgtccagccgacccccggcttcagttggggagctgagcagcagtgggctgggggacccccgcctccagaagggacaccccacaggaagccggctggctgaccctcgcctcagccgggaccccagactcacccgccatgtggaggcttctggcgggtctggcccaggtgattcgggaccctccgatcctcggctggctcgcgccctgcccacctccaagcccgaaggcagccttcattccagccctgtgggccccagcagttccaaggggtctgggccgcccccaacggaggaggaggaaggggagcgggccctgcgggagaaggccgtgaacattcccctggacccactccccgggcaccctctgcgggacccacggtcacagctgcagcagttcagccacatcaagaaggacgtgaccctgagcaagcccagcttcgcccgcaccgtgctctggaatcccgaggacctgatccccctacccatccccaagcaggacgcagtgccccccgtgcccgcggccctgcaatccatgcccaccctggacccccggctgcaccgcgctgccacggcagggccccccaacgcccggcagcgcccgggcgcctccacggattccagcacacagggcgccaacctccccgactttgaacttctgtctcgcatcctcaagacagtcaatgccaccggctcctcggccgcccccggttccagcgacaaacccagtgacccccgggtgcggaaggcccccaccgaccctcggctgcagaaacccacagactctacggcctcctcccgggctgccaagcccggccctgctgaggcgccctctcccaccgccagcccgagtggggatgcctccccaccagccaccgctccctacgacccccgcgtgctggcggccggtggactgggccagggcggagggggcgggcagagcagtgtgctgagcggtatcagcctctacgacccgaggactcccaacgcggggggcaaagccacagagccggctgctgacacgggtgcccagcccaagggtgctgagggcaatggcaagagctcggcctccaaggctaaggagcccccgttcgtccgcaagtctgccctggaacagccagagacagggaaggccggtgctgatgggggcacccccacggacagatacaacagctacaaccggccccggcccaaggctgctgcagcccccgctgccaccaccgccaccccaccccccgagggtgccccaccccagcccggggtgcacaacctgcccgtgcccaccctcttcgggacggtgaagcagacacccaagacgggctcaggaagcccatttgctgggaacagtccggcccgcgagggtgagcaggatgcggcatccctgaaggatgtttttaaaggcttcgaccccacggcctcccccttttgccagtagtgtccagccagagctgcggctccagccacccttcctagggtggcattcagggcagcacccagggtagggaacttgggggcaaggggaggcaggctgggtgttccttttttcttttctttttcttttgctttccgtctcttttatttttttttaaagtagtactttctttgagatttgtaaattgtatataaccatcttaagttctggtcagtgtggcgggctcaggggctcctgctgagcaaaccgactcatgcccgcaaacctgtgaactttcgccagtgcctggcctcagactctgtgggctctgcgtggccgggccttgctggaggcccagtgggttttctgggcaaagcatggccccttttccccaggacaaagggaacagttggtgtctgggaaggtattgaacgctcctcaccctgtgcccgaagagacccggaaccaagaccatggcagggcctgcgtggaagcaggtccaggcgtttctagaaccctagggtgcaccatcactgtcttttcagtgcaggctgtaacaacccactcaggagacagtgagagtgaaaaggtattaaggaaaaagcccccagcggcactatgggggctccctggcgcatgcctgctcctgtccctggattaccacacgtgccctccctgccaccctccgtctagagcaagcggatgccccccagcctgcagcagaagcctccacagtgagaactggacccaaaggtagtgggggccggtgtggggcagagtcctgaagagccacctctaggaggcagcccctaggagcacgcacgttctgtcagtattaccccacctgtcctatcaggtgggccacaccctgcttgcccacaccagggtctgtcctggtcctcaagccacgcacccgctatgcctgcactgcagcccagccccggacagctccaggatccgtgcagtggctgcgccgccaggccccaacaatggggaccctggggtggctcctggccaagtgttctctgttttcctcgcacctccttacactgtgtgacctgcagggcatgaggtattgatgtgttcgggtttcctttcccaagccagcagatgcaggtgttccaaggtgtgttgctctgtgggatttgtggacacttaagaaacggactgagtgggaaccctgcagccaggggatggggagcctctgctccccccatgctcccaccctggctgagggccagcctcatctgcagagccctggaggaggcccacctatggacacagcccgagagatgggcgcaaggggtgctgggggaggcctgctatcctgcctctgggccacttgaggggcctcaggaagtgtgtgcttgtggctgcatctgcccgtctccctggcccaccatgtggctgcaggccaagctcttcattgctgaccatgaagagacctagttacctgccaagggattccccttccctcctcctcagggtggggtgaacaaggctcctatcccaccccaccccaaaaagagaaaaatgaaaaactcatagtttggagccaggaggcagggtgtcctacagggctgcacagccctgaggggtcagtgctgggatctggttggttggtttgtctttttgtctttttttttttttttttttttacacaatcattaatgagatttgtcttcagccaccagtgttggccttgaagcagagggcacagcccttggtgtttgtaaaataagtatgaatacacagtgtggcagtgttgtggtttttgttgttgtttttcctctccttttgagaattttcttttgtaaaagaaaaatattttttaaaccgaaatctgtggatgaaatagaagctggagccctcctcttggaatattcagcctaagaacctcataggactatgaattcacccgaaattctcatttgccatcaggccgagcttttaaagaaaaattgttctctaaccaggattgtaacaaaagtgtaaatactgtttcagagttgagagttggtggtgcaaatatgtatataatgaactgtatttttacaatgatcgccgcatgactatttcacaccctttttatactccatatctgtcttccagaaacgtcacctgcctttctcctgtggtctcttaatccagtaattgtattactgccattaaaggatgcagttattttaaaaaaaaaaaaaaaaa

SEQ ID NO: 132 - DNA, 4892 bp, Homo sapiens
cacgccgcccctcctttccctttccgctctctccgcctccggaagcgcgggcgcgcggcgccgggagcccgttcagggccgcgggagtgcgccagcgccgcgcgtggggctgtggtggccgcggctctcagatatatttttgccatcatggatcagtttggagatatattagaaggtgaagtggaccattctttctttgacagtgactttgaagaaggaaagaaatgtgaaactaactcagtttttgacaagcaaaatgatgacccaaaggaaagaatagataaagatacaaaaaatgtaaattcgaacactggaatgcaaacaacagaaaattatcttactgagaagggaaatgaaagaaacgtgaaatttcccccagaacaccccgtagagaatgatgttacacaaactgtaagttctttctcattgccagcctcttcaagatcaaaaaaattgtgtgatgttacaacaggacttaaaatacacgtgtccattccaaatagaattcccaaaattgtaaaagaaggtgaagatgattactacacagatggagaggaaagcagtgatgatgggaagaaataccatgtgaagtccaagtccgctaaaccatctactaacgttaaaaaaagcataaggaaaaagtattgcaaagttagctcctcttcctcctcctctttatcttcctcatcttcaggttcaggtacagattgtttagatgcagggtctgatagccatctatctgattcgtctccgtcatctaagtcatctaagaaacatgtatctggtataaccctcctgtcaccaaaacacaagtataaatcaggaataaaatcgacagaaacacagccttcaagtactacaccaaaatgtggccactaccctgaggagtctgaagatactgtgactgacgtaagtcccttatcaactccagacattagccctcttcagtcttttgaactgggcatagcaaatgatcaaaaagtgaaaattaaaaagcaagaaaatgtgagccaagaaatatatgaagatgttgaggatttgaaaaataattcaaaatatttgaaagcagccaaaaaagggaaagaaaaacatgagcctgatgtctcctcaaagtcgtcttcagtgttagactccagtttagaccacagacataaacagaaagtcttacatgacacaatggatctgaatcatctcttgaaagcttttctgcaattagataaaaaaggaccacaaaaacatcactttgatcagccttcagtagcacccgggaaaaactactctttcacaagagaagaggtgagacagatcgatcgggaaaatcagaggcttttgaaagaactgtcaagacaggcggaaaagccgggaagcaaaagtacaattcctagatcggctgatcatcccccaaagttatatcacagtgctctcaacagacagaaggaacaacaaaggattgagagagaaaacttggctttattgaaaaggcttgaggccgtgaaaccaacagttggtatgaaacgttcagaacaactgatggactatcatcgcaatatgggctatctcaactcatcaccattgtcaagacgggccagatccactcttggccaatatagcccattaagagcttccaggacatccagtgctacgagtggtctcagttgtaggagtgagcgatcagcggttgacccctccagtggccaccctcgaagaagacctaaaccccctaatgtccgtacagcttggttataaaacacttttttactttaaacattgttcacacaacttttcttgaagtgctcgtgcatattcctataattctctgtgtaaacatctagaataccgttttagcaattgaaggtgtacaacagtgagttgtaatgtattgttattcagtgcaaaattattgtcaaaaaacgatttaatgtaaaaagtgtttcctgaggatgtatttatatgagatgtatgtgttcttaatagagaaatagtggtatgcatgtgtatcttctaattattcagttgtcatgctgtcaaaatagtagtgatagtatcattgcatgtcgtacccaagatggtcactatagttttcaatttgtgttattttcatttctttataagtgttataccatgagctcagctcctaaatttggctggctttgtttttcatttgctttagaacttatgtggcatgacatagctcgattgtatgagatttaacacttattatgaaacaaatcttgaaatttgttttcactggtagagctgatctagatttgatgagcagattgagagacgcttctattggagcaattcttgtaattcaccagaggtatctagttgttgtataagtgcactgagttagatcttaaaagtttgatcaatcattccaaaccatttcaaatatgaatattagaaagttcaacttagaagcttcccttttgtgttttcatgcattcaggtaaagtcctatttatgactcttagaaatgaggtaggttttagagctagtcttctaactcgtgaatagtttaaggaccacatcagttgcaaaccaagtatttgttgaatcagtaaaatggcactatcagcatgaagagccgagaagcagatcctgaggtgtgtcggacagtgttttaggtaattcagtatttattatgttattagagaagacttagcgtaagaaagaaatgctttagaaattccagcaagtatttataaggcctgaaaaaactaaaatttgaacaaaaagggaaataaatcacacatttgttgaggctgctgctttaagtattagctaattttcttgttttggtttactttgatttatttgattgaaccctggaagtattatttttaccagtcccacatttaaagacctagtaatcttgatggaattggtaatggcagatagggtttaatgttgttgacttttcaaattagaattttttttctatgaaacttaggcttttatataacaaagtatgttatctcattgttaccgccgtttttcactactagccttttcagtatgcccttaaaatgtattggaaaggttccatgttaaatgacaaatatctttttcagtatgaatttttgatctgtagccacaataagattttcatttttcagtgctcaggctctgtagcatgtatgctgaatgcttttttccttagcacctttaacagtaaggtgttactgcattttacaaaacatcaattcaacatgtcttggatttagtttgtattcatagattttgttaaggatcaggcctgtgaacatgggcacagtgaggttaaataattcacctggccacaacctggctttgaggttctgcttttgggggccataattttcattcaatgttattcgatgctctgttctggtgaacaagttactatacctgctgtgtatataagcttgagctatactaaaccaaggctttattttcttttgttttttaacttactgtacttttactgtttatataacctatatttcaagagagagaagataatgctgaattttaaatttagcatttgaacatctgtccaatagtgaattaatcagtttttttaaaattgatttttaagttgttccagaaaaaaatatatatatatgttttatatatataatctttttatatatataaaaaatcttttttataaattttattttaaaaatcttttaaaaaagatttttatatatatataaagattatatatttatatatagtcttttctgttcataatggcattagtctctgagtacattttatattatctttgttgccacatgtcaggatacaacacaatagttttataaataacaactgtactttgattttaggtactaatttaatgctttagtttttactactttgaaaggggaggttcatctaagtattactgataacagggatccaacttaaagtaaaagttggaaagggcaagagtcttttaagcggaacttcaattctgttgtcatggttacttatatataatgaatataaatagacaaataggacttggaaaatgtggtatattctaagtcaagatgctatgaagttgttatagtttaagggacattcttaagaaggaattacttaagtacattactcttctcatttcaaggacatgaaaaagtaaaggatggtctggcactattttattttattttttttttgacttgtactgaatatatgtactttgaagatttacatctataactagaaagtatgctaatccgaggtctggttattaggccagattcttgttcagttattttttaaactaatgttgcctcctttttagtaagattctacgaaagttttttaactattaaagttatttcttaaggggtagctcttatacttcctctgctctctttttcagtttccataaaaagctgttttcttagctattactccaaataaagttctttggtttgcttaaaaatgtagtgtaatgcagttgttttctctccgctaacctgagtgtccacagtagatatcgagacagctttaggaaggtgacggtcatgggtaagtgacggctgtgcctgtcatcttcagattcttcaaatactgcaaactatgaaacatgttaagaccaatataaacccaaagatgtgagttgattatactttaataattacattcataaaaagtttactatcttcaatgccaaaaaagtcttaacctataaatgattatatacaatcttagaaaatgtgcttaacagttatggcttctttatttctatgaggaaagattagtaagaaacttaaaattaacatccttttaagattttctgttccattttaatgtattttaggcatttagaactagcctggcataatgaagaaagaaataaaagaattaaagataaaaaaaaaaaaaaa

SEQ ID NO: 133 - DNA, 5420 bp, Homo sapiens
ggcggggagcgcgggctgcggagaggcgggccgggccaagcggagccgagcgagcgggagcgcggcgtccgggaggcggcggagacgcggggctcggagggtcagcctcttatcgtagcaggtctcctcggcacgccccccttgtttcgccccacggccaagcccgccgcgggccggcgtgcgctggtcactgaggcccaggtcgccgccgcggcgcgtttttgaaatcatgaatcctgtttatagtcctggatcttctggggttccctatgcaaatgccaaaggaattggttatccagctggttttcccatgggctatgcagcagcagctcctgcctattctcctaacatgtatcctggagcgaatcctaccttccaaacaggttacactcctggcacaccttacaaagtgtcctgttcccccaccagcggggctgtgccaccgtactcctcctccccgaacccctaccagactgccgtgtaccctgtgcgaagtgcctacccccagcagagcccgtatgcacagcaaggcacgtactacacacagccgctgtatgcagcacctcctcacgtcatccaccacaccacggtggtgcagcccaacggcatgcctgcaacggtgtaccctgctcccatcccccctcctagaggcaacggggtcaccatgggcatggtggctgggaccaccatggccatgtcagcaggtaccctgctgactgctcactccccaactcctgtcgccccccacccggtcactgtgcccacgtaccgggccccaggaacgcccacttacagctatgtgccccctcagtggtgatcacctgcaaatgtttgaggacggagctgtgcagtcacattattggggattccacagctggtgctgcaggccttgcgcctccaaccaggactttcttcttaatgctctcgacacttagctaaacacgactatatcccggcccagcaggccccagcgccgttagtctccagctgactctgtgggttggtcttaaagcaaattctgttttgtggactgcctggcaattttttagctaactgtaatgataaaaagggagtattaatctattctgaatcatatctagttgaatgcatgtttaaaaaaacaaacacaaaaagcttgctcaatctacctgcagtgactgatgcaaaaccatcatatgcaaaatccaaaggaatggaaacgtattttacaacttgtatcactaatgcactgttgtaatgtatgcaaagtcttacagttataagtgttaaagtgaatttcttcatagagcatctgaaatatcttagatgattcttcaaccttttggggttgatgtgggttgccagttagggatgtggacattttagttttcagcgacctgtttctttggcactgactgtcctgggagggagttgcgaggtttgggagagagtagggaagccacagctgcttgggtgcagctggttcatggacatccctttgagtttaggcttggtggagtcagtggaacagggacatgcttaaagctcatcatgaaagattatggtagtgtggccagtgaaatttggggcgaggggggtggtttatgtgtcagcaaagcagtttctctgctatctaaattttcattacagttctcttagagagatgatgtttttatatgtctttattggaaaagtcctatgtaaaactaaattattttcctgggatggaagaaatgtgaaagagaaacagccatgcttgcaggaggtattgtcctctgctttatttagcttagaaaatcattcctttttttttttttttcctggagaaatgtttgaatcagctgaaaacaggtaggcattgctgtttttccccaacaaagaagggcaaaggtttcagctgtatgttatgaagaaagtggtatatttaagaatgagttaaaagggaacaaaactttatttaaaattcctccaatttcttgtagaaaggcagggccgtggatatgtctggaaatgtagaaacctgtagctgcttctgggatcacccacctgatggtggtgacttgtctgctgaggccggctggaggggacgcttagagatggggcgagggggaggccagtgtctgttgtctgcgagcctctcctgccatctcttttgcagctgagggcacttggcacaaagcaagcacagacagcagagagggccaggcagtgctggagtgcgtttgctggcagggtttattgtgggagaggaatttgagtttaggatctgaatcttgtacataagaaatgaaaaggcttccctccaccccgcccccaaaatatgcccgtcctgtatccatgagagtgcatgtcggttctcagtgagggtcagagtgggggtgggggatgtaaggcctgggtccctttcagcggcctctagggcaggagcgtgcgtcttcttcccagtgcagtggtggtttgagtgctgtgggctctggtggggagggggctctgctcctcttcatttgctggtagctgggtcagggcactggcgcccagtgaggatgcccactagcattctgccccagttggttggtggggggacacctgaccactgagcctttggtaaccttattttttatagtaagtacttcttataattttctttttcacatcttattttataagaagtttaggaatatttttgcatggatcattgtaaacagcagattctttattcctgatgtctattaacatagaatgtttactgataagtactttaaattgcttcatgagcacttaatccatcttagtgtctatgtgtgggggcagacatttaccgcaggcacacagtttgtgcccctttcttcaagtctctgttggtaattccatctgtttagtggatggttggcaggattagtaggttctaacggttccaggggttcagctgaccaagtagcagagaagtcttttttcccaattgagtgctaatagattaggataaaatactcacaatgttagtgtatgcttttaaaaagccccacacaacaaaatggaaacatacatgtacaactcctgtgagggctggagtcttggggttcaggaggaggttagaagttacaggcatctcttcaggcttgcttggtacttggcacacacaggatggtgttttaaagagtgggctgcaccccccacacgccatttacatcagcttcataaacacttttcttcctccctgtaacttaaccttttttcccttttatgaagttgagaggctttatgaaataagtttgcattgcacatccgtgcagaaatctttctgactttgaaatttttaggacgtcagctgtcagatacgaaaggtagatatcaggtaagaatctggacttaggaaatagtcacaaaactgtcataggttgtaattttatcaacattcgcttctagtaaaattaaagtcaattaagaaatagaacttgggtcaaaattctgttacaaagcttcataatttgtcccgaagcatatggtggagcattctgagaaatttgctttttgtgtgtttgacattcctaatttgggagtccttcagctgaattactattcttttagaagttgagacagcaggtaagcaaaggacctagttcatgtaaacatggacatcatgatggctatttaaaaaatatttgttctacaccttctcccctgaggcttggggagtgtgttcagccgctgcagtttctctgctcatggaggtcttgtttggatctgtgctggcggctgagcatttagtgtgagccagtgacccatgaacttgccgctctgtgagggccagagtcagggccagtcatggtaatgggcctgaaggcacttccagaaccttttatgtctctcgtgagccatctgttaagaacgttcttcttggtgtggtttgtaggcctacctgtcgctattctgggaaaccttcttgagtgctatgcaaatgtgttcacaggcaatgggggtggtcctgagccttggggtgggcacctggtcagtgagtgtcttgcccttccccagctgggcatacagtaccttgctctttctggtggcatcatctggctgtgatgaatgaggtctaggaaataatttgcatgtgtcttgggggacacaacagtaacgagaggaaatacattattacagcaacttgcgacgtactaatacctgtcagtgttggcccccgtaaggtatgtaaggcacctgtgagtgccagtgagtgctggtgaaaggccaacatgtactagttatgtaagtattggtgtctgctttaaaaaaggagacccagacttcacctgtcctttttaaacatttgagaacagtgttactctgagcagttgggccaccttcaccttatccgacagctgactgttggatgtgtccattgtcgccagtttggctgttgcccggacaggacaggacctccattgggcgcagcagcaggtggcaggggtgtggcttgaggtgggtggcagcgtctggtcctcctctctggtgctttctgagagggtctctaaagcagagtgtggttggcctgggggaaggcagagcacgtatttctcccctctagtacctctgcatttgtgagtgttccctctggctttctgaagggcagcagactcttgagtatactgcagaggacatgctttatcagtaggtcctgagggctccaggggctcaactgaccaagtaacacagaagttggggtatgtggcctatttgggtcggaaactgcatgtttcagaaagtttgtctctttttcctactcattatttgaagaagagagagcctgtggggatagccttatcttggtccacaggactgaagtgaagtcacttgggaaggggagacagaggatgagcggcgtctgtgtagggacccccccccgggcctgcagaagggtggtgtgctcccaggactggcatgacaggtgtctcctcctcaccacaggctgtgcccatgagtccctgtgcagaccagtgggcaaggcagctgggccagatctcaggccagccgtttgtgctcctagcagggttgctgtgctggccacacggagaggccctagagagcctcatggattgtaactaaagaagaaacggttcctttttgtttttttaaaaatgatttttaaataccgttttttacaccgttctctcggtactttttttaagctaagtcagcattgtcttccagtgttaaaggcatccctcacctctgcattgaacttacgtatccatgccaaggaatggaatttccatcctgagccagttcagttaggtgtcaattgatactattttaattttttatgcaatctgatgagatgagctcagatttaaaaatctcaaaagcacgtttattgtaacaagaattgttatgtattaatactgcagttttcaataaagattgacttgtgttgcaaaaaaaaaaaaaaa

SEQ ID NO: 134 - DNA, 560 bp, Homo sapiens
ggaagaggacggacctaagatggcggcctccagggggctgggaatagccgctcatgtcggctaacggagcggtgtggggccgcgtgcgaagccgcctccgcgccttccccgagcggctggccgcctgcggggccgaggccgcggcgtacggcaggtgcgtgcaggcctccacggccccgggcggccgcctgagtaaggacttctgcgcgcgggagttcgaggccctgcggagctgcttcgccgctgcggccaagaagacgctggagggaggctgttaggagggactctgagcttcacacctgtctgctgccatgggtgcagagccctagtcctgatggcccctggtggcacatatcgaatgcctagggcagaaaggaagtgggaatggcgaagatgtgacattcctcggtgttagatcctgttttttcttaacaagttgaggcgtgggtagagcaggaattggttttccagcattgtgtccgtaaacctgagttagaataagatgtaacggaagccacgataaagactcggtcaaatcctgcagcctggggcttactgtg

SEQ ID NO: 135 - DNA, 2017 bp, Homo sapiens
gtgaccctgccgggccaggagccgggaagaagagccggtgttctctctgttcctcgctagcagcttgggacattagcgccgagatgtgccccatatctgagctgccttcagctccttggaaacggatccaggtttttcctaccccttccgactgccccatccctctccagagaatccttttctgccctggtgatcaaggcgcgtcaattcaactctccctagagtggccacagtactggagatccaaagatgacttaagacatggtacctacgctggctctagtggaggagacagacacgtagtcaactgaactattttacaaacctgaaatatgtgccacattgagataaatccaaaatgcctagaatctttgttcagctcccctttacttagttcttaagcccaaaagaggtcttcattctgcttacagctcctggtgctctaactccagagcattttgcatacatctttagggttattctcacattgaactgtatttttgtgaatgcctttctgtccgaatccaataccagtggtctaacaattcacaaaagaaaacagaaatattggaaactgtactatggagaaattagggacaaaaggtaacagtatattgatattaacattgctgctagtcctttgcactagtaaataactgctatttgataaatgatcacaatgtgtaaaacactgtagttacaagatctcatttaatccgcctaacaaccttgccaagtattaataaaacccgttttaggcgctgacactgacctacagcgcctcagctccagcgccatggcgccctccaggaagttcttcgtggggaggaactggaagatgaacgggcggaagaaatgtctgggggagctcatcggcactcagaacgcggccactgtgcctgccgacaccaaggtgatttgtgctctcgccactgcgtataacgagttggcccggcagaagctagctcccaagattgctgtggctccgcagaactgctacaaagtgactaatggggcctttactggggagatcagccctggcatggtcaaagacttaggagtcacgtgggtggtgtggtcctggggcactcagaaggcgtgtctttggggagtcagatgagctgattgggcagaaaagtggcccatgctctggcagagagactcggagtaatcgcctgcattggggagaagctagatgaaagggaagctggcatcactgagaaggttgtttttgagcagacaaaggtcatcgcagataatgtgaaggactggagcaaggtcatcttggcctatgatcccgtgtgggccactggtactggcaagactgcaacaccccaacaggcccaggaggtacacaagaagctccgaggatggcttaagtccaacatctctgatgcagtggctcagagcactggtatcatttatggaggctctgtgaccaaggcaacctgcaaggagctggccagccagcctgacgtggctgccttcctcatgagtggtgtttccctcaagcccgaattcgtagacatcatcaatgccaaacaatgagccccatccgtcttccctacccttcctgccaacccaggaactaagcagcccagaagctgagtgactgcccctcccctgcacatgcttctgatggtgtcatctgcaccctattgtggcctcatccaaactgtatcttcctttactatgtataccttcaccgtgtaatggtcgggaccagaccaatcccttctccacttactgtaattgttggaactaaatgtcaccaatgtggcttctccttggctgagaggtgaaagggatggaatttgctcctgggtcccctaggccctagtgaggggaggagagagaacccatcctctcccttcttacactgtgaggccaagcagaagccaggggtgctgccctctcccacggtgccaacgcctttgtgtgttgtgtatgtgagccatcccacatgtgagggaaataaacccctggcacttaaaaaacaaacaaaaaaacgcatttt

SEQ ID NO: 136 - DNA, 681 bp, Homo sapiens
atggtcgatgatgctggtgccgctgagtcccagcggggcaaacagactccggcccactccctggagcagctgcgtaggttaccacttccgccgccacagattcgcatccggccctggtggtttccggtgcaggaactgagagaccctttggtgttctacctagaggcatggctggcagacgagctctttggcccagaccgagccataattccagaaatggagtggacgagccaggccctgctgacagtggacatagttgactcagggaacctagtcgaaatcaccgttttcgggcggccccgtgtacagaatcgggtgaagagcatgctcctgtgcctggcatggtttcaccgagaacatcgtgcccgagctgagaagatgaaacaccttgagaagaacttgaaggcccatgcatcagacccccactctccccaggatcctgttgcttaagacaacatagttactgttgggaacatcttaactttctaacttttgctgctaaagttgaagaaaagcaagtatagcattcttaaatccccgtattcctttttcctgtgtcttgatggattgtggtttattttgttgcaagagtgagtttgaactattctaataaagaaatggctattttgccaaaagcattaagatcttcacacacttataataaagcaaatttataaaaga

SEQ ID NO: 137 - DNA, 259 bp, Homo sapiens
atgacagacactgaaaatcacgactcatccccctccagcacctctacctgttgcccgccgatcacagccggaatgcagctgaaagattccctggggcctggttccaactgcccactgtggactctgaggcctctgcatttgcgggtggtctgcctgtgatattttggtcatgggctggtctggtcggtttcccatttgtctggccagtctctgtgtgtcttaatcccttgtccttcattaaaagcaaaactaaagaaaa

SEQ ID NO: 138 - DNA, 3568 bp, Homo sapiens
gtgactgggaagatggccgtctttccttggcactccaggaataggaactacaaagctgaatttgcatcatgccgactggaggctgtaccattggagtttggggactatcaccctctgaaacccataactgtcacagagtcaaagacaaagaaagtgaaccggaaaggaagcacttcttccacgtcctcctcctcctccagctccgtggtggacccgctgagcagcgtcctcgatgggactgaccccctctccatgtttgcagccactgctgaccccgcagccttggcagctgccatggacagctccagaaggaaacgtgatagagatgataactccgttgtaggatcggattttgagccttggaccaacaaacggggagaaatccttgcccggtacaccactaccgaaaagctgtctattaatctgtttatgggatctgaaaaaggcaaagctgggactgccacattggcaatgtcagagaaggtgcggacccggctggaggagctggatgactttgaggagggttcccaaaaggagctgttgaacttgactcagcaggattacgtgaaccgcatagaggagctcaaccaatcgctgaaggatgcctgggcctcagaccagaaagtgaaggctctaaaaatagtcatccagtgttcaaagcttctttcagacaccagtgttattcagttctacccaagcaaatttgtccttatcaccgacatacttgatacatttggaaagctcgtgtacgagcgcatcttttccatgtgtgtggatagccgcagcgtcttaccagatcacttttctccagagaatgcaaatgacacggccaaggaaacatgcctaaattggtttttcaagattgcctccatcagggaactcattccaagattttacgtggaggcatccatcctgaaatgtaacaaattcctctccaaaacgggaatttcagagtgcctgccccggttgacatgcatgatcagagggatcggagacccactagtgtcggtgtatgcccgtgcctacctgtgccgggtgggaatggaagtggccccacatctcaaagaaaccctaaataagaacttttttgacttcctccttacgttcaaacagattcatggggatacggtccagaaccagctggtggtccaaggagtggagctcccatcttacctccccttgtacccgcctgccatggactggatcttccagtgcatctcctaccatgcccccgaggctctgctgaccgagatgatggaaaggtgtaagaaactaggaaacaatgccttgctgttgaattctgtgatgtctgccttccgggctgagttcatcgccacaaggtctatggatttcattggcatgattaaagagtgtgatgaatctggtttccccaagcatcttctttttcgatcactgggattaaacttggccttggctgatcctcctgagagtgaccgacttcagattctcaacgaagcttggaaagtcatcactaagctgaagaacccacaggactacattaattgtgccgaagtgtgggtggaatacacctgcaagcatttcacgaaacgagaggtgaataccgttttggcagatgtcatcaagcacatgactccagatcgtgcatttgaagattcctacccccagcttcagttaataattaagaaagttattgcccacttccatgacttctcagttcttttctcagtggaaaaatttctgccgtttctggacatgttccaaaaagagagtgtgcgggtggaggtttgcaaatgcatcatggacgcctttatcaagcatcaacaagagcccaccaaggacccggtcatcttgaatgcccttttgcatgtttgcaagaccatgcatgactctgtgaatgcactcactcttgaggatgagaaaagaatgctgtcatatttgattaatggatttataaaaatggtttcctttggccgtgattttgaacaacagctgagtttttatgttgagtccaggtcgatgttttgcaatctggagcctgttcttgtgcagttgattcatagtgtgaaccggttggcaatggagacaagaaaagtaatgaaaggaaatcattccagaaagacagctgcatttgtccgggcctgtgttgcctactgcttcatcaccatcccctccctggcgggcatcttcacacgtctcaatctctacctgcattctggtcaggtggccttggccaaccagtgcctctcccaagctgatgcttttttcaaagccgctataagccttgttccggaagttccaaagatgattaatattgatgggaagatgcggccatcggaatcgttccttctggaattcctctgcaatttcttttctactttattaatagttccggatcatcctgaacatggggtcctgtttcttgttcgagagcttctcaacgtgatccaggactacacctgggaggacaacagcgatgagaaaatccgcatctacacctgcgtcctgcatctcctctccgccatgagccaggagacgtacctttaccacatagacaaagtggactccaacgacagcctctacgggggagactccaagttcctggcagaaaacaacaagctgtgtgagacggtgatggctcagatcctagagcatctgaaaaccctggccaaggacgaggccctgaagcgccagagctcgttgggcctttccttctttaacagcatcttggcccatggggacctacgcaacaacaagctcaaccagctctccgtcaacctgtggcacctggcacagaggcacggctgtgcagacaccaggaccatggtgaaaacgctagaatacatcaagaagcaaagcaaacaaccagacatgactcatctgacggagctggccctcagactccctctgcaaacaaggacctgacccccgggcccatccccaggctcagggactctggtgccaaatccagaaagatctgctctgctgccctgaactcttacggcaatttaggtttctcatttttcttttctttttacatatgtacaaattgttttaagctttggcctctatccaggttattctgacaatgaagaaatgggagttgtcagagcattaaaatgcaatcttcactaagaagcagtctctgtgttgtctttgcacaagtggccttcggtctactcagcccgatctgatgggcctttttagcaagagagaaacaagaatgcaagtaacatctttcttctctggaaggtgtttgttttttcatagtttagaaataaggactttaaaagtggactgcttttcaaagtgccactgttccagacccattccattccagactttgtaccttaaagttagagcacacccaaagtctggaactgtgttacctgaacccctatggaggatttataaaaggcagaaatagcactccattaactctttttcctatcaaaagcagctcttgattggacttagaatctgtgttggtggatcaaaggagaaagcgaggtcaaatttgagattctctgtggcttcagtatacagtaactgaataaatgtcctgaaggagaaaaaaaaaaaaaaaaaa

SEQ ID NO: 139 - DNA, 902 bp, Homo sapiens
gggtagccgactggggtctcctggcgacgaccatggcgggggatgtgggcggtcgcagctgcacggactcggaactgctgctgcacccggagctgctgtcccaggagttccttctcctcactctggagcagaagaacatagctgttgaaactgatgtaagagtaaacaaagacagtcttactgacctttatgtccaacatgcaataccattgcctcagagggatttgccgaagaatagatgggggaaaatgatggaaaagaaaagagaacaacatgagattaaaaatgagactaaaaggagtagcactgtagatgggttaaggaaaagacccctcatcgtatttgatggaagttcaacaagtacaagcataaaagtgaaaaagacagagaatggagataatgatcgactgaagcctcccccgcaggcaagctttaccagtaatgcctttagaaaattatcaaattcctcttcgagtgtttcacccctaattttgtcttccaatttgcctgtgaacaataaaacggaacacaataataatgacgctaaacagaaccatgacttaacgcataggaaaagtccttcaggccctgtgaagtcgccaccattgtcccctgttggaactactccagtgaagttaaagagagctgctcctaaagaagaggcagaggccatgaataacctgaagcccccacaagcaaaaaggaagatacaacatgttacttggccctgaagaaaagtttccaaaaatgtaaatatactgtaactgtagtttttcaaatatgttcatatatattgacaatatttacagaaatcctgattattgtggaattttcttaagaggtttcaaataggtttaaaaaaataaaggatttattttccttcccttaaaaaaaaaaaaaaa

SEQ ID NO: 140 - DNA, 755 bp, Homo sapiens
ttccccaggagcagttttggtttcagacggcgccgtctcccgcgaaagtcctgagaggagcccagccttttccgcctgccgcccccggatgggatggttgaggccggggccacgccccctctgcccccctgcgagggcatcctgggctttctcccaccgctttccgagcccgcttgcacctcggcgatccccgactcccttctttatggcgtcgctcctgtgctgtgggccgaagctggccgcctgcggcatcgtcctcagcgcctggggagtgatcatgttgataatgctcggaatatttttcaatgtccattccgctgtgttgattgaggacgttcccttcacggagaaagattttgagaatggcccccagaacatatacaacctttacgagcaagtcagctacaactgtttcatcgctgcaggcctttacctcctcctcggaggcttctctttctgccaagttcggctcaataagcgcaaggaatacatggtgcgctagggccccggcgcgtttccccgctccagcccctcctctatttaaagactccctgcaccgtgtcacccaggtcgcgtcccacccttgccggcgccctctgcgggactgggtttcccgggcgagagactgaatcccttctcccatctctggcatccggcccccgtggagagggctgaggctggggggctgttccgtttctccacccttcgctgtgtcccgtatctcaataaagagaatctgctctcttcaaaaaaaa

SEQ ID NO: 141 - DNA, 1514 bp, Homo sapiens
agagttgaggccaatggcggctccaccatcctgtggctatgattcctgaacttgtggtctcctcagttgatgaggtgaagaaagaaagcctggagaattatgcacgagcttctcactgtctcagcccacaagtaacacacctctttccctcacagcccacactggctggaaccaatcacatagccctgcctaactgcaagggaggccagaaagtgcaacactttctcatggtcacgggtgagcacgaaacatctctccaaatatgggttgggaagattaagagactagtccagaagaaaacagtctccagggagaaatatatctgggaataggaatcgagaaagcaaccaagagagcagaactgagtggatgagaggaattgtcagaccacatctgcagctctgattagtcaaatccgaaatgtgcccgttgatctattattccgtggaaatactggaatcaccagctcaataataagaggcgctggatccagggcaaaaatgaagacataagggaccttaggtgacaaggaagaagctcccaggcagcatgtgggatggacccaaggagaacgtcagagagaaagagttcagtctggtgtctcctgaagcaggttaaaattaagctcggcaggctcgacgtaaatgtgcagacaaagcaaagcaggaagcctcccacactaaaaagggaagataagaatcacagaagctgggatggtttttattgaagggcatttcaaagcaaatacagacatctacagcatatttgtaaatcctccgtatgtgtatggaaacacacaccttcacacatcaaaactggatttgttcctgagtaacaaaacgtcctaagaacaaaacgaaagaaaaccaaatagcagaaaaatacttccaatgtatataccacaaaggattatttatcaactcaacacaccaagagttaagaaaataatgcaacaggaacaaagggcaaaggatatgtactgggaagaccacagagaaagaaaatgcaaatggctaataatataaaatatttcctaggttcaatagtaatcagggaaagacaaaattacacttttgacctcttagactggcagtgacaaagattatttgagtgaccaagctttagtcaggctcctggatcttctaggcccatctgggcacttcctcgtaaaatacagttttaacaaaagccctgctaaattggtttaccgagaactcccaccttcaatcgagttccttagcccttcacctttcctcaggtgagggctgatcatcctgtcctgtcttcagcaagactgctctaacgctgacgtttccttttagtaattttccatccactgacacccacttgtaatttatagccaaggagcaagatacactgctccttggctataagttattttccctgttacattcagagttgagcccaatcccactccccgactacaaaatcgatagcagtggtccctatacctattgcaatggtcctaaataaaacctgccttaccgtgcttg

SEQ ID NO: 142 - DNA, 471 bp, Homo sapiens
gagctctctctggtccgtgcctccaagatgacaaagaaaagaaggaacaatggtcgtgccaaaaagggccgcggccacgtgcagcctattcgctgcactaactgtgcccgatgcgtgcccaaggacaaggccattaagacattcgtcattcgaaacatagtgaaggccgcagcagtcagggacatttctgaagcgagcgtcttcgatgcctatgtgcttcccaagctgtatgtgaagctacattactgtgtgagttgtgcaattcacagcaaagtagtcaggaatcgatctcgtgaagcccgcaaggaccgaacacccccatcccgatttagacctgcgggtgctgccccacgtcccccaccaaagcccatgtaaggagctgagttcttaaagactgaagacaggctattctctggagaaaaataaaatggaaattgtacttaaaaaaaaaaaaaaaagaatgcacatgag

SEQ ID NO: 143 - DNA, 932 bp, Homo sapiens
ctgcgcctgcgcatgccacacgcgcactcgcgtggccttcgcgaaggtgtcgctgccaagaaacgtgtcctgcgcgctacgccgtctgtttctagggcaacgccggcgtctcttagcaaccgcgcgcggcctaggtgggtccccccggcacccccagacctgccatggcgaccgcgagtcctagcgtctttctactcatggtcaacgggcaggtggagagcgcccagtttccagagtatgatgacctctactgcaagtactgctttgtgtacggccaggactgggcccccacagcgggtctggaggaggggatctcacagatcacatccaagagccaagatgtgcggcaagcactggtgtggaacttccccattgatgtcacctttaaaagcaccaacccctacggctggccacagatcgtgctcagcgtgtatggaccagatgtgttcgggaacgatgtggttcgaggctatggggccgtgcacgtgcccttctcacctggccggcacaaaaggaccatccccatgtttgtcccagaatctacgtctaaactgcagaagtttacaagctggttcatggggcggcggcccgagtacacagaccccaaggtggtggctcagggtgaaggccgggaagtgacccgtgtccgttctcagggctttgtcaccctcctcttcaacgtggtgaccaaggacatgaggaaactgggctatgacactgggccttctgatacacagggtgtgttggggcccagcccaccccagagcttcccccagtgaaggctccacaggctgcacagtctctgataatgaagggctgccttcccgaagtcagccgctgcccatcggcctgaggggcagcctggtggccagagctgggggcacacagaatagttttgtataataaagtctcattttcagagagcctaaaaa

SEQ ID NO: 144 - DNA, 441 bp, Homo sapiens
  misc_feature (10)..(10): n is a, c, g, or t
cctctggtgncccttctgaaggatcccgtgagccaggcagaaatggtttgctaggggacccagcgagctcacaagtctttcctcattgcttcctctgcccctgtattttgctaggctctctaaattgactcggcttcaggtatcaagacgctcaccttctaagaggcttgcctaactggagtgctggagtctgaactttctttgaacatcgtttgatctcagatgcagccagtcctgtgcacagcctgatggggatgggaatgttcagggatcatcatgtgattcccnggggtgggcctaggttgaggggacacaaattttccccagggcagaggaaggacagcacagggagggaaggaagcttccaatattcgaggagttgaggaggggntggcaaatttcntttcaaggagcttgggcaccttnccaaccaaaantgtt

SEQ ID NO: 145 - DNA, 485 bp, Homo sapiens
  misc_feature (4)..(4): n is a, c, g, or t
caangggtgctgctaaacatccggcaataccgggccaacctcccccctcctcctgcccacagtaaagaattaccccgcctggattgtcagtagtgcagaggtagaggaaaccctacacctgtgaaactcatctgacctttactatgaattcctggatctcctgggtgcatatcatttctgttgccctggctgacatttccaccttttctcttttctagattttattacaagaactcaatctgttggtctctagggaatcacctattgccttgctatgttttgttggaacctgtttttggaatctggttttcttacattggggggaaatagggtatacgttttgttaaacctttaaaaggntctggttatggntattaaggggaaattcaccaggacangtggggataaggnttttgccaggagggttgagggggtcgttcttcccaccagaggaggacccngaagggagttcagggtttaagncc

SEQ ID NO: 146 - DNA, 503 bp, Homo sapiens
  misc_feature (366)..(366): n is a, c, g, or t
ggaattctaggagagttacacaactagtggaagtccatgtttagaaaataaatggcttgtttaaggaaaagtttttgtgtccaaagctccttaaagtcagagagatttctacctggtacttaacatcatatggaaattgatgctttagtgagggtgttggctatcctattgtcaatttcctgcatccttttttcttctttatttttgtatagagacaaggtctcgctatgttgcccagggtggtcttgttcctggggctcaagcagtcctcccgcctcgggtctcccaaagtgccgggattacaggtgtggaggccactgttgcccagctttattcctttttttcatttacacaaaaagactggantttgggttagtttctaagtttgggaaggataaaggngggtatggcacagggagggcccttgggnagccccttcagataacttttcntcattcnttcccaaaattcaggtntggggttgcattcctggtaaaattttt

SEQ ID NO: 147 - DNA, 318 bp, Homo sapiens
  misc_feature (274)..(274): n is a, c, g, or t
agcaagctttagaaatatgtcggcacagtttcgttctctccatcagtatgctgcccagaggatcatcagtttattttctttgctgtctaaaaaacacaacaaagttctggaacaagccacacagtccttgagaggttcgctgagttctaatgatgttcctctaccagattatgcacaagacctaaatgtcattgaagaagtgattcgaatgatgttagagatcatcaactcctgcctgacaaattccctttcaccacaacccaaacttggggtntacggcctgcttttacaaacgcggntcttnttttggaanaattt

SEQ ID NO: 148 - DNA, 450 bp, Homo sapiens
gcggccgcgggcgactcctggtacccccgaggccccgcgaactcaccttcacaaagctgtcggcgtccgggaagcctggcagcaccatctctccgtcggatttggttgcgctggtcgccgacaggaccctgggctcccgggtaagctcctgaagaaagcgtctagctccaactgtgcttcctccctccagtaccctctgaactcctccaagcagacgttgtttcctgcagacatcgctggaaccattctggttaacacagagtgggaactcagtacacatttgtgaagtgaactcctggaagagctcttgtgagggaggcaccgaattatcaggcagctcaagagatagattcactctcctgaaattagagatgggatgcccttaatacaattcattccacccattaagtcttcaataaatgttcagcatatccagttaaaaaaaaaaaa

SEQ ID NO: 149 - DNA, 2012 bp, Homo sapiens
aaagaagccaggagacaacatcggaggcaggagctgtgctgtattcatcctgaaaagttctggaggaggaagccacctacgggtctctgagtgggtggtggggggcagatggaagtggaggggcaaggctaaaaccttaaggaactgcctgtcagtgagcactcctgggagaatcagaacactgggaggaaagaggtcaaggagacagccttcctgccacatagatagaacattctggtggatggaaatttccacgagtgcttcagcctttctgcctggcttacacagaaatggatcttagagctactggccagaagatcacctttaccgaaagcatcttgcagtcatcctcctttccaagccgccttccagtaagactcacaatggaaggtccccaacctggagcaaggatacaagccaagggctttgatcaactcggccttcctgggcctcggggagagagacggactgcctccgggtgctcatgacctttccagcagtaagccaataatgtatgactcctgcgttgccgttgctcgtttgagctttgaatgatgcattggcccacgtggaccaccttccatttcccaagactttttgaaggcaatgaatgagaagaaagcagagaaaacagtgggcatgaggtcagatgactcaggcttgagtactgatcacatcaaggattggacgcactgctcttaggaaggcacaacttctactactttcagtcttatcaactgtgagaggaagaagaagcaaagcacaccattgatgtgtgtgggtttgatgtagcatcttctaagaggtctacaaagagacattgcaaagccaggcctctaaatgattcctctgtgaaacattgggccaaacattccgtaaccccttcctgctctggtgatcatgtaagtagatattctggaagcacttgctctacagatggagaattgtcaccatctgccaggacagctcctgctcatctctcatgactcagctcacatgtcataacttcaaggggccttcagctctctggcccttcctgccccatgaaggctctacccagtgcccccaactttgctccttcggctgcatttgaagcatttacctgtgcttgttaattgttggtggaattgtctgtagactgtgaccttgagtgtgcagagtcttgtagagtcatctctggattccccactctgagaaccacgcttggccattgcaggctctcaacaaaggcttgctgaggggatgaatgaatgaatgtatggatggcatgaatgtatgggctctggtgaattttttgcccacattttgctggccagcggctgcattgggtctgcacactgaaccctttggctcaaggtatcagagctgctctccttccagtgaagacttggatggaagacttgagatgcaggttatggggatacgtgtcacctctattcctcattcagaaggatacatacctcactggaccataaaaaatgagaactgaattatctgagtgtttgaataaacttccaccttgttactccactacagtctgagaaaacttcactgcacaaatagaggcagtgtacacccctcacagacactttcctttctcctgttgagaggtgaagccagctggacttctgggtcgggtgaggacttgaagaacttttttgtcttacaagaggtttgtaaaatgcaccaatcagtgctctgtaaaaacgcaccaatcagtgccctgtggctagctagcggtttgtaaaatgcacccatcagtgccctgtaaaaacgcagcaatcagcactctgtggctagctagaggtttgtaaaatggaccaatcagcactctgtaaaacagaccaatcagcactctgtaaaatggaccaatcagcaggacatgggtggggacaaataagggaataaaagctggccaccccagccagcagcagcaacaacacggtcgcgtccctttccacggtttggaagctttgttcttttgctctttccaataaatcttgctacccttc

SEQ ID NO: 150 - DNA, 2194 bp, Homo sapiens
taatgtagtgaaccctaagacatgcaagatacccaataagttatgaagagaattatattggcagagacactgccagcttggactgaaagggacagagccagtgcaaaaggacaagacggctttgcacacccaaacttttacaggcaggaagcaggcagatgaaactgtggagctgcaaacatctttcatggaagaggtgggacgactcagggagagccatggagaatcattcatgaacaggagtaggcatcaaagagttcctagcatttctccaactggattacagaatttccacacaccagtgacttatatgtatgacttatatgtgcctcctgtcccctgccccactttttttttttttttgagacagagtcttgctatgttgcccaggctggtctcgaactcctgggctcaagtaatcctcccaccttggcctcctgagtagctgggatgacaggcacacgctaccacgcccggcttcccctgcttttgagcaagaatgtccttactggttatcctgtgcctgcctctaccactgcacactgggagtgtggagatgacctgtctctttagctcacaggtctgcagataggaaatgcacttaagggctggatgcggtggctcgcgcctgtaatcccaacactttgggaggccgaggcaggtggatcacctgaggtcaggagttaaagacgggcctggccaacatggtgaaatcccgtctctactaaagaaaattggctgggatggtggtgcatgcctgtagtcccagctacttgggatgctgaggcaggagaatcacttgaacctgggaggcagagttttcagtgaggtgagatggtggtggctctgcacttcagcttgggagacagagcaagactccatttcaaaaaaaaaaaaaaaaagaaatgcacttaaggagccatagttacggaactgcatgctagagccacatccccacctggacctgactgagatgatgagattctgtactttgagctgatgctgtaatgggatgatgggggatcctggaaggtggtgagtatatttggcatgtgggaggggggaaatcactgagagccagcggtggcctgtggagccagccaccaaggcagcctgatgattctcgtcccctggtgctcgttcctgtgtgtcatctccttcctcactggataggaccaacagacctaggtcataaaagacaatggaggggcccagcgctgtggctcacgtctgtaatcccagcactttgggaggctgaggcgagtggatcacctgaggtcgggagtttgagaccagcctggccaacatggtgaaaccctgtctctactaaaaatacaaaaattagctgggcgtgatggcacacctcggtagtcccagctacttgggtggttgaggcaggaaaatcacttgaacctgggaggcggaggttgcagtgagccgatattacgccactgcactccagcctgggcgacacagtgagactccatctcaaaaataaaaaagacaatgaggcttccaccttgccctctctggaattatttgctctggagaagccagttgccatgctgcgaggatactcaagcaaccctgtggagagatctacatggcagggatttgttgtttcctgccagcagctagtgcgaacttgccagccacatgaacaagctttctcagagtgcatcctctaggggcggtcatgcctttgatgcccacagccccactgacatcttggccacagcctcagaagagaccccgtgccggagccacccagccgagccactgctgagatcctgaccctcagaaacagaagtaataaatattgttaatttaagctcctaaattgggggcagtttgttacacagtgatagataacgaattcatactccgtaattcccctcgcacatttgtggtgtctttttaaaaataccaattctcatatacacagtggtctgtttctgatcgctctgttgtgtttctcaggtatttttgtttctgtgcccgaactgataccgttctcactgttgtggctttgcagcctatcttaatatctggcagggtcggcctttcctctttgctcttatttttaaaaactgacctaagtttgggaggatggcttgagcccaggaggtcaaggctgctgtaagctgtggtcatgccactgcactcagcctggatgacagcaagaccctgtctc

SEQ ID NO: 151 - DNA, 2934 bp, Homo sapiens
gttactggaaatgaggagagcctcccaggtcccagcagagctgctgctggtgcctcccactgtgggctccacagaggcagaggatgagggagccactgatacctccctgtaaggcagcctccctgagcgagcaggagcaggtcaggggtgagtgtggaatgatgacagcccagggcatcctaggcccccggggcaagagcaagctccctgtgcttcctgacccccgcctcatttgcagatacatgtgctcatgtcgggagatacctggagtcatctcaactcctgtcccccattccgcaggtccacaagtcaccaggttccattccccttcccctccacatatctcagagctgtcacagactctccatgccccatttcaggggtcttggcccaggccttgtcatgccttatctggaccaagagcaatggtccccaaccagctccacccaggtaccccctgccccgcccatgtcccccacagttgcaccatgcctgctggcacctgccctctcttcactggtgctccagcagctgcagaggcagggctgaaccctgggcccgctctcagggcccccacctcctgctcatcctccacatggcccaagcccttcatgactgtttgcctcttgcccacatcttccatgttgacgtcgcctgggactggaggatgcatcagagcacggatccttgagccagcttaaaaacggtgtgtgaggccgggcacggtggctcacgcctgtaatcccagcactttgggaggccgagggtctacaggagttagtgcatgcagtgtgcttaggacagggcctgctggaggaatgtttactactattattaatgactggtacccggggctagtgatccagcatttcctgcctagcgccttgtccattccatgagaaccggccctcctcagcctcaccagggacatcgacatgtctgaccccctgcagcctgcagggggaggacacggagaccctgagcaggcagcgacttctggagttcaacagccccctagggcagacctgtggatgcaaagccgggctccaggtacgcagtgcccttgggggccgccaggtgctggcagcagggccacggggacctgggactgagccctctctgcctccaggaaaaagcaaaatgggatcatggtcccaggcgtccattcgaggtcctctccccttggcaggagacgaaggacactgaccccagcactcagcatgggctggcaaacctaatcagtgcccatggattatgtttgccatccactgactggtggacggattgaggaccttggagttatcttcctgaatcttccctccttgatggcaactcacctccgctcaccaaggggtccaggaaggcacctgactctcaggaccgctggcgtcttggccagcctgggagagaaattcatctggcttatggcacagggacagaagcaggccggctgaggacaaggtaacagaagggctagggcttttagaagacaaagcttccttcacggatgcagaaggagaatgctgtttctctgcgctgggcaacctgggtttgaatcccatctctgggataagtgtgcagggcgcacagcaggtgttcggcacacagagcccttactgaagatactgctcaacaaggcagggtctgcctggcgctggcttcaggcccttggcaggggctgggcagccactcttgcccatgccctggtggccccgagatgccagcactcctctgggtggttggggaacccacccagggtggaggaggtgggcaaggcctgggattccacatctccagggcagcagaccccacggtctccagggatcttgtctgtgcccaaactcctgagacttccctgtcctttcccacaagggccctggctctggtggctgcagaagtgggaggaggggatgtgggggagtctggggaaggccaccttccatgacgctctgccaagccaggaggagcgaaggaataacctggtccccgtgctggaccctggactccaggaagcagagcctacagccggctcagccttgggtcccctgcctctgtggagctggccctaccccagatggccactgtgcctgaggcccggcatctgaccctctgtcctccctgccgaggggccagaacagaggccccggcaacaggcaaggcgcagtgcacggagctgaccccgtgcctcggtccaggcctagcaacaggaagtgagaggccaatgccggtaaacgagatccgaaagattagaggcctcgctgccgccccaagttagaggacaagttcctggaaggaggaggaagctgccgcagtaaataacgccagaccccagcgtcccagctgtcagcagccagtgcggccgcaggctgatatggggtttcgctgtgttgaccagactggtctcgaactgctgaccttgtgatccgcccgccttggactctcaaagtgctgggattgaaggcatgagccaccgcgcccagccctggggtgaaatctttgcaacgcacagaacccgcaaggatcagtatgcttgcacataccaaccagcctgaccagtcaatgagaaaagaccagaacatgctcttcacaaagaggaattcaaacggctaaaacataagaaaaggggctgggcgcggtggctcacgcctgtaatcccagcactttgggaggccaagacgggtgtatcacgaggtcaggagatcgagaccatcctggctaacacggtgaaaccccgtctctactaaaaatacaagaaaaattagccgggcatggtggcgggcgcctgtagtcccagctactcgggaggctgaggcaggagaatggtgtgaacttgggaggcggagcttgcagtgagccgagatcgcgccactgcactccagcctgggcgatagagcgagactccgtctcaaaaaataaataaataaataaataaat

SEQ ID NO: 152 - DNA, 370 bp, Homo sapiens
  misc_feature (287)..(287): n is a, c, g, or t
tttaatttttcaaattgatttaaaaattatttgaaaattacaacagatagatttcttttttttttccacttaacatgattattttcccagtcatttcatgttcttcaaaaaccccgatgataggaataattttctctttcctgaaatctctcttcttgtgattggatctgtttgaggtcctggacggaaaggcttggacgaagtccttgcagactcagcatggttgtaagcagtataatttcagagggctgtcccatgggctcactctgcaaccaggcggaacccangagagggcgagctgtggtgcagtcctcataaaggggagaactgtagttttcagagttctaatgaacatcccaatgggcctaaa

SEQ ID NO: 153 - DNA, 2343 bp, Homo sapiens
acctctgcaaccctccctatagcaagatgggtcaaagcctgatccctacccccgaaaatgggtgtcaagtgtctccgtcaaggacagacttaagactcacccgaaagaaaacgagacttctggtaaagccctagagtcctgatctggcatcaccggctttgctctatttgctttgaggaagccatccttccctggtcgtcttcactgcatctgctctgtgtacagaatccttctcattctaatccaccttggtggcagtaagtgcttagataagcaaacttggctcatcaataccatgaggttctttggtgggtgaagagatgtttccttttcccaggtccccttacttcctctccattcccccactaaactcccatcttctctctgtacttctctcaccttgtactcctgcaccacctcctccaatggcaccacgctgtgctgtttgtggctcctggattctcggcacaccacacagatggcctcttcgtctacctcgcagaagagcttcagggcttcttggtgtttgggacagatgccctgatcggtcacgcggctccctcgaccaggggttgggtgcatctgccgaatcacctggaccatattggccagctgcaggttggggcggaagctccgccgaggaaagctctttcggcactgagggcatgtgaagcacctccgaggggctggaggcgggggcagtggggtgacggggtctagatcctcttcctcaacctcctccagcacttcctcctcgtcctcctcatcctcccccctcaggtcctcctcctccatgtcccccaagtagtagtccaggtcttcctcctcgtcctcctcctcccacacatagtccatgttgtcccagctggacctgctcatgccactggtccagaacacaccctcttcttcctcctcgacctcctcctccatgtcaccctcgtagtcttcatcccgcatgggggtgtcccaccccgcgccagcccccacagcctccacttcctcctcctctccgtcctcctcctcctcctcccgatctaactcatctctgtcctcctcatcctccccaccccacaactgggttacacaaactcggcagaagttgtgcccgcagccgatggacacggggtccgtgaagtaatcgaggcagatggcgcacaccgcctcctcctgaagggtctgcacagggttgggtgtcatggcaacggcagccatcttagtgtccagccagccagtgtagaggttcggtgggggggcgaggggcgggggtcttccctaccgacgccctggcgacccggctccacccccagccctgcccctccacacctcgccccaagagcagccagagagatgtcctgccgacaacccaccccaacacagtgttccctactcccaaacgacaaccgtgtctctacgaggggaggggacagtgctgggcgccaccgccaagtccctcaggtggctctgagtacaagtctgccccaatgctcccttggactcctcataaacccccgcccctcctcacttcctggccccgcccctagcttccggcctccttcccaacacttccggcgtctacacaccacctaagctcgcgacttccctccgctgtcctgctactccccctttttccccgcggggccccagggcgacaggaaatggcgaggagacgctctagtccgcactagagaacagggcgggagggctaggacggtggaggcccgcgtctctgtggtaagaggccgcggggaacgcagaaagaacagaggaaccgcctacccccatcccccgccgctggggagaaacttcggggggtagggggagcgcctggcggccgtctgctttcggtgctatcaccgcatcggccagacgccatcctaccctcccggcacagccgactgcagccggtactccccacagccgatgccggaagcgagggggtgggtccgcggcgcgccctgaagtacttccggcccttctaggcagacgacttcgttgtggaggactcagagggtcgtccgggtcggaccgggggcggggcctgagaaagcgcttcgcgggttagggcagcagacgccccacccttccccaactgtagtcggcgtttcgcatccgaggtagaaactgccaaccggacactgtctcatggtttagatgaacttccctaactgcccagcagtgaagaagtggtgacgaatcattccatctacattctcacagcatctctgcaaaaatagatgctttgtgcggtagaaataatacttgatgcttaaatagctctttaaattttatttttatagagatttgatgtcgatacataaataaataaatgttgtttgatgcgag

SEQ ID NO: 154 - DNA, 1022 bp, Homo sapiens
gatccggtgggagggaacatctggaacattagacaggatgctgatcttctggacaatcacacttttcctgctgggagcagccaaaggaaaagaagtttgctatgaggacctcgggtgcttttctgacactgagccctggggcgggacagcaatcaggcccctgaaaattctcccctggagccctgagaagatcggcacccgcttcctgctgtacaccaatgaaaacccaaacaactttcaaattctcctcctctctgatccatcaacaattgaggcatcaaattttcaaatggacagaaagacccggttcatcatccatggcttcatagacaaaggagatgagagctgggtgacagacatgtgcaaggtaggagccagctctgatccctgtggccagctgaggccaacacttctgctaacatctctgcatcactttatgcactcaagaaatctttacatattaggtaactttatgcaattaaaatgcttctcttcacaaaaattaaaatgcctttccatgtttccgcactacatttgcacactgaagcaaccacatttgctgttagaaaagtactcctactacctaatttctggttaaaccaaggcctgatgttttctgcttccatttgtagtgagggtactttgtatcctataagcgagggactataggggtttctttgttcaaatttttcccacatccctgagaggctgacatgtgttgctgtgaccacttaattgatcccagcactttgggaggccaaggtggatggatcacctgaggtcaggagttcgagaccagcctggcgaacatggtgaaaccctatctctactaatgatacaaaaatcagcctgttgtggtggcaggctcttgtagtcccagctacttgggaggctgaggcaggaaaattgcttgaacccaggaggcaaaggttgcaatgagccaatattgtgccactacactccagcctgggcaacagagtgagactccatcccaaaaaaaaaaaaaaaaaaaaaaaaaaaaa

SEQ ID NO: 155 - DNA, 1413 bp, Homo sapiens
ggctcggcagctccaggctattccgaggaacagtcttcagagagcctggggtgctgatggggaaaagctgttgctgagatttgacaagtttggtcctgagatgtcagaaaggatttcagctcttctgccaccccaaggacggtggcgcgtaaatcacagtgagctggcttatgtcaggtcaactgtggaatttgatcctagcgcattgctccctcctcttccttgcctgcgtctctccctctctcagctctagtttgagatcagcacagattccctgggtgatttttgagaaaactgcctcctctttaggacttggcatcccctaacctgcatcaggaaagggtggcgacagatactctcacacccagggaggctgcaaggcccaccctctaccctccacctgggttctctggcgtcgagacttgtgccctacagttctgggccgttggggtgatgtcccagtgggaccagggtgagggtgcaggtatctgagggagtagtaggaccagaacctctaaccttagggaggcttcctgggagccaagcttggtgacttagaatgtgaatgtctgacccatatcccactaccttttggtgggtgctgcagaggacaacggagagagagtcaactttgaagagtggtcttgtggaagaggcagctggctggttcagtgtggcactggcagcctccctaaggttcccaggtagaagtttctgacaggtagatttcagctgtgtttaaggaagaactttgaagttgtcagaatgagctgtcttgtgatagtgagctccctgtctccagaggcatgcaagcaggacactttcacttatgcattcaagctgacctatgcaaggcgttgtgctaagtgctaggagtgcagaggagaacgagacccagtggggaaacaggcacttgcaatgctgctaggacaatgaatccaggcagtgatccaccaatgataactaacatcataaaaagatacatggccggatatgatgttccttctgacggaagaccacaacacctgctgaaaaatcaaatctgaatcggatcaagcctctagaggaggcagcacccagggtcattcctggagcataaacgatccctgcagaacatgggctagaagaccccacagtggggcagaggcaggtcctggactgggatcccagcagtgagaagacctggacatcaaccctatttcattgtgcctctgcaaatctcccaacttttggagctgtagctgcaagctcagacttgtggtttcctgtacctggaggaggcccaggtcattctgttaagtcccttgtctatttcctgcttttatctggatgctgagttaattatttgatccaacaataaaactaacaaggttttaaaaaaaaaaaaaaaaaaaaagaaaaaaaaaaaaaaaa

SEQ ID NO: 156 - DNA, 471 bp, Homo sapiens
ccttagattatacttggattttctacatcacaatgcacttcctgccgtgcttgtgttgggaaatctgccagaacccacaggagggaaagtctaatgaaagcacgcgcctgctggaaaacgaaccgggcttttgaaaagagaggggctgcgtttgtgctgacacgagcaagagacctgtattgccttaacactcccagcaatgaccacctgcaagcttgcgctgcgactcccgtcctaagacatgcgggccagtatgagcggagaggttcccagcaccgtcacaagaccctgtgctattattttagactcacctgtggctgttgacaacaccacacacatgaaatgatgctcaccagaatcaaaatactcagctaaacaaagaattgtgttggtcatgaaattattaccaggagggataaaactccagggtgagccattaaagaatctgaattcaattcaaaaaaaaaaaaa

SEQ ID NO: 157 - DNA, 2831 bp, Homo sapiens
ggccggggatacgtgcttaatcctggtgcagggggcgagcatggccgctccgcgagtattcccactttcctgtgcggtgcagcagtatgcctgggggaagatgggttccaacagcgaagtggcgcggctgttggccagcagtgatccactggcccagatcgcagaggacaagccttatgcagagttgtggatggggactcacccccgaggggatgccaagatccttgacaaccgcatctcacagaagaccctaagccagtggattgctgagaaccaggacagcttgggctcaaaggtcaaggacacctttaatggcaacctgcccttcctcttcaaagtgctctcagttgaaacacccctgtccatccaggcacaccctaacaaggagctggcagagaagctgcacctccaggctccgcagcactaccccgatgccaaccacaagccagagatggccattgccctcacccccttccagggcttgtgtggcttccggccagttgaggagattgtaacctttctaaagaaggtgcctgagtttcagttcctgattggagatgaggcagcaacacacctgaagcagaccatgagccatgactcccaggctgtggcctcctctctgcagagctgtttctcccacctgatgaagagtgagaagaaggtggtggtggaacagctcaacctgttggtgaagcggatctcccagcaagcggctgccggaaacaacatggaggacatctttggggagcttttgctacagctgcaccagcagtacccaggtgatatcggctgctttgccatctacttcctgaacctgcttaccctgaagcctggggaggccatgtttctggaggccaacgtaccccatgcctacctgaaaggaggtccctggctctgtcactgaatacaaggtcttggcactggactctgccagcatcctcctgatggtacaggggacagtgatagccagcacacccacaacccagacaccaatccctctgcaacgtggtggcgtgctcttcattggggccaatgagagtgtctcactgaagcttactgagccgaaggacctgctgatattccgtgcctgctgtctgctgtaaaggctgcagcctccccagctctcctctgccagccaccctaaattccagccaacctcacctcctcgggcccagctcaagcccccttccttgctctggaccccttaggtataccctggaagagctggggtgggggaggagggagcgtgaaggtagtgactcctgaacacacccaggtggaaccatctttggggaggagaggcccgtgtgaggggtctgatactccctttgtcttccctctctactcctcgctacacctgagccaggctcttgccaactctgttccagcctatggctttaggctagctgttaaatatgtgacccagcattagctcagcatctgtcagagcaagagaccaggtaatttctaagaacagggttctagcgatgggactgcccatttcctcagctgcagaggaggaaagggaaagggtaggcctgtagactaacgctgtttacacccttgttctgtcaaagcaattaaagatcacttgtgttgaggctgtggggtaatgagcactcagcctttggggtacctgttcctaaagtgggccaaaagagccctccctacatgatgccccagtttttgctttattcctatttcatacagcttctcgggggggtgagcaggctacactccagaacaccggtatgggaaggagtgggagaggaagccagctttggcctcacaggcacagcttgcaagcaggccttgggtctgcccagaggcacagcttgcaagcagccctacagagaaggtgactcaaaggatacaccagtcaccagtgcaagactcttcgctcctgtttttctcttttttttttttttgagacggagtctcggtctgtagtccaggctggagtgcagtggcacgatctcggctcattgcaagctccgcctcccgggttcacgccactcttctgcctcagcctcccaagtagctgggaccacaggcgcccaccaccacgcccagccaatttttttggtatttttagtagagatggggtttcactgtgttagccaggatggtctcgatcttctgacctcgtgatccgcccacctcagcctcccaaagtgctgggattacaggcgtgagccaccacgcctggcccggctcctgtttttcaactggccctagaggaggggtccgcaaaccaggctggcaggccagttctggccatacctgttcatttccatactgtctggctgctttcaagccacagtggcaaagttgaagagttgcactagactgtacggcctgcaaaactgaaaccattgactgtcaaccacttctctacaacatactcaactgttgcagattacagggactcaggaaccgaattagacaattttcatggcgaggagaagcccagttagctttcctaaacgggcaggaagtgtgaaggagggaatctcctgatgccctgctccaggggtggcacacacctgcaagaggctgtcggctgtctgctgctgctgaggtttctgacctgcaatcgtagatcctgtcaccacagactaatcacttagtccactggctccttcctgtgggataaaggtttaaattcatgcaaaagaatctttctgggcttctgcccacactagagtccctatacaatggagttccaggaaaccaccttcaaaaatctcctgggttttcttgcctccaaattttcttcagctaaaaaacaataaagatgagctggaaagaaaaaaaaaaaaaaaaaaaca

SEQ ID NO: 158 - DNA, 3189 bp, Homo sapiens
gaactgtattcagcggcgacagcggcgactgcggcggccgcgggagggcatcccgttggggatccttccgcacactgaagagtacgtcttcgggtctacccctaatcacataatggctgtgtttaatcagaagtctgtctcggatatgattaaagagtttcgaaaaaattggcgtgctctttgtaactctgagagaactactctatgtggtgcagactccatgctcttggcattgcagctttctatggcggagaacaacaaacaggagagacggggtttcaccatgttagccaggatggtctcgatctcctgacttcgtgatccacccgcctcggcctcccaaagtgctaaaattacaggcgtgaaccaccaccacagtggagaatttacagtctctctcagtgatgttttattgacatggaaatacttgctccatgagaaattgaacttaccagttgaaaacatggacgtgactgaccattatgaggacgttaggaagatttatgatgatttcttgaagaacagtaatatgttagatctgattgatgtttatcaaaaatgtagggctttgacttctaattgtgaaaattataacacagtatctcctagtcaactactggattttctgtctggcaaacagtatgcagtaggtgatgaaactgatctttctataccaacatcaccaacaagtaaatacaaccgtgataatgaaaaggtgcagctgctagcaaggaaaattatcttttcatatttaaatctgctagtgaattcaaagaatgacctggctgtggcttatattctcaatattcctgatagaggactaggaagagaagccttcactgatttgaaacatgctgctcgagagaaacaaatgtctatctttttggtggccacgtcttttattagaacaatagagcttggagggaaaggatatgcaccaccaccatcagatcctttaaggacacatgtaaagggattgtctaattttattaatttcattgacaaattagatgagattcttggagaaataccaaacccaagcattgcagggggtcaaatactgtcagtgataaagatgcaactgattaaaggccaaaacagcagggatcctttttgcaaagcaatagaggaagttgctcaggatttggatttgaggattaaaaatattatcaattctcaagaaggtgttgtagctcttagcaccactgacatcagtcctgctcggccaaaatctcatgccataaaccatggtactgcatactgtggcagagatactgtgaaagccttattagttcttttggacgaagaagcagctaatgctcctaccaaaaacaaagcagagcttttatatgatgaggaaaacacaatccatcatcatggaacgtctattcttacactttttaggtctcccacacaggtgaataattcgataaaacccctaagagaacgcatctgtgtgtcaatgcaagagaaaaaaattaagatgaagcaaactttaattagatcccaatttgcttgtacttataaagatgactacatgataagcaaggataattggaataatgttaatttagcatcaaagcctttgtgtgttctttacatggaaaatgacctttctgagggtgtaaatccatctgttggaagatcaacaattggaacgagttttggaaatgttcatctggacagaagtaaaaatgaaaaagtatcaagaaaatcaaccagtcagacaggaaataaaagctcaaaaaggaaacaggtggatttggatggtgaaaatattctctgtgataatagaaatgaaccacctcaacataaaaatgctaaaatacctaagaaatcaaatgattcacagaatagattgtacggcaaactagctaaagtagcaaaaagtaataaatgtactgccaaggacaagttgatttctggccaggcaaagttaactcagttttttagactataaatttgtgtcttatatgctttaggtttatgtatctataaaccattcaccaaagacatgcttaatttttaagagatcaaggtgtaaattatgatgatttattattttggtctacagtgtatgtaaggttagtatgttaagcattgtttaaaaatactagtaagtcataattatgcagaattttcacaaagtttaatgcacagagaaagcatatcatttcagttactgatacatcttaacactactttcttttaaaacagacatttaacatacacaagttatagtagcagtatgggcttctcctcccattggcaattaaatgcttttattttcttctgaaaagatgatgtggaccaacaggtatcagacttgccaacaaggtcggtagactcttcccagcatacatctgagcactgaaggaagaagaaagtttaaattgtttaaaggactataattatcacacaaaatttattaagaaaaaaagaatggatctagtataactaattctgagtaaaccaaaatgataataattaattgttgctatttaatcccacatttttggcaggtgtaattgagccatggtcttatttgattttgttatgattgcatccaaattcactttaactcagagttctgtttaatggtggtaggatgtaagaattgaattttgaaaagactactcactgtcaaaatctctccttcctataggaaatttagctgagttttcttcatccccaatttctctcttttcttgtgttgattcagtattctgaactccattctcagctgggaaagctacagatccttttagtgcaagataaggttttatagccagattcagtggcagaccatgatttaagaaattatgtttggagcctgtgttctgtaaagagaaggttgatttggtttttagctatcgtattcggagtggaactataatacaattgtataatattcttgttgatcaattcaaagttactctgcactgtttttgactttttaaaaataccttagatgcaaatttataggagaaaaaacactttcagataagaggtgtttgctgggatggaagaactacctggcatgtaagaaatatcgtcagtcgtcctaatgcatattgtgactgtttgcatatacttctgtttataaaagtatcagttttacttttcagaggatttgtaagaatcatttaaattttcattgaaataaacgacaagtcacattgccactta

SEQ ID NO: 159 - DNA, 1012 bp, Homo sapiens
cgccggtgcctgcgcctcccgctccacctcgcttcttctctcccggccgaggcccgggggaccagagcgagaagcggggaccatgttccgacgcaagttgacggctctcgactaccacaaccccgccggcttcaactgcaaagatgaaacagaatttagaaacttcatcgtttggcttgaagaccagaaaatcaggcactacaagattgaagacagagggaatttaagaaacatccacagcagcgactggcccaagttctttgaaaagtatctcagagatgttaactgtcctttcaagattcaagatcgacaagaagctattgactggcttcttggtttagctgttagacttgaatatggagataatgctgaaaaatacaaggatttagtacctgataattcaaaaactgctgacaatgcaactaaaaatgcagaaccattgatcaatttggatgtaaataatcctgattttaaggctggtgtgatggctttggctaacctgcttcagattcagcgtcatgatgattacctggtaatgcttaaggcaattcggattttggttcaggagcgcctgacacaggatgcagttgctaaggcaaatcaaacaaaagagggcttacctgttgctttagacaaacatattcttggttttgacacaggagatgcagttcttaatgaagctgctcaaattctgcgattgctgcacatagaggagctcagagagctacagacaaaaatcaacgaagccatagtagctgttcaggcaattattgctgatccaaagacagaccacagactgggaaaagttggaagatgaacacttgaggacttcagcttctcacctacttagtacagttgggaaccatacacttctggcatgtttggaaatcaaaatgtcacattctcgggggaggaagcccagaaaattgggtatgttctagagatttaccaccattgcttattgcttttttctttaataaagtttaggaaagtaaaaaaaaaaaaaaaaaaa

SEQ ID NO: 160 - DNA, 4430 bp, Homo sapiens
cggcggagtggcgagaggcgagaggcggcggaggcggcggagctggggggggtgggaggggggggagagtgagtgagtggcagagtgagtttacccctatgagactgtgagaggcccggggcctacctcaaaggagcggggtcgcgaagctagctagcagcggcccccctccaggtccccgggcccggcggcgcggcggcggcttggttgtgaagaggcggggaagcgggtgtccggtccccgccatggagggcatggacgtagacctggacccggagctgatgcagaagttcagctgcctgggcaccaccgacaaggacgtgctcatctccgagttccagaggctgctcggcttccagctcaatcctgccggttgcgccttcttcctggacatgaccaactggaacctacaagcagcaattggcgcctattatgactttgagagcccaaacatcagtgtgccctctatgtcctttgttgaagatgtcaccataggagaaggggagtcaatacctccggatactcagtttgtaaaaacatggcggatccagaattctggggcagaggcctggcctccaggggtttgtcttaaatatgtcgggggagaccaatttggacatgtgaacatggtgatggtgagatcgctagagccccaagagattgcagatgtcagcgtccagatgtgcagccccagcagagcaggaatgtatcagggacagtggcggatgtgcactgctacaggactctactatggagatgtcatctgggtgattctcagtgtggaggtgggtggacttttaggagtaacgcagcagctgtcatcttttgaaacggagttcaacacacagccgcatcgtaaggtagaaggaaacttcaacccttttgcctctccccaaaagaaccgacaatcagatgaaaacaacttaaaagaccctgggggctccgagttcgactcgatcagcaaaaacacatgggctcctgctcctgacacatgggctcctgctcctgaccaaactgagcaagaccagaatagactgtcacagaactctgtaaatctgtctcccagcagtcacgcaaacaacttatcagtagtgacttacagtaaggggctccatgggccttaccccttcggccagtcttaaacgggtgtcagcaagaagaaaaattaacaaaagacagaaggcctgactttggggggttagggcaaggggttcctctggattgcagaccacatcgcacagacccctggctctgaccccctctcatcccggaagaagaggaagaagcagaacagactagttttgagtaaactcagtatgcatgtgtgaatgctgaatcacaggaatggtgttgaggctaccaagaagaaatccatgcagccactttgggttttgttataggcatcagtctaacaagtcattaggtcactcgggaagggggaaaaagtttaaaatgggggaaaaaaagccatctttttaaacaaaaattattttgcctacagaaaggttgtagttttgagcacatgttaatttttttccctctttccccacttttatttttttaaataagggataacatattctttatagaatagtgcttgttctggaagagattcaggtgaaaagttggcgtggcatgtttgaggactctgcggatcagtgctacaggagtacatctgccctgccacatgactccagaagtctctgaccccatttgtttttaatggcatcagccaatgagttatcacccttttcctcctcttttctctttaatcttctgttgatttacacctttgacatttgtattcgtcaaccctgtggcttgttagcatcagaacctctctgaagaccaaacttccctgtgtggccagcagaggaagccttgaggatagatctcgggtgacgtgggattttctaagcctgagaggtgtccttctgcacacccttgtaacaattaaaattgcttttcttccatgtttctcttggcagagagaaatgccatcatgcttactgctcttttggattcttcatgcagtggcttcccatttgctctgggaacagtgcctctgtgctggttatatgtatgcaccacatgtgcacacacgggtgtcggtgcaactcaccagcaggtgtgcagtaggcaagcttgaaggtggcccatgcttctctgttgtcacacaacacctttccctgtttctcttcagttgtcctctgatattttcacagcctgttagtggcggctgatctgtagaggtatgaaaatacagccccaaagggggaatttgtcatggggaagggccagccactgactacttgatcttggagaccacatttagatggaaatgagaggacctccacagccccgcctccctgccaggatgctagattattggctgggaggtgggcaagtggcagcccagctgaagcaagaggctgattgactggctgctcactcaggcaggtggcctgccctggcatcctgtagattctgagcaggttgagatgtggatatctccatgggaagcttgaaccaggctagaacagctgtctgccaaagatacaagaatatgcaaagtccctcttacccccctccttccctcaagtcttagttggtttgagagccagggatatggatccagggtgctgttgcagggtcacctgcctgttaccacacccccatccagctgggtggtgggtgagggtgtgagacaggcagggagaccaggtaatgagtggctgcaaggagtcatatagcctgggtgtgggtgagggtctgcctgcctgtttctgtctgggtgtctgagttccaaaaaatgtgtgttgtttgttcctcctcatcctcttctgagactgttgtttttttagagcttgattgtgggagaaaagcttgtgtgaaattccctcttcacctccccaccccccaaaaaataaaagggggcataaactttattccactggagaagccctgggggtggctggagccagcctgctgagaagcgtggtgcagctgggtctgggacttcactagagcttactctggagcacctattttctgtgcacatgggagtgctccactccctagcagagcagagggaagtctctgccaggtacccacgccacaggcctcaggatggctttttgtctgagggcctcattaggcccagactgctgctgctggtaccagtctcctgagacactgcctccctggagcccatgcatgcccagctgttcttactgcttagtagccttgaagcagcacatctccactgtccctggcaggattgtgggaggcttcacaacccctgctttcctgacttcctcttctgcccaacactgggtgccccttcccagtttcccagcaggggcttcatgggccaccgtcagtgggtctgggcctgccagagtcgtcttgctcttcctcttgcttctaggcaaccacacttggcaagttaaatgtcccaagcaccttgtctcccttcccaagaacaaaccatttctgtgcacatccttggaggcgagatgagctccttgcagagggccagagtaagtgcaagtcatggagtgcaggcagaggggtccatggcttccccagcccagggagggtatcagtatcattcattctctctcccctcactggtatgggttcagagcaatgcctagggcccctctctgctcccctgcctagtggggtggtagtggctacctctcagccccaatagtccctgctcctcttagaatcttcaccaagtggcaggccacctctgggcaggaccacctgcgtctggcacccaggaggtgaggcagacaccatggttaggtgatagggtctgaacccagttggggagacagaggggttcagacttgggaagaatctgtcagtgtgcatggagtgaggtgtgtgtgtgtggatggatgtgcacgtgtgcatgctttttttctttttgccgtgaggacagttgaatttggggcatttttctacaagaagcagtcagccttctctcccccataaggactggctgtaaccttaagcccatcaaagttacttccagcctggagagaacctcaccaaacagctggtgtggcctgagggtggccatggtgggaaatgggcatgagaataaatacctccccaaattcaatctgagcccagcaggagaggatggtagggttgccagggctcagaagtgcaagctgattactcaccccaccctgcctcgccgcacctttcctttgtttccttgggtgaatgcaggagcagcagtggctgcccttcccacctggacagtggtgtgtgtagaaagccagctggatgtttgtggtggggcctcatggtgcctaggaggagataaagatgaggaggtttttccttattgtataaatgaatatttgtatgattaaattaacacacacaccaaaaaaaaaaaaaaaaaa

SEQ ID NO: 161 - DNA, 1126 bp, Homo sapiens
tggtaaaggactagcagttctctgcggagggccggttgatacagttccggtgggagaacgcggctgcgaggttttcggctttggctcctgatatgcagcgacagaattttcggcccccaactcctccttaccctggtccgggtggaggaggttggggtagcggaagcagcttccggggaaccccgggcgggggcggaccacggccgccctcccctcgagacgggtacgggagtccgcaccacacgccgccgtacgggccccggtctaggccgtacgggagcagtcactctccgcgacacggcggcagcttcccggggggccggttcgggtctccgtcccctggcggctaccctggctcctactccaggtcccccgcggggtcccagcagcaattcggctactccccagggcagcagcagacccacccccagggttctccaaggacatctacaccatttggatcagggcgtgttagagaaaaaagaatgtctaatgagttggaaaattatttcaagccttcaatgcttgaagatccttgggctggcctagaaccagtatctgtagtggatataagccaacaatacagcaatactcaaacattcacaggcaaaaaaggaagatacttttgttaacatttctgaaattcaactggaagcttcatgtgtcaggaacatcttggacaaaactttaagttgtgttgatataaatttacccaaagatgatgactttgattggataattagtaaggtctttttgttatttttcatcgtatcaggtattgttgatattagagaaaaaagtaggataacttgcaacatttagctctggaagtacctaccacattttagagatttaccgtttccatatatttaacattcctggttacataatggacatttgtcttttaatgttttttcaatgttttaaaataaaacattttgtcttctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 162 - DNA, 700 bp, Homo sapiens
atctaatcttcaaacagctcatttcaacagatagggaaaccaaagcaaaaagaagttaaataattgttcaatcatgacattgtgaaaaatgatactttgccctttcctcagttgccaccaaggtgcttggtccttccgaggaagctaaggccacattggggtgaggccatcacttcatccagtgactagcaccacctctggcaatgtcagccccacactcgcccgcgccatggcctccatctccgagcttgcctgtgtctacttggccctcattctgcacgatgacgaggtgatcatcatggaggttaatatcaataccctcattaaagcagccagtgtaaatgttgaaccttttggcctggcttgtttggaaaggccctggccaacgtcaacattggaagcctcatctgcaatgtaggggctggtggacctgctctagcagctggtgctgcaccagcaggaggtcctgccccctccattgctgctgcttcagctgaggagaagaaaatggaagcaaagaaagaagaatctgaggagtctgatgatgacatgggctttggtctttttactaaacctgttttataatgtgttcaataaaaagctgactttactgctgttggtcttgcccatagtttgggaatgtgctctgcaaaaatggtctcagttttgtaatgttggcttttaacctattctgccatga

SEQ ID NO: 163 - DNA, 428 bp, Homo sapiens
  misc_feature (292)..(292): n is a, c, g, or t
tagctcttcatcaaggaaattttatttgagggagtttacattgttctctacagtaacaacaaaaatagggcaacttacagagaagttagtaagctaagtaaacattttgtctttagcaaagcttatcatttgcatatttcttatttcttgattaagaagactcttgcaaccaaccttttaaagccttcctttacctccttgttcctaagtgtatatataaggggattcagcatgggtgcaatgattccatagaagagagaaaccatctttccttggtccttggagctgggcnaaggtggttgcaggtacacagagacggctgtacttttcccgccgtcttccctcctaatggaggccggccggagtccacccaaaagacttacaagcaaaatgcacaggctctttctctggaagcgtgagtggctatg

SEQ ID NO: 164 - DNA, 31737 bp, Homo sapiens
aggaacctggtaaaagaatctgccttctaagattttgtagagtagctgtgctgtgctgatgatatatcccttctgccccggtaggtatggattctccagagcccgcgggctgtaacgactaagttgcacaaacagcaaagatggctccttgctcttgcgcgtgggaactcattccaggaattcaaatctctgtcagcctgagaacaccggtgtgggtggctggaggccccagctgaaaggaccctcattgggctggacctccaggcctccatgccagggagaactcaaatctctgtcagccccagaacactggtgggggtggctggaggccccagttgggaggtcccccactgggccagaccccaagacctccatgccagagagaattcaaatctctgtcagccccagaacactggtgggggtggctggaggccccggttgggaggtccctcactggaagggaccccaagaaccgcatcccaaagataattcaaatatctgtcagccccagaacactggcgagggtggctggaagccacggtagggaggtccctcactaagcaggacctcaagacctccataccagggggaattcaaacctctgtcagccacagaacactggcaggggtggctggaggccctagttgggaggtccctcactgcgccagaccccaagacctccatgccagagagaattcaaatctctgtcagccccagaacactggcgggagtgtctcaaggcccctattgggaggtccctcgctgaatggaacctcaagacctccatcccaaagagaattcaaatctctgtcagcctgagaacaccagcgggggtggctggaggcccctgttgggaggtccctcactgggcgggacctcgagacctccatgctggggagaattcaaatctctgtcagccccagaacgctggcgggggtggctggaggccccggttgggaggtgcctcactgggcaggacctccagaactccatgccagggagaattcaaatctctgtcagccccagaacactggtgggggtggctggaggccccggttgggaggtcccgcccagtgaggaggaatggatcacagaccttcttaaagacccagtatggccacattttggtagagcaccttgctatgcccaccccgccagtgttctcaggatgacagatttggattctccctggcatggaggtcttgaggtcctgccaagagagggacctcccaaccgaggcctccagcaacccctgccagtgttctggggctgacagagatttgaattctccctgccatgaaggcctcagggtctggcccagtgagggacctcccaaccatggcctccagccacccccgctggtgttccagggctgacagagatctgaattctccctggcacgtagttctcaaggtcctgcccaatgagggtccttttacctggggtccccagccaccccctccggtgttctcaggctgacagagatttgagttctccctggcatggaggtctcaggatcttgcccagtgagggacctcccaactggggcctccagccaccccctccagtgttctggggctgacagagatttgaattctctctggcatggaggtcttggggtgtggcccagtgaggggcctcccaactggggcctccagccacccccaccagtgttctggggctgacagagatttgaattctccctggaatggaggtctccaggtcccacccagtgagggacctcccaactggggcctgcaaccacccctgtcagtgttctgggactgacagagattcgaattctccctggcatggaggtctttaagtcctgccaagtgagtgacctcccaatcgggcctccagccacccccgccggtgttcttgggctgacagagatttgaattctccctggtatggatgtctcagggtctggcccagtgaaggacctcggcatccagccacccctgccagtgttttggggctgacagagatttgaattctccctggcatggaggtcttgaggtcctgcccagtgagggacctctcaaccgtggcctccagccacccccaccggtgttcttgggctgacagatatctgaattctccctggcacctagttctcaaagtcccgcccaatgagggtccttttagctggggtctccagccaatcccaccggtgttctcaggctgacagtgatttgagttctccctggcatggaggtctttaggtcctgcccagtgagggatctcccaactggggcctccagccacccccgccagtgttctggggctgacagagatttgaattctcccaggtatgcagttcttggttcttgaagtcccgcccagtaagggttctcctaactggggcctccggccaccctcacctgtgttctcatgctgacagagatttgaactcttcctgacatggaggtctccacttcctgtccgctgtgggtcttttcaattcaggcctccagccacccacccctggtgttctcaggctgacagagattcgaattctccctggcacttagttcttgaggtcctacccaatgagggtccttttagctggggtctccagccaaccccactggtgttctggggctgacagaggttggagttctccctggcatggcggccttggggtcccgcccagtgagggacctcccaactggggcctgcagccacccctgccagtgttctggggctgacagagatttgaattctccctggaatggaggtctcagggtctggcccagtgagggacctcccaactggggcctccagctaaccccaccagtgttctggggctgacagagatttgaattctccctggcatggaggtctttaggtcctgcttagtgagggacctccctaccgtggcttccagccaccctcgccagtgttctggggctgacagaggtttgaattctccctggaatggaggtctcagggtctggcccagtgagggacctcccaactggggcctccagctaaccccgccagtgttctggggctgacagagatttgaattctccctggcatggaggtcttcaggtcctgccaagtgagtgacctcccaattgggtctccagccacccccaccggtgttcttgggatgacagagatttgaattctccctggtatggaggtctcagcgtctggcccagtgaaggacttcggcctccagccacccctgccagtgttttggggctgacagaaatttgaattctccctggcatggaggtcttgaggtcctgcccagtgagggacctctcaaccgtggcctccagccacccccaccggtgttctcgggctgacagagatctgaattctccctggcacgtagttctcaaggtcccgcccaatgagtgtccttttagctggggtctccagccaatcccactggtgttctcaggctgacagtgatttgagttctccctggcatggaggtctcggggtcttgcccagtgaggaacccccccaaccggggcctgcagccaccctcgctggtgttctggggctgacagagatttcaattctcccaggcatggagtttttgaggtcccacccagttagggttctcctaactgagacctctggccacccacacctgtgttctcatgctgaaagagatttgaattcttcctggcatggaggtctccggttcctgcccagtgagggtccttccaactcaggcctccagccaactccccccacccccgccaatattctggggctgacagagatttcaattctccctggcatcgaggtatcggggtcctgcccagtgagggacctcccaacgggggcctgcagccacccccgccagtgttctggggctgacagagatttgaattctccctagtgtggaggtcttgaggtcctgcccagtgagggacctcccaagcggggcctccagccaaccccactggtgttctcatgctgagagagatttgaattctccctggcatgtaattcacaaggtcccacccaatgagggtccttttagctgcggtgtccagccacccccactggtgttcccaggctgacagagatttgagttctccctggcatggaggtcttggggtatcgcccagtgagggacctcccaaccggggcctccggccacccccgcctttattctgggactgacagagatttgaattctccctggcatggaggtctcggggtctggcccagtgagcgacctcccaactggggtctctggccacccctgccagtgttctggggctgacagagatttgaattctccctggcatggaggtctcggggtcccgcccagtgaggacctcccaactagggcctgcaaccaccccggtagtgttctggggctgacagagatgtgaattctctctggcacggaggtctcggtgtctgacccagtgagggacctcccatatggggcctccagccacccccgccagtgtcctggggctgacagagatttgaattttccctagcatggagttcttgaggtcccacccagtgagggttctcctaactgggccctccggccacccctacctgtgttctcatgctgacagagattcgagttctccctggcatggaggtctccagttcctgcccagtgaagttcctttcaactcgggcccccagccaccaccccaccccctcctccagtgtcctcaggctgccagagatttcaattatccctggcatggcggtctcaaagtcctgcctggtgagggatcaccgcactgggacctccagtcactcctgctggtgttctcgggcccaaagatatttgaattcttgggatgaaatggccaaagatgagctgccatctttgctgtttgtgcaacttagccactccagcctgagggttttgtggaatccatacctaccggggcagaagggatctcccagcacaacacagctactctacaaaatcttggtcaggcagattcttttagcaggttcctgacccatttcttcttaattggcaggaccttgacaactctatccaagggagaatgtaaatctctgtcagctctagaactaaggggtggaagccccacttgggaggtctcacccagtgaggaacggatcggggatctacttaaagaatcttgccatgatttgatagagcagttgtgctgttctggggatcccctccaccctagtcactttggactctccaaagcccacagactggaataactgagtcacccaaacagcaaagatggcggcccccttctcccccagggaactcatcccagggagaattcaaatctccgtcagcctgagagcacctgcaggagtagctgcaggccccagttgggaggtcccgcccagcgaggaggaatggattggggatccacttaaagaggcagtctggccacattttggtagagcagctgtgctgcgtgggggatcccttctgccctcagttggtttgggttctcaaaagcccaacaggctggaatggctaagttacccaaacaacaaagatggtggcctgccctgtcccctaggaagtcagtctcaggtaggtaaaacactgttgctggtggctggctggagttgtttccttgattatgtgagtaatgcgagtacctggttgtttcagttgaaggtgctgtattgacttgcccttttcattcctctccatgagagccgtgcaccctagcttcttctagtcagtcatcttggccacacacccccataatcgtattttttaactaaatcattctttaaaactctaacaaaatatttaaacatttaaaaagtgtgagctttagaaatgcctaattacatcttaggtttgagacacgtagcagttatgtacattgtcaattccagattttgcatcttacaaggaaaggacttagatcttattcagtctttgtgtgtctaaactatcttcgcctgtaaaatgagtacaatgaagtactttatagagttgtaaatgttatatgtaaaaatatagcagtgagggggcagtgggctggccaaggtggccgagttgaagcagctagtgtgtttggctctcacagagaggaacacaaggggagagtcaatactgcaccttcaactgaaacatccaggtactcacattgggactaaccaaggaaacaacttgacccagggagaatgaagaaaagaaaggcaaaacgacagcccacctgggagtaccacagagccagggggagctctctcacccagggaagcagtgagtgaatgtgtgaccctggaaacccatgctttttccatggatctttgcaatccttgggtcgagagttctcatgaacccactttaccagggccttcagtctgacagagctacgtggaatcttggcacagcagccactcaggcacacatggagacctgggagccttaggtacctgggctttcctgcaaaagtagctgcaactgtggcaaagtgggaggtgagaccctcatacatatccctagggaagaggctgaattcagggagctgagcagcaacagcctgcaggccccacttccacagcacctcacaggataagagccactggcttggaattccagccagccaccagcaacagtgttgagcctccctgagacagagctcctgagagaaggggtaggccaccatctttgctgtttgggcaacttagctgctccagccttcgggatttggagagtctcagcggaccagggacagagggatccattagcacagcacagtgctactctaccaaaatatggccggactactgctttaagaaggtccccaatcccattcctcctcactgggcaggacctcccaaccagggcctctagctacccactctggtattctcaggctgacagagatttgaattctccctgagatggagtgccctgagggaggcgtgggccgccatatttgatgttttggcaacttagccattccagcctttgggctttaaggagtcccagctgacttagggcggatatggccccccagcacagcacagctgctgtacaaaagcatagccagactgcttctttaagtaggtcccggattcattcctcctcactgtttgggacttcccaactggggcctccaaccacccccactggtgttctcctgctgacagtgatttcaattttttctgggctggagctccctgatggaggggcaggccaccatctttgctgtttttgcaacttagccacttcaaccttcagtctttggagtgtccaaggagaccagggggtgatgtggaccctcagcatagcacagctgctctataaaaatgaggccagactgcttttttaagcactttcccaatgccattcctcctcactgggcaaaacctccaaactggggtctccagccacctcctacaggtgtgtttgggccagcaacaagttcattcatacctccctagggcaaagcttccaaagggagcggtaggctgccatctttgctgtttcacaggcttcactgatgataactccaggtactggaaaatctgaggctactagagactggagcgggccctgggcatactgcagcagccctatggaaaagtggccagactgttacctgggttcccattcctatatcttctcactaggcaagtcttgcaggcctggacctctacctaaccccccctaccagaactgttgagccagtagcaactcagccactccctggagagagcctccaggggcaactgaaagcctctctgccactgcttctgcagtggaactgtccttgctaccctcagactgatgaaggagctaacacccttatctacaccttcaacaagctttaattgaccaaagcccatctctcatgggttctacacactccccactgctcatgacagggaacccctggattggcccccacagcacgaattctccatcctgattgctgattgcagtaaacagttgctgtattctccaggggtggtggaactctgaggagacaaacaaaagacccttggctacaaccactactaatgtcccttcctcttctgcctcaaagttaggaaagaaatataaacactgagattgccccagagctgcagtgggcagcctaggagtgccaagccatgacctacagccagcactcaagggggagagaagcacattttcagatcattgagagggaacatggctgcaactgtaaggaaacataggggagccacatgaccaagcaagagtctaccaactgaccagtaagcccaagtgccacctactggatcacatcccaaagcttcagcatcaaaaataccttactaatatactcccctctgaaaccagaaatgagaagtcagcttcaaataaagaccctgcacaaagcctcagcctggtgaaaacatccgaaaataagtctacggactgtactcaatctacactgcaattaaaggaaaacccataggtggaaatgagaagaaaccaatgcaagaactccagtaactcaaatggcctctgtgtcatatgtccttctaacaaccacaccagttctccaacaagagttcttaacctggatgaactgtctggaattacataaatataattcagaatatggataggaaaaaaaatcatcaagactcaggagaatggcaaaacccaatccaaggaaaataagaataacagtaaagtgttacaggagctgaaggataaagtagctggtataataaaaaagaacctaaccgatctgaaagcgccgaagaacacaatacaagaattccacaatgcaatcacaagtattaacagcagaaaaaaaaacctgaggaacgaatctcagaacttgaagattggttctctaaaataagatggacaaaaataaaaaagaatgaacaaaaccttcaaggaggatgggattatataaagaggccaattctacaaatcactggcatctctgaaagggaggtggagaaatcaaacaacttggaaaacgtagttcaggatatcatctttgaaagcttccctaaccttgctagaaaggccaacagtcaaattcaggaaatacaaagaactcctacaagattctacacaagaccatcctcaagacacataaacatcaggttttccaaggtcgaaatgagggaaaatatgttaaaggcagccagagagaaagggcaggccatctacaaagggaaccccattaggctaacagcagatctctcagctgaaatcctacaagccagaagggattgggggactatatttaacattattaaagaaaatcttcaaccaagaatttcatatacagctaaactaagctttctaaatgaaggagaaatgagatcatttacagacaagcaaattctgaggtaattcattaccaccacatctgccttacaagagattttagaaaggaggactaaatatagaaaggaaagaccactacacgctaatgcaaaaacatacttaaacacacagaccggtgacactataaagcaaccacacaaaaaagccaacataataaccagccaacagcacaatgacaagatcaaatctacacaaatcgatactagccttgaatgtaaatgggcaaatgccccacttaaaaggcacagagtggcaagctagatttaaaaaaaaaaaaagtgagacccaatggtatgtcgtcttcaagagacccatctcacacataatgacactcatcgtctcaaactaaaaggatggagaaaaatctaccagacaaatagaaaacagaaaaaaagcagaggttgcaatcctaatttcagacaaaacagatttcaaatgaacaataatttttcaaaaggacaagggggcaggggcaagatagccgactagaagcagctgcagtttgaggctcccactgagaagaactaaaagagtgtgcaaatcctgcaccagcaactgacatatccaggttctatgatcaggactgactaggtggttgccgtgacccatagagaacaaggaaagatgggctggtggattggcccacctgggagccacatggggcaaggggagccgtcaccctcagccagccaagggaggcagtgagtgagcatgctacccagcctgggaaactgctttttccatggatctttgcaatccacagatcagaagatcccactcatgagaccacaccacgagggccttgggtgccaaccacagagccatgcagattctcaacagccactcagctggagtctgcctaaaactaccgagttcccaagttggggaggggtggtcatcatcactgtggctgcctgctgcctaaaccctctgagttccctgggggagggggagcaatcatcactgtggttgctggctgcctaagacaactgagcttcccaagagaggggcagtcatcatcactgcagctgcctgctgcctgaggaaactgagctccctaagaagggacagcagccatcactgtggctgctagctgcctaagacactgaactcctggggaggaagggcggcagccatttctacagatccaggctgctgtttttcctttgctgatgccaggaagactggacggcttggtcccaagaggtattccccacagcgcagcatactggctgtggcagatcatggccagactgcctctttaggctgaccctgacccatccctcctcactgggtggggcctccctgcaggaactccagcaactcaagccagggaattagggagagaactctgatctctctaggtctgagtccctagtgggaggggtggctggctgttgtctccacaaaccggaagacttgttctttccccctgctcactctgaggattccaggcagcccagatgagtgggattttccccggcacagcatacccccttcccaaagggacaatcaaagtgcttcattaagcaagtcctggatcctgtgcccctcaactgggtgaaacaccccagtgggtcaccagacaccttatacaggagcatttctactggcatcaggtgggtgcccctcaaggacagagatcccagaggaaggagtggggtcccatctttgctgttctccagcaccctctggtgacatcttcaggtgtgggagggacccagataaatagggcttgaagtgaatccccagcaaaccacagcagccctacagaagaggtacctgactgtcgaaagaaaaacagaaagcaacaacaacatcaaccaaaaagtccccacgaaaacctcatctaaaggtcagcagcctcaaagatcaaaatgagacaaactcatgaagatgagaaaggaatgaaaaacccctaacaactcaaaaggccagagtgacttgtttactccaaatgatcacaacacctctacagcaagggcacagtcctgggtggaggttgagatggatgaattgacagaagtaggcttcagaaggtgggtagtagcaaacttcactgagctaaaggagtacgctctaacccaacatattggaacgaatcccagaacttaaagattggttctctaaaataagacagacaaaaataaaaaagaataaaacggaaggaagaaaacctccaataagtatggggttatatatagaggccaattctacaaatcactggcatccctgaaagggaggtggagaaatcaatgcattgggttagaacatgctcatttagctctgtgaaatttgttattacccaccttctgaaccctacttctgtcagcaaagaagctaagaaccatgttaaaagattacagaagatgctaactagaataaccagtttagagaggaacataaatgaccagagccagctgtaaagcacataaggggaactttgtgatgcaaacacaagtatcaacagctgaattgatcaagcagaaaaaagaatatcagagcttgaagactgtcttgccaaaataaggcaggcagagaagattagagaaaaaagaatgaaaaggaatgaacaaaacctcaaagaactgtggaactatgtaaaagaccaaacctatgactgattggactacctgaaagagacaaggagaatggagccacgttgggaaaacacacttcaagatatcatccagaagaacttccccaacctagcaagacaggccaacattcaaattcaggaaatccagagaaccccagtaagatactccacaagaagatcaaccctcaagacacatagtcatcagattctccaagatcaaaatgaaggaaaaaatgttaaaggcagacagagacaaagggcaggtcacctacaacgggaagcccatctgactaagagtgggcctctcagcagaaaccccacaagccaggagacagttgggtccaatggtcaacattcttagagaaaagaatttctaacctagaatttcatatctggccaaactaaccttcataagtgaaggagaaatccttttcagacaagcaaatgctgagggaatttatcaccaccaggcctgccttgcaagacctcctgaaggaagtgctaaagatggaaaggaaaaactggtaccagccactgcaaaaatgtactgaagtacaaagaccaatgacactatgaagaaactgcatcaactagtgagcaaaataaccagcaagcatcatggtgacaggatcaaattcacacataaccatattaaccttaagtgtcaatgggctaaatgtaccaattaaaagacacagaccagcaaattggatataaagagtcaagattcaggtgtgctgtattcagggttcctatctcatgtgcaaagacacacataggctcaaagcgatggaggaaaatttaccaagcaaatggaaagcagaaaaaagcaggggttgcaatcttagtttctgacaaaacagactttgaatcaacaaagataaaaaaagacaaagaagaacatcacaatgataaaggaatcaattcaacaagaagagctaactatcctaaatatgtatgcacccaatacaggagaacccagattcacaaaacaagttcttagagacctacaaagagactcaggctaccacacaacaatagtgggagacattaacaccccactgtcaatattggatcatctaggcagaaaattgacaagaaaggacttgaacacagctctggatcaagtgaatctcatagatatctacagaactctccaacacgaaacaacagactatatattattcttagtggcatatgacactctaaaattgatcacaaaattagaattaaaacactcctcagcaaatgcaaaataactgaaatcataacaatctctcagaccacagtgcaatcaaattagaactcaagattaagaaactcactcaaaaccacacaactacatggatattgaacaacgtgctcctgagtgacttctgggtaaataataaaattaaggcagaaatcaagaagctctttgaaaccaatgagaacaaagagacaatgtaccagaatctctgggaggcaactaaagcagtgttaagaggtaaatttatagcactaaatgcccacatcataaagctagacatatctcaaattgacaccctaacatcacaactaaaagaactagagaagcaagagcaaacaaatccaaaagttagcagaagagaaaaaaaaaaatgactaagatcaaagtggaactgaaggagacagagacatgaaaaacccttcaaaaaaaaaaagaaagaaataaaacatgttcaaatagcaagagataaaatcaaattgtctgtgtttgcagaaaatgtgattctatatctagaaaaccacatcgtcacagcccaaaaactccttaagctgataagcaagttcagcaaagtctgaagatacaaaatcaatgtgcaaaaatcacaagcattcttatacaccagcaatagacaatattcttcaattcctatacaccaacaataggcaagagagccaaatcatgaatgaactcccattcacaattgttacaaagagaataaaatacctaggaatacagctaacgatggatgtgaagaacctcttcaaggagaactacaagccactgctcaaggaaataagagaggacacaaacaaatggaaaaatattccatgctcatgggtgggaagaatcaatctcatgaaaatggccatactgcccaaggtaatttatagattcagtgctattcacatcaaactaccattgacattcttcacataattaaaaaaaaactactttaagtttcatatgcaaccaaaaaagagactgaatagtcgagacaatcctaagccaaaagaacaaagctggaggcatcatgctatatgacttcaaactatactacaaggccacactaatcaaaacagcatggtactgttaccaaaacagacacacagaccaatggagcagaatagagatctcagaaataagaccacacatctacaaccatctgatctttgacaaacctgacaaaaacaagcaatgggggaaggaatacctatttatttatttatttatttattttgagacaaagtctcactctgtcaccaggctggagtgcagcggcatgatctcagctcactgcaacctctgcctcccggattcaagtgattctcctgcctcagcctcctgagtagctgggactacaggttcgagccaccacgcccagctagtttttgtatttttagaagagacggggtttcaccatgttggccaggatggtcttgatctcttgacctcaagatccacctgcatcagcctcccaaagtgctgagattatagacatgagccactgcacttggccaggattccctatttaaatggtgctgggaaaactgactagccatatgcagaaaactgaaactggacctcatccttacatcttatgcaaaaattaactcaagatggattaaaaacttaaatgtgaaaccccgaactgtaaaaaaccctagaagaaaatctaggaagttccattcaggacataggcatgagcaaagattttatgatgaaatcatcaatagcaattgcaacaaaagcaaaaattgataaatgggatctaattaaacgtaagcacttctgcacagggaaagaaactatcatcagagtgaacaagcaacctacagaatgggagaatatttttgcaatctaccaatctgacaaaggtctaatatccagaatctacaaggaacttaaacaaatttacaagaaaataacaaccccatcaaaacatgggcaaaggccacgaacagacattctgaaaagaagacatttatgcgtccgacaaacatatgaaaaaaaaagctcaacactagtgtttattagagaaatgcaaatcaaaaccacaatgagataccatctcatgccagtcagaatggcaattattaaaaagtcaagaaacaacagatgctagagaggctgtggagaaacaggaacacttttacactgttggtgggaatgtaaactagttcaaccattgtggaagacagtgtggcaattcctggaggatctagaagcagaaataccatttgacccagcaatcccattactaggtttatatccaaagaaatataaatcattctgttttaaagatacatgcacacttatgtttattgcagcactattcacaatagcaaagacatggaatcagcccaaatgtccatcaatgatagactggataaagaaaatgtgatacatatacaccatggaatactatgcagccataaaaaggaatgagatcatgtcttttgcagggacatggatgaagctggaagccataaacttcagctaattaacacaggaacaggaaaccaaacaccacatgttctcataagtgggaaccgaacaatgagaacacatggacacagggaggggcagaacacacaccggagcctgttgggaaggtagggggaaggagagcatcacgataaatagctaatgcacgtggggcttaatacctaggtgataggttgataggtgcagcaaaccaccatggcacatgtttaactatgtaacaaacctgcacatcctgtacatgtatcctggaacttaaaataaaatagaaaagacaaagaaggatattacataatggcaaaggcttcaattcaacgagaagacctaactatcctaaatatatattcatccaatgcaggagcacccagattcataagtaaagttcttagagacctacaaagtatgtttcacagtaatagtgggagatttccacactccaatgacagtattagacagatgattgaggcaaaaaaatgaacaaagatattcaggacctgaactcaacattggatcaaatggatctgatagacctttacagaactctgcactcaaaaacaacagaatatgcattcctcacatcatcatatgccacatactctaaaatcaaccacataattggacataaagcaatcctcagcaaatgcaaaacaactgaaatcataccaaatacatactgagatcacagtgcagtaaaaatagaagactaagaaaattgctgaaaatcatgcaattacatggaaatcaatcaacatgctcctgaatgactttttaataaataatgaaattaaggcagaaatcaagaagctctttgaaaataatgagaacaaagttacaacatactagagcctctggacacagctaagacaatgttaggagggaaatttatagcactaaatcccacatcaaaaagttaggaagaactcaaattaataacctaacatcacaactgaaagaactagagaagcaagacaaaaccccaaagctagaggaagacaagaaataactgaaaatctgagctgaactgaaagaaaccgagacatgaaaaaaagaaattcaaaagatctatgaattccgggtaggtttcttgaaaatattaataagaaagtctgctagcagactaatacagaggatgattgaaagaaacacaattagaaatgacaaagggaatgttaccactgaccccacagaaatagaaacagccatcagaaactactgcaaacacttctatgcatacaaactagaaaacttcaaagagatggataaattcatggagaaatacaccctcccacaactgagccaggaagaaattgatttgctgtaaacagaccaataacaagctccgaaattgaatcagtaataaataacctaccaaccaaaaaaagcccagaacatgatggattcccagtcatattctactagaaatacaaagaagagctggtaccatttttacaggaactatttgaaaatattgaggaggaggaactcctccccaactcattctatgaggccaacatcatcttgataccaaaatctggcagatacacacacacacacacacacacacacacacacacaatcttcaggccaataccctcgatgaacatcaatgcaaaaatcctcaacaaaatactggcaaaccaaatccagcagcacatcaaaaagttaatccatcatgatcaagtatgcttcatccccaggatgcaaggttgcctcaacatacacatatcaattaatctgattcatcacatgaacaaaactaaagataaaaaccatgtggttatctcaatataagcagaaaaggctttcaataaaattcaatgcctctccatattaaaaactctaaaaaatctgggtattgaagaaacatagctcaaaatgatgagctgtttttgtatcagtatcatgctgttttggttactgtagccctgtagtatggtttgaagttgggtaacatgatgcctccagcttcgttctttttgctgaggattgcttggctattagggctcttttttttggttccatatgaattttgaaatagtttgctctagttctgtgaggaatgccgttggtaatttaatagggataacttgcatctgtaaattactttgggcagtatagccattttaatgacattaattcttcctatccatgagcatgacatgtttttccatttgtttctgtcttctctgatttctttgagcagtgttttgtaattcttctagagatctcctagagatctttcacctccctggttagctgtattcctaggtatcttattttttctgtgtgtagcaattatgaatgggattgtgttcttcatttgactctctgcttgacttgatgtataggactgctagtaatttttgcacattgattttgtatgctaagactttgctaaagtttatcagcagaagaagctttggggccaagactatggggctttctagatatagaaacatgtcatctgtaaacagagacagtttgacttcctgtctattcctctcttccctcctctgtttggatgcccttccagttttgcacattcagtgtaatgttggctgtgggtttgtcatggctagctctcatcattttggcacttctgactagaagggcaagaggggccagtgttgttgttacctgaaaggtaagtgcagcccacaaaaatgcagtgaagaagaagatgtattatgcatatgtttaagttaatacaattgagatatatttagcagacagaatcaatagagttgatgagtgactgactggatgtgtggggagtttatatcactcccaggtttttgacttgggcaaccgggcactcatgaaggaaaaagaggatccagggagaggaacttttctgaggactggggtagggctgaacagctgcattcgaggctggtggagggtgggctgggcatgggatgcacaaatggaaattccactgggtctgcagctcacacataggcatgaccagcatagagatagagaggccccagtgctgctgagtaactgtgattccccaggtgatggcatcagctgagaagggaaggaagcccatgagaggacactgaagaaggagtgagcagacaataagaagcccacagaagacagagaaggaacaactagagggagaagccaaggcaggcgtgtgtggtaacacataggaactgagggagaggacatttcaagatggtggggatgccatacaacaggactatgtgatgatttttggctgtgtccataggaagtcacaacaggcaagggaaagaaaccagaacccagtcatggagctaagaagtgagtcagagagtagaggggtagggacagtgaggtaagtcctctttctaaggaagtttggctgaaggatagactagctggacacatgctggctgtgtggggtagagggaggaatgatggagggtaggagagccttgagcctgcgagaagagtctcttagaatagagaagctgaagttaaagttgtggaagagagtggggataactgagtgacagataatcaggagaagaaaaggagatccagactcatgacagagagatgacctttgccaagagtacagccgtctttcacggtcacagagaggtaggacaaaatgagtggtgttcaagaattggtttgtagcacaatatttcaactatgtcctttaaaaagtttctccacagacactacccaaagcagtgcttcactacagtggcagacagacctgaaaattttcatctgaagcagcagagtgaactgcagaggcaggtaatttctagaaggcttgctttgttacattgaaactgaagattattcatgaggccagtcttctgagatttctgtcatttctctcatgtcaggtcaccaaccagtgtggaggctaaaagcgggcctctttggggattccaggtggaagtgttggacatctgtagtattcctggtctcgaatcatcctgatattcttcattttgttattcacattggactagggtgaggaaatgagttctggtcagagtcaatgtttcccaacaatgttgatttatttggtcctaaatatttaaacacattttataaatagcttttccacaccacatatatagggaaaaacagggagttaacttggcccagcagcactgtttgtggggctaacaccctgtctgtgtatctcacaccaaccctaagctttgtttctctcctgtcactttgctcctatttctttcatgtgaaagaatgttcattttctttgaaaatgagctcatttcctcattttctttccatatttcagcaggaacaaagatcaccacaactggctcgccttcaactatgttagatggcaacttgccttcagtatggtgaaacacatcagttaagaccggggttgtgcatggcaggactttctacaaggacacccagtctccttaataaacatgagatgctctctttccagaatttctcttgcctgacacagcataggaagatgctgaacggccacacagtgatccattggtcagtggtgacataaggaggtcagaggggaggagtgaggagaagtagggaagactaggtggttgtaggcctccttcatctgttcattggctgtggcattaggccagctactctttgcacttctgtaaagtgagacggtcgatcttgtctgcctctctagaggatggttgcaggtgtcaaatggggtagttaggtgggagggcatttcacaaagttaaaaaatatgactttggaggcttgttatattgatgaggattataatccctgagaattcctggtatgaagaagggaaaagaagataatttgtgaaagaaatgtgtccagttactagtctttgaaaagggtcagtctgtagctcttcttaatgagaataggcagctttcagttgctcagggtcagatttccttagtggtgtatctaatcacaggaaacatcgtggttccctccagtctctttctgggggacttgggcccacttctcatttcatttaattagaggaaatagaactcaaagtacaatttactgttgtttaacaatgccacaaagacatggttgggagctatttcttgatttgtgtaaaatgctgtttttgtgtgctcataatggttccaaaaattgggtgctggccaaagagagatactgttacagaagccagcaagaagacctctgttcattcacacccccggggatatcaggaattgactccagtgtgtgcaaatccagtttgcctatcttctcaagttagggttaattggataattctggagaagtacacattgaaaactagaactaagccaagcaattaaatacgtttcctgcctattacatgccttggtactgtgcaaaagagctcacagggcatctgaggaaagattactaacacacacctcaaatgactgtgtctgatttcctagaaggactcagaaagggagtgatcactgtggattggactggatagtgcctcatgctggaggtgggctttgagtggagccgtgaaggtcaagaaaaagtgatataaacactggctccttccttttgaagggacaccacactctttgggcttagtggttatagatgcctttagcgcagcccaggagcaagcatttgttgtctacctaccacgtgcttggctttggaaatgcaaataaaaaaggaggtcactattttatgaaaggatgattttattctacctgatgtagccagctgcaagcttcagaaggcagcacatacaataatgatgggctggtgagatgagagctaatggagagatgtacaaagtgccatgggatcaccaagcagtggtcaggtgaaggcatctgattttctttaagatcttgctcaaatgtcatctcctctagaagaaaatgtagggcttcatcagtcagacaacatggggaaattgatctcagttcaagaaacatgtattgattatctaataggttcaagcagttgtgcactggggatagaaaaatattatcatcatcatcatcatcatcaacaacaacaacaatcttgctgtatgccaggcacataattttacatgtattatctcatttaatctcaaacaactctatgctataggtgtttattattatccctatttagatgaaacatttaaccttcagagaatttaaataagttgcacttgcaagcttagcaggtgaggaatcaaatttggaataggacctagatcagtgatcccctagatctacaatcttaaccagtaccctgttctctctgcttagagagcacctagagaaaatctgtaagcaaataatcacaaaacaagggatgacacaatatatgtaaatgtaggattttacattaagtggaagaaggaagaaggtcattatttactttttctgggtgagagatgaaaacacttctgcaagagtatttccaatttcaccaaagtatggaaagatgtttatcctaataacaatactcagtaatggtatttgatttaatttcagcctagtacatgatttgattctaaaacaacatttccttagaatttcaatatctggaattctatagctcaatggttttggtgactattaatattttatatacttcttttgatagttttggggctaagtgaatcaaatctattcataactctaagttttcaaattctgaaatcttttagagttgtttagttaaaactggttcttttcttccagtttgttagcttaccaaatgagccaaaataaagaaaaaacaaaaatttattttcagtctgatatagagagttatttatagttacatggtgctatctcttcctttaaaaacatttcttctttctttttttctccctctctctttttgaggggattataaacactgccagtattttttgcctaaagggtaaatcctcttaaactcttttgggatctatattaggtgccagatggtcataagaaaattatgtgagatactggtgaataaacaagcaaaccagaagctaggttttcagaatagatgctagccagtagttcacaggcattatccagcaactggtggggatctacttggccagcccgtgaccactgagatgtgaacttccctggaccttggcagtcaagagggagagaggcagagaaagctccattatcatcactaattcccttttgaccacacgtgacagcatgacctgcttcaaagaaataagataaatggtggttaggtgcccccacaggcccacagtagcccattcaacaaagagcaaaagtaaaactactcctgatggatttagcaacatcatagttcacttgtgaggcaacaggcttttattgcttgctttgggactggcttcttctcattttctgtaggaaagtgagcctacctgacttagaaacaaaattttggaacacaaaccctttgtaagttgggagatccctaaactaaaatttccagtgagatgaaaagccccttgagaaagaaatttctggctgggcatggtggctgatacctctaattccagcacgttggaaggttgaggcaggaagatcacctgaggccaggagtttgagaccagcctaagcaacatagggaaacccctgtgatatagtttggatctgtgtccccacccaaatctcatgttgaactgtaatccccagtgtggtaggtggggtctggagggaggtgactggattaggagggtggatttctcatgaatggtttagtactatctccttggtactcctcatgatagcaggtgagctctcaaaagatctggttgtttaaaagtgtgtggtatctcccccctcactctcttgttcctgctttcactgtgtgacatgcctgctcccgcttcacaatctgccagtaaattgtggcaggttatgagtgtgtgtaatttatgagtgctgataggatgaaagatagaaaatgattttagatagaaagaaaatcattctggaagctttgagtcttccttttgtaatctatgttggttcttttttaacatcccctgtttcagcacttactgacctggagtctgtctcacaatgactaaatgtaatataagattctaggatgtacactagaacagaaaatggaaactaaataaaaactgaggaattctgaacaagtatggtctttagataataataatatgacaatattgatgccttaattgtaatgaatgcaccacactaatataaaattttagtaatagagaaagcagagtttgggtacttaccaggaatattatactattttcacgatgtttctgtaaaccttaaagtgttcaaaaattaaaattttatttaagtcaattgcaatatataagattaattgaacccaaattgtggaaaatataacttctgttataatgttccttggaagcttctaaagacaaggcctttcttttctatttttagtagaaattacacgtattttcaagtcaaactgttaatatgtgacaatatttgcagtactgaaagcttaataacttatagattcatggtaacgaaaagcaaagcatagtgttgtttttggttttggaatactgtatgaaatatgtagtctttttttttttttaagatggattctcactcatttgcccaggctggagtgcagtgggatggtcttagctcactgcaacttctgccttctgagttcaagcaattcttctgcctcagcctccccagtagctgggactacaggcatgtgccaccatacctggctaatttttttttttttttttttttttttttttgtattttgagtagagatggggtttcaccattttggccaggctggtctcgaattcctgacctcaggtgatctgcccaccttggcctccaaaattgctgggattacaggcatgagccaccgtgcctggccaaaacatgtaatctcttattcaagatttataataacagttatgtgatactcagtaagggatggtgatctacataaaataaaagtacaggcacagtggcttatgcctgtaatctcagcattttgggaggccaagacacgaggactacttgagttcaaaaccagctttgtcagcatagtgagacctcatctctacaaaaaaatcaaaaacattagctgggggtggtgtcacacactggtagtcccagatatttgggaggatgaggtaggaggatcctacctgagcctggaagacaaaggccactgcacttcagcctgggtgacaaagtaggatcctatctcaaaatagatagatagataaatagatagatcaatgttatttatctcaatatttgaaagaaaagttgaaaaacctccgagctcaactaagaatcagtttctagaataaacagaagtataaatcattatacctctttactttaaaaatattacagacattataagtattttaataacaaaaaatcaaatactttatatatgctttgtaatctgtcctttacaaaatcatttaacctttatgacaggagctactatggtgctcattccacagatgaggtgctgaggtttagtgaactgaatagcttgttcacagacagctggagacagagactcagtttgcccatacaaagataattttaaccaatagcattttacaatagaatatcataatgcctatgaattctaaaaacattttattcaaatctctcttttaatattagcacctcttcaacctcttcttgactctaccaccaaaattatcttcctaatctctcttaccctgtaaatttctgtgtgtgtgtgagtgtgtgtgtgtgtgtgtgtgagagagagatagagacagggaaagagtggatttaccaaaaaataagataggaaggttgtcataacatcttcccttttatttctcaattctgaagtccccattttcatccctgggagagttgttctaggatagggtagctaaaggaacagagaaaatcagagtaatggaattagctaaaagggaaagtggcgacctgaagaccaaacactaaagaaggggccagtgaatggcaaagaaagtggagagcaaaacatgcaggtagatagggtcctagttaatgaaggctcagaggctggagggtaaacccatgcaagggtaaagtttaatacagtgaaaatataagtaagatgggagacctaccacagagaaccaacacttggcatgtttctgatgacagtgtttagattgcagtggttctaaaaattaagtgattttggaattggcattccatgtgactttaaaagataaatgataaagtaaatgttttattcagctctctctctccctccccacccaccttcttccctccattcttctgtttttaatggggatgtttaaaatccaatctcacattaattttttcttaagtgtaaataatgggaaaacttcacttcgagaggggactcctcagtagacctccataatttcctggcttttactgccagttagtcatcaggaatagccacggctaagagagtaccaactgggcaagccagtgaattataccagtgcatctcagatttactgatttgtttaacttttgttttttgttctttttttttttcagagatagaatctcactatgttgaccaggctggtcttgaactcttggcacccaagcgatccttttgcctggaatcccaaagtgtcagatttactgaagaatatttcattgtaattactttttatactttataggtcaagagctctgttttaaatacaaaatttattgaatatactttttcaaacaaagtttcatgttctaatcattgtctgatttcagcattaaatgaaacacagtaaagaaagttgggctgatgttgttgttggtggttgtttttgtattctgattacagaactgtattttgagtgggctgaacatagagtaaatgcatcctcagctgctggtctagacactttttttccatgagtgaaactgcacattggggatggtgggtcggggctgtgagtccagagagttaagcagtaaccccaaagattttaactgggatccagaacagttttatcatcgctcttctttatgtctatatttatctttagtaattgtaccatatgctgcattttccactggttacataggtaagcctagtcctccccacaacattcttataccaatgatggtgacaatatgctgtatttcttttgtatcaactccttacttactgtagctactcaaattgtactaagaccactggtactaatctgcaaactaattttgaagtacctggaagtacagatttgaaaaccttttatagcaatctgacattgccacaatattttacattttacaaaaaataaaagtctttcactctgggtagtttgaaaagcaccactgcagagaaaatagttgacacttcacaaatgcttatttgattttcctgaaggtgataagtaaattagatattctataatgtgttatctttaacacaaaaattatagtaactatttaaacaattaaataacagaaattcagattcatggtaaaatcaaagaatataaactaaaaattccctatattttctatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaatatagaaataaatatattttctgagaaaattaaattaaataaatatattttctgagaaaatataaataaaatttccctatattttctgagcttgagtaactctttaacaaaatgttgacatagataagcacttcagcattcatggataagcatactttcataaaatctgaagaaaaatatatttgataattccaatgcctgtctcagagctactttttctgctggtacctctgactggaatgctttctctctcaactcatacttttaaattctagccccctttcaggatccaaatgctccattttgtagaacatgtttattaaaatagtttatactctcttattgtattattatatgatgccttaattcatggcaacttgttaatatgtcatatttcctcttaagcttcttaagacgagaccatttattatcactttgtatatttttaatctttcccagaataggtgctctataaatgcttactcagcattacatcattaaataaggcaacacaatgtaattttcactcttaataatgactgcattagcagggcaaggactctgaggtatttgtctgacaagcattcaaaattgctagccaatgttagaactagaaattttggaaaaggtagtgaggtcaagtcattgactgaccttggctttactcatacatactctaaccagatggatacacatcagagcctcagagtctccgagtttaaatgggccataggcaccacctaaactaatagtcaaaccggaaaaagtatacgaggacacttggaagatgtattgagttgttaacctaaaagttaagagaactaagaatctaaatggtggttgcttaagaaaaataccatctcacaaaagaatactcctaaccactactgcaaaaaacacacttttggggaaagtacacccatatggtttgtacacattctcaaatatctaaaagtgacttgggcttgacatgtagttctgaatgcttctgttagatttccaatttatctctcttttggtaccagtaccatgctgttttggttactgtagccttgtagtatagtttgaagtcaggtagcatgatgcctccaggtttgttcttttggcttaggattgacttggcaatgcgggctcttttttggttccatatgaactttaaagtagtttttcccaattctgtgaagaaagtctttggtagcttgatggggatggcattgaatctataaattaccttgggcagtatggccattttcacaatgttgattcttcccatccatgagcatggaatgttcttccatttgtttgtgtcctcttttatttcattgagcagtggtttgtagttctcctcgaagaggtcctttacgtctcttgtgagttggattcctaggtattttattctctttgaagcaattgtgaatgggagttcactcatgatttggctctctgtctgttattggtgtataagaatgcttgtgatttttgcacattgattttgtatcctgagactttgctgaagttgctgatcagcttaaggagattttgggccgagacaatggggttttctagatatacaatcatgtcatctgcaaacagggactatttgacttcctcttttcctaattgaataccctttatttctttctcctgcctgattgccctggccagaacttccaacactatgttgaataggagtggcgagagagggcatccctgtcttatgccagttttcaaagggaatgcttctagtttttgcccattcagtatgatattggctgtgggtttgtcataaatagctcttattattttgagatacgtcccatcagagatatagaccaatggaacagaacagagccctcagaaataataccacacatgtacaaccatctgatctttgacaaacctgacaagaacaagaaatggggaaaggattccctattaaataaatggtgctgggaaaactggctagccatatgtagaaagctgaaactgaatcccttcctgacaccttatacaaaaattaattcaagatggactaaagacttaaatgttagacctaaaaccataaaaaccctcgaagaaaacttaggcaatatcattcagtatataggcatgggcagagacttcatgtctaaaacaccaaaagcaatggcaacaaaagccaaaattgacaaatgggatctaattaaactaaagagcttctgcacagcaaaagaaactaccatcagagtgaacaggcaacctacaaaatgggagaaaatttttgcaatctactcatctgacaaagggctaatatccagaatctacaaagagctcaaacaaatttacaagaaaaaaacaaacaaccccatcaaaaagtgggcaaaggatatgaacagacacttctcaaaagaagacatttatgcagccaacagacacatgaaaaaatgctcatcatcactggccatcagagaaatgcaaatcaaaacgacaatgagataccatctcacaccagttacaatgacgatcattaaaaagtcaggaaacaacaggtgttggagaggatgtggagaaataggatcacttttacactgttggtgggactgtaaactagttgaaccattgtggaagacagtgtggtgattcctcaaggatctaggactagaaataccatttgacccagccatcccattactgggtatatacccaaaggattagaaatcatgctgctataaagacacatgcacacgtatgtttattgtggcactattcacaatagcaaagacttggaaccaacccaaatgtccatcaatgatagattggattaagaagatgtggcacatatacaccatggaatactatgcagccataaagaatgataagttcatgtcctatttagggacatgcatgaagctggaaaccatcattctcagcaaactatcacaaggaaaaaaaaccaaacaccacatgttctcactcataggtgggaattgaacaatgagaacacttggacacagggtggggaacatcacacactgggtcctgttgtgggctggaggtatgggggagggatagcattaggagatatacctaatgtaaatgacgagttaatgggtgcagcacaccaacatggcacatgtatacatatgtaacaaacgtgcacgtcgtgcacatgtaccctagaacttaaagtataataaaaaatatataaaaaaataacaaaaaagtgctaattgtaaaaaacaacaaaaaaaggatttcaaatttagtttgaaccttcaatgtataccttaagcaagtgacttgaaggaaatttgaatgctgcgtgccttctcccagctctgcctcactgaggatgggaacccagtggcacctgagactcctggatgtagtgcctgggtgacattcctgtggagaaaagcactttagggctagtctctagatgtcttctcatgagtcttctgctttcacatgaagctctttagaagacagaaggaaaaaaaatgtgagaagaaataccttgcccttccacaagatagacctgttgtgcagaggtgcatacaattgaggacagagttcaacattttaaattaaatttccaagtagtttctgtgacttcatttaagagaccgttttttgaattccatggttccaatttgtgtctattttcctgttcacataaatttataggaatatacatgccagctgtgagagatgactttatttcactgttgctcttatatccccctacagttgtcacaaggacaccgatatcacacagtgacatgaacctagacatatagtacacttggcagaagaattttccaggtctagcccagcagtccattcaatgatctaaaatggtgatacaga

SEQ ID NO: 165 - protein, 614 aa, Homo sapiens
MSGYSSDRDRGRDRGFGAPRFGGSRAGPLSGKKFGNPGEKLVKKKWNLDELPKFEKNFYQEHPDLARRTAQEVETYRRSKEITVRGHNCPKPVLNFYEANFPANVMDVIARQNFTEPTAIQAQGWPVALSGLDMVGVAQTGSGKTLSYLLPAIVHINHQPFLERGDGPICLVLAPTRELAQQVQQVAAEYCRACRLKSTCIYGGAPKGPQIRDLERGVEICIATPGRLIDFLECGKTNLRRTTYLVLDEADRMLDMGFEPQIRKIVDQIRPDRQTLMWSATWPKEVRQLAEDFLKDYIHINIGALELSANHNILQIVDVCHDVEKDEKLIRLMEEIMSEKENKTIVFVETKRRCDELTRKMRRDGWPAMGIHGDKSQQERDWVLNEFKHGKAPILIATDVASRGLDVEDVKFVINYDYPNSSEDYIHRIGRTARSTKTGTAYTFFTPNNIKQVSDLISVLREANQAINPKLLQLVEDRGSGRSRGRGGMKDDRRDRYSAGKRGGFNTFRDRENYDRGYSSLLKRDFGAKTQNGVYSAANYTNGSFGSNFVSAGIQTSFRTGNPTGTYQNGYDSTQQYGSNVPNMHNGMNQQAYAYPATAAAPMIGYPMPTGYSQ

SEQ ID NO: 166 - protein, 1148 aa, Homo sapiens
MDIRKFFGVIPSGKKLVSETVKKNEKTKSDEETLKAKKGIKEIKVNSSRKEDDFKQKQPSKKKRIIYDSDSESEETLQVKNAKKPPEKLPVSSKPGKISRQDPVTYISETDEEDDFMCKKAASKSKENGRSTNSHLGTSNMKKNEENTKTKNKPLSPIKLTPTSVLDYFGTGSVQRSNKKMVASKRKELSQNTDESGLNDEAIAKQLQLDEDAELERQLHEDEEFARTLAMLDEEPKTKKARKDTEAGETFSSVQANLSKAEKHKYPHKVKTAQVSDERKSYSPRKQSKYESSKESQQHSKSSADKIGEVSSPKASSKLAIMKRKEESSYKEIEPVASKRKENAIKLKGETKTPKKTKSSPAKKESVSPEDSEKKRTNYQAYRSYLNREGPKALGSKEIPKGAENCLEGLIFVITGVLESIERDEAKSLIERYGGKVTGNVSKKTNYLVMGRDSGQSKSDKAAALGTKIIDEDGLLNLIRTMPGKKSKYEIAVETEMKKESKLERTPQKNVQGKRKISPSKKESESKKSRPTSKRDSLAKTIKKETDVFWKSLDFKEQVAEETSGDSKARNLADDSSENKVENLLWVDKYKPTSLKTIIGQQGDQSCANKLLRWLRNWQKSSSEDKKHAAKFGKFSGKDDGSSFKAALLSGPPGVGKTTTASLVCQELGYSYVELNASDTRSKSSLKAIVAESLNNTSIKGFYSNGAASSVSTKHALIMDEVDGMAGNEDRGGIQELIGLIKHTKIPIICMCNDRNHPKIRSLVHYCFDLRFQRPRVEQIKGAMMSIAFKEGLKIPPPAMNEIILGANQDIRQVLHNLSMWCARSKALTYDQAKADSHRAKKDIKMGPFDVARKVFAAGEETAHMSLVDKSDLFFHDYSIAPLFVQENYIHVKPVAAGGDMKKHLMLLSRAADSICDGDLVDSQIRSKQNWSLLPAQAIYASVLPGELMRGYMTQFPTFPSWLGKHSSTGKHDRIVQDLALHMSLRTYSSKRTVNMDYLSLLRDALVQPLTSQGVDGVQDVVALMDTYYLMKEDFENIMEISSWGGKPSPFSKLDPKVKAAFTRAYNKEAHLTPYSLQAIKASRHSTSPSLDSEYNEELNEDDSQSDEKDQDAIETDAMIKKKTKSSKPSKPEKDKEPRKGKGKSSKK

SEQ ID NO: 167 - DNA, 37 bp, Artificial sequence
  Single strand DNA oligonucleotide
ggccacgcgtcgactagtacttttttttttttttttt

SEQ ID NO: 168 - DNA, 20 bp, Artificial sequence
  Single strand DNA oligonucleotide
ggccacgcgtcgactagtac

SEQ ID NO: 169 - DNA, 20 bp, Artificial sequence
  Single strand DNA oligonucleotide
gcagaagaacggcatcaagg

SEQ ID NO: 170 - DNA, 21 bp, Artificial sequence
  Single strand DNA oligonucleotide
cgcgatcacatggtcctgctg

SEQ ID NO: 171 - DNA, 20 bp, Artificial sequence
  Single strand DNA oligonucleotide
gtggtgaccgtgacccagga

SEQ ID NO: 172 - DNA, 20 bp, Artificial sequence
  Single strand DNA oligonucleotide
gcggatgtaccccgaggacg

SEQ ID NO: 173 - DNA, 20 bp, Artificial sequence
  Single strand DNA oligonucleotide
gactacaccatcgtggaaca

SEQ ID NO: 174 - DNA, 20 bp, Artificial sequence
  Single strand DNA oligonucleotide
ggatcactctcggcatggac

SEQ ID NO: 175 - RNA, 19 nt, Artificial sequence
  DDX5 SiRNA targeting oligonucleotide
gcaugucgcuugaagucua

SEQ ID NO: 176 - RNA, 19 nt, Artificial sequence
  DDX5 SiRNA targeting oligonucleotide
cucuuuauauuguguguua

SEQ ID NO: 177 - RNA, 19 nt, Artificial sequence
  DDX5 SiRNA targeting oligonucleotide
gcugcaccuaugauugguu

SEQ ID NO: 178 - RNA, 19 nt, Artificial sequence
  DDX5 SiRNA targeting oligonucleotide
gcucuaaguggauuggaua